SEQUENCE LISTING (302 sequences)

SEQ ID NO: 1 - DNA, 864 bp, Canis sp.
  source 1..864: mol_type = genomic DNA; note = Canis familiaris
atggggagccggcgggggccctggccgctcgtctgggccgtgctgcagctgggctggtggccaggatggctcctagactcccctgacaggccctggagcccgctcaccttctccccggcgcagctcacggtgcaggagggagagaacgccacgttcacctgcagcctggccgacatccccgacagcttcgtgctcaactggtaccgcctgagcccccgcaaccagacggacaagctggccgccttccaggaggaccgcatcgagccgggccgggacaggcgcttccgcgtcacgcggctgcccaacgggcgggacttccacatgagcatcgtcgctgcgcgcctcaacgacagcggcatctacctgtgcggggccatctacctgccccccaacacacagatcaacgagagtccccgcgcagagctctccgtgacggagagaaccctggagccccccacacagagccccagccccccacccagactcagcggccagttgcaggggctggtcatcggcgtcacgagcgtgctggtgggtgtcctgctactgctgctgctgacctgggtcctggccgctgtcttccccagggccacccgaggtgcctgtgtgtgcgggagcgaggacgagcctctgaaggagggccccgatgcagcgcccgtcttcaccctggactacggggagctggacttccagtggcgagagaagacgccggagcccccggcgccctgtgccccggagcagaccgagtatgccaccatcgtcttcccgggcaggccggcgtccccgggccgcagggcctcggccagcagcctgcagggagcccagcctccgagccccgaggacggacccggcctgtggcccctc

SEQ ID NO: 2 - protein, 288 aa, Canis sp.
  source 1..288: note = Canis familiaris
MGSRRGPWPLVWAVLQLGWWPGWLLDSPDRPWSPLTFSPAQLTVQEGENATFTCSLADIPDSFVLNWYRLSPRNQTDKLAAFQEDRIEPGRDRRFRVTRLPNGRDFHMSIVAARLNDSGIYLCGAIYLPPNTQINESPRAELSVTERTLEPPTQSPSPPPRLSGQLQGLVIGVTSVLVGVLLLLLLTWVLAAVFPRATRGACVCGSEDEPLKEGPDAAPVFTLDYGELDFQWREKTPEPPAPCAPEQTEYATIVFPGRPASPGRRASASSLQGAQPPSPEDGPGLWPL

SEQ ID NO: 3 - DNA, 352 bp, Canis sp.
  source 1..352: mol_type = genomic DNA; note = canis familiaris
gaggtgcagctggtggagtctgggggagacctggtgaagcctggggggtccctgagactttcctgtgtggcctctggattctccttcagtagataccacatgagctgggtccgccaggctccagggaagggacttcagtgggtcgcatacattatcagtggtggaattaccacaagctatgcagacgctgtgaagggccgattcaccatctccagagacaacgccaagaacacgctgtttcttcagatgaacagcctgagagccgaggacacggccgtgtattactgtgcgacgtacggtagttatttttttgacaagtggggccagggaaccctggtcaccgtctcctcag

SEQ ID NO: 4 - protein, 117 aa, Canis sp.
  source 1..117: note = canis familiaris
EVQLVESGGDLVKPGGSLRLSCVASGFSFSRYHMSWVRQAPGKGLQWVAYIISGGITTSYADAVKGRFTISRDNAKNTLFLQMNSLRAEDTAVYYCATYGSYFFDKWGQGTLVTVSS

SEQ ID NO: 5 - DNA, 334 bp, Canis sp.
  source 1..334: mol_type = genomic DNA; note = canis familiaris
gatattgtcatgacacagaccccactgtccctgtctgtcagccctggagagactgcctccatctcttgcaaggccagtcagagcctcctgcacagtgatggaaagtcgtatttgaattggttccgacagaagccaggccagtctccacagcgtttgatctatagggtctccaacagagaccctggggtcccagacaggttcagtggcagcgggtcagggacagatttcaccctgagaatcagcagagtggaggctgacgatactggactttattactgcgggcaagttatacaagatccgacgttcggcgcaggaaccaaggtggagctcaaac

SEQ ID NO: 6 - protein, 111 aa, Canis sp.
  source 1..111: note = canis familiaris
DIVMTQTPLSLSVSPGETASISCKASQSLLHSDGKSYLNWFRQKPGQSPQRLIYRVSNRDPGVPDRFSGSGSGTDFTLRISRVEADDTGLYYCGQVIQDPTFGAGTKVELK

SEQ ID NO: 7 - protein, 7 aa, Canis sp.
  source 1..7: note = canis familiaris
GFSFSRY

SEQ ID NO: 8 - protein, 6 aa, Canis sp.
  source 1..6: note = canis familiaris
ISGGIT

SEQ ID NO: 9 - protein, 12 aa, Canis sp.
  source 1..12: note = canis familiaris
CATYGSYFFDKW

SEQ ID NO: 10 - protein, 11 aa, Canis sp.
  source 1..11: note = canis familiaris
CKASQSLLHSD

SEQ ID NO: 11 - protein, 7 aa, Canis sp.
  source 1..7: note = canis familiaris
PQRLIYR

SEQ ID NO: 12 - protein, 10 aa, Canis sp.
  source 1..10: note = canis familiaris
CGQVIQDPTF

SEQ ID NO: 13 - DNA, 358 bp, Canis sp.
  source 1..358: mol_type = genomic DNA; note = canis familiaris
gaggtgcagctggtggagtctgggggagacctggtgaagcctgcggggtccctgagactgtcctgtttggcctctggattcaccttcagtagctacagcatgagctgggtccgccaggctcctgagaaggggctgcagtcggtcgcgggtattaacagcgatggagatagaacatactacacagacgctgtgaagggccgattcaccatctccagagacaacgccaagagcacagtgtatctgcagatgaacagcctgcgagccgaggacacggccatgtattattgtgtacgggggttttcctctttcaatgcttttggttactggggccagggcaccctggtcactgtctcctcag

SEQ ID NO: 14 - protein, 119 aa, Canis sp.
  source 1..119: note = canis familiaris
EVQLVESGGDLVKPAGSLRLSCLASGFTFSSYSMSWVRQAPEKGLQSVAGINSDGDRTYYTDAVKGRFTISRDNAKSTVYLQMNSLRAEDTAMYYCVRGFSSFNAFGYWGQGTLVTVSS

SEQ ID NO: 15 - DNA, 337 bp, Canis sp.
  source 1..337: mol_type = genomic DNA; note = canis familiaris
gatattgtcatgacacagaccccactgtccctgtctgtcagtcctggagagactgcctccatctcttgcaaggccagtcagagcctcctgcacagtgatggaaacacgtatttgagttggttccgacagaagccaggccagtctccacaccgtttgatctatgcggtctcccacagacaccctggggtcccagacaggttcagtggcagcgggtcagggacagatttcaccctgagaatcagcagagtggaggctgacgatgctggactttattactgcggacaagttatacaagatccgtggacgttcggagcaggaaccaaggtggaactcaagc

SEQ ID NO: 16 - protein, 112 aa, Canis sp.
  source 1..112: note = canis familiaris
DIVMTQTPLSLSVSPGETASISCKASQSLLHSDGNTYLSWFRQKPGQSPHRLIYAVSHRHPGVPDRFSGSGSGTDFTLRISRVEADDAGLYYCGQVIQDPWTFGAGTKVELK

SEQ ID NO: 17 - protein, 7 aa, Canis sp.
  source 1..7: note = canis familiaris
GFTFSSY

SEQ ID NO: 18 - protein, 6 aa, Canis sp.
  source 1..6: note = canis familiaris
NSDGDR

SEQ ID NO: 19 - protein, 14 aa, Canis sp.
  source 1..14: note = canis familiaris
CVRGFSSFNAFGYW

SEQ ID NO: 20 - protein, 11 aa, Canis sp.
  source 1..11: note = canis familiaris
CKASQSLLHSD

SEQ ID NO: 21 - protein, 7 aa, Canis sp.
  source 1..7: note = canis familiaris
PHRLIYA

SEQ ID NO: 22 - protein, 11 aa, Canis sp.
  source 1..11: note = canis familiaris
CGQVIQDPWTF

SEQ ID NO: 23 - DNA, 352 bp, Canis sp.
  source 1..352: mol_type = genomic DNA; note = canis familiaris
gaggtgcagctggtggagtctgggggagacctggtgaagcctggggggtccctgagactttcctgtgtggcctctggattctccttcagtagataccacatgagctgggtccgccaggctccagggaagggacttcagtgggtcgcatacattgtcagtggtggaatttccacaagctatgcagacgctgtgaagggccgattcaccatctccagagacaacgccaagaacacgctgtttcttcagatgaacagcctgagagccgaggacacggccgtgtattactgtgcgacgtacggtagttatttttttgacacctggggccagggaaccctggtcaccgtctcctcag

SEQ ID NO: 24 - protein, 117 aa, Canis sp.
  source 1..117: note = canis familiaris
EVQLVESGGDLVKPGGSLRLSCVASGFSFSRYHMSWVRQAPGKGLQWVAYIVSGGISTSYADAVKGRFTISRDNAKNTLFLQMNSLRAEDTAVYYCATYGSYFFDTWGQGTLVTVSS

SEQ ID NO: 25 - DNA, 334 bp, Canis sp.
  source 1..334: mol_type = genomic DNA; note = canis familiaris
gatattgtcatgacacagaccccactgtccctgtctgtcagccctggagagactgcctccattttttgtaaggccagtcagagcctcctgcacagtgatggaaagtcgtatttgaattggttccgacagaagccaggccagtctccacagcgtttgatctatagggtctccaacagggaccctggggtcccagacaggttcagtggcagcgggtcagggacagatttcaccctgagaatcagcagagtggaggctgacgatactggactttattactgcgggcaagttatacaagatccgacgttcggcgcaggaaccaaggtggagctcaaac

SEQ ID NO: 26 - protein, 111 aa, Canis sp.
  source 1..111: note = canis familiaris
DIVMTQTPLSLSVSPGETASIFCKASQSLLHSDGKSYLNWFRQKPGQSPQRLIYRVSNRDPGVPDRFSGSGSGTDFTLRISRVEADDTGLYYCGQVIQDPTFGAGTKVELK

SEQ ID NO: 27 - protein, 7 aa, Canis sp.
  source 1..7: note = canis familiaris
GFSFSRY

SEQ ID NO: 28 - protein, 6 aa, Canis sp.
  source 1..6: note = canis familiaris
VSGGIS

SEQ ID NO: 29 - protein, 12 aa, Canis sp.
  source 1..12: note = canis familiaris
CATYGSYFFDTW

SEQ ID NO: 30 - protein, 11 aa, Canis sp.
  source 1..11: note = canis familiaris
CKASQSLLHSD

SEQ ID NO: 31 - protein, 7 aa, Canis sp.
  source 1..7: note = canis familiaris
PQRLIYR

SEQ ID NO: 32 - protein, 10 aa, Canis sp.
  source 1..10: note = canis familiaris
CGQVIQDPTF

SEQ ID NO: 33 - DNA, 361 bp, Canis sp.
  source 1..361: mol_type = genomic DNA; note = canis familiaris
caggtgcagctggtggagtctgggggagacctggtgaagccgggggggtccctgagactttcctgtgtggcctctggattccccttcagtagcttccacatgacctgggtccgccaggttcctgggatggggcttcagtgggtcgcatacgttaagactggtggaagtagtacaagctatgcagacgctgtgaagggccgattcaccatctccagagacaatgccaagaacacactgtatcttcacatgaatagcctgagagccgaggacacggccttgtattactgtgcgactggggtggtccccactacggtagcttcttcctactggggccagggaaccctggtcaccgtctcctcag

SEQ ID NO: 34 - protein, 120 aa, Canis sp.
  source 1..120: note = canis familiaris
QVQLVESGGDLVKPGGSLRLSCVASGFPFSSFHMTWVRQVPGMGLQWVAYVKTGGSSTSYADAVKGRFTISRDNAKNTLYLHMNSLRAEDTALYYCATGVVPTTVASSYWGQGTLVTVSS

SEQ ID NO: 35 - DNA, 337 bp, Canis sp.
  source 1..337: mol_type = genomic DNA; note = canis familiaris
gatattgtcatgacacagaccccactgtccctgtccgtcagccctggagagccggcctccatctcctgcaaggccagtcagagcctcctgcacagtagtgggtacacctatttgtattggttccgacagaagccaggccagtctccacagcgtttggtctataaggtcgtctacagagaccctggggtcccagacaggttcagtggcagcgggtcagggacagatttcaccctgagaatcagcagagtggaggctgatgatgctggagtttattactgcgggcaaggtataaaagatccgttcacttttggtcaagggaccaaactggagatcaaac

SEQ ID NO: 36 - protein, 112 aa, Canis sp.
  source 1..112: note = canis familiaris
DIVMTQTPLSLSVSPGEPASISCKASQSLLHSSGYTYLYWFRQKPGQSPQRLVYKVVYRDPGVPDRFSGSGSGTDFTLRISRVEADDAGVYYCGQGIKDPFTFGQGTKLEIK

SEQ ID NO: 37 - protein, 7 aa, Canis sp.
  source 1..7: note = canis familiaris
GFPFSSF

SEQ ID NO: 38 - protein, 6 aa, Canis sp.
  source 1..6: note = canis familiaris
KTGGSS

SEQ ID NO: 39 - protein, 15 aa, Canis sp.
  source 1..15: note = canis familiaris
CATGVVPTTVASSYW

SEQ ID NO: 40 - protein, 11 aa, Canis sp.
  source 1..11: note = canis familiaris
CKASQSLLHSS

SEQ ID NO: 41 - protein, 7 aa, Canis sp.
  source 1..7: note = canis familiaris
PQRLVYK

SEQ ID NO: 42 - protein, 11 aa, Canis sp.
  source 1..11: note = canis familiaris
CGQGIKDPFTF

SEQ ID NO: 43 - DNA, 352 bp, Canis sp.
  source 1..352: mol_type = genomic DNA; note = canis familiaris
gaactcacactgcaggagtcagggccaggattagtgaagccctcacagaccctctctctcacctgtgttgtgtccggaggctccgtcaccaacatattctactggaactggatccgtcagcgccctaagaggggactggaatggatgggatactggtcaggttacacacactacaacccggcattccagggacgcctctccatcgatgctgacacggcccagaaccacttctccctgcagctgagctccatgaccaccgaggacacggccgtgtattattgtgttagaggatatatatggaccccagattatgactactggggccagggaatcctggtcaccgtctcctcag

SEQ ID NO: 44 - protein, 117 aa, Canis sp.
  source 1..117: note = canis familiaris
ELTLQESGPGLVKPSQTLSLTCVVSGGSVTNIFYWNWIRQRPKRGLEWMGYWSGYTHYNPAFQGRLSIDADTAQNHFSLQLSSMTTEDTAVYYCVRGYIWTPDYDYWGQGILVTVSS

SEQ ID NO: 45 - DNA, 337 bp, Canis sp.
  source 1..337: mol_type = genomic DNA; note = canis familiaris
gatattgtcatgacacagaccccactgtccctgtccgtcagccctggagagccggcctccatcgcctgcaaggccagtcagagcctcctgcatagtaatgggaacacctatttgtattggttccgacagaagccaggccagtctccacagcgtttgatctataaggtctccaacagagaccctggggtcccggataggttcagtggcagcgggtcagggacagatttcaccctgagaatcaccagagtggaggctgatgatgctggagtttattactgcgggcaaggtatacaagatccgtggacgttcggagaaggaaccaaggtggagctcaaac

SEQ ID NO: 46 - protein, 112 aa, Canis sp.
  source 1..112: note = canis familiaris
DIVMTQTPLSLSVSPGEPASIACKASQSLLHSNGNTYLYWFRQKPGQSPQRLIYKVSNRDPGVPDRFSGSGSGTDFTLRITRVEADDAGVYYCGQGIQDPWTFGEGTKVELK

SEQ ID NO: 47 - protein, 5 aa, Canis sp.
  source 1..5: note = canis familiaris
GGSVT

SEQ ID NO: 48 - protein, 6 aa, Canis sp.
  source 1..6: note = canis familiaris
GYWSGY

SEQ ID NO: 49 - protein, 14 aa, Canis sp.
  source 1..14: note = canis familiaris
CVRGYIWTPDYDYW

SEQ ID NO: 50 - protein, 11 aa, Canis sp.
  source 1..11: note = canis familiaris
CKASQSLLHSN

SEQ ID NO: 51 - protein, 7 aa, Canis sp.
  source 1..7: note = canis familiaris
PQRLIYK

SEQ ID NO: 52 - protein, 11 aa, Canis sp.
  source 1..11: note = canis familiaris
CGQGIQDPWTF

SEQ ID NO: 53 - DNA, 358 bp, Canis sp.
  source 1..358: mol_type = genomic DNA; note = canis familiaris
gaggtgcagctggtggagtctgggggagacctggtgaagcctgcggggtccctgagactgtcctgtttggcctctggattcaccttcagtagctacagcatgagctgggtccgccaggctcctgagaaggggctgcacttggtcgcaggtatcaacagcgatggagatagaacatactacaaagacgctgtgaagggccgattcaccatctccagagacaacgccaagagtatagtgtatctccagatgaatagcctgcgagtcgaggacacggccatgtattactgtgtacgggggttttcctctttcaatggtcttggttactggggccagggcaccctggtcactgtctcctcag

SEQ ID NO: 54 - protein, 119 aa, Canis sp.
  source 1..119: note = canis familiaris
EVQLVESGGDLVKPAGSLRLSCLASGFTFSSYSMSWVRQAPEKGLHLVAGINSDGDRTYYKDAVKGRFTISRDNAKSIVYLQMNSLRVEDTAMYYCVRGFSSFNGLGYWGQGTLVTVSS

SEQ ID NO: 55 - DNA, 337 bp, Canis sp.
  source 1..337: mol_type = genomic DNA; note = canis familiaris
gatgttgtcatgacacagaccccactgtccctgtctgtcagccctggagagactgcctccatctcttgcaaggccagtcagagcctcctccacagtgatggaaacacgtatttgacttggttccgacagaagccaggccagtctccacaccgtttggtctatgcggtctcccacagacaccctggggtcccagacaggttcagtggcagcgggtcagggacagatttcaccctgagaataagcagagtggaggctgacgatactggactttattactgcggacaagttatacacgatccgtggacgttcggagcaggaaccaaggtggagctcaaac

SEQ ID NO: 56 - protein, 112 aa, Canis sp.
  source 1..112: note = canis familiaris
DVVMTQTPLSLSVSPGETASISCKASQSLLHSDGNTYLTWFRQKPGQSPHRLVYAVSHRHPGVPDRFSGSGSGTDFTLRISRVEADDTGLYYCGQVIHDPWTFGAGTKVELK

SEQ ID NO: 57 - protein, 7 aa, Canis sp.
  source 1..7: note = canis familiaris
GFTFSSY

SEQ ID NO: 58 - protein, 6 aa, Canis sp.
  source 1..6: note = canis familiaris
NSDGDR

SEQ ID NO: 59 - protein, 14 aa, Canis sp.
  source 1..14: note = canis familiaris
CVRGFSSFNGLGYW

SEQ ID NO: 60 - protein, 11 aa, Canis sp.
  source 1..11: note = canis familiaris
CKASQSLLHSD

SEQ ID NO: 61 - protein, 7 aa, Canis sp.
  source 1..7: note = canis familiaris
PHRLVYA

SEQ ID NO: 62 - protein, 11 aa, Canis sp.
  source 1..11: note = canis familiaris
CGQVIHDPWTF

SEQ ID NO: 63 - DNA, 355 bp, Canis sp.
  source 1..355: mol_type = genomic DNA; note = canis familiaris
gaggtgcagctggtggagtctgggggagacctggtgaagcctggggggtccctgagactttcctgtgtggcctctggattcaccttcagtttctacctccttaactgggtccgccaggctccagggaagggacttcaatgggtcgcatacattaacagtggtggaagtaccacatactatgcagacgctgtgaagggccgattcaccatctccagagacaacgccaagaacactctctatcttcagatgaacagcctgagagccgaggacacggccgtatactactgtgcgtctgggggtacttggggtttttttgactactggggccagggaaccctggtctccgtctcctcag

SEQ ID NO: 64 - protein, 118 aa, Canis sp.
  source 1..118: note = canis familiaris
EVQLVESGGDLVKPGGSLRLSCVASGFTFSFYLLNWVRQAPGKGLQWVAYINSGGSTTYYADAVKGRFTISRDNAKNTLYLQMNSLRAEDTAVYYCASGGTWGFFDYWGQGTLVSVSS

SEQ ID NO: 65 - DNA, 319 bp, Canis sp.
  source 1..319: mol_type = genomic DNA; note = canis familiaris
tcctatgtgctgtctcagccgccatcagtgactgtgactctgaggcagacggcccgcctcacctgtgggggagacagcattggaagtaaaagtattgaatggtaccagcagaagccgggccagccccccgtgctcattatctatggtgaaagtagcaggccgtcagggatccctgagcgattctccggcgccaactcggggaacacggccaccctgaccatcagcggggccctggccgaggacgaggctgactattactgccaggtgtgggacagcagtacttgggtattcggtgaagggacccagctgaccgtcctcg

SEQ ID NO: 66 - protein, 106 aa, Canis sp.
  source 1..106: note = canis familiaris
SYVLSQPPSVTVTLRQTARLTCGGDSIGSKSIEWYQQKPGQPPVLIIYGESSRPSGIPERFSGANSGNTATLTISGALAEDEADYYCQVWDSSTWVFGEGTQLTVL

SEQ ID NO: 67 - protein, 7 aa, Canis sp.
  source 1..7: note = canis familiaris
GFTFSFY

SEQ ID NO: 68 - protein, 6 aa, Canis sp.
  source 1..6: note = canis familiaris
NSGGST

SEQ ID NO: 69 - protein, 13 aa, Canis sp.
  source 1..13: note = canis familiaris
CASGGTWGFFDYW

SEQ ID NO: 70 - protein, 11 aa, Canis sp.
  source 1..11: note = canis familiaris
GGDSIGSKSIE

SEQ ID NO: 71 - protein, 7 aa, Canis sp.
  source 1..7: note = canis familiaris
GESSRPS

SEQ ID NO: 72 - protein, 11 aa, Canis sp. [sequence omitted]

SEQ ID NO: 73 - DNA, 352 bp, Canis sp.
  source 1..352: mol_type = genomic DNA; note = canis familiaris
gaggtgcacctggtggagtctgggggagacctggtgaagccgggggggtccctgagactttcctgtgtggcctctggattcaccttcagaaactattacatgagttgggtccgccaaggtccagggaaggggcttcagtgggtcgcatacatcaacagtggtggaattagtacaagatatgcagacgctgtgaagggccgattcaccatctccagagacaacgccaagaatacggtgtatcttcagatgaacagcctgagagtcgaggacacggccgtgtattactgtgcgacctacggtagttattactacgactactggggccagggaaccctggtcaccgtcacctcag

SEQ ID NO: 74 - protein, 117 aa, Canis sp.
  source 1..117: note = canis familiaris
EVHLVESGGDLVKPGGSLRLSCVASGFTFRNYYMSWVRQGPGKGLQWVAYINSGGISTRYADAVKGRFTISRDNAKNTVYLQMNSLRVEDTAVYYCATYGSYYYDYWGQGTLVTVTS

SEQ ID NO: 75 - DNA, 334 bp, Canis sp.
  source 1..334: mol_type = genomic DNA; note = canis familiaris
gatattgtcatgacacagaccccactgtccctgtctgtcagccctggagagactgcctccatctcttgcaaggccagtcagagcctcctgcacaatgatgacaacacgtatttgaattggttccgacagaggccaggccagtctccacagcgtttgatctatagggtctccaacagagaccctggggtcccagacaggttcagtggcagcgggtcagggacagatttcaccctgaaaatcagcagagtggaggctgacgatactggagtttattactgcgggcaagttatacaagatcctacgtttggcaaagggacacatctggagattaaac

SEQ ID NO: 76 - protein, 111 aa, Canis sp.
  source 1..111: note = canis familiaris
DIVMTQTPLSLSVSPGETASISCKASQSLLHNDDNTYLNWFRQRPGQSPQRLIYRVSNRDPGVPDRFSGSGSGTDFTLKISRVEADDTGVYYCGQVIQDPTFGKGTHLEIK

SEQ ID NO: 77 - protein, 7 aa, Canis sp.
  source 1..7: note = canis familiaris
GFTFRNY

SEQ ID NO: 78 - protein, 6 aa, Canis sp.
  source 1..6: note = canis familiaris
NSGGIS

SEQ ID NO: 79 - protein, 12 aa, Canis sp.
  source 1..12: note = canis familiaris
CATYGSYYYDYW

SEQ ID NO: 80 - protein, 11 aa, Canis sp.
  source 1..11: note = canis familiaris
CKASQSLLHND

SEQ ID NO: 81 - protein, 7 aa, Canis sp.
  source 1..7: note = canis familiaris
PQRLIYR

SEQ ID NO: 82 - protein, 10 aa, Canis sp.
  source 1..10: note = canis familiaris
CGQVIQDPTF

SEQ ID NO: 83 - DNA, 352 bp, Canis sp.
  source 1..352: mol_type = genomic DNA; note = canis familiaris
gatctcacactgcaggagtcagggccaggactagtgaagccctcacagaccctctctctcacctgtgttgtgtccggaggctccgtcaccaacatattctactggaactggatccgtcagcgccctaataggggactggaatggatggggtactggtcaggttacacacactacaacccggcattccagggacgcatctccatcgatgctgacacggcccagaaccacttctccctgcagctgagctccatgaccaccgaggacacggccgtatattactgtctaagaggatatatatggaccccagattatgactactggggccagggaatcctggtcaccgtctcctcag

SEQ ID NO: 84 - protein, 117 aa, Canis sp.
  source 1..117: note = canis familiaris
DLTLQESGPGLVKPSQTLSLTCVVSGGSVTNIFYWNWIRQRPNRGLEWMGYWSGYTHYNPAFQGRISIDADTAQNHFSLQLSSMTTEDTAVYYCLRGYIWTPDYDYWGQGILVTVSS

SEQ ID NO: 85 - DNA, 337 bp, Canis sp.
  source 1..337: mol_type = genomic DNA; note = canis familiaris
gatattgtcatgacacagaccccactgtccctgtccgtcagtcctggagagccggcctccatcgcctgtaaggccagtcagagcctcctgcacagtaatgggaacacctatttgtattggttccgacagaagccaggccagtctccacagcgtttgatctataaggtctccaacagagaccctggggtcccggataggttcagtggcagcgggtcagggacagatttcaccctgagaatcaccagagtggaggctgatgatgctggagtttattactgcgggcaaggtatacaagatccgtggacgttcggagcaggaaccaaggtggggctcaaac

SEQ ID NO: 86 - protein, 112 aa, Canis sp.
  source 1..112: note = canis familiaris
DIVMTQTPLSLSVSPGEPASIACKASQSLLHSNGNTYLYWFRQKPGQSPQRLIYKVSNRDPGVPDRFSGSGSGTDFTLRITRVEADDAGVYYCGQGIQDPWTFGAGTKVGLK

SEQ ID NO: 87 - protein, 5 aa, Canis sp.
  source 1..5: note = canis familiaris
GGSVT

SEQ ID NO: 88 - protein, 6 aa, Canis sp.
  source 1..6: note = canis familiaris
GYWSGY

SEQ ID NO: 89 - protein, 14 aa, Canis sp.
  source 1..14: note = canis familiaris
CLRGYIWTPDYDYW

SEQ ID NO: 90 - protein, 11 aa, Canis sp.
  source 1..11: note = canis familiaris
CKASQSLLHSN

SEQ ID NO: 91 - protein, 7 aa, Canis sp.
  source 1..7: note = canis familiaris
PQRLIYK

SEQ ID NO: 92 - protein, 11 aa, Canis sp.
  source 1..11: note = canis familiaris
CGQGIQDPWTF

SEQ ID NO: 93 - DNA, 352 bp, Canis sp.
  source 1..352: mol_type = genomic DNA; note = canis familiaris
gaggtacaactggtggagtctgggggagacctagtgaagccgggggggtccctgagactttcctgtgtggcctctggattcactttcaatacttaccacatgagctgggtccgccaggctccagggaaggggcttcagtgggtcgcatacattaccagtggtggaagtagtacaagccatgcagacgctgtgaagggccgattcaccatctccagagacaacgccaagaaaacgctgtatcttcagatgaacagcctgagagccgaggacacggccgtgtattactgtgcgacttatggtagttatttctttgactcttggggccagggaaccctggtcaccgtctcctcag

SEQ ID NO: 94 - protein, 117 aa, Canis sp.
  source 1..117: note = canis familiaris
EVQLVESGGDLVKPGGSLRLSCVASGFTFNTYHMSWVRQAPGKGLQWVAYITSGGSSTSHADAVKGRFTISRDNAKKTLYLQMNSLRAEDTAVYYCATYGSYFFDSWGQGTLVTVSS

SEQ ID NO: 95 - DNA, 340 bp, Canis sp.
  source 1..340: mol_type = genomic DNA; note = canis familiaris
agtggggatattgtcatgacacagaccccactgtccctgtctgtcagccctggcgagactgcctccatctcttgcaaggccagtcagagcctcctgcacattgatggaaacacgtatttgaattggttccgacagaagccaggccagtctccacagcgtttgatctatagggtctccaccagagaccctggggtcccagacaggttcagtggcagcgggtcagggacagatttcaccctgagaatcagcagagtggaggctgacgatactggagtttattactgcgggcaagttatacaagatcctacgttcggccaagggaccaaggtggagatcaaac

SEQ ID NO: 96 - protein, 113 aa, Canis sp.
  source 1..113: note = canis familiaris
SGDIVMTQTPLSLSVSPGETASISCKASQSLLHIDGNTYLNWFRQKPGQSPQRLIYRVSTRDPGVPDRFSGSGSGTDFTLRISRVEADDTGVYYCGQVIQDPTFGQGTKVEIK

SEQ ID NO: 97 - protein, 7 aa, Canis sp.
  source 1..7: note = canis familiaris
GFTFNTY

SEQ ID NO: 98 - protein, 6 aa, Canis sp.
  source 1..6: note = canis familiaris
TSGGSS

SEQ ID NO: 99 - protein, 12 aa, Canis sp.
  source 1..12: note = canis familiaris
CATYGSYFFDSW

SEQ ID NO: 100 - protein, 11 aa, Canis sp.
  source 1..11: note = canis familiaris
ISCKASQSLLH

SEQ ID NO: 101 - protein, 7 aa, Canis sp.
  source 1..7: note = canis familiaris
QSPQRLI

SEQ ID NO: 102 - protein, 10 aa, Canis sp.
  source 1..10: note = canis familiaris
CGQVIQDPTF

SEQ ID NO: 103 - DNA, 352 bp, Canis sp.
  source 1..352: mol_type = genomic DNA; note = canis familiaris
gaggtacaactggtggagtctgggggagacctagtgaagccgggggggtccctgagactttcctgtgtggcctctggattcactttcaatacttaccacatgagctgggtccgccaggctccagggaaggggcttcagtgggtcgcatacattaccagtggtggaagtagtacaagtcatgcagacgctgtgaagggccgattcactatctccagagacaacgccaagaaaacgctgtatcttcagatgaacaacctgagagccgaggacacggccgtgtattactgtgcgacttatggtagttatttctttgactcttggggccagggaaccctggtcaccgtctcctcag

SEQ ID NO: 104 - protein, 117 aa, Canis sp.
  source 1..117: note = canis familiaris
EVQLVESGGDLVKPGGSLRLSCVASGFTFNTYHMSWVRQAPGKGLQWVAYITSGGSSTSHADAVKGRFTISRDNAKKTLYLQMNNLRAEDTAVYYCATYGSYFFDSWGQGTLVTVSS

SEQ ID NO: 105 - DNA, 340 bp, Canis sp.
  source 1..340: mol_type = genomic DNA; note = canis familiaris
agtggggatattgtcatgacacagaccccactgtccctgcctgtcagccctggcgagactgcctccatctcttgtaaggccagtcagagcctcctgcacattgatggaaacacgtatttgaattggttccgacagaagccaggccagtctccacagcgtttgatctatagggtctccaccagagaccctggggtcccagacaggttcagtggcagcgggtcagggacagatttcaccctgagaatcagcagagtggaggctgacgatactggagtttattactgcgggcaagttatacaagatcctacgttcggccaagggaccaaggtggagatcaaac

SEQ ID NO: 106 - protein, 113 aa, Canis sp.
  source 1..113: note = canis familiaris
SGDIVMTQTPLSLPVSPGETASISCKASQSLLHIDGNTYLNWFRQKPGQSPQRLIYRVSTRDPGVPDRFSGSGSGTDFTLRISRVEADDTGVYYCGQVIQDPTFGQGTKVEIK

SEQ ID NO: 107 - protein, 7 aa, Canis sp.
  source 1..7: note = canis familiaris
GFTFNTY

SEQ ID NO: 108 - protein, 6 aa, Canis sp.
  source 1..6: note = canis familiaris
TSGGSS

SEQ ID NO: 109 - protein, 12 aa, Canis sp.
  source 1..12: note = canis familiaris
CATYGSYFFDSW

SEQ ID NO: 110 - protein, 11 aa, Canis sp.
  source 1..11: note = canis familiaris
ISCKASQSLLH

SEQ ID NO: 111 - protein, 7 aa, Canis sp.
  source 1..7: note = canis familiaris
QSPQRLI

SEQ ID NO: 112 - protein, 10 aa, Canis sp.
  source 1..10: note = canis familiaris
CGQVIQDPTF

SEQ ID NO: 113 - DNA, 346 bp, Canis sp.
  source 1..346: mol_type = genomic DNA; note = canis familiaris
gaggtgccactggtggagtctgggggagacctggtgaagcctggggggtccctgagactttcctgtctggcctctggattcaccttcagtagctaccacatgagctgggtccgccaggctccagggaaggggcttcagtgggtcgcatacattaacagtggtggaaatatcataagctatgtagacgctgtgaagggccgattcaccatctccagagacaacgccaagaacacgctgtatcttcagatgaacagcctgagagtcgaggacacggccgtttattactgtgcgagtcacttccttagctacttctggggccagggaaccctggtcaccgtctcctcag

SEQ ID NO: 114 - protein, 115 aa, Canis sp.
  source 1..115: note = canis familiaris
EVPLVESGGDLVKPGGSLRLSCLASGFTFSSYHMSWVRQAPGKGLQWVAYINSGGNIISYVDAVKGRFTISRDNAKNTLYLQMNSLRVEDTAVYYCASHFLSYFWGQGTLVTVSS

SEQ ID NO: 115 - DNA, 325 bp, Canis sp.
  source 1..325: mol_type = genomic DNA; note = canis familiaris
tcctatgtgctgtctcagccgccatcagcgactgtgactctgaggcagacggcccgcctcacctgtgggggagacagcattggaagaaaaagtgttgaatggtaccaacagaagccgggccagccccccgtgctcattatctatggtgatagcagcaggccgtcagggatccctgagcgattctccggcgccaactcggggaacacggccaccctgaccatcagcggggccctggccgaggacgaggctgactattactgccaggtgtgggacagcagtactaaggctagggtattcggtgaagggacccagctgaccgtcctcg

SEQ ID NO: 116 - protein, 108 aa, Canis sp.
  source 1..108: note = canis familiaris
SYVLSQPPSATVTLRQTARLTCGGDSIGRKSVEWYQQKPGQPPVLIIYGDSSRPSGIPERFSGANSGNTATLTISGALAEDEADYYCQVWDSSTKARVFGEGTQLTVL

SEQ ID NO: 117 - protein, 7 aa, Canis sp.
  source 1..7: note = canis familiaris
GFTFSSY

SEQ ID NO: 118 - protein, 6 aa, Canis sp.
  source 1..6: note = canis familiaris
NSGGNI

SEQ ID NO: 119 - protein, 10 aa, Canis sp.
  source 1..10: note = canis familiaris
CASHFLSYFW

SEQ ID NO: 120 - protein, 11 aa, Canis sp.
  source 1..11: note = canis familiaris
GGDSIGRKSVE

SEQ ID NO: 121 - protein, 7 aa, Canis sp.
  source 1..7: note = canis familiaris
GDSSRPS

SEQ ID NO: 122 - protein, 13 aa, Canis sp.
  source 1..13: note = canis familiaris
CQVWDSSTKARVF

SEQ ID NO: 123 - DNA, 346 bp, Canis sp.
  source 1..346: mol_type = genomic DNA; note = canis familiaris
gaggtgcacctggtggagtctgggggagacctggtgaggccgggggggtccctgagactttcctgtgtggcctctggattcaccttcagtagctaccacatgagctgggtccgccaggctccagggaaggggcttcagtgggtcgcatacattagcaatgatggaagaatcataagttatgcagacgctgtgaagggccgattcaccatctccagagacaacgccaagaacacgttgtatcttcagatgaacagcctgaacgtcgaggacacggccgtgtattactgtgcgagtcacttcgatagttacttctggggccagggaaccctggtcaccgtctcctcag

SEQ ID NO: 124 - protein, 115 aa, Canis sp.
  source 1..115: note = canis familiaris
EVHLVESGGDLVRPGGSLRLSCVASGFTFSSYHMSWVRQAPGKGLQWVAYISNDGRIISYADAVKGRFTISRDNAKNTLYLQMNSLNVEDTAVYYCASHFDSYFWGQGTLVTVSS

SEQ ID NO: 125 - DNA, 325 bp, Canis sp.
  source 1..325: mol_type = genomic DNA; note = canis familiaris
tcctatgtgctgtctcagccgccatcagcgactgtgactctgaggcagacggcccgcctcacctgtgggggagacagcattggacgtaaaagtgttgaatggtaccagcagaagccgggccagccccccgtgctcattatatatggtgatagtagtaggccgtcagggatccctgagcgattctccggcgccaactcggggaacacggccaccctgaccatcagcggggccctggccgaggacgaggcggactattactgccaggtgtgggacagcagaactaaggctagggtattcggtgaagggacccagctgaccgtcctcg

SEQ ID NO: 126 - protein, 108 aa, Canis sp.
  source 1..108: note = canis familiaris
SYVLSQPPSATVTLRQTARLTCGGDSIGRKSVEWYQQKPGQPPVLIIYGDSSRPSGIPERFSGANSGNTATLTISGALAEDEADYYCQVWDSRTKARVFGEGTQLTVL

SEQ ID NO: 127 - protein, 7 aa, Canis sp.
  source 1..7: note = canis familiaris
GFTFSSY

SEQ ID NO: 128 - protein, 6 aa, Canis sp.
  source 1..6: note = canis familiaris
SNDGRI

SEQ ID NO: 129 - protein, 10 aa, Canis sp.
  source 1..10: note = canis familiaris
CASHFDSYFW

SEQ ID NO: 130 - protein, 11 aa, Canis sp.
  source 1..11: note = canis familiaris
GGDSIGRKSVE

SEQ ID NO: 131 - protein, 7 aa, Canis sp.
  source 1..7: note = canis familiaris
GDSSRPS

SEQ ID NO: 132 - protein, 13 aa, Canis sp.
  source 1..13: note = canis familiaris
CQVWDSRTKARVF

SEQ ID NO: 133 - DNA, 346 bp, Canis sp.
  source 1..346: mol_type = genomic DNA; note = canis familiaris
gaggtgcagctggtggagtctgggggagacctggtgaagcctggggggtccctgagactttcctgtttggcctctggattcaccttcagtaacttccacatgaactgggtccgccaggctccagggaaggggcttcagtgggtcgcattcgtaaacgttggtggaattagaacagcctatgcagacggtgtgaagggccgattcaccatctccagagacgacgccaaaaacacgctctatcttcagatggacagcctgcgagccgacgacacggccgtatattactgtgcgagggctttcccgggggactactggggccagggaaccctggtcaccgtctcctcag

SEQ ID NO: 134 - protein, 115 aa, Canis sp.
  source 1..115: note = canis familiaris
EVQLVESGGDLVKPGGSLRLSCLASGFTFSNFHMNWVRQAPGKGLQWVAFVNVGGIRTAYADGVKGRFTISRDDAKNTLYLQMDSLRADDTAVYYCARAFPGDYWGQGTLVTVSS

SEQ ID NO: 135 - DNA, 325 bp, Canis sp.
  source 1..325: mol_type = genomic DNA; note = canis familiaris
tcctatgtgctgtctcagccgccatcagcgactgtgactctgaggcagacggcccgcctcacctgtgggggagacagcattgggagaaaaagtgttgaatggtatcagcagaagccgggccagccccccgtgctcattatctttggtgatagcagcaggccgtcagggatccctgagcgattctccggcgccaactcggggaacacggccaccctgaccatcagcggggccctggccgaggacgaggctgactattactgccaggtgtgggacagcagtactaaggcttgggtattcggtgaagggacccagctgaccgtcctcg

SEQ ID NO: 136 - protein, 108 aa, Canis sp.
  source 1..108: note = canis familiaris
SYVLSQPPSATVTLRQTARLTCGGDSIGRKSVEWYQQKPGQPPVLIIFGDSSRPSGIPERFSGANSGNTATLTISGALAEDEADYYCQVWDSSTKAWVFGEGTQLTVL

SEQ ID NO: 137 - protein, 7 aa, Canis sp.
  source 1..7: note = canis familiaris
GFTFSNF

SEQ ID NO: 138 - protein, 6 aa, Canis sp.
  source 1..6: note = canis familiaris
NVGGIR

SEQ ID NO: 139 - protein, 10 aa, Canis sp.
  source 1..10: note = canis familiaris
CARAFPGDYW

SEQ ID NO: 140 - protein, 11 aa, Canis sp.
  source 1..11: note = canis familiaris
GGDSIGRKSVE

SEQ ID NO: 141 - protein, 7 aa, Canis sp.
  source 1..7: note = canis familiaris
GDSSRPS

SEQ ID NO: 142 - protein, 13 aa, Canis sp.
  source 1..13: note = canis familiaris
CQVWDSSTKAWVF

SEQ ID NO: 143 - DNA, 352 bp, Canis sp.
  source 1..352: mol_type = genomic DNA; note = canis familiaris
gaggtacaactggtggagtctgggggagacctagtgaagccgggggggtccctgagactttcctgtgtggcctctggattcactttcaatacttaccacatgagctgggtccgccagactccagggaaggggcttcagtgggtcgcatacattaccagtggtggaattagtacaagccattcagacgctgtgaagggccgattcaccatctccagagacaacgccaagaaaacgctatatcttcagatgaacagcctgagagccgaggacacggccgtgtattactgtgcgacttatggtagttatttctttgactcttggggccagggaaccctggtcaccgtctcctcag

SEQ ID NO: 144 - protein, 117 aa, Canis sp.
  source 1..117: note = canis familiaris
EVQLVESGGDLVKPGGSLRLSCVASGFTFNTYHMSWVRQTPGKGLQWVAYITSGGISTSHSDAVKGRFTISRDNAKKTLYLQMNSLRAEDTAVYYCATYGSYFFDSWGQGTLVTVSS

SEQ ID NO: 145 - DNA, 340 bp, Canis sp.; identical to SEQ ID NO: 95
  source 1..340: mol_type = genomic DNA; note = canis familiaris

SEQ ID NO: 146 - protein, 113 aa, Canis sp.; identical to SEQ ID NO: 96
  source 1..113: note = canis familiaris

SEQ ID NO: 147 - protein, 7 aa, Canis sp.
  source 1..7: note = canis familiaris
GFTFNTY

SEQ ID NO: 148 - protein, 6 aa, Canis sp.
  source 1..6: note = canis familiaris
TSGGIS

SEQ ID NO: 149 - protein, 12 aa, Canis sp.
  source 1..12: note = canis familiaris
CATYGSYFFDSW

SEQ ID NO: 150 - protein, 11 aa, Canis sp.
  source 1..11: note = canis familiaris
ISCKASQSLLH

SEQ ID NO: 151 - protein, 7 aa, Canis sp.
  source 1..7: note = canis familiaris
QSPQRLI

SEQ ID NO: 152 - protein, 10 aa, Canis sp.
  source 1..10: note = canis familiaris
CGQVIQDPTF

SEQ ID NO: 153 - DNA, 346 bp, Canis sp.; identical to SEQ ID NO: 133
  source 1..346: mol_type = genomic DNA; note = canis familiaris

SEQ ID NO: 154 - protein, 115 aa, Canis sp.; identical to SEQ ID NO: 134
  source 1..115: note = canis familiaris

SEQ ID NO: 155 - DNA, 325 bp, Canis sp.
  source 1..325: mol_type = genomic DNA; note = canis familiaris
tcctatgtgctgtctcagccgccatcagcgactgtgactctgaggcagacggcccgcctcacctgtgggggagacagcattgggagaaaaagtgttgaatggtatcagcagaagccgggccagccccccgtgctcattatctttggtgatagcagcaggccgtcagggatccctgagcgattctccggcgccaactcggggaacacggccaccctgaccatcagcggggccctggccgaggacgaggctgactattactgccaggtgtgggacagcaatactaaggcttgggtattcggtgaagggacccagctgaccgtcctcg

SEQ ID NO: 156 - protein, 108 aa, Canis sp.
  source 1..108: note = canis familiaris
SYVLSQPPSATVTLRQTARLTCGGDSIGRKSVEWYQQKPGQPPVLIIFGDSSRPSGIPERFSGANSGNTATLTISGALAEDEADYYCQVWDSNTKAWVFGEGTQLTVL

SEQ ID NO: 157 - protein, 7 aa, Canis sp.
  source 1..7: note = canis familiaris
GFTFSNF

SEQ ID NO: 158 - protein, 6 aa, Canis sp.
  source 1..6: note = canis familiaris
NVGGIR

SEQ ID NO: 159 - protein, 10 aa, Canis sp.
  source 1..10: note = canis familiaris
CARAFPGDYW

SEQ ID NO: 160 - protein, 11 aa, Canis sp.
  source 1..11: note = canis familiaris
GGDSIGRKSVE

SEQ ID NO: 161 - protein, 7 aa, Canis sp.
  source 1..7: note = canis familiaris
GDSSRPS

SEQ ID NO: 162 - protein, 13 aa, Canis sp.
  source 1..13: note = canis familiaris
CQVWDSNTKAWVF

SEQ ID NO: 163 - DNA, 346 bp, Canis sp.
  source 1..346: mol_type = genomic DNA; note = canis familiaris
gaggtgcaactggtggagtctgggggagacctggtgaagcctggggggtccctgagactttcctgtgtggcctctggattcaccttcagtacctaccacatgagctgggtccgccaggctccagggaggggacttcagtgggtcgcatacattaacagtggtggaactatgataagctatacagacactgtgaagggccgattcaccatctccagagacgacgccaagagcacgctgtatcttcaaatgaacagcctgagagccgaggacacggccgtttattactgtgcgagtcacttcagtagctactactggggccagggaaccctggtcaccgtctcctcag

SEQ ID NO: 164 - protein, 115 aa, Canis sp.
  source 1..115: note = canis familiaris
EVQLVESGGDLVKPGGSLRLSCVASGFTFSTYHMSWVRQAPGRGLQWVAYINSGGTMISYTDTVKGRFTISRDDAKSTLYLQMNSLRAEDTAVYYCASHFSSYYWGQGTLVTVSS

SEQ ID NO: 165 - DNA, 325 bp, Canis sp.
  source 1..325: mol_type = genomic DNA; note = canis familiaris
tcctatgtgctgtctcagccgccatcagcgactgtgactctgaaacagacggcccgcctcacctgtgggggagacagcattggaaggaaaagtgttgaatggtaccagcagaagccgggccagccccccgtgctcattatctatggtgatagcagcaggccgtcagggatccctgagcgattctccggcgccaactcggggaacacggccaccctgaccatcagcggggccctggccgaggacgaggctgactattactgccaggtgtgggacagcagtagtaaggctagggttttcggtgaagggacccagctgaccgtcctcg

SEQ ID NO: 166 - protein, 108 aa, Canis sp.
  source 1..108: note = canis familiaris
SYVLSQPPSATVTLKQTARLTCGGDSIGRKSVEWYQQKPGQPPVLIIYGDSSRPSGIPERFSGANSGNTATLTISGALAEDEADYYCQVWDSSSKARVFGEGTQLTVL

SEQ ID NO: 167 - protein, 7 aa, Canis sp.
  source 1..7: note = canis familiaris
GFTFSTY

SEQ ID NO: 168 - protein, 6 aa, Canis sp.
  source 1..6: note = canis familiaris
NSGGTM

SEQ ID NO: 169 - protein, 10 aa, Canis sp.
  source 1..10: note = canis familiaris
CASHFSSYYW

SEQ ID NO: 170 - protein, 11 aa, Canis sp.
  source 1..11: note = canis familiaris
GGDSIGRKSVE

SEQ ID NO: 171 - protein, 7 aa, Canis sp.
  source 1..7: note = canis familiaris
GDSSRPS

SEQ ID NO: 172 - protein, 13 aa, Canis sp.
  source 1..13: note = canis familiaris
CQVWDSSSKARVF

SEQ ID NO: 173 - DNA, 346 bp, Canis sp.
  source 1..346: mol_type = genomic DNA; note = canis familiaris
gaggtgcagctggtggagtctgggggagacctggtgaagcctggggggtccctgagactttcctgtttggcctctggattcaccttcagtaacttccacatgaactgggtccgccaggctccagggaaggggcttcagtgggtcgcattcgtaaacgttggtggaattagaacagcctatgcagacgctgtgaagggccgattcaccatctccagagacgacgccaaaaacacgctctatcttcagatggacagcctgcgagccgacgacacggccgtatattactgtgcgagggcattcccgggggactactggggccagggaattctggtcaccgtctcctcag

SEQ ID NO: 174 - protein, 115 aa, Canis sp.
  source 1..115: note = canis familiaris
EVQLVESGGDLVKPGGSLRLSCLASGFTFSNFHMNWVRQAPGKGLQWVAFVNVGGIRTAYADAVKGRFTISRDDAKNTLYLQMDSLRADDTAVYYCARAFPGDYWGQGILVTVSS

SEQ ID NO: 175 - DNA, 325 bp, Canis sp.; identical to SEQ ID NO: 135
  source 1..325: mol_type = genomic DNA; note = canis familiaris

SEQ ID NO: 176 - protein, 108 aa, Canis sp.; identical to SEQ ID NO: 136
  source 1..108: note = canis familiaris

SEQ ID NO: 177 - protein, 7 aa, Canis sp.
  source 1..7: note = canis familiaris
GFTFSNF

SEQ ID NO: 178 - protein, 6 aa, Canis sp.
  source 1..6: note = canis familiaris
NVGGIR

SEQ ID NO: 179 - protein, 10 aa, Canis sp.
  source 1..10: note = canis familiaris
CARAFPGDYW

SEQ ID NO: 180 - protein, 11 aa, Canis sp.
  source 1..11: note = canis familiaris
GGDSIGRKSVE

SEQ ID NO: 181 - protein, 7 aa, Canis sp.
  source 1..7: note = canis familiaris
GDSSRPS

SEQ ID NO: 182 - protein, 13 aa, Canis sp.
  source 1..13: note = canis familiaris
CQVWDSSTKAWVF

SEQ ID NO: 183 - DNA, 346 bp, Canis sp.
  source 1..346: mol_type = genomic DNA; note = canis familiaris
gaggtgcagctggtggagtctgggggagacctggtgaagcctggggggtccctgagactttcctgtttggcctctggattcaccttcagtaacttccacatgaactgggtccgccaggctccagggaaggggcttcagtgggtcgcattcgtaaacgttggtggaattagaacagcctatgcagacgctgtgaagggccgattcaccatctccagagacgacgccaaaaacacgctctatcttcagatggacagcctgcgagccgacgacacggccatatattactgtgcgagggcattcccgggggactactggggccagggaaccctggtcaccgtctcctcag

SEQ ID NO: 184 - protein, 115 aa, Canis sp.
  source 1..115: note = canis familiaris
EVQLVESGGDLVKPGGSLRLSCLASGFTFSNFHMNWVRQAPGKGLQWVAFVNVGGIRTAYADAVKGRFTISRDDAKNTLYLQMDSLRADDTAIYYCARAFPGDYWGQGTLVTVSS

SEQ ID NO: 185 - DNA, 325 bp, Canis sp.; identical to SEQ ID NO: 135
  source 1..325: mol_type = genomic DNA; note = canis familiaris

SEQ ID NO: 186 - protein, 108 aa, Canis sp.; identical to SEQ ID NO: 136
  source 1..108: note = canis familiaris

SEQ ID NO: 187 - protein, 7 aa, Canis sp.
  source 1..7: note = canis familiaris
GFTFSNF

SEQ ID NO: 188 - protein, 6 aa, Canis sp.
  source 1..6: note = canis familiaris
NVGGIR

SEQ ID NO: 189 - protein, 10 aa, Canis sp.
  source 1..10: note = canis familiaris
CARAFPGDYW

SEQ ID NO: 190 - protein, 11 aa, Canis sp.
  source 1..11: note = canis familiaris
GGDSIGRKSVE

SEQ ID NO: 191 - protein, 7 aa, Canis sp.
  source 1..7: note = canis familiaris
GDSSRPS

SEQ ID NO: 192 - protein, 13 aa, Canis sp.
  source 1..13: note = canis familiaris
CQVWDSSTKAWVF

SEQ ID NO: 193 - DNA, 346 bp, Canis sp.
  source 1..346: mol_type = genomic DNA; note = canis familiaris
gaggtgcaactggtggagtctgggggagacctggtgaagcctggggggtccctgagactttcctgtgtggcctctggattcaccttcagtacctaccacatgagctgggtccgccaggctccagggaaggggcttcagtgggtcgcatacattagcagtgatggaagaatcataagttatgcagacgctgtgaagggccgattcaccatctccagagacaacgccaagaaaatgttgtatcttcagatgaacagcctgagcgtcgaggacacggccgtatattactgtgcgagtcacttcgctagttacttctggggccagggaaccctggtcaccgtctcctcag

SEQ ID NO: 194 - protein, 115 aa, Canis sp.
  source 1..115: note = canis familiaris
EVQLVESGGDLVKPGGSLRLSCVASGFTFSTYHMSWVRQAPGKGLQWVAYISSDGRIISYADAVKGRFTISRDNAKKMLYLQMNSLSVEDTAVYYCASHFASYFWGQGTLVTVSS

SEQ ID NO: 195 - DNA, 325 bp, Canis sp.
  source 1..325: mol_type = genomic DNA; note = canis familiaris
tcctatgtgctgtctcagccgccatcagcgactgtgactctgaggcagacggcccgcctcacctgtgggggagacagcattggacgtaaaagtgttgaatggtaccagcagaagccgggccagccccccgtgctcattatatatggtgatagtagtaggccgtcagggatccctgagcgattctccggcgccaactcggggaacacggccaccctgaccatcagcggggccctggccgaggacgaggctgactattactgccaggtgtgggacagcagaactaaggctagggtattcggtgaagggacccagctgaccgtcctcg

SEQ ID NO: 196 - protein, 108 aa, Canis sp.; identical to SEQ ID NO: 126
  source 1..108: note = canis familiaris

SEQ ID NO: 197 - protein, 7 aa, Canis sp.
  source 1..7: note = canis familiaris
GFTFSTY

SEQ ID NO: 198 - protein, 6 aa, Canis sp.
  source 1..6: note = canis familiaris
SSDGRI

SEQ ID NO: 199 - protein, 10 aa, Canis sp.
  source 1..10: note = canis familiaris
CASHFASYFW

SEQ ID NO: 200 - protein, 11 aa, Canis sp.
  source 1..11: note = canis familiaris
GGDSIGRKSVE

SEQ ID NO: 201 - protein, 7 aa, Canis sp.
  source 1..7: note = canis familiaris
GDSSRPS

SEQ ID NO: 202 - protein, 13 aa, Canis sp.
  source 1..13: note = canis familiaris
CQVWDSRTKARVF

SEQ ID NO: 203 - DNA, 379 bp, Canis sp.
  source 1..379: mol_type = genomic DNA; note = canis familiaris
gaggtgcaactggtggagtctgggggagacctggtgaagtcgggggggtccctgagactttcctgtgtggcctctggattcaccttcaatacctattctatgaactgggtccgccaggctccagggaaggggcttcagtgggtcgcacgcattaaaagtagtggaagtagcacaacctatgcagacgctgtgaagggccgattcaccgtctccagagacatcgccaagaacactctatttcttcagatgaacagcctgagagccgaggacacggccctgtattactgtgcgagtgaaccgggggcacggtacgaaatatactttgtggagagtttctttgactactggggccagggaaccctggtcaccgtctcctcag

SEQ ID NO: 204 - protein, 126 aa, Canis sp.
  source 1..126: note = canis familiaris
EVQLVESGGDLVKSGGSLRLSCVASGFTFNTYSMNWVRQAPGKGLQWVARIKSSGSSTTYADAVKGRFTVSRDIAKNTLFLQMNSLRAEDTALYYCASEPGARYEIYFVESFFDYWGQGTLVTVSS

SEQ ID NO: 205 - DNA, 325 bp, Canis sp.
  source 1..325: mol_type = genomic DNA; note = canis familiaris
tcctatgtgctgacacagctgccatccaaaaatgtgaccctgaagcagccggcccacatcacctgtgggggagacaacattggaagtaaaagtgttcactggtaccagcagaagctgggccaggcccctgtactgattatctattatgatagcagcaggccgacagggatccctgagcgattctccggcgccaactcggggaacacggccaccctgaccatcagcggggccctggccgaggacgaggctgactattactgccaggtgtgggacagcagtgctcaggcttggatattcggtgaagggacccagctgaccgtcctcg

SEQ ID NO: 206 - protein, 108 aa, Canis sp.
  source 1..108: note = canis familiaris
SYVLTQLPSKNVTLKQPAHITCGGDNIGSKSVHWYQQKLGQAPVLIIYYDSSRPTGIPERFSGANSGNTATLTISGALAEDEADYYCQVWDSSAQAWIFGEGTQLTVL

SEQ ID NO: 207 - protein, 7 aa, Canis sp.
  source 1..7: note = canis familiaris
GFTFNTY

SEQ ID NO: 208 - protein, 6 aa, Canis sp.
  source 1..6: note = canis familiaris
KSSGSS

SEQ ID NO: 209 - protein, 21 aa, Canis sp.
  source 1..21: note = canis familiaris
CASEPGARYEIYFVESFFDYW

SEQ ID NO: 210 - protein, 11 aa, Canis sp.
  source 1..11: note = canis familiaris
GGDNIGSKSVH

SEQ ID NO: 211 - protein, 7 aa, Canis sp.
  source 1..7: note = canis familiaris
YDSSRPT

SEQ ID NO: 212 - protein, 13 aa, Canis sp.
  source 1..13: note = canis familiaris
CQVWDSSAQAWIF

SEQ ID NO: 213 - DNA, 379 bp, Canis sp.
  source 1..379: mol_type = genomic DNA; note = canis familiaris
gaggtgcagatggtggagtctgggggagacctggtgaagccgggggggtccctaagactttcctgtgtggcctctggattcaccttcagtaactaccacatgaactgggtccgccaggctccagggaaggggcttcagtgggtcgcatacattaacagaggtggaagtaggacaagctatgtagacgctgtgaagggccgattcaccatctccagagacaacgccaggaacacgctgtttcttcagatgaacagcctgagagccgaggacacggccgtgtattactgtgcgagtgaaccgggggcacgatatgatatatactacgtggagagtttctttgagtcctggggccagggaaccctggtcaccgtctcctcag

SEQ ID NO: 214 - protein, 126 aa, Canis sp.
  source 1..126: note = canis familiaris
EVQMVESGGDLVKPGGSLRLSCVASGFTFSNYHMNWVRQAPGKGLQWVAYINRGGSRTSYVDAVKGRFTISRDNARNTLFLQMNSLRAEDTAVYYCASEPGARYDIYYVESFFESWGQGTLVTVSS

SEQ ID NO: 215 - DNA, 325 bp, Canis sp.
  source 1..325: mol_type = genomic DNA; note = canis familiaris
tcctatgtgctgacacagctgccatccaaaaatgtgaccctgaagcagccggcccacatcacctgtgggggagacaacattggaagtaaaagtgttcactggtaccagcagaagctgggccaggcccctgtactgattatctattatgatagcagcaggccgacagggatccctgagcgattctccggcgccaactcggggaacacggccaccctgaccatcagcggggccctggccgaggacgaggctgactattactgccaggtgtgggacagcagtgctaaggcttggatattcggtgaagggacccagctgaccgtcctcg

SEQ ID NO: 216 - protein, 108 aa, Canis sp.
  source 1..108: note = canis familiaris
SYVLTQLPSKNVTLKQPAHITCGGDNIGSKSVHWYQQKLGQAPVLIIYYDSSRPTGIPERFSGANSGNTATLTISGALAEDEADYYCQVWDSSAKAWIFGEGTQLTVL

SEQ ID NO: 217 - protein, 7 aa, Canis sp.
  source 1..7: note = canis familiaris
GFTFSNY

SEQ ID NO: 218 - protein, 6 aa, Canis sp.
  source 1..6: note = canis familiaris
NRGGSR

SEQ ID NO: 219 - protein, 21 aa, Canis sp.
  source 1..21: note = canis familiaris
CASEPGARYDIYYVESFFESW

SEQ ID NO: 220 - protein, 11 aa, Canis sp.
  source 1..11: note = canis familiaris
GGDNIGSKSVH

SEQ ID NO: 221 - protein, 7 aa, Canis sp.
  source 1..7: note = canis familiaris
YDSSRPT

SEQ ID NO: 222 - protein, 13 aa, Canis sp.
  source 1..13: note = canis familiaris
CQVWDSSAKAWIF

SEQ ID NO: 223 - DNA, 379 bp, Canis sp.
  source 1..379: mol_type = genomic DNA; note = canis familiaris
gaggtgcagctggtggagtctgggggagacctggtgaagtcgggggggtccctgagactttcctgtgtggcctctggattcaccttcagtacctactctatgaactgggtccgccaggctccagggaaggggcttcagtgggtcgcacgcattaatagtggtggaagtagcacaagctatgcagacgctgtgaagggccgattcaccgtctccagagacatcgccaagaacactctatttcttcagatgaacagcctgagagccgaggacacggccctgtattactgtgcgagtgaaccgggggcacggtacgaaatatactttgtggagagtttctttgactactggggccagggaaccctggtcaccgtctcctcag

SEQ ID NO: 224 - protein, 126 aa, Canis sp.
  source 1..126: note = canis familiaris
EVQLVESGGDLVKSGGSLRLSCVASGFTFSTYSMNWVRQAPGKGLQWVARINSGGSSTSYADAVKGRFTVSRDIAKNTLFLQMNSLRAEDTALYYCASEPGARYEIYFVESFFDYWGQGTLVTVSS

SEQ ID NO: 225 - DNA, 325 bp, Canis sp.
  source 1..325: mol_type = genomic DNA; note = canis familiaris
tcctatgtgctgacacagctgccatccaaaaatgtgaccctgaagcagccggcccacatcacctgtgggggagacaacattggaagtaaaagtgttcactggtaccagcagaagctgggccaggcccctgtattgattatctattatgatagcagcaggccgacagggatccctgagcgattctccggcgccaactcggggaacacggccaccctgaccatcagcggggccctggccgaggacgaggctgactattactgccaggtgtgggacagcagtgctaaggcttggatattcggtgaagggacccagctgaccgtcctcg

SEQ ID NO: 226 - protein, 108 aa, Canis sp.; identical to SEQ ID NO: 216
  source 1..108: note = canis familiaris

SEQ ID NO: 227 - protein, 7 aa, Canis sp.
  source 1..7: note = canis familiaris
GFTFSTY

SEQ ID NO: 228 - protein, 6 aa, Canis sp.
  source 1..6: note = canis familiaris
NSGGSS

SEQ ID NO: 229 - protein, 21 aa, Canis sp.
  source 1..21: note = canis familiaris
CASEPGARYEIYFVESFFDYW

SEQ ID NO: 230 - protein, 11 aa, Canis sp.
  source 1..11: note = canis familiaris
GGDNIGSKSVH

SEQ ID NO: 231 - protein, 7 aa, Canis sp.
  source 1..7: note = canis familiaris
YDSSRPT

SEQ ID NO: 232 - protein, 13 aa, Canis sp.
  source 1..13: note = canis familiaris
CQVWDSSAKAWIF

SEQ ID NO: 233 - DNA, 379 bp, Canis sp.
  source 1..379: mol_type = genomic DNA; note = canis familiaris
gaggtgcagctggtggagtctgggggagacctggtgaagccgggggggtccctgagactttcctgtgtggcctctggattcaccttcagtacctaccacatgaactgggtccgccaggctccagggaaggggcttcagtgggtcgcacacattaacagaagtggaagtattacaagttatgtggacgctgtgaaggaccgattcaccatctccagagacaacgccaagaacacgctgtatcttcagctgaacagcctgagagccgaggacacggccgtgtattactgtgcgagtgaaccgggggcacggtacgatatatactacgtggagagtttctttgactactggggccagggaaccctggtcaccgtctcctcag

SEQ ID NO: 234 - protein, 126 aa, Canis sp.
  source 1..126: note = canis familiaris
EVQLVESGGDLVKPGGSLRLSCVASGFTFSTYHMNWVRQAPGKGLQWVAHINRSGSITSYVDAVKDRFTISRDNAKNTLYLQLNSLRAEDTAVYYCASEPGARYDIYYVESFFDYWGQGTLVTVSS

SEQ ID NO: 235 - DNA, 325 bp, Canis sp.
  source 1..325: mol_type = genomic DNA; note = canis familiaris
tcctatgtgctgacacagctgccagccaaaaatgtgaccctgaagcaggcggcccacatcacctgtgggggagacaacattggaagtaaaagtgttcactggtaccagcagaagctgggccaggcccctgtactgattatctattatgatagcagcaggccgacagggatccctgagcgattctccggcgccaactcggggaacacggccaccctgaccatcagcggggccctggccgaggacgaggctgactattactgccaggtgtgggacagcagtgctaaggcttggatattcggtgaagggacccagctgaccgtcctcg

SEQ ID NO: 236 - protein, 108 aa, Canis sp.
  source 1..108: note = canis familiaris
SYVLTQLPAKNVTLKQAAHITCGGDNIGSKSVHWYQQKLGQAPVLIIYYDSSRPTGIPERFSGANSGNTATLTISGALAEDEADYYCQVWDSSAKAWIFGEGTQLTVL

SEQ ID NO: 237 - protein, 7 aa, Canis sp.
  source 1..7: note = canis familiaris
GFTFSTY

SEQ ID NO: 238 - protein, 6 aa, Canis sp.
  source 1..6: note = canis familiaris
NRSGSI

SEQ ID NO: 239 - protein, 21 aa, Canis sp.
  source 1..21: note = canis familiaris
CASEPGARYDIYYVESFFDYW

SEQ ID NO: 240 - protein, 11 aa, Canis sp.
  source 1..11: note = canis familiaris
GGDNIGSKSVH

SEQ ID NO: 241 - protein, 7 aa, Canis sp.
  source 1..7: note = canis familiaris
YDSSRPT

SEQ ID NO: 242 - protein, 13 aa, Canis sp.
  source 1..13: note = canis familiaris
CQVWDSSAKAWIF

SEQ ID NO: 243 - DNA, 379 bp, Canis sp.
  source 1..379: mol_type = genomic DNA; note = canis familiaris
gaggtgcaactggtggagtctgggggagacctggtgaagtcgggggggtccctgagactctcctgtgtggcctctggattcaccttcagtacctactctatgaactgggtccgccaggctccagggaaggggcttcagtgggtcgcacgcattaaaagtagtggaagtagtacaagctatgcagacgctgtgaagggccgattcaccgtctccagagacatcgccaagaacactctatttcttcagatgaacagcctgagagccgaggacacggccctgtatttctgtgcgagtgaaccgggggcacggtacgaaatatactttgtggagagtttctttgactactggggccagggaaccctggtcaccgtctcctcag

SEQ ID NO: 244 - protein, 126 aa, Canis sp.
  source 1..126: note = canis familiaris
EVQLVESGGDLVKSGGSLRLSCVASGFTFSTYSMNWVRQAPGKGLQWVARIKSSGSSTSYADAVKGRFTVSRDIAKNTLFLQMNSLRAEDTALYFCASEPGARYEIYFVESFFDYWGQGTLVTVSS

SEQ ID NO: 245 - DNA, 325 bp, Canis sp.; identical to SEQ ID NO: 205
  source 1..325: mol_type = genomic DNA; note = canis familiaris

SEQ ID NO: 246 - protein, 108 aa, Canis sp.; identical to SEQ ID NO: 206
  source 1..108: note = canis familiaris

SEQ ID NO: 247 - protein, 7 aa, Canis sp.
  source 1..7: note = canis familiaris
GFTFSTY

SEQ ID NO: 248 - protein, 6 aa, Canis sp.
  source 1..6: note = canis familiaris
KSSGSS

SEQ ID NO: 249 - protein, 21 aa, Canis sp.
  source 1..21: note = canis familiaris
CASEPGARYEIYFVESFFDYW

SEQ ID NO: 250 - protein, 11 aa, Canis sp.
  source 1..11: note = canis familiaris
GGDNIGSKSVH

SEQ ID NO: 251 - protein, 7 aa, Canis sp.
  source 1..7: note = canis familiaris
YDSSRPT

SEQ ID NO: 252 - protein, 13 aa, Canis sp.
  source 1..13: note = canis familiaris
CQVWDSSAQAWIF

SEQ ID NO: 253 - DNA, 379 bp, Canis sp.
  source 1..379: mol_type = genomic DNA; note = canis familiaris
gaggtgcaactggtggagtctgggggagacctggtgaagtcgggggggtccctgagactttcctgtgtggcctctggattcaccttcagtacctactctatgaactgggtccgccaggctccagggaaggggcttcagtgggtcgcacgcattaaaagtagtggaagtagcacaagctatgcagacgctgtgaagggccgattcaccgtctccagagacatcgccaagaacactctatttcttcagatgaacagcctgagagccgaggacacggccttctattactgtgcgagtgaaccgggggcacggtacgaaatatactttgtggagagtttctttgactactggggccagggaaccctggtcaccgtctcctcag

SEQ ID NO: 254 - protein, 126 aa, Canis sp.
  source 1..126: note = canis familiaris
EVQLVESGGDLVKSGGSLRLSCVASGFTFSTYSMNWVRQAPGKGLQWVARIKSSGSSTSYADAVKGRFTVSRDIAKNTLFLQMNSLRAEDTAFYYCASEPGARYEIYFVESFFDYWGQGTLVTVSS

SEQ ID NO: 255 - DNA, 325 bp, Canis sp.
  source 1..325: mol_type = genomic DNA; note = canis familiaris
tcctatgtgctgacacagctgccatccaaaaatgtgaccctgaagcagccggcccacatcacctgtgggggagacaacattggaagtaaaagtgttcactggtaccagcagaagctgggccaggcccctatactgattatctattatgatatcagcaggccgacagggatccctgagcgattctccggcgccaagtcggggaacacggccaccctgaccatcagcggggccctggccgaggacgaggctgactattactgccaggtgtgggacagcagtgctcaggcttggatattcggtgaagggacccagctgaccgtcctcg

SEQ ID NO: 256 - protein, 108 aa, Canis sp.
  source 1..108: note = canis familiaris
SYVLTQLPSKNVTLKQPAHITCGGDNIGSKSVHWYQQKLGQAPILIIYYDISRPTGIPERFSGAKSGNTATLTISGALAEDEADYYCQVWDSSAQAWIFGEGTQLTVL

SEQ ID NO: 257 - protein, 7 aa, Canis sp.
  source 1..7: note = canis familiaris
GFTFSTY

SEQ ID NO: 258 - protein, 6 aa, Canis sp.
  source 1..6: note = canis familiaris
KSSGSS

SEQ ID NO: 259 - protein, 21 aa, Canis sp.
  source 1..21: note = canis familiaris
CASEPGARYEIYFVESFFDYW

SEQ ID NO: 260 - protein, 11 aa, Canis sp.
  source 1..11: note = canis familiaris
GGDNIGSKSVH

SEQ ID NO: 261 - protein, 7 aa, Canis sp.
  source 1..7: note = canis familiaris
YDISRPT

SEQ ID NO: 262 - protein, 13 aa, Canis sp.
  source 1..13: note = canis familiaris
CQVWDSSAQAWIF

SEQ ID NO: 263 - DNA, 379 bp, Canis sp.; identical to SEQ ID NO: 203
  source 1..379: mol_type = genomic DNA; note = canis familiaris

SEQ ID NO: 264 - protein, 126 aa, Canis sp.; identical to SEQ ID NO: 204
  source 1..126: note = canis familiaris

SEQ ID NO: 265 - DNA, 325 bp, Canis sp.
  source 1..325: mol_type = genomic DNA; note = canis familiaris
tcctatgtgctgacacagctgccatccaaaagtgtgaccctgaagcagccggcccacatcacctgtgggggagacaacattggaagtaaaagtgttcactggtaccagcagaagctgggccaggcccctgtactgattatctattatgatagcagcaggccgacagggatccctgagcgattctccggcgccaactcggggaacacggccaccctgaccatcagcggggccctggccgaggacgaggctgactattactgccaggtgtgggacagcagtgctcaggcttggatattcggtgaagggacccagctgaccgtcctcg

SEQ ID NO: 266 - protein, 108 aa, Canis sp.
  source 1..108: note = canis familiaris
SYVLTQLPSKSVTLKQPAHITCGGDNIGSKSVHWYQQKLGQAPVLIIYYDSSRPTGIPERFSGANSGNTATLTISGALAEDEADYYCQVWDSSAQAWIFGEGTQLTVL

SEQ ID NO: 267 - protein, 7 aa, Canis sp.
  source 1..7: note = canis familiaris
GFTFNTY

SEQ ID NO: 268 - protein, 6 aa, Canis sp.
  source 1..6: note = canis familiaris
KSSGSS

SEQ ID NO: 269 - protein, 21 aa, Canis sp.
  source 1..21: note = canis familiaris
CASEPGARYEIYFVESFFDYW

SEQ ID NO: 270 - protein, 11 aa, Canis sp.
  source 1..11: note = canis familiaris
GGDNIGSKSVH

SEQ ID NO: 271 - protein, 7 aa, Canis sp.
  source 1..7: note = canis familiaris
YDSSRPT

SEQ ID NO: 272 - protein, 13 aa, Canis sp.
  source 1..13: note = canis familiaris
CQVWDSSAQAWIF

SEQ ID NO: 273 - DNA, 367 bp, Canis sp.
  source 1..367: mol_type = genomic DNA; note = canis familiaris
gaggtgcagctggtggagtctgggggagacctggtgaagcctggggggtccctgagactttcctgtgtggcctctggattcaccttcagtagatactacatgaactgggtccgccaggctccagggaaggggcctcagtgggtcgcatacattaacaatgctggaagtgatagaaattatgtagacgctgtgaagggccgattcaccatctccagagacaacgccaagaacacgctgtaccttcagatgaacagcctgagagccgaggacacggccgtgtattattgtgcgagtgaaataaggggggttgtagtagttggacatcttgactactggggccagggaaccctggtcaccgtctcctcag

SEQ ID NO: 274 - protein, 122 aa, Canis sp.
  source 1..122: note = canis familiaris
EVQLVESGGDLVKPGGSLRLSCVASGFTFSRYYMNWVRQAPGKGPQWVAYINNAGSDRNYVDAVKGRFTISRDNAKNTLYLQMNSLRAEDTAVYYCASEIRGVVVVGHLDYWGQGTLVTVSS

SEQ ID NO: 275 - DNA, 325 bp, Canis sp.
  source 1..325: mol_type = genomic DNA; note = canis familiaris
tcctatgtgctgacacagctgccatccaaaagtgtgaccctgaaccagccggcccacatcacctgtgggggagacaacattggaagtaaaagtgttcactggtaccagcagaagctgggccaggcccctgtactgattatctattatgatagcatcaggccgacagggatccctgagcgattctccggcgccacctcggggaacacggccaccctgaccatcagcggggccctggccgaggacgaggctgactattactgccaggtgtgggacagcagtgctaaggcttgggtattcggtgaagggacccagctgaccgtcctcg

SEQ ID NO: 276 - protein, 108 aa, Canis sp.
  source 1..108: note = canis familiaris
SYVLTQLPSKSVTLNQPAHITCGGDNIGSKSVHWYQQKLGQAPVLIIYYDSIRPTGIPERFSGATSGNTATLTISGALAEDEADYYCQVWDSSAKAWVFGEGTQLTVL

SEQ ID NO: 277 - protein, 7 aa, Canis sp.
  source 1..7: note = canis familiaris
GFTFSRY

SEQ ID NO: 278 - protein, 6 aa, Canis sp.
  source 1..6: note = canis familiaris
NNAGSD

SEQ ID NO: 279 - protein, 17 aa, Canis sp.
  source 1..17: note = canis familiaris
CASEIRGVVVVGHLDYW

SEQ ID NO: 280 - protein, 11 aa, Canis sp.
  source 1..11: note = canis familiaris
GGDNIGSKSVH

SEQ ID NO: 281 - protein, 7 aa, Canis sp.
  source 1..7: note = canis familiaris
YDSIRPT

SEQ ID NO: 282 - protein, 13 aa, Canis sp.
  source 1..13: note = canis familiaris
CQVWDSSAKAWVF

SEQ ID NO: 283 - DNA, 367 bp, Canis sp.
  source 1..367: mol_type = genomic DNA; note = canis familiaris
gaggtgcagctggtggagtctgggggagacctggtgaagcctggggggtccctgagactttcctgtgtggcctctggattcaccttcagtagatactacatgaactgggtccgccaggctccagggaaggggcctcagtgggtcgcatacattaacaatggtggaagtgatagaaattatgtagacgctgtgaagggccgattcaccatctccagagacaacgccaagaacacgctgtaccttcagatgaacagcctgagagccgaggacacggccgtgtattattgtgcgagtgaaataaggggggttgtagtagttggacatcttgactactggggccagggaaccctggtcaccgtctcctcag

SEQ ID NO: 284 - protein, 122 aa, Canis sp.
  source 1..122: note = canis familiaris
EVQLVESGGDLVKPGGSLRLSCVASGFTFSRYYMNWVRQAPGKGPQWVAYINNGGSDRNYVDAVKGRFTISRDNAKNTLYLQMNSLRAEDTAVYYCASEIRGVVVVGHLDYWGQGTLVTVSS

SEQ ID NO: 285 - DNA, 325 bp, Canis sp.
  source 1..325: mol_type = genomic DNA; note = canis familiaris
tcctatgtgctgacacagctgccatccaaaaatgtgaccctgaaccagccggcccacatcacctgtgggggagacaacattggaagtaaaagtgttcactggtaccagcagaagctgggccaggcccctgtactgattatctattatgatagcatcaggccgacagggatccctgagcgattctccggcgccacctcggggaacacggccaccctgaccatcagcggggccctggccgaggacgaggctgactattactgccaggtgtgggacagcagtgctaaggcttgggtattcggtgaagggacccagctgaccgtcctcg

SEQ ID NO: 286 - protein, 108 aa, Canis sp.
  source 1..108: note = canis familiaris
SYVLTQLPSKNVTLNQPAHITCGGDNIGSKSVHWYQQKLGQAPVLIIYYDSIRPTGIPERFSGATSGNTATLTISGALAEDEADYYCQVWDSSAKAWVFGEGTQLTVL

SEQ ID NO: 287 - protein, 7 aa, Canis sp.
  source 1..7: note = canis familiaris
GFTFSRY

SEQ ID NO: 288 - protein, 6 aa, Canis sp.
  source 1..6: note = canis familiaris
NNGGSD

SEQ ID NO: 289 - protein, 17 aa, Canis sp.
  source 1..17: note = canis familiaris
CASEIRGVVVVGHLDYW

SEQ ID NO: 290 - protein, 11 aa, Canis sp.
  source 1..11: note = canis familiaris
GGDNIGSKSVH

SEQ ID NO: 291 - protein, 7 aa, Canis sp.
  source 1..7: note = canis familiaris
YDSIRPT

SEQ ID NO: 292 - protein, 13 aa, Canis sp.
  source 1..13: note = canis familiaris
CQVWDSSAKAWVF

SEQ ID NO: 293 - protein, 335 aa, Canis sp.
  source 1..335: note = Canis familiaris
ASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPESLGGPSVFIFPPKPKDTLLIARTPEVTCVVVDLGREDPEVQISWFVDGKQMQTAKTQPREEQFNGTYRVVSVLPIGHQDWLKGKQFTCKVNHIGLPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQKSLSHSPGK

SEQ ID NO: 294 - protein, 106 aa, Canis sp.
  source 1..106: note = Canis familiaris
GQPKASPSVTLFPPSSEELGANKATLVCLISDFYPSGVTVAWKADGSPITQGVETTKPSKQSNNKYAASSYLSLTPDKWKSHSSFSCLVTHEGSTVEKKVAPAECS

SEQ ID NO: 295 - protein, 110 aa, Canis sp.
  source 1..110: note = Canis familiaris
RNDAQPAVYLFQPSPDQLHTGSASVVCLLNSFYPKDINVKWKVDGVIQDTGIQESVTEQDKDSTYSLSSTLTMSSTEYLSHELYSCEITHKSLPSTLIKSFQRSECQRVD

SEQ ID NO: 296 - protein, 455 aa, Canis sp.
  source 1..455: note = Canis familiaris
EVQLVQSGGDLVKPGGSVRLSCVASGFNIKNTYMHWVRQAPGKGLQWIGRIAPANVDTKYAPKFQGKATISADTAKNTAYMQLNSLRAEDTAVYYCVLIYYDYDGDIDVWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVALDPEDPEVQISWFVDGKQMQTAKTQPREEQFAGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 297 - protein, 218 aa, Canis sp.
  source 1..218: note = Canis familiaris
DIVMTQTPLSLSVSLGEPASISCHASQNINVWLSWYRQKPGQIPQLLIYKASHLHTGVPDRFSGSGSGTDFTLRISRVEADDAGVYYCQQGQSWPLTFGQGTKVEIKRNDAQPAVYLFQPSPDQLHTGSASVVCLLNSFYPKDINVKWKVDGVIQDTGIQESVTEQDSKDSTYSLSSTLTMSSTEYLSHELYSCEITHKSLPSTLIKSFQRSECQRVD

SEQ ID NO: 298 - protein, 451 aa, Canis sp.
  source 1..451: note = Canis familiaris
EVQLVETGGGLVQPGRSLKLSCVASGFTFNNYWMSWTRQAPGKGLEWVASITNSGVSTYYPDSVKGRFTISRDNAQNTLYLQMNSLRSEDTATYFCTSALNWGYWYFDFWGPGTMVTVSSASTTAPSVFPLAPSCGSTSGSTVALACLVSGYFPEPVTVSWNSGSLTSGVHTFPSVLQSSGLYSLSSTVTVPSSRWPSETFTCNVVHPASNTKVDKPVPKESTCKCISPCPVPESLGGPSVFIFPPKPKDILRITRTPEITCVVLDLGREDPEVQISWFVDGKEVHTAKTQPREQQFNSTYRVVSVLPIEHQDWLTGKEFKCRVNHIGLPSPIERTISKARGQAHQPSVYVLPPSPKELSSSDTVTLTCLIKDFFPPEIDVEWQSNGQPEPESKYHTTAPQLDEDGSYFLYSKLSVDKSRWQQGDTFTCAVMHEALQNHYTDLSLSHSPGK

SEQ ID NO: 299 - protein, 223 aa, Canis sp.
  source 1..223: note = Canis familiaris
DIVMTQTPSSQAVSAGEKVTMSCKSSQSLLYSENKKNYLAWYQRKPGQSPKLLIYWASTRESGVPDRFIGSGSGTDFTLTISSVQAEDLAVYYCQQYYNFPLTFGSGTKLEIKRNDAQPAVYLFQPSPDQLHTGSASVVCLLNSFYPKDINVKWKVDGVIQDTGIQESVTEQDKDSTYSLSSTLTMSSTEYLSHELYSCEITHKSLPSTLIKSFQRSECQRVD

SEQ ID NO: 300 - protein, 453 aa, Canis sp.
  source 1..453: note = Canis familiaris
EVQLVQSGAEVKKPGASVKVSCKASGYTFTSFWMNWVRLAPGAGLEWIGRVDPYDSETHYNQKFKDRAILTVDTSTSTAYMELSSLRAGDIAVYYCATQFGFSWLAYWGQGTLVTVSAASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPESLGGPSVFIFPPKPKDTLLIARTPEVTCVVVDLGREDPEVQISWFVDGKQMQTAKTQPREEQFNGTYRVVSVLPIGHQDWLKGKQFTCKVNHIGLPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQKSLSHSPGK

SEQ ID NO: 301 - protein, 224 aa, Canis sp.
  source 1..224: note = Canis familiaris
EIVMTQSPGSLAGSAGESVSINCKSSQSLLYSSNQKNYLAWYQQKPGESPKLLIYWASTRESGVPDRFSGSGSGTDFTLTINNLQAEDVGVYYCQQYYSNPYTFGQGTKLEIKRNDAQPAVYLFQPSPDQLHTGSASVVCLLNSFYPKDINVKWKVDGVIQDTGIQESVTEQDSKDSTYSLSSTLTMSSTEYLSHELYSCEITHKSLPSTLIKSFQRSECQRVD

SEQ ID NO: 302 - protein, 124 aa, Canis sp.
  source 1..124: note = canis familiaris
LDSPDRPWSPLTFSPAQLTVQEGENATFTCSLADIPDSFVLNWYRLSPRNQTDKLAAFQEDRIEPGRDRRFRVTRLPNGRDFHMSIVAARLNDSGIYLCGAIYLPPNTQINESPRAELSVTERT

(19) United States
(12) Patent Application Publication
(10) Pub. No.: US 2024/0254231 A1
(43) Pub. Date: Aug. 1, 2024

(54) THERAPEUTIC ANTIBODIES

(71) Applicant: PETMEDIX LTD., Cambridge (GB)

(72) Inventors: Daniel George William Alanine, Cambridge (GB); Michelle Rodrigues Goulart, London (GB)

(21) Appl. No.: 18/417,116

(22) Filed: Jan. 19, 2024

(30) Foreign Application Priority Data
Jan. 20, 2023 (GB) 2300904.6
Jul. 14, 2023 (GB) 2310818.6

(51) Int. Cl. C07K 16/28; A61K 39/00; A61P 35/00; G01N 33/68

(52) U.S. Cl. CPC C07K 16/2818; A61P 35/00; G01N 33/6854; A61K 2039/505; C07K 2317/20; C07K 2317/33; C07K 2317/565; C07K 2317/569; C07K 2317/622; C07K 2317/76; C07K 2317/94

(57) ABSTRACT

The disclosure provides an isolated canine antibody or antigen-binding portion thereof which binds to an epitope of the extracellular domain of canine PD-1, compositions comprising the same and methods of their use.

### CROSS REFERENCE TO RELATED APPLICATIONS

This application claims the benefit of and priority from United Kingdom Patent Application No. 2300904.6, filed Jan. 20, 2023, and United Kingdom Patent Application No. 2310818.6, filed Jul. 14, 2023, the contents of which are incorporated herein by reference in their entirety.

### SEQUENCE LISTING

The instant application contains a Sequence Listing which has been submitted electronically in XML format and is hereby incorporated by reference in its entirety. Said XML copy, created on Mar. 28, 2024 is named ZP000485A_SL.xml, and is 343,415 bytes in size.

### INTRODUCTION

The present invention relates to antibodies which modulate the PD-1 signalling pathway to treat inflammatory diseases in companion animals.

The human Programmed Death receptor 1 (PD-1) protein is encoded by the PDCD1 gene and expressed as a 32 kDa type I transmembrane protein (Agata 1996 Int Immunol 8(5):765-72). PD-1 is an immunoglobulin superfamily member (Ishida 1992 EMBO 11(11):3887-95) and it is an inhibitory member of the extended CD28/CTLA-4 family of T cell regulators. Other members of this family include CD28, CTLA-4, ICOS and BTLA. PD-1 exists as a monomer, lacking the unpaired cysteine residue characteristic of other CD28 family members (Zhang 2004 Immunity 20:337-47). Its cytoplasmic domain contains an immunoreceptor tyrosine-based inhibitory motif (ITIM) and an immunoreceptor tyrosine-based switch motif (ITSM) that are phosphorylated during signal transduction (Riley 2009 Immunol Rev 229(1):114-25).

PD-1 is expressed on B cells, T cells, and monocytes (Agata 1996). The role of PD-1 in maintaining immunologic self-tolerance was demonstrated in PDCD1−/− mice, which develop autoimmune disorders (Nishimura 1999 Immunity 11:141-51, Nishimura 2001 Science 291(5502):319-22). The PD-1 pathway therefore regulates antigen responses, balancing autoimmunity and tolerance.

There are two ligands for PD-1 that mediate its regulatory function. PD-L1 (B7-H1) is normally expressed on dendritic cells, macrophages, resting B cells, bone marrow-derived mast cells and T cells as well as non-hematopoietic cell lineages (reviewed in Francisco 2010 Immunol Rev 236:219-42). PD-L2 (B7-DC) is largely expressed on dendritic cells and macrophages (Tseng 2001 J Exp Med 193(7):839-45). Ligand expression is influenced by local mediators and can be upregulated by inflammatory cytokines.

PD-1 binds to its corresponding ligand, PD-L1 and attenuates T-cell responses. Antibodies which bind PD-1 and block binding of PD-L1 are used in human cancer therapies to enhance tumour-specific CD8+ T-cell immunity and the clearance of tumour cells by the immune system. PD-1 is known as an immunoinhibitory protein that negatively regulates TCR signals. The interaction between PD-1 and PD-L1 can act as an immune checkpoint, which can lead to, e.g., a decrease in tumour infiltrating lymphocytes, a decrease in T-cell receptor mediated proliferation, and/or immune evasion by cancerous cells. Immune suppression can be reversed by inhibiting the local interaction of PD-1 with PD-L1 or PD-L2; the effect is additive when the interaction of PD-1 with both PD-L1 and PD-L2 is blocked.

As T cells become activated and co-stimulated by antigen-presenting cells (APCs), T cell expression of PD-1 is induced. PD-1 engagement with ligand on the APC cross-links PD-1 and clusters it into the T cell receptor (TCR) complex within the immunological synapse (Yokosuka 2012 J Exp Med 209(9):1201-17). Within the T cell cytoplasm, PD-1 signalling domains ITIM and ITSM are phosphorylated. This induces Src-homology-2 domain-containing tyrosine phosphatase (SHP1/2) that attenuates various components of the T cell receptor (TCR) signalling. T cell activation is dampened, which leads to a reduction in cytokine response, proliferation and cytolytic activity. This downregulation of T cell function serves to prevent overstimulation, tolerising cells against weakly immunogenic self-antigen.

The PD-1 pathway can be exploited in cancer or infection, whereby tumours or viruses can evade effective immune recognition and T cells demonstrate an ‘exhausted’ phenotype. PD-L1 has also been shown to be expressed in many tumour types including urothelial, ovarian, breast, cervical, colon, pancreatic, gastric, melanoma, glioblastoma and non-small cell lung carcinoma (reviewed in Callahan 2014 J Leukoc Biol 94(1):41-53). The cytokines produced by cancer stromal cells can further upregulate PD-L1 in the tumour microenvironment (He 2015 Nature Scientific Reports 5:13110). As a result, tumour-specific T cells become unresponsive through PD-1 signalling and therefore fail to eliminate their target. T regulatory cells (T regs) have also been shown to express high levels of PD-1 and they suppress the anti-tumour response further (Lowther 2016 JCI Insight 1(5):85935).

Disruption of the PD-1:PD-L1 interaction enhances T cell activity. An anti-PD-1 monoclonal antibody demonstrates blockade of the interaction between PD-1 and its ligands (Wang 2014 Cancer Immunol Res 2(9):846-56). T cell function in-vitro can be enhanced by PD-1 blockade, as demonstrated by improved proliferation and cytokine responses in mixed lymphocyte reactions of T cells and dendritic cells. Cytotoxic lymphocytes (CTLs) derived from human melanoma patients has also been shown to be enhanced by PD-1 blockade in vitro using the antibody OPDIVO (nivolumab), and can become resistant to Treg suppression (Wang 2009 Int Immunol 21(9):1065-1077). This antibody has been tested in human clinical dose escalation studies in melanoma, non-small cell lung carcinoma (NSCLC), renal cell cancer (RCC) and others. It shows improved overall survival rates compared to chemotherapy in NSCLC patients. Another PD-1 blocking antibody, KEYTRUDA® (pembrolizumab), demonstrates responses in human NSCLC patients refractory to CTLA-4 blockade. OPDIVO® and KEYTRUDA® both functionally block the interaction of human PD-1 with its ligands.

It is possible to induce human PD-1 signalling by cross-linking it on the membrane with a combination of anti-PD-1 plus anti-CD3 antibodies (Bennett 2003 J Immunol 170:711-18, Keir 2005 J Immunol 175:7372-7379). This function could be detrimental during an anti-tumour response because T cell activity would be suppressed. If suppression of T cell responses were desired, agonistic anti-PD-1 antibodies or those with effector functions could be used to treat immune-related diseases such as rheumatoid arthritis.

Antibodies which recognised canine PD-1 have been described in WO2015/091910, WO2015/091911 and WO2015091914. Mice were immunised with canine PD-1 to generate monoclonal antibodies from which CDR sequences were extracted and placed into a canine immunoglobulin backbone in a process of “caninization”.

Caninization of antibodies is an artificial process, combining mouse CDR repertoires with the canine immunoglobulin backbone. The process assumes that the mouse CDR sequences will function comparably in canine sequence when introduced in vivo i.e. it assumes that the antibody specificity of the CDR regions will be maintained with respect to binding when these regions are taken out of their naturally generated antibody molecule. Moreover, the use of these chimeric antibodies assumes that the presence of mouse CDR sequences within an otherwise canine molecule does not cause any anti-mouse antibody responses in a dog into which the chimeric antibody is introduced. Immunogenicity of antibody therapeutics can lead to attenuation of efficacy over time. This is especially relevant in chronic diseases requiring repeated dosing.

Igase et al. (Nature Research Scientific Reports 2020; https://doi.org/10.1038/s41598-020-75533-4) describes rat-dog chimeric and caninised anti-PD1 antibodies which have been evaluated in vitro and used in a pilot study to determine their safety profiles and clinical efficacy in spontaneously occurring canine cancers such as advanced oral malignant melanoma. These antibodies are rat monoclonal antibodies [see also WO2016/006241] with the heavy and light chain variable regions of 4F12-E6 fused to the constant regions of dog IgG type A.

A successful anti-canine PD1 antibody must have a degree of efficacy in the target population in that the antibody helps to clear and/or reduce the size of certain types of tumour. Whilst being able to cause a reduction in tumour size the antibody must also have an acceptable safety profile. The safety profile will involve a balance between the efficacy of the antibody in tumour reduction and the severity of any off target effects. In addition to the safety profile the pharmacokinetic (pK) profile of the antibody must also enable the antibody to remain in the body long enough to be active. Even if the above criteria are met the antibody must also be able to me manufactured at scale at a cost that is economically viable.

To the inventors' knowledge, an anti-canine PD1 antibody with a high degree of efficacy in the target population and an acceptable safety profile with any sufficient and robust statistical power has yet to be published. On top of this, to date there are no published pK profiles of fully caninised anti-canine PD1 antibodies in the literature. In addition, there is no published caninised anti-canine PD1 antibody that is manufactured economically at scale.

WO2018/189520 and WO2020/074874 describe a transgenic mouse model for the expression of a canine antibody repertoire. Advantageously, this approach allows highly evolved in vivo mechanisms such as hypermutation in germinal centres to be exploited to generate high-affinity antibodies with optimal biophysical properties. The canine mice respond to antigen challenge and produce high-affinity antibodies with canine-like CDRH3 lengths which exhibit broad epitope coverage. In vivo maturation enables antibodies with favourable and desirable biophysical properties to be obtained e.g higher stability and lower tendency to aggregate.

There is a need for improved treatments as well as for drugs that treat the underlying cause of the disease rather than the symptoms. In particular, there is a need for a treatment that is safe, has a long duration of action, and has efficacy to cover a wider spectrum of patients, particularly non-responders.

### SUMMARY

The invention relates to an isolated canine antibody or antigen-binding portion thereof which binds to one of the following epitopes of the extracellular domain of canine PD-1:
i) an epitope comprising T76, Q83, E84, L128, P129, P130 according to SEQ ID NO: 2
ii) an epitope comprising D58, D61, N74, T76, Y127 according to SEQ ID NO: 2
   wherein the epitope is determined using an epitope mapping technique.

The invention also relates to and isolated canine antibody or antigen binding portion thereof which binds to the extracellular domain of canine PD-1, wherein said antibody comprises an HC CDR1 sequence comprising or consisting of SEQ ID NO: 17 or an amino acid sequence with at least 70%, 80%, 85%, 90% or 95% sequence identity to SEQ ID NO: 17 and an HC CDR2 sequence comprising or consisting of SEQ ID NO: 18 or an amino acid sequence with at least 70%, 80%, 85%, 90% or 95% sequence identity to SEQ ID NO: 18 and an HC CDR3 sequence comprising or consisting of SEQ ID NO: 19 or an amino acid sequence with at least 70%, 80%, 85%, 90% or 95% sequence identity to SEQ ID NO: 19 and an LC CDR1 sequence comprising or consisting of SEQ ID NO: 20 or an amino acid sequence with at least 70%, 80%, 85%, 90% or 95% sequence identity to SEQ ID NO: 20 and an LC CDR2 sequence comprising or consisting of SEQ ID NO: 21 or an amino acid sequence with at least 70%, 80%, 85%, 90% or 95% sequence identity to SEQ ID NO: 21 and an LC CDR3 sequence comprising or consisting of SEQ ID NO: 22 or an amino acid sequence with at least 70%, 80%, 85%, 90% or 95% sequence identity to SEQ ID NO: 22; or an HC CDR1 sequence comprising or consisting of SEQ ID NO: 27 or an amino acid sequence with at least 70%, 80%, 85%, 90% or 95% sequence identity to SEQ ID NO: 27 and an HC CDR2 sequence comprising or consisting of SEQ ID NO: 28 or an amino acid sequence with at least 70%, 80%, 85%, 90% or 95% sequence identity to SEQ ID NO: 28 and an HC CDR3 sequence comprising or consisting of SEQ ID NO: 29 or an amino acid sequence with at least 70%, 80%, 85%, 90% or 95% sequence identity to SEQ ID NO: 29 and an LC CDR1 sequence comprising or consisting of SEQ ID NO: 30 or an amino acid sequence with at least 70%, 80%, 85%, 90% or 95% sequence identity to SEQ ID NO: 30 and an LC CDR2 sequence comprising or consisting of SEQ ID NO: 31 or an amino acid sequence with at least 70%, 80%, 85%, 90% or 95% sequence identity to SEQ ID NO: 31 and an LC CDR3 sequence comprising or consisting of SEQ ID NO: 32 or an amino acid sequence with at least 70%, 80%, 85%, 90% or 95% sequence identity to SEQ ID NO: 32; or an HC CDR1 sequence comprising or consisting of SEQ ID NO: 207 or an amino acid sequence with at least 70%, 80%, 85%, 90% or 95% sequence identity to SEQ ID NO: 207 and an HC CDR2 sequence comprising or consisting of SEQ ID NO: 208 or an amino acid sequence with at least 70%, 80%, 85%, 90% or 95% sequence identity to SEQ ID NO: 208 and an HC CDR3 sequence comprising or consisting of SEQ ID NO: 209 or an amino acid sequence w with at least 70%, 80%, 85%, 90% or 95% sequence identity to SEQ ID NO: 209 and an LC CDR1 sequence comprising or consisting of SEQ ID NO: 210 or an amino acid sequence with at least 70%, 80%, 85%, 90% or 95% sequence identity to SEQ ID NO: 210 and an LC CDR2 sequence comprising or consisting of SEQ ID NO: 211 or an amino acid sequence with at least 70%, 80%, 85%, 90% or 95% sequence identity to SEQ ID NO: 211 and an LC CDR3 sequence comprising or consisting of SEQ ID NO: 212 or an amino acid sequence with at least 70%, 80%, 85%, 90% or 95% sequence identity to SEQ ID NO: 212; or an HC CDR1 sequence comprising or consisting of SEQ ID NO: 97 or an amino acid sequence with at least 70%, 80%, 85%, 90% or 95% sequence identity to SEQ ID NO: 97 and an HC CDR2 sequence comprising or consisting of SEQ ID NO: 98 or an amino acid sequence with at least 70%, 80%, 85%, 90% or 95% sequence identity to SEQ ID NO: 98 and an HC CDR3 sequence comprising or consisting of SEQ ID NO: 99 or an amino acid sequence with at least 70%, 80%, 85%, 90% or 95% sequence identity to SEQ ID NO: 99 and an LC CDR1 sequence comprising or consisting of SEQ ID NO: 100 or an amino acid sequence with at least 70%, 80%, 85%, 90% or 95% sequence identity to SEQ ID NO: 100 and an LC CDR2 sequence comprising or consisting of SEQ ID NO: 101 or an amino acid sequence with at least 70%, 80%, 85%, 90% or 95% sequence identity to SEQ ID NO: 101 and an LC CDR3 sequence comprising or consisting of SEQ ID NO: 102 or an amino acid sequence with at least 70%, 80%, 85%, 90% or 95% sequence identity to SEQ ID NO: 102.

The invention also relates to an isolated canine antibody or antigen-binding portion thereof which binds canine PD-1 wherein said antibody comprises
a) an HC CDR1 sequence comprising or consisting of SEQ ID NO: 17 or an amino acid sequence which has 1, 2, 3, 4, or 5 amino acid differences compared to SEQ ID NO: 17 and an HC CDR2 sequence comprising or consisting of SEQ ID NO: 18 or an amino acid sequence which has 1, 2, 3, 4, 5, or 6 amino acid differences compared to SEQ ID NO: 18 and an HC CDR3 sequence comprising or consisting of SEQ ID NO: 19 or an amino acid sequence which has 1, 2, 3, 4, 5, 6, 7, 8, or 9 amino acid differences compared to SEQ ID NO: 19 and an LC CDR1 sequence comprising or consisting of SEQ ID NO: 20 or an amino acid sequence which has 1, 2, 3, 4, 5, 6, 7, 8, 9, 10, 11, 12, 13 or 14 amino acid differences compared to SEQ ID NO: 20 and an LC CDR2 sequence comprising or consisting of SEQ ID NO: 21 or an amino acid sequence which has 1, 2, 3, 4, or 5 amino acid differences compared to SEQ ID NO: 21 and an LC CDR3 sequence comprising or consisting of SEQ ID NO: 22 or an amino acid sequence which has 1, 2, 3, 4, 5, 6, 7, 8, 9, or 10 amino acid differences compared to SEQ ID NO: 22; or
b) an HC CDR1 sequence comprising or consisting of SEQ ID NO: 207 or an amino acid sequence which has 1, or 2 amino acid differences compared to SEQ ID NO: 207 and an HC CDR2 sequence comprising or consisting of SEQ ID NO: 208 or an amino acid sequence which has 1, 2, 3, 4, or 5 amino acid differences compared to SEQ ID NO: 208 and an HC CDR3 sequence comprising or consisting of SEQ ID NO: 209 or an amino acid sequence which has 1, 2, 3, 4, 5, 6, 7, 8, 9, 10, 11, 12, 13, 14, 15, or 16 amino acid differences compared to SEQ ID NO: 209 and an LC CDR1 sequence comprising or consisting of SEQ ID NO: 210 or an amino acid sequence which has 1, 2 or 3 amino acid differences compared to SEQ ID NO: 210 and an LC CDR2 sequence comprising or consisting of SEQ ID NO: 211 or an amino acid sequence which has 1 or 2 amino acid differences compared to SEQ ID NO: 211 and an LC CDR3 sequence comprising or consisting of SEQ ID NO: 212 or an amino acid sequence which has 1, 2, 3, or 4 amino acid differences compared to SEQ ID NO: 212; or
c) an HC CDR1 sequence comprising or consisting of SEQ ID NO: 97 or an amino acid sequence which has 1, 2, or 3 amino acid differences compared to SEQ ID NO: 97 and an HC CDR2 sequence comprising or consisting of SEQ ID NO: 98 or an amino acid sequence which has 1, 2, 3, or 4 amino acid differences compared to SEQ ID NO: 98 and an HC CDR3 sequence comprising or consisting of SEQ ID NO: 99 or an amino acid sequence which has 1, 2, 3, 4, 5, 6, 7, 8, 9, 10, 11, 12, 13, or 14 amino acid differences compared to SEQ ID NO: 99 and an LC CDR1 sequence comprising or consisting of SEQ ID NO: 100 or an amino acid sequence which has 1, 2, 3, 4, 5, 6, 7, 8, 9, 10, 11, 12, 13, 14 or 15 amino acid differences compared to SEQ ID NO: 100 and an LC CDR2 sequence comprising or consisting of SEQ ID NO: 101 or an amino acid sequence which has 1, 2, 3, 4, or 5 amino acid differences compared to SEQ ID NO: 101 and an LC CDR3 sequence comprising or consisting of SEQ ID NO: 102 or an amino acid sequence which has 1, 2, 3, 4, 5, 6, 7, 8, 9, or 10 amino acid differences compared to SEQ ID NO: 102.

The invention also relates to an antibody or antigen-binding portion thereof wherein said antibody or antigen-binding portion thereof has
i) a HC CDR1 sequence comprising or consisting of SEQ ID NO: 17, a HC CDR2 sequence comprising or consisting of SEQ ID NO: 18, a HC CDR3 sequence comprising or consisting of SEQ ID NO: 19, a LC CDR1 sequence comprising or consisting of SEQ ID NO: 20, a LC CDR2 sequence comprising or consisting of SEQ ID NO: 21 and a LC CDR3 sequence comprising or consisting of SEQ ID NO: 22,
ii) a HC CDR1 sequence comprising or consisting of SEQ ID NO: 27, a HC CDR2 sequence comprising or consisting of SEQ ID NO: 28, a HC CDR3 sequence comprising or consisting of SEQ ID NO: 29, a LC CDR1 sequence comprising or consisting of SEQ ID NO: 30, a LC CDR2 sequence comprising or consisting of SEQ ID NO: 31 and a LC CDR3 sequence comprising or consisting of SEQ ID NO: 32
iii) a HC CDR1 sequence comprising or consisting of SEQ ID NO: 7, a HC CDR2 sequence comprising or consisting of SEQ ID NO: 8, a HC CDR3 sequence comprising or consisting of SEQ ID NO: 9, a LC CDR1 sequence comprising or consisting of SEQ ID NO: 10, a LC CDR2 sequence comprising or consisting of SEQ ID NO: 11 and a LC CDR3 sequence comprising or consisting of SEQ ID NO: 12,
iv) a HC CDR1 sequence comprising or consisting of SEQ ID NO: 37, a HC CDR2 sequence comprising or consisting of SEQ ID NO: 38, a HC CDR3 sequence comprising or consisting of SEQ ID NO: 39, a LC CDR1 sequence comprising or consisting of SEQ ID NO: 40, a LC CDR2 sequence comprising or consisting of SEQ ID NO: 41 and a LC CDR3 sequence comprising or consisting of SEQ ID NO: 42
v) a HC CDR1 sequence comprising or consisting of SEQ ID NO: 47, a HC CDR2 sequence comprising or consisting of SEQ ID NO: 48, a HC CDR3 sequence comprising or consisting of SEQ ID NO: 49, a LC CDR1 sequence comprising SEQ ID NO: 50, a LC CDR2 sequence comprising or consisting of SEQ ID NO: 51 and a LC CDR3 sequence comprising or consisting of SEQ ID NO: 52,
vi) a HC CDR1 sequence comprising SEQ ID NO: 57, a HC CDR2 sequence comprising SEQ ID NO: 58, a HC CDR3 sequence comprising SEQ ID NO: 59, a LC CDR1 sequence comprising SEQ ID NO: 60, a LC CDR2 sequence comprising SEQ ID NO: 61 and a LC CDR3 sequence comprising SEQ ID NO: 62,
vii) a HC CDR1 sequence comprising SEQ ID NO: 67, a HC CDR2 sequence comprising SEQ ID NO: 68, a HC CDR3 sequence comprising SEQ ID NO: 69, a LC CDR1 sequence comprising SEQ ID NO: 70, a LC CDR2 sequence comprising SEQ ID NO: 71 and a LC CDR3 sequence comprising SEQ ID NO: 72,
viii) a HC CDR1 sequence comprising SEQ ID NO: 77, a HC CDR2 sequence comprising SEQ ID NO: 78, a HC CDR3 sequence comprising SEQ ID NO: 79, a LC CDR1 sequence comprising SEQ ID NO: 80, a LC CDR2 sequence comprising SEQ ID NO: 81 and a LC CDR3 sequence comprising SEQ ID NO: 82,
ix) a HC CDR1 sequence comprising or consisting of SEQ ID NO: 87, a HC CDR2 sequence comprising or consisting of SEQ ID NO: 88, a HC CDR3 sequence comprising or consisting of SEQ ID NO: 89, a LC CDR1 sequence comprising or consisting of SEQ ID NO: 90, a LC CDR2 sequence comprising or consisting of SEQ ID NO: 91 and a LC CDR3 sequence comprising or consisting of SEQ ID NO: 92,
x) a HC CDR1 sequence comprising or consisting of SEQ ID NO: 97, a HC CDR2 sequence comprising or consisting of SEQ ID NO: 98, a HC CDR3 sequence comprising or consisting of SEQ ID NO: 99, a LC CDR1 sequence comprising or consisting of SEQ ID NO: 100, a LC CDR2 sequence comprising or consisting of SEQ ID NO: 101 and a LC CDR3 sequence comprising or consisting of SEQ ID NO: 102,
xi) a HC CDR1 sequence comprising or consisting of SEQ ID NO: 107, a HC CDR2 sequence comprising or consisting of SEQ ID NO: 108, a HC CDR3 sequence comprising or consisting of SEQ ID NO: 109, a LC CDR1 sequence comprising or consisting of SEQ ID NO: 110, a LC CDR2 sequence comprising or consisting of SEQ ID NO: 111 and a LC CDR3 sequence comprising or consisting of SEQ ID NO: 112,
xii) a HC CDR1 sequence comprising or consisting of SEQ ID NO: 117, a HC CDR2 sequence comprising or consisting of SEQ ID NO: 118, a HC CDR3 sequence comprising or consisting of SEQ ID NO: 119, a LC CDR1 sequence comprising or consisting of SEQ ID NO: 120, a LC CDR2 sequence comprising or consisting of SEQ ID NO: 12 and a LC CDR3 sequence comprising or consisting of SEQ ID NO: 122,
xiii) a HC CDR1 sequence comprising or consisting of SEQ ID NO: 127, a HC CDR2 sequence comprising or consisting of SEQ ID NO: 128, a HC CDR3 sequence comprising or consisting of SEQ ID NO: 129, a LC CDR1 sequence comprising or consisting of SEQ ID NO: 130, a LC CDR2 sequence comprising or consisting of SEQ ID NO: 131 and a LC CDR3 sequence comprising or consisting of SEQ ID NO: 132,
xiv) a HC CDR1 sequence comprising or consisting of SEQ ID NO: 137, a HC CDR2 sequence comprising or consisting of SEQ ID NO: 138, a HC CDR3 sequence comprising or consisting of SEQ ID NO: 139, a LC CDR1 sequence comprising or consisting of SEQ ID NO: 140, a LC CDR2 sequence comprising or consisting of SEQ ID NO: 141 and a LC CDR3 sequence comprising or consisting of SEQ ID NO: 142,
xv) a HC CDR1 sequence comprising or consisting of SEQ ID NO: 147, a HC CDR2 sequence comprising or consisting of SEQ ID NO: 148, a HC CDR3 sequence comprising or consisting of SEQ ID NO: 149, a LC CDR1 sequence comprising or consisting of SEQ ID NO: 150, a LC CDR2 sequence comprising or consisting of SEQ ID NO: 151 and a LC CDR3 sequence comprising or consisting of SEQ ID NO: 152,
xvi) a HC CDR1 sequence comprising or consisting of SEQ ID NO: 157, a HC CDR2 sequence comprising or consisting of SEQ ID NO: 158, a HC CDR3 sequence comprising or consisting of SEQ ID NO: 159, a LC CDR1 sequence comprising or consisting of SEQ ID NO: 160, a LC CDR2 sequence comprising or consisting of SEQ ID NO: 161 and a LC CDR3 sequence comprising or consisting of SEQ ID NO: 162,
xvii) a HC CDR1 sequence comprising or consisting of SEQ ID NO: 167, a HC CDR2 sequence comprising or consisting of SEQ ID NO: 168, a HC CDR3 sequence comprising or consisting of SEQ ID NO: 169, a LC CDR1 sequence comprising or consisting of SEQ ID NO: 170, a LC CDR2 sequence comprising or consisting of SEQ ID NO: 171 and a LC CDR3 sequence comprising or consisting of SEQ ID NO: 172,
xviii) a HC CDR1 sequence comprising or consisting of SEQ ID NO: 177, a HC CDR2 sequence comprising or consisting of SEQ ID NO: 178, a HC CDR3 sequence comprising or consisting of SEQ ID NO: 179, a LC CDR1 sequence comprising or consisting of SEQ ID NO: 180, a LC CDR2 sequence comprising or consisting of SEQ ID NO: 181 and a LC CDR3 sequence comprising or consisting of SEQ ID NO: 182,
xix) a HC CDR1 sequence comprising or consisting of SEQ ID NO: 187, a HC CDR2 sequence comprising or consisting of SEQ ID NO: 188, a HC CDR3 sequence comprising or consisting of SEQ ID NO: 189, a LC CDR1 sequence comprising or consisting of SEQ ID NO: 190, a LC CDR2 sequence comprising or consisting of SEQ ID NO: 191 and a LC CDR3 sequence comprising or consisting of SEQ ID NO: 192,
xx) a HC CDR1 sequence comprising or consisting of SEQ ID NO: 197, a HC CDR2 sequence comprising or consisting of SEQ ID NO: 198, a HC CDR3 sequence comprising or consisting of SEQ ID NO: 199, a LC CDR1 sequence comprising or consisting of SEQ ID NO: 200, a LC CDR2 sequence comprising or consisting of SEQ ID NO: 201 and a LC CDR3 sequence comprising or consisting of SEQ ID NO: 202,
xxi) a HC CDR1 sequence comprising or consisting of SEQ ID NO: 207, a HC CDR2 sequence comprising or consisting of SEQ ID NO: 208, a HC CDR3 sequence comprising or consisting of SEQ ID NO: 209, a LC CDR1 sequence comprising or consisting of SEQ ID NO: 210, a LC CDR2 sequence comprising or consisting of SEQ ID NO: 211 and a LC CDR3 sequence comprising or consisting of SEQ ID NO: 212,
xxii) a HC CDR1 sequence comprising or consisting of SEQ ID NO: 217, a HC CDR2 sequence comprising or consisting of SEQ ID NO: 218, a HC CDR3 sequence comprising or consisting of SEQ ID NO: 219, a LC CDR1 sequence comprising or consisting of SEQ ID NO: 220, a LC CDR2 sequence comprising or consisting of SEQ ID NO: 221 and a LC CDR3 sequence comprising or consisting of SEQ ID NO: 222,
xxiii) a HC CDR1 sequence comprising or consisting of SEQ ID NO: 227, a HC CDR2 sequence comprising or consisting of SEQ ID NO: 228, a HC CDR3 sequence comprising or consisting of SEQ ID NO: 229, a LC CDR1 sequence comprising or consisting of SEQ ID NO: 230, a LC CDR2 sequence comprising or consisting of SEQ ID NO: 231 and a LC CDR3 sequence comprising or consisting of SEQ ID NO: 232,
xxiv) a HC CDR1 sequence comprising or consisting of SEQ ID NO: 237, a HC CDR2 sequence comprising or consisting of SEQ ID NO: 238, a HC CDR3 sequence comprising or consisting of SEQ ID NO: 239, a LC CDR1 sequence comprising or consisting of SEQ ID NO: 240, a LC CDR2 sequence comprising or consisting of SEQ ID NO: 241 and a LC CDR3 sequence comprising or consisting of SEQ ID NO: 242,
xxv) a HC CDR1 sequence comprising or consisting of SEQ ID NO: 247, a HC CDR2 sequence comprising or consisting of SEQ ID NO: 248, a HC CDR3 sequence comprising or consisting of SEQ ID NO: 249, a LC CDR1 sequence comprising or consisting of SEQ ID NO: 250, a LC CDR2 sequence comprising or consisting of SEQ ID NO: 251 and a LC CDR3 sequence comprising or consisting of SEQ ID NO: 252,
xxvi) a HC CDR1 sequence comprising or consisting of SEQ ID NO: 257, a HC CDR2 sequence comprising or consisting of SEQ ID NO: 258, a HC CDR3 sequence comprising or consisting of SEQ ID NO: 259, a LC CDR1 sequence comprising or consisting of SEQ ID NO: 260, a LC CDR2 sequence comprising or consisting of SEQ ID NO: 261 and a LC CDR3 sequence comprising or consisting of SEQ ID NO: 262,
xxvii) a HC CDR1 sequence comprising or consisting of SEQ ID NO: 267, a HC CDR2 sequence comprising or consisting of SEQ ID NO: 268, a HC CDR3 sequence comprising or consisting of SEQ ID NO: 269, a LC CDR1 sequence comprising or consisting of SEQ ID NO: 270, a LC CDR2 sequence comprising or consisting of SEQ ID NO: 271 and a LC CDR3 sequence comprising or consisting of SEQ ID NO: 272,
xxviii) a HC CDR1 sequence comprising or consisting of SEQ ID NO: 277, a HC CDR2 sequence comprising or consisting of SEQ ID NO: 278, a HC CDR3 sequence comprising or consisting of SEQ ID NO: 279, a LC CDR1 sequence comprising or consisting of SEQ ID NO: 280, a LC CDR2 sequence comprising or consisting of SEQ ID NO: 281 and a LC CDR3 sequence comprising or consisting of SEQ ID NO: 282, or
xxix) a HC CDR1 sequence comprising or consisting of SEQ ID NO: 287, a HC CDR2 sequence comprising or consisting of SEQ ID NO: 288, a HC CDR3 sequence comprising or consisting of SEQ ID NO: 289, a LC CDR1 sequence comprising or consisting of SEQ ID NO: 290, a LC CDR2 sequence comprising or consisting of SEQ ID NO: 291 and a LC CDR3 sequence comprising or consisting of SEQ ID NO: 292.

The invention also relates to an antibody or antigen-binding portion thereof wherein said antibody or antigen-binding portion thereof comprises a HC variable region sequence comprising SEQ ID NO: 4 or a sequence with at least 45%, 50%, 60%, 70%, 75%, 80%, 85% or 90% sequence identity thereto and a LC variable region sequence comprising SEQ ID NO: 6 or a sequence with at least 35%, 40%, 50%, 60%, 70%, 75%, 80%, 85%, 90% or 95% sequence identity thereto for example a LC variable region sequence comprising SEQ ID NO: 6.

The invention also relates to an antibody or antigen-binding portion thereof wherein said antibody or antigen-binding portion thereof has
a) a HC variable region sequence comprising SEQ ID NO: 4 or a sequence with at least 45%, 50%, 70%, 75%, 80%, 85% or 90% sequence identity thereto and a LC variable region sequence comprising SEQ ID NO: 6 or a sequence with at least 35%, 40%, 70%, 75%, 80%, 85% or 90% sequence identity thereto; or
b) a HC variable region sequence comprising SEQ ID NO: 14 or a sequence with at least 40%, 45%, 70%, 75%, 80%, 85% or 90% sequence identity thereto and a LC variable region sequence comprising SEQ ID NO: 16 or a sequence with at least 35%, 45%, 55%, 65%, 70%, 75%, 80%, 85% or 90% sequence identity thereto; or
c) a HC variable region sequence comprising SEQ ID NO: 24 or a sequence with at least 45%, 50%, 70%, 75%, 80%, 85% or 90% sequence identity thereto and a LC variable region sequence comprising SEQ ID NO: 6 or a sequence with at least 35%, 40%, 70%, 75%, 80%, 85% or 90% sequence identity thereto; or
d) a HC variable region sequence comprising SEQ ID NO: 34 or a sequence with at least 45%, 50%, 70%, 75%, 80%, 85% or 90% sequence identity thereto and a LC variable region sequence comprising SEQ ID NO: 6 or a sequence with at least 35%, 40%, 70%, 75%, 80%, 85% or 90% sequence identity thereto; or
e) a HC variable region sequence comprising SEQ ID NO: 44 or a sequence with at least 40%, 50%, 60%, 70%, 75%, 80%, 85% or 90% sequence identity thereto and a LC variable region sequence comprising SEQ ID NO: 46 or a sequence with at least 35%, 45%, 55%, 65%, 70%, 75%, 80%, 85% or 90% sequence identity thereto; or
f) a HC variable region sequence comprising SEQ ID NO: 54 or a sequence with at least 40%, 45%, 70%, 75%, 80%, 85% or 90% sequence identity thereto and a LC variable region sequence comprising SEQ ID NO: 56 or a sequence with at least 35%, 40%, 70%, 75%, 80%, 85% or 90% sequence identity thereto; or
g) a HC variable region sequence comprising SEQ ID NO: 64 or a sequence with at least 45%, 65%, 70%, 75%, 80%, 85% or 90% sequence identity thereto and a LC variable region sequence comprising SEQ ID NO: 36 or a sequence with at least 35%, 40%, 70%, 75%, 80%, 85% or 90% sequence identity thereto; or
h) a HC variable region sequence comprising SEQ ID NO: 74 or a sequence with at least 45%, 50%, 70%, 75%, 80%, 85% or 90% sequence identity thereto and a LC variable region sequence comprising SEQ ID NO: 6 or a sequence with at least 35%, 40%, 70%, 75%, 80%, 85% or 90% sequence identity thereto; or
i) a HC variable region sequence comprising SEQ ID NO: 84 or a sequence with at least 40%, 45%, 50%, 70%, 75%, 80%, 85% or 90% sequence identity thereto and a LC variable region sequence comprising SEQ ID NO: 86 or a sequence with at least 35%, 40%, 70%, 75%, 80%, 85% or 90% sequence identity thereto; or
j) a HC variable region sequence comprising SEQ ID NO: 94 or a sequence with at least 45%, 50%, 70%, 75%, 80%, 85% or 90% sequence identity thereto and a LC variable region sequence comprising SEQ ID NO: 6 or a sequence with at least 35%, 40%, 70%, 75%, 80%, 85% or 90% sequence identity thereto; or
k) a HC variable region sequence comprising SEQ ID NO: 104 or a sequence with at least 45%, 50%, 70%, 75%, 80%, 85% or 90% sequence identity thereto and a LC variable region sequence comprising SEQ ID NO: 6 or a sequence with at least 35%, 40%, 70%, 75%, 80%, 85% or 90% sequence identity thereto; or
l) a HC variable region sequence comprising SEQ ID NO: 114 or a sequence with at least 45%, 50%, 70%, 75%, 80%, 85% or 90% sequence identity thereto and a LC variable region sequence comprising SEQ ID NO: 6 or a sequence with at least 35%, 40%, 70%, 75%, 80%, 85% or 90% sequence identity thereto; or
m) a HC variable region sequence comprising SEQ ID NO: 124 or a sequence with at least 45%, 50%, 70%, 75%, 80%, 85% or 90% sequence identity thereto and a LC variable region sequence comprising SEQ ID NO: 6 or a sequence with at least 35%, 40%, 70%, 75%, 80%, 85% or 90% sequence identity thereto; or
n) a HC variable region sequence comprising SEQ ID NO: 134 or a sequence with at least 45%, 50%, 70%, 75%, 80%, 85% or 90% sequence identity thereto and a LC variable region sequence comprising SEQ ID NO: 136 or a sequence with at least 35%, 40%, 70%, 75%, 80%, 85% or 90% sequence identity thereto; or
o) a HC variable region sequence comprising SEQ ID NO:144 or a sequence with at least 45%, 50%, 70%, 75%, 80%, 85% or 90% sequence identity thereto and a LC variable region sequence comprising SEQ ID NO: 6 or a sequence with at least 35%, 40%, 70%, 75%, 80%, 85% or 90% sequence identity thereto; or
p) a HC variable region sequence comprising SEQ ID NO: 154 or a sequence with at least 45%, 50%, 70%, 75%, 80%, 85% or 90% sequence identity thereto and a LC variable region sequence comprising SEQ ID NO: 156 or a sequence with at least 35%, 40%, 70%, 75%, 80%, 85% or 90% sequence identity thereto; or
q) a HC variable region sequence comprising SEQ ID NO: 164 or a sequence with at least 45%, 50%, 70%, 75%, 80%, 85% or 90% sequence identity thereto and a LC variable region sequence comprising SEQ ID NO: 176 or a sequence with at least 35%, 40%, 70%, 75%, 80%, 85% or 90% sequence identity thereto; or
r) a HC variable region sequence comprising SEQ ID NO: 174 or a sequence with at least 45%, 50%, 70%, 75%, 80%, 85% or 90% sequence identity thereto and a LC variable region sequence comprising SEQ ID NO: 6 or a sequence with at least 35%, 40%, 70%, 75%, 80%, 85% or 90% sequence identity thereto; or
s) a HC variable region sequence comprising SEQ ID NO: 184 or a sequence with at least 45%, 50%, 70%, 75%, 80%, 85% or 90% sequence identity thereto and a LC variable region sequence comprising SEQ ID NO: 186 or a sequence with at least 35%, 40%, 70%, 75%, 80%, 85% or 90% sequence identity thereto; or
t) a HC variable region sequence comprising SEQ ID NO: 194 or a sequence with at least 45%, 50%, 70%, 75%, 80%, 85% or 90% sequence identity thereto and a LC variable region sequence comprising SEQ ID NO: 6 or a sequence with at least 35%, 40%, 70%, 75%, 80%, 85% or 90% sequence identity thereto; or
u) a HC variable region sequence comprising SEQ ID NO: 204 or a sequence with at least 40%, 45%, 70%, 75%, 80%, 85% or 90% sequence identity thereto and a LC variable region sequence comprising SEQ ID NO: 206 or a sequence with at least 35%, 40%, 70%, 75%, 80%, 85% or 90% sequence identity thereto; or
v) a HC variable region sequence comprising SEQ ID NO: 214 or a sequence with at least 40%, 45%, 70%, 75%, 80%, 85% or 90% sequence identity thereto and a LC variable region sequence comprising SEQ ID NO: 216 or a sequence with at least 35%, 40%, 70%, 75%, 80%, 85% or 90% sequence identity thereto; or
w) a HC variable region sequence comprising SEQ ID NO: 224 or a sequence with at least 40%, 45%, 70%, 75%, 80%, 85% or 90% sequence identity thereto and a LC variable region sequence comprising SEQ ID NO: 226 or a sequence with at least 35%, 40%, 70%, 75%, 80%, 85% or 90% sequence identity thereto; or
x) a HC variable region sequence comprising SEQ ID NO: 234 or a sequence with at least 45%, 50%, 70%, 75%, 80%, 85% or 90% sequence identity thereto and a LC variable region sequence comprising SEQ ID NO: 236 or a sequence with at least 35%, 40%, 70%, 75%, 80%, 85% or 90% sequence identity thereto; or
y) a HC variable region sequence comprising SEQ ID NO: 244 or a sequence with at least 40%, 45%, 70%, 75%, 80%, 85% or 90% sequence identity thereto and a LC variable region sequence comprising SEQ ID NO: 246 or a sequence with at least 35%, 40%, 70%, 75%, 80%, 85% or 90% sequence identity thereto; or
z) a HC variable region sequence comprising SEQ ID NO: 254 or a sequence with at least 40%, 45%, 70%, 75%, 80%, 85% or 90% sequence identity thereto and a LC variable region sequence comprising SEQ ID NO: 256 or a sequence with at least 35%, 40%, 70%, 75%, 80%, 85% or 90% sequence identity thereto; or
aa) a HC variable region sequence comprising SEQ ID NO: 264 or a sequence with at least 40%, 45%, 70%, 75%, 80%, 85% or 90% sequence identity thereto and a LC variable region sequence comprising SEQ ID NO: 266 or a sequence with at least 35%, 40%, 70%, 75%, 80%, 85% or 90% sequence identity thereto; or
bb) a HC variable region sequence comprising SEQ ID NO: 274 or a sequence with at least 45%, 50%, 70%, 75%, 80%, 85% or 90% sequence identity thereto and a LC variable region sequence comprising SEQ ID NO: 276 or a sequence with at least 35%, 40%, 70%, 75%, 80%, 85% or 90% sequence identity thereto; or
cc) a HC variable region sequence comprising SEQ ID NO: 284 or a sequence with at least 45%, 50%, 70%, 75%, 80%, 85% or 90% sequence identity thereto and a LC variable region sequence comprising SEQ ID NO: 286 or a sequence with at least 35%, 40%, 70%, 75%, 80%, 85% or 90% sequence identity thereto; or
dd) a HC variable region sequence comprising SEQ ID NO: 24 or a sequence with at least 45%, 50%, 70%, 75%, 80%, 85% or 90% sequence identity thereto and a LC variable region sequence comprising SEQ ID NO: 26 or a sequence with at least 35%, 40%, 70%, 75%, 80%, 85% or 90% sequence identity thereto.

The invention also relates to a pharmaceutical composition comprising an antibody or antigen-binding portion thereof as described herein.

The invention also relates to an antibody or antigen-binding portion thereof as described herein or the pharmaceutical composition as described herein for use in the treatment of disease.

The invention also relates to a method of treating a disease in a canine subject in need thereof comprising administering an effective amount of the antibody or antigen-binding portion thereof as described herein or a pharmaceutical composition as described herein.

In one embodiment, the disease is a cancer or tumor.

The invention also relates to a nucleic acid sequence that encodes an antibody or antibody antigen-binding portion thereof as described herein.

The invention also relates to a vector comprising a nucleic acid sequence as described herein.

The invention also relates to a host cell comprising the nucleic acid sequence as described herein or a vector as described herein.

The invention also relates to a kit comprising an antibody or antigen-binding portion thereof as described herein or a pharmaceutical composition as described herein.

The invention also relates to a method for making a canine antibody that binds PD-1 comprising culturing the isolated host cell as described herein and recovering said antibody.

The invention also relates to a method for making a canine antibody that binds PD-1 comprising the steps of
a) immunising a transgenic mouse that expresses a nucleic acid construct comprising canine heavy chain V genes and canine light chain V genes with PD-1 antigen,
b) generating a library of antibodies from said mouse and
c) isolating an antibody from said library.

The invention also relates to a method for detecting a PD-1 protein or an extracellular domain of a PD-1 protein in a biological sample from a canine subject, comprising contacting a biological sample with the antibody or antigen-binding portion thereof as described herein wherein said antibody or antigen-binding portion thereof is linked to a detectable label.

The invention also relates to a method of inhibiting tumor growth or metastasis comprising contacting a tumor cell with an effective amount of the antibody or antigen-binding portion thereof as described herein or pharmaceutical composition as described herein.

The invention also relates to a method of killing a tumor cell expressing PD-1, comprising contacting the cell with the antibody as described herein or pharmaceutical composition as described herein, such that killing of the cell expressing PD-1 occurs.

The invention also relates to a binding agent comprising the antibody or antigen-binding portion thereof as described herein wherein said antibody or antigen-binding portion thereof is linked to a second antibody or antigen-binding portion thereof that binds to a second target.

The invention also relates to an immunoconjugate comprising the antibody or antigen-binding portion thereof as described herein or the binding agent as described herein.

The invention also relates to a method of modulating an immune response comprising administering an antibody or fragment thereof as described herein, or a pharmaceutical composition as described herein or a binding agent as described herein, or an immunoconjugate as described herein.

The invention also relates to a combination therapy comprising an antibody or fragment thereof as described herein, or a pharmaceutical composition as described herein, or a binding agent as described herein, or an immunoconjugate as described herein and a further therapeutic moiety.

### BRIEF DESCRIPTION OF THE DRAWINGS

The invention is further described in the following non-limiting figures.

FIG. 1 shows the section of each expression plasmid encoding PD-1 ECD-mFc, PD-1 ECD-6His, PD-L1 ECD-mFc and PD-L2 ECD-mFc (ECD=extracellular domain). PD-1 ECD, PD-L1 ECD and PD-L2 ECD refer to the predicted extracellular domains of PD-1, PD-L1 and PD-L2, respectively. The “-mFc” suffix refers to the Fc portion of murine IgG2a comprising the genetic annotations Hinge-Constant Heavy2-Constant Heavy3-Constant Heavy Secreted (H-CH2-CH3-CHS) and “6His” refers to a hexahistidine tag.

FIG. 2 is a graph that shows the binding intensity of anti-PD-1 antibodies to HEK293 plasma membrane-embedded full-length canine PD-1 protein measured using flow cytometry. Cells stably transfected with canine PD-1 were incubated with anti-canine PD-1 mAb, followed by detection with an anti-canine IgG FITC secondary antibody.

FIGS. 3A-3B. FIG. 3A shows the binding of AlexaFluor 647-labelled PD-L1 ECD-mFc binding to PD-1-expressing HEK293 cells which were previously incubated with 2 μg/mL of anti-PD-1 antibodies in a flow cytometry-based assay. FIG. 3B shows the binding of AlexaFluor 647-labelled PD-L2 ECD-mFc binding to PD-1-expressing HEK293 cells which were previously incubated with 2 μg/mL of anti-PD-1 antibodies in a flow cytometry-based assay.

FIGS. 4A-4B. FIG. 4A is a table showing the SPR-based affinity and kinetics of anti-PD-1 antibodies conducted as multi-cycle kinetic experiments either in monovalent format (PD-1 analyte) or bivalent format (mAb analyte). FIG. 4B shows a table showing the apparent affinity of anti-PD-1 antibodies using a flow cytometry-based affinity assay. Apparent affinities were derived by reporting the concentration at which signal is 50% maximal after fitting the data to a “[Agonist] vs. response” 4-parameter logistic curve in Prism 8.3.0 (GraphPad).

FIGS. 5A-5C. FIG. 5A is a table that shows the fraction of mAb2 binding when mAb1 was prebound to saturating conditions using an “in tandem” SPR-based method. Values lower than 15% indicate complete blockade of a pair of mAbs whereas values above 75% indicate negligible blockade of mAb 2 binding by mAb 1. The darkest squares are −5 to 15%. FIG. 5B shows a correlation matrix of each mAb's binding profile against every other mAb's profile. To construct this table, the Pearson Product-Moment correlation coefficient is calculated between each mAb pair's relative binding values. Pearson Product-Moment correlation coefficients ≥0.97 were highlighted. FIG. 5C shows a visual representation of the resulting epitope bins.

FIG. 6 is a graph showing the binding of anti-PD-1 antibodies to concanavalin A-activated CD5⁺ canine T cells isolated from a healthy dog. PBMC Fc receptors were blocked with canine gamma globulin and anti-PD-1 mAbs were detected by flow cytometry with anti-canine IgG FITC.

FIG. 7 is a graph showing the functional in vitro assay of IFN-γ secretion carried out using supernatants collected from PBMCs which had been stimulated with concanavalin A in the presence of 10 μg/mL anti-PD-1 mAbs or an isotype control mAb. After incubation with each anti-PD-1 antibody for 72 h, IFN-γ secretion was evaluated by sandwich ELISA using a canine IFN-gamma ELISA.

FIGS. 8A-8B. FIG. 8A shows PD-1/PD-L1 receptor blockade in a luciferase reporter assay using anti-PD-1 mAbs at 3 μg/mL. FIG. 8B shows receptor blockade of selected anti-PD-1 mAbs in a luciferase reporter assay using at a concentration range spanning 666 nM to 0.03 nM. IC₅₀ concentrations are indicated in nM. Reporter Jurkat cells express chimeric canine (ectodomain) and human (transmembrane domain and intracellular domain) PD-1, a TCR specific for a known MHC-peptide complex and transcribe luciferase under the control of an NFAT-regulated promoter. Raji antigen presenting cells express an MHC-peptide complex bound by then Jurkat TCR and full-length canine PD-L1. In the absence of inhibition, TCR signalling is repressed by PD-1 signalling leading to low levels of luciferase transcription. High levels of PD-1 receptor blockade lead to unabated TCR signalling, leading to high levels of luciferase transcription.

FIGS. 9A-9B. FIG. 9A shows a structural representation using canine PD-1 and PD-L1 extracellular domain (PD-1 ECD and PD-L1 ECD, respectively) homology models to highlight the residue positions identified in the PMX-126 binding epitope on PD-1 in black. FIG. 9B A sequence-based representation of the PD-1 extracellular domain with secondary structure features highlighted. Residue positions identified in the PMX-126 binding epitope on PD-1 in grey.

FIGS. 10A-10B. FIG. 10A shows a structural representation using canine PD-1 and PD-L1 extracellular domain (PD-1 ECD and PD-L1 ECD, respectively) homology models to highlight the residue positions identified in the PMX-127 binding epitope on PD-1 in black. FIG. 10B A sequence-based representation of the PD-1 extracellular domain with secondary structure features highlighted. Residue positions identified in the PMX-127 binding epitope on PD-1 in grey.

FIG. 11 shows a sequence representation of full-length canine PD-1. The predicted extracellular domain (ECD) is highlighted in grey.

FIGS. 12A-12B. FIG. 12A shows flow cytometric and histogram plots demonstrating the binding of PMX-127 antibody to PD-1 receptor expressed on the cell surface of T cells from dogs with lymphoma (n=2). Pre-gated on live, single CD5⁺ T cells. FIG. 12B flow cytometric and histogram plots demonstrating the binding of PMX-127 antibody to PD-1 receptor expressed on the cell surface of T cells from dogs with transitional cell carcinoma (n=2). Pre-gated on live, single CD5⁺ T cells.

FIG. 13 shows augmentation of IFN-γ production in PBMCs from dogs with cancer following in-vitro treatment with canine anti-PD-1 antibodies PMX-126 and PMX-127. Patient PBMC samples from dogs with rectal adenocarcinoma and lymphoma were stimulated with ConA in the presence of 10 μg/ml of anti-PD-1 mAbs or Ritux-def2 isotype control. After 96 h supernatants collected and the amount of canine IFN-γ was measured in triplicate using a canine IFN-gamma ELISA.

FIGS. 14A-14C. FIG. 14A shows pharmacokinetic analysis showing linear antibody concentration time curves of canine anti-PD-1 antibody in healthy beagle dogs after a single intravenous administration of PMX-126 at low and high dosages (mean±SD, n=3) The presence of antibodies in the plasma was assessed by direct ELISA using purified canine PD-1 Fc-tag protein. FIG. 14B Pharmacokinetic analysis showing antibody concentration time curves of canine anti-PD-1 antibody in healthy beagle dogs after a single intravenous administration of PMX-127 at low and high dosages (mean±SD, n=3). The presence of antibodies in the plasma was assessed by direct ELISA using purified canine PD-1 Fc-tag protein. FIG. 14C shows a table of results of the non-compartmental analysis of PMX-126 or PMX-127 antibodies demonstrating similar half-life of 5 days. VD (L): volume of distribution. Cmax: maximum observed plasma concentration, AUC: area under the plasma concentration vs. time curve from time 0 to 28 days,

FIG. 15 is a summary table showing potential adverse events following administration of a single intravenous dose of canine PD-1 PMX-126 and PMX-127 antibodies to laboratory beagle dogs. Changes were mild, transient and may not be specific to the antibody treatment.

FIGS. 16A-16B. FIG. 16A shows SPR-based assay graphs demonstrating that there is no cross-reactivity of the anti-canine mAbs PMX-126 and PMX-127 to recombinant human PD-1 protein at a concentration of 900 nM. FIG. 16B ELISA assay graphs showing that PMX-126 and PMX-127 do not cross-react with human PD-1 protein up to a concentration of 100 μg/mL. Anti-human PD-1 mAb pembrolizumab was included as a control to demonstrate that the recombinant human PD-1 protein is active.

FIG. 17 is a receptor occupancy graph demonstrating that canine anti-PD-1 antibodies PMX-126 and PMX-127 remain bound to circulating CD5⁺ T cells up to 21 days after a single intravenous dose were given to laboratory dogs (n=3 dogs per group). Data are shown as the mean±SD.

FIGS. 18A-18B. FIG. 18A is a volcano plot showing genes that are differentially expressed at timepoint Day 07 as compared to pre-treatment following treatment with canine anti-PD-1 antibody PMX-126 in laboratory dogs. Highlighted displaying genes met significance criteria of log 2 fold change >1.5 and p-value <0.05. Horizontal lines on the plot describe statistical significance; thus, differentially expressed (DE) genes with high statistical significance are shown from the bottom to the top. Highly DE genes are at the horizontal extremes of the plot, thus, the more the right, the greater the gene expression in Day 07 timepoint compared to pre-treatment. FIG. 18B is a volcano plot showing genes that are differentially expressed at timepoint Day 07 as compared to pre-treatment following treatment with canine anti-PD-1 antibody PMX-127 in laboratory dogs. Highlighted displaying genes met significance criteria of log 2 fold change >1.5 and p-value <0.05. Horizontal lines on the plot describe statistical significance; thus, differentially expressed (DE) genes with high statistical significance are shown from the bottom to the top. Highly DE genes are at the horizontal extremes of the plot, thus, the more the right, the greater the gene expression in Day 07 timepoint compared to pre-treatment.

Table 1. Amino Acid Residues and Examples of Conservative Amino Acid Substitutions

Table 2. Sequence IDs of amino acid and nucleotide sequences for each anti-PD-1 antibody included within the specification.

### DETAILED DESCRIPTION

The embodiments of the invention will now be further described. In the following passages, different embodiments are described. Each aspect so defined may be combined with any other aspect or aspects unless clearly indicated to the contrary.

Generally, nomenclatures used in connection with, and techniques of, cell and tissue culture, pathology, oncology, molecular biology, immunology, microbiology, genetics and protein and nucleic acid chemistry and hybridization described herein are those well-known and commonly used in the art. The methods and techniques of the present disclosure are generally performed according to conventional methods well-known in the art and as described in various general and more specific references that are cited and discussed throughout the present specification unless otherwise indicated. See, e.g., Green and Sambrook et al., Molecular Cloning: A Laboratory Manual, 4th ed., Cold Spring Harbor Laboratory Press, Cold Spring Harbor, N.Y. (2012); Therapeutic Monoclonal Antibodies: From Bench to Clinic, Zhiqiang An (Editor), Wiley, (2009); and Antibody Engineering, 2nd Ed., Vols 1 and 2, Ontermann and Dubel, eds., Springer-Verlag, Heidelberg (2010).

Enzymatic reactions and purification techniques are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

The inventors have developed fully canine antibodies that bind specifically to canine PD-1. These antibodies were generated in transgenic rodents expressing canine V, D, J genes. Therefore, the antibodies are less likely to be immunogenic for administration to canine subjects than caninized antibodies or chimeric antibodies. Furthermore, as these antibodies can be used directly, with no further modifications to their variable regions, there is no risk of reducing the affinity or otherwise compromising the antibody. Other technologies risk introducing development or efficacy liabilities through the ex vivo combination of antibody sequences of canine origin with that from another species, typically rodent. Thus, preferably, the invention relates to an isolated canine antibody or antigen-binding portion thereof which binds canine PD-1.

The invention thus provides isolated antibodies that bind canine PD-1 and block or reduce the interaction of PD-1 with PD-L1 and/or the interaction of PD-1 with PD-L2, pharmaceutical compositions comprising such binding molecules, as well as isolated nucleic acids, isolated recombinant expression vectors and isolated host cells for making such binding proteins. Also provided are methods of using the antibodies disclosed herein to detect canine PD-1 and methods of treating disease. In another aspect, the invention provides binding molecules comprising an antibody or antigen-binding portion that binds canine PD-1 and block the interaction of PD-1 with PD-L1 and/or the interaction of PD-1 with PD-L2 as described herein. The properties of the antibodies and antigen binding portions thereof of the invention can be exploited in therapeutic methods and uses as well as in pharmaceutical formulations as described herein.

In one embodiment of the invention an isolated canine antibody or antigen binding fragment thereof which binds to canine PD-1 is provided wherein said antibody comprises an HC CDR1 sequence comprising or consisting of SEQ ID NO: 17 or an amino acid sequence which has 1, 2, 3, 4, or 5 amino acid differences compared to SEQ ID NO: 17 and an HC CDR2 sequence comprising or consisting of SEQ ID NO: 18 or an amino acid sequence which has 1, 2, 3, 4, 5, or 6 amino acid differences compared to SEQ ID NO: 18 and an HC CDR3 sequence comprising or consisting of SEQ ID NO: 19 or an amino acid sequence which has 1, 2, 3, 4, 5, 6, 7, 8, or 9 amino acid differences compared to SEQ ID NO: 19 and an LC CDR1 sequence comprising or consisting of SEQ ID NO: 20 or an amino acid sequence which has 1, 2, 3, 4, 5, 6, 7, 8, 9, 10, 11, 12, 13 or 14 amino acid differences compared to SEQ ID NO: 20 and an LC CDR2 sequence comprising or consisting of SEQ ID NO: 21 or an amino acid sequence which has 1, 2, 3, 4, or 5 amino acid differences compared to SEQ ID NO: 21 and an LC CDR3 sequence comprising or consisting of SEQ ID NO: 22 or an amino acid sequence which has 1, 2, 3, 4, 5, 6, 7, 8, 9, or 10 amino acid differences compared to SEQ ID NO: 22; or an HC CDR1 sequence comprising or consisting of SEQ ID NO: 207 or an amino acid sequence which has 1, or 2 amino acid differences compared to SEQ ID NO: 207 and an HC CDR2 sequence comprising or consisting of SEQ ID NO: 208 or an amino acid sequence which has 1, 2, 3, 4, or 5 amino acid differences compared to SEQ ID NO: 208 and an HC CDR3 sequence comprising or consisting of SEQ ID NO: 209 or an amino acid sequence which has 1, 2, 3, 4, 5, 6, 7, 8, 9, 10, 11, 12, 13, 14, 15, or 16 amino acid differences compared to SEQ ID NO: 209 and an LC CDR1 sequence comprising or consisting of SEQ ID NO: 210 or an amino acid sequence which has 1, 2 or 3 amino acid differences compared to SEQ ID NO: 210 and an LC CDR2 sequence comprising or consisting of SEQ ID NO: 211 or an amino acid sequence which has 1 or 2 amino acid differences compared to SEQ ID NO: 211 and an LC CDR3 sequence comprising or consisting of SEQ ID NO: 212 or an amino acid sequence which has 1, 2, 3, or 4 amino acid differences compared to SEQ ID NO: 212; or an HC CDR1 sequence comprising or consisting of SEQ ID NO: 97 or an amino acid sequence which has 1, 2, or 3 amino acid differences compared to SEQ ID NO: 97 and an HC CDR2 sequence comprising or consisting of SEQ ID NO: 98 or an amino acid sequence which has 1, 2, 3, or 4 amino acid differences compared to SEQ ID NO: 98 and an HC CDR3 sequence comprising or consisting of SEQ ID NO: 99 or an amino acid sequence which has 1, 2, 3, 4, 5, 6, 7, 8, 9, 10, 11, 12, 13, or 14 amino acid differences compared to SEQ ID NO: 99 and an LC CDR1 sequence comprising or consisting of SEQ ID NO: 100 or an amino acid sequence which has 1, 2, 3, 4, 5, 6, 7, 8, 9, 10, 11, 12, 13, 14 or 15 amino acid differences compared to SEQ ID NO: 100 and an LC CDR2 sequence comprising or consisting of SEQ ID NO: 101 or an amino acid sequence which has 1, 2, 3, 4, or 5 amino acid differences compared to SEQ ID NO: 101 and an LC CDR3 sequence comprising or consisting of SEQ ID NO: 102 or an amino acid sequence which has 1, 2, 3, 4, 5, 6, 7, 8, 9, or 10 amino acid differences compared to SEQ ID NO: 102.

In one embodiment of the invention the amino acid differences are selected from conservative amino acid substitutions and/or non-conservative amino acid substitutions.

In another related embodiment of the invention an isolated canine antibody or antigen binding fragment thereof which binds to canine PD-1 is provided wherein said antibody or antigen-binding fragment thereof is provided which has i) a HC CDR1 sequence comprising or consisting of SEQ ID NO: 17, a HC CDR2 sequence comprising or consisting of SEQ ID NO: 18, a HC CDR3 sequence comprising or consisting of SEQ ID NO: 19, a LC CDR1 sequence comprising or consisting of SEQ ID NO: 20, a LC CDR2 sequence comprising or consisting of SEQ ID NO: 21 and a LC CDR3 sequence comprising or consisting of SEQ ID NO: 22, ii) a HC CDR1 sequence comprising or consisting of SEQ ID NO: 47, a HC CDR2 sequence comprising or consisting of SEQ ID NO: 48, a HC CDR3 sequence comprising or consisting of SEQ ID NO: 49, a LC CDR1 sequence comprising SEQ ID NO: 50, a LC CDR2 sequence comprising or consisting of SEQ ID NO: 51 and a LC CDR3 sequence comprising or consisting of SEQ ID NO: 52, iii) a HC CDR1 sequence comprising SEQ ID NO: 57, a HC CDR2 sequence comprising SEQ ID NO: 58, a HC CDR3 sequence comprising SEQ ID NO: 59, a LC CDR1 sequence comprising SEQ ID NO: 60, a LC CDR2 sequence comprising SEQ ID NO: 61 and a LC CDR3 sequence comprising SEQ ID NO: 62, iv) a HC CDR1 sequence comprising or consisting of SEQ ID NO: 37, a HC CDR2 sequence comprising or consisting of SEQ ID NO: 38, a HC CDR3 sequence comprising or consisting of SEQ ID NO: 39, a LC CDR1 sequence comprising or consisting of SEQ ID NO: 40, a LC CDR2 sequence comprising or consisting of SEQ ID NO: 41 and a LC CDR3 sequence comprising or consisting of SEQ ID NO: 42, v) a HC CDR1 sequence comprising or consisting of SEQ ID NO: 87, a HC CDR2 sequence comprising or consisting of SEQ ID NO: 88, a HC CDR3 sequence comprising or consisting of SEQ ID NO: 89, a LC CDR1 sequence comprising or consisting of SEQ ID NO: 90, a LC CDR2 sequence comprising or consisting of SEQ ID NO: 91 and a LC CDR3 sequence comprising or consisting of SEQ ID NO: 92, vi) a HC CDR1 sequence comprising or consisting of SEQ ID NO: 137, a HC CDR2 sequence comprising or consisting of SEQ ID NO: 138, a HC CDR3 sequence comprising or consisting of SEQ ID NO: 139, a LC CDR1 sequence comprising or consisting of SEQ ID NO: 141, a LC CDR2 sequence comprising or consisting of SEQ ID NO: 142 and a LC CDR3 sequence comprising or consisting of SEQ ID NO: 143, vii) a HC CDR1 sequence comprising or consisting of SEQ ID NO: 157, a HC CDR2 sequence comprising or consisting of SEQ ID NO: 158, a HC CDR3 sequence comprising or consisting of SEQ ID NO: 159, a LC CDR1 sequence comprising or consisting of SEQ ID NO: 160, a LC CDR2 sequence comprising or consisting of SEQ ID NO: 161 and a LC CDR3 sequence comprising or consisting of SEQ ID NO: 162, viii) a HC CDR1 sequence comprising or consisting of SEQ ID NO: 177, a HC CDR2 sequence comprising or consisting of SEQ ID NO: 178, a HC CDR3 sequence comprising or consisting of SEQ ID NO: 179, a LC CDR1 sequence comprising or consisting of SEQ ID NO: 180, a LC CDR2 sequence comprising or consisting of SEQ ID NO: 181 and a LC CDR3 sequence comprising or consisting of SEQ ID NO: 182, ix) a HC CDR1 sequence comprising or consisting of SEQ ID NO: 187, a HC CDR2 sequence comprising or consisting of SEQ ID NO: 188, a HC CDR3 sequence comprising or consisting of SEQ ID NO: 189, a LC CDR1 sequence comprising or consisting of SEQ ID NO: 190, a LC CDR2 sequence comprising or consisting of SEQ ID NO: 191 and a LC CDR3 sequence comprising or consisting of SEQ ID NO: 192, x) a HC CDR1 sequence comprising or consisting of SEQ ID NO: 207, a HC CDR2 sequence comprising or consisting of SEQ ID NO: 208, a HC CDR3 sequence comprising or consisting of SEQ ID NO: 209, a LC CDR1 sequence comprising or consisting of SEQ ID NO: 210, a LC CDR2 sequence comprising or consisting of SEQ ID NO: 211 and a LC CDR3 sequence comprising or consisting of SEQ ID NO: 212, xi) a HC CDR1 sequence comprising or consisting of SEQ ID NO: 217, a HC CDR2 sequence comprising or consisting of SEQ ID NO: 218, a HC CDR3 sequence comprising or consisting of SEQ ID NO: 219, a LC CDR1 sequence comprising or consisting of SEQ ID NO: 220, a LC CDR2 sequence comprising or consisting of SEQ ID NO: 221 and a LC CDR3 sequence comprising or consisting of SEQ ID NO: 222, xii) a HC CDR1 sequence comprising or consisting of SEQ ID NO: 227, a HC CDR2 sequence comprising or consisting of SEQ ID NO: 228, a HC CDR3 sequence comprising or consisting of SEQ ID NO: 229, a LC CDR1 sequence comprising or consisting of SEQ ID NO: 230, a LC CDR2 sequence comprising or consisting of SEQ ID NO: 231 and a LC CDR3 sequence comprising or consisting of SEQ ID NO: 232, xiii) a HC CDR1 sequence comprising or consisting of SEQ ID NO: 237, a HC CDR2 sequence comprising or consisting of SEQ ID NO: 238, a HC CDR3 sequence comprising or consisting of SEQ ID NO: 239, a LC CDR1 sequence comprising or consisting of SEQ ID NO: 240, a LC CDR2 sequence comprising or consisting of SEQ ID NO: 241 and a LC CDR3 sequence comprising or consisting of SEQ ID NO: 242, xiv) a HC CDR1 sequence comprising or consisting of SEQ ID NO: 247, a HC CDR2 sequence comprising or consisting of SEQ ID NO: 248, a HC CDR3 sequence comprising or consisting of SEQ ID NO: 249, a LC CDR1 sequence comprising or consisting of SEQ ID NO: 250, a LC CDR2 sequence comprising or consisting of SEQ ID NO: 251 and a LC CDR3 sequence comprising or consisting of SEQ ID NO: 252, xv) a HC CDR1 sequence comprising or consisting of SEQ ID NO: 257, a HC CDR2 sequence comprising or consisting of SEQ ID NO: 258, a HC CDR3 sequence comprising or consisting of SEQ ID NO: 259, a LC CDR1 sequence comprising or consisting of SEQ ID NO: 260, a LC CDR2 sequence comprising or consisting of SEQ ID NO: 261 and a LC CDR3 sequence comprising or consisting of SEQ ID NO: 262, xvi) a HC CDR1 sequence comprising or consisting of SEQ ID NO: 267, a HC CDR2 sequence comprising or consisting of SEQ ID NO: 268, a HC CDR3 sequence comprising or consisting of SEQ ID NO: 269, a LC CDR1 sequence comprising or consisting of SEQ ID NO: 270, a LC CDR2 sequence comprising or consisting of SEQ ID NO: 271 and a LC CDR3 sequence comprising or consisting of SEQ ID NO: 271, xvii) a HC CDR1 sequence comprising or consisting of SEQ ID NO: 117, a HC CDR2 sequence comprising or consisting of SEQ ID NO: 118, a HC CDR3 sequence comprising or consisting of SEQ ID NO: 119, a LC CDR1 sequence comprising or consisting of SEQ ID NO: 120, a LC CDR2 sequence comprising or consisting of SEQ ID NO: 121 and a LC CDR3 sequence comprising or consisting of SEQ ID NO: 122, xviii) a HC CDR1 sequence comprising or consisting of SEQ ID NO: 127, a HC CDR2 sequence comprising or consisting of SEQ ID NO: 128, a HC CDR3 sequence comprising or consisting of SEQ ID NO: 129, a LC CDR1 sequence comprising or consisting of SEQ ID NO: 130, a LC CDR2 sequence comprising or consisting of SEQ ID NO: 131 and a LC CDR3 sequence comprising or consisting of SEQ ID NO: 132, xxix) a HC CDR1 sequence comprising or consisting of SEQ ID NO: 167, a HC CDR2 sequence comprising or consisting of SEQ ID NO: 168, a HC CDR3 sequence comprising or consisting of SEQ ID NO: 169, a LC CDR1 sequence comprising or consisting of SEQ ID NO: 170, a LC CDR2 sequence comprising or consisting of SEQ ID NO: 171 and a LC CDR3 sequence comprising or consisting of SEQ ID NO: 172, xx) a HC CDR1 sequence comprising or consisting of SEQ ID NO: 197, a HC CDR2 sequence comprising or consisting of SEQ ID NO: 198, a HC CDR3 sequence comprising or consisting of SEQ ID NO: 199, a LC CDR1 sequence comprising or consisting of SEQ ID NO: 200, a LC CDR2 sequence comprising or consisting of SEQ ID NO: 201 and a LC CDR3 sequence comprising or consisting of SEQ ID NO: 202, xxi) a HC CDR1 sequence comprising or consisting of SEQ ID NO: 97, a HC CDR2 sequence comprising or consisting of SEQ ID NO: 98, a HC CDR3 sequence comprising or consisting of SEQ ID NO: 99, a LC CDR1 sequence comprising or consisting of SEQ ID NO: 100, a LC CDR2 sequence comprising or consisting of SEQ ID NO: 101 and a LC CDR3 sequence comprising or consisting of SEQ ID NO: 102, xxii) a HC CDR1 sequence comprising or consisting of SEQ ID NO: 277, a HC CDR2 sequence comprising or consisting of SEQ ID NO: 278, a HC CDR3 sequence comprising or consisting of SEQ ID NO: 279, a LC CDR1 sequence comprising or consisting of SEQ ID NO: 280, a LC CDR2 sequence comprising or consisting of SEQ ID NO: 281 and a LC CDR3 sequence comprising or consisting of SEQ ID NO: 282, xxiii) a HC CDR1 sequence comprising or consisting of SEQ ID NO: 287, a HC CDR2 sequence comprising or consisting of SEQ ID NO: 288, a HC CDR3 sequence comprising or consisting of SEQ ID NO: 289, a LC CDR1 sequence comprising or consisting of SEQ ID NO: 290, a LC CDR2 sequence comprising or consisting of SEQ ID NO: 291 and a LC CDR3 sequence comprising or consisting of SEQ ID NO: 292, xxiv) a HC CDR1 sequence comprising or consisting of SEQ ID NO: 107, a HC CDR2 sequence comprising or consisting of SEQ ID NO: 108, a HC CDR3 sequence comprising or consisting of SEQ ID NO: 109, a LC CDR1 sequence comprising or consisting of SEQ ID NO: 110, a LC CDR2 sequence comprising or consisting of SEQ ID NO: 111 and a LC CDR3 sequence comprising or consisting of SEQ ID NO: 112, xxv) a HC CDR1 sequence comprising or consisting of SEQ ID NO: 147, a HC CDR2 sequence comprising or consisting of SEQ ID NO: 148, a HC CDR3 sequence comprising or consisting of SEQ ID NO: 149, a LC CDR1 sequence comprising or consisting of SEQ ID NO: 150, a LC CDR2 sequence comprising or consisting of SEQ ID NO: 151 and a LC CDR3 sequence comprising or consisting of SEQ ID NO: 152, xxvi) a HC CDR1 sequence comprising or consisting of SEQ ID NO: 7, a HC CDR2 sequence comprising or consisting of SEQ ID NO: 8, a HC CDR3 sequence comprising or consisting of SEQ ID NO: 9, a LC CDR1 sequence comprising or consisting of SEQ ID NO: 10, a LC CDR2 sequence comprising or consisting of SEQ ID NO: 11 and a LC CDR3 sequence comprising or consisting of SEQ ID NO: 12, xxvii) a HC CDR1 sequence comprising or consisting of SEQ ID NO: 27, a HC CDR2 sequence comprising or consisting of SEQ ID NO: 28, a HC CDR3 sequence comprising or consisting of SEQ ID NO: 29, a LC CDR1 sequence comprising or consisting of SEQ ID NO: 30, a LC CDR2 sequence comprising or consisting of SEQ ID NO: 31 and a LC CDR3 sequence comprising or consisting of SEQ ID NO: 32, xxviii) a HC CDR1 sequence comprising or consisting of SEQ ID NO: 67, a HC CDR2 sequence comprising or consisting of SEQ ID NO: 68, a HC CDR3 sequence comprising or consisting of SEQ ID NO: 69, a LC CDR1 sequence comprising or consisting of SEQ ID NO: 70, a LC CDR2 sequence comprising or consisting of SEQ ID NO: 71 and a LC CDR3 sequence comprising or consisting of SEQ ID NO: 72, xxix) a HC CDR1 sequence comprising or consisting of SEQ ID NO: 77, a HC CDR2 sequence comprising or consisting of SEQ ID NO: 78, a HC CDR3 sequence comprising or consisting of SEQ ID NO: 79, a LC CDR1 sequence comprising or consisting of SEQ ID NO: 80, a LC CDR2 sequence comprising or consisting of SEQ ID NO: 81 and a LC CDR3 sequence comprising or consisting of SEQ ID NO: 82.

Also within the scope of the invention an isolated canine antibody or antigen binding fragment thereof which binds to canine PD-1 is provided wherein said antibody or antigen-binding fragment thereof is has the VH and LC CDRs with at least 90% or 95% sequence identity to the CDRs as shown in i) to xxviii) above.

In an embodiment of the invention the antibody or antigen-binding portion thereof which binds to canine PD-1 comprises a HC variable region sequence comprising SEQ ID NO: 4 or a sequence with at least 45%, 50%, 60%, 70%, 75%, 80%, 85% or 90% sequence identity thereto and a LC variable region sequence comprising SEQ ID NO: 6 or a sequence with at least 35%, 40%, 50%, 60%, 70%, 75%, 80%, 85%, 90% or 95% sequence identity thereto for example a LC variable region sequence comprising SEQ ID NO: 6.

In another related embodiment of the invention an antibody or antigen-binding portion thereof which binds to canine PD-1 is provided which has: a) a HC variable region sequence comprising SEQ ID NO: 14 or a sequence with at least 40%, 45%, 70%, 75%, 80%, 85% or 90% sequence identity thereto and a LC variable region sequence comprising SEQ ID NO: 16 or a sequence with at least 35%, 45%, 55%, 65%, 70%, 75%, 80%, 85% or 90% sequence identity thereto; or b) a HC variable region sequence comprising SEQ ID NO: 44 or a sequence with at least 40%, 50%, 60%, 70%, 75%, 80%, 85% or 90% sequence identity thereto and a LC variable region sequence comprising SEQ ID NO: 46 or a sequence with at least 35%, 45%, 55%, 65%, 70%, 75%, 80%, 85% or 90% sequence identity thereto; or c) a HC variable region sequence comprising SEQ ID NO: 54 or a sequence with at least 40%, 45%, 70%, 75%, 80%, 85% or 90% sequence identity thereto and a LC variable region sequence comprising SEQ ID NO: 56 or a sequence with at least 35%, 40%, 70%, 75%, 80%, 85% or 90% sequence identity thereto; or d) a HC variable region sequence comprising SEQ ID NO: 34 or a sequence with at least 45%, 65%, 70%, 75%, 80%, 85% or 90% sequence identity thereto and a LC variable region sequence comprising SEQ ID NO: 36 or a sequence with at least 35%, 40%, 70%, 75%, 80%, 85% or 90% sequence identity thereto; or e) a HC variable region sequence comprising SEQ ID NO: 84 or a sequence with at least 40%, 45%, 50%, 70%, 75%, 80%, 85% or 90% sequence identity thereto and a LC variable region sequence comprising SEQ ID NO: 86 or a sequence with at least 35%, 40%, 70%, 75%, 80%, 85% or 90% sequence identity thereto; or f) a HC variable region sequence comprising SEQ ID NO: 134 or a sequence with at least 45%, 50%, 70%, 75%, 80%, 85% or 90% sequence identity thereto and a LC variable region sequence comprising SEQ ID NO: 136 or a sequence with at least 35%, 40%, 70%, 75%, 80%, 85% or 90% sequence identity thereto; or g) a HC variable region sequence comprising SEQ ID NO: 154 or a sequence with at least 45%, 50%, 70%, 75%, 80%, 85% or 90% sequence identity thereto and a LC variable region sequence comprising SEQ ID NO: 156 or a sequence with at least 35%, 40%, 70%, 75%, 80%, 85% or 90% sequence identity thereto; or h) a HC variable region sequence comprising SEQ ID NO: 174 or a sequence with at least 45%, 50%, 70%, 75%, 80%, 85% or 90% sequence identity thereto and a LC variable region sequence comprising SEQ ID NO: 176 or a sequence with at least 35%, 40%, 70%, 75%, 80%, 85% or 90% sequence identity thereto; or i) a HC variable region sequence comprising SEQ ID NO: 184 or a sequence with at least 45%, 50%, 70%, 75%, 80%, 85% or 90% sequence identity thereto and a LC variable region sequence comprising SEQ ID NO: 186 or a sequence with at least 35%, 40%, 70%, 75%, 80%, 85% or 90% sequence identity thereto; or j) a HC variable region sequence comprising SEQ ID NO: 204 or a sequence with at least 40%, 45%, 70%, 75%, 80%, 85% or 90% sequence identity thereto and a LC variable region sequence comprising SEQ ID NO: 206 or a sequence with at least 35%, 40%, 70%, 75%, 80%, 85% or 90% sequence identity thereto; or k) a HC variable region sequence comprising SEQ ID NO: 214 or a sequence with at least 40%, 45%, 70%, 75%, 80%, 85% or 90% sequence identity thereto and a LC variable region sequence comprising SEQ ID NO: 216 or a sequence with at least 35%, 40%, 70%, 75%, 80%, 85% or 90% sequence identity thereto; or 1) a HC variable region sequence comprising SEQ ID NO: 224 or a sequence with at least 40%, 45%, 70%, 75%, 80%, 85% or 90% sequence identity thereto and a LC variable region sequence comprising SEQ ID NO: 226 or a sequence with at least 35%, 40%, 70%, 75%, 80%, 85% or 90% sequence identity thereto; or m) a HC variable region sequence comprising SEQ ID NO: 234 or a sequence with at least 45%, 50%, 70%, 75%, 80%, 85% or 90% sequence identity thereto and a LC variable region sequence comprising SEQ ID NO: 236 or a sequence with at least 35%, 40%, 70%, 75%, 80%, 85% or 90% sequence identity thereto; or n) a HC variable region sequence comprising SEQ ID NO: 244 or a sequence with at least 40%, 45%, 70%, 75%, 80%, 85% or 90% sequence identity thereto and a LC variable region sequence comprising SEQ ID NO: 246 or a sequence with at least 35%, 40%, 70%, 75%, 80%, 85% or 90% sequence identity thereto; or o) a HC variable region sequence comprising SEQ ID NO: 254 or a sequence with at least 40%, 45%, 70%, 75%, 80%, 85% or 90% sequence identity thereto and a LC variable region sequence comprising SEQ ID NO: 256 or a sequence with at least 35%, 40%, 70%, 75%, 80%, 85% or 90% sequence identity thereto; or p) a HC variable region sequence comprising SEQ ID NO: 264 or a sequence with at least 40%, 45%, 70%, 75%, 80%, 85% or 90% sequence identity thereto and a LC variable region sequence comprising SEQ ID NO: 266 or a sequence with at least 35%, 40%, 70%, 75%, 80%, 85% or 90% sequence identity thereto; or q) a HC variable region sequence comprising SEQ ID NO: 114 or a sequence with at least 40%, 45%, 70%, 75%, 80%, 85% or 90% sequence identity thereto and a LC variable region sequence comprising SEQ ID NO: 116 or a sequence with at least 35%, 40%, 70%, 75%, 80%, 85% or 90% sequence identity thereto; or r) a HC variable region sequence comprising SEQ ID NO: 124 or a sequence with at least 45%, 50%, 70%, 75%, 80%, 85% or 90% sequence identity thereto and a LC variable region sequence comprising SEQ ID NO: 126 or a sequence with at least 35%, 40%, 70%, 75%, 80%, 85% or 90% sequence identity thereto; or s) a HC variable region sequence comprising SEQ ID NO: 164 or a sequence with at least 45%, 70%, 75%, 80%, 85% or 90% sequence identity thereto and a LC variable region sequence comprising SEQ ID NO: 166 or a sequence with at least 35%, 40%, 70%, 75%, 80%, 85% or 90% sequence identity thereto; or t) a HC variable region sequence comprising SEQ ID NO: 194 or a sequence with at least 45%, 50%, 70%, 75%, 80%, 85% or 90% sequence identity thereto and a LC variable region sequence comprising SEQ ID NO: 196 or a sequence with at least 35%, 40%, 70%, 75%, 80%, 85% or 90% sequence identity thereto; or u) a HC variable region sequence comprising SEQ ID NO: 94 or a sequence with at least 45%, 50%, 70%, 75%, 80%, 85% or 90% sequence identity thereto and a LC variable region sequence comprising SEQ ID NO: 96 or a sequence with at least 35%, 40%, 70%, 75%, 80%, 85% or 90% sequence identity thereto; or v) a HC variable region sequence comprising SEQ ID NO: 274 or a sequence with at least 45%, 50%, 70%, 75%, 80%, 85% or 90% sequence identity thereto and a LC variable region sequence comprising SEQ ID NO: 276 or a sequence with at least 35%, 40%, 70%, 75%, 80%, 85% or 90% sequence identity thereto; or w) a HC variable region sequence comprising SEQ ID NO: 284 or a sequence with at least 45%, 50%, 70%, 75%, 80%, 85% or 90% sequence identity thereto and a LC variable region sequence comprising SEQ ID NO: 286 or a sequence with at least 35%, 40%, 70%, 75%, 80%, 85% or 90% sequence identity thereto; or x) a HC variable region sequence comprising SEQ ID NO: 104 or a sequence with at least 40%, 45%, 70%, 75%, 80%, 85% or 90% sequence identity thereto and a LC variable region sequence comprising SEQ ID NO: 106 or a sequence with at least 35%, 40%, 70%, 75%, 80%, 85% or 90% sequence identity thereto; or y) a HC variable region sequence comprising SEQ ID NO: 144 or a sequence with at least 45%, 50%, 70%, 75%, 80%, 85% or 90% sequence identity thereto and a LC variable region sequence comprising SEQ ID NO: 146 or a sequence with at least 35%, 40%, 70%, 75%, 80%, 85% or 90% sequence identity thereto; or z) a HC variable region sequence comprising SEQ ID NO: 4 or a sequence with at least 45%, 50%, 70%, 75%, 80%, 85% or 90% sequence identity thereto and a LC variable region sequence comprising SEQ ID NO: 6 or a sequence with at least 35%, 40%, 70%, 75%, 80%, 85% or 90% sequence identity thereto; or aa) a HC variable region sequence comprising SEQ ID NO: 24 or a sequence with at least 45%, 50%, 70%, 75%, 80%, 85% or 90% sequence identity thereto and a LC variable region sequence comprising SEQ ID NO: 26 or a sequence with at least 35%, 40%, 70%, 75%, 80%, 85% or 90% sequence identity thereto; or bb) a HC variable region sequence comprising SEQ ID NO: 64 or a sequence with at least 45%, 50%, 70%, 75%, 80%, 85% or 90% sequence identity thereto and a LC variable region sequence comprising SEQ ID NO: 66 or a sequence with at least 35%, 40%, 70%, 75%, 80%, 85% or 90% sequence identity thereto; or cc) a HC variable region sequence comprising SEQ ID NO: 74 or a sequence with at least 45%, 50%, 70%, 75%, 80%, 85% or 90% sequence identity thereto and a LC variable region sequence comprising SEQ ID NO: 76 or a sequence with at least 35%, 40%, 70%, 75%, 80%, 85% or 90% sequence identity thereto.

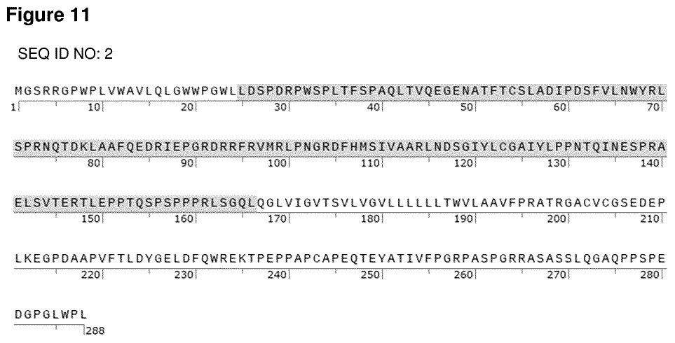

The antibody or antigen-binding portion thereof according to the invention may bind to one of the following epitopes of the extracellular domain of canine PD-1:
i) T76, Q83, E84, L128, P129, P130, or
ii) D58, D61, N74, T76, Y127
   wherein the epitope is determined using epitope mapping techniques. The epitope residues are provided with reference to canine PD-1 sequence. SEQ ID NO: 2 provides the sequence of canine PD-1 and FIG. 11 shows the residue numbering of canine PD-1.

The antibody or antigen-binding portion thereof according to the invention may bind to one of the following epitopes of the extracellular domain of canine PD-1:
i) T76, Q83, E84, L128, P129, P130 according to SEQ ID NO: 2
ii) D58, D61, N74, T76, Y127 according to SEQ ID NO: 2
   wherein the epitope is determined using epitope mapping techniques.

Epitope mapping techniques that may be used to determine whether an antibody binds to a specific epitope include site directed mutagenesis epitope mapping, shotgun mutagenesis epitope mapping, oligopeptide scanning (e.g., pepscan analysis), hydrogen-deuterium exchange optionally in conjunction with mass spectrometry analysis, cross linking mass spectrometry analysis, x-ray crystallographic analysis, electron microscopy analysis, NMR analysis, in silico methods e.g. using computer generated structural predictions or a combination of the aforementioned techniques. In an embodiment the epitope mapping technique is site directed mutagenesis epitope mapping. In one embodiment the epitope mapping technique is alanine scanning mutagenesis epitope mapping. In one embodiment the epitope mapping technique is site directed mutagenesis epitope mapping such as the technique described in Example 17 herein.

In one embodiment of the invention an isolated canine antibody or antigen binding fragment thereof which binds to canine PD-1 is provided wherein said antibody binds to one of the following epitopes of the extracellular domain of canine PD-1:
i) T76, Q83, E84, L128, P129, P130 according to SEQ ID NO: 2
ii) D58, D61, N74, T76, Y127 according to SEQ ID NO: 2
   wherein the epitope is determined using an epitope mapping technique,
   and wherein said antibody comprises an HC CDR1 sequence comprising or consisting of SEQ ID NO: 17 or an amino acid sequence with at least 70%, 80%, 85%, 90% or 95% sequence identity to SEQ ID NO: 17 and an HC CDR2 sequence comprising or consisting of SEQ ID NO: 18 or an amino acid sequence with at least 70%, 80%, 85%, 90% or 95% sequence identity to SEQ ID NO: 18 and an HC CDR3 sequence comprising or consisting of SEQ ID NO: 19 or an amino acid sequence with at least 70%, 80%, 85%, 90% or 95% sequence identity to SEQ ID NO: 19 and an LC CDR1 sequence comprising or consisting of SEQ ID NO: 20 or an amino acid sequence with at least 70%, 80%, 85%, 90% or 95% sequence identity to SEQ ID NO: 20 and an LC CDR2 sequence comprising or consisting of SEQ ID NO: 21 or an amino acid sequence with at least 70%, 80%, 85%, 90% or 95% sequence identity to SEQ ID NO: 21 and an LC CDR3 sequence comprising or consisting of SEQ ID NO: 22 or an amino acid sequence with at least 70%, 80%, 85%, 90% or 95% sequence identity to SEQ ID NO: 22; or an HC CDR1 sequence comprising or consisting of SEQ ID NO: 27 or an amino acid sequence with at least 70%, 80%, 85%, 90% or 95% sequence identity to SEQ ID NO: 27 and an HC CDR2 sequence comprising or consisting of SEQ ID NO: 28 or an amino acid sequence with at least 70%, 80%, 85%, 90% or 95% sequence identity to SEQ ID NO: 28 and an HC CDR3 sequence comprising or consisting of SEQ ID NO: 29 or an amino acid sequence with at least 70%, 80%, 85%, 90% or 95% sequence identity to SEQ ID NO: 29 and an LC CDR1 sequence comprising or consisting of SEQ ID NO: 30 or an amino acid sequence with at least 70%, 80%, 85%, 90% or 95% sequence identity to SEQ ID NO: 30 and an LC CDR2 sequence comprising or consisting of SEQ ID NO: 31 or an amino acid sequence with at least 70%, 80%, 85%, 90% or 95% sequence identity to SEQ ID NO: 31 and an LC CDR3 sequence comprising or consisting of SEQ ID NO: 32 or an amino acid sequence with at least 70%, 80%, 85%, 90% or 95% sequence identity to SEQ ID NO: 32; or an HC CDR1 sequence comprising or consisting of SEQ ID NO: 207 or an amino acid sequence with at least 70%, 80%, 85%, 90% or 95% sequence identity to SEQ ID NO: 207 and an HC CDR2 sequence comprising or consisting of SEQ ID NO: 208 or an amino acid sequence with at least 70%, 80%, 85%, 90% or 95% sequence identity to SEQ ID NO: 208 and an HC CDR3 sequence comprising or consisting of SEQ ID NO: 209 or an amino acid sequence w with at least 70%, 80%, 85%, 90% or 95% sequence identity to SEQ ID NO: 209 and an LC CDR1 sequence comprising or consisting of SEQ ID NO: 210 or an amino acid sequence with at least 70%, 80%, 85%, 90% or 95% sequence identity to SEQ ID NO: 210 and an LC CDR2 sequence comprising or consisting of SEQ ID NO: 211 or an amino acid sequence with at least 70%, 80%, 85%, 90% or 95% sequence identity to SEQ ID NO: 211 and an LC CDR3 sequence comprising or consisting of SEQ ID NO: 212 or an amino acid sequence with at least 70%, 80%, 85%, 90% or 95% sequence identity to SEQ ID NO: 212; or an HC CDR1 sequence comprising or consisting of SEQ ID NO: 97 or an amino acid sequence with at least 70%, 80%, 85%, 90% or 95% sequence identity to SEQ ID NO: 97 and an HC CDR2 sequence comprising or consisting of SEQ ID NO: 98 or an amino acid sequence with at least 70%, 80%, 85%, 90% or 95% sequence identity to SEQ ID NO: 98 and an HC CDR3 sequence comprising or consisting of SEQ ID NO: 99 or an amino acid sequence with at least 70%, 80%, 85%, 90% or 95% sequence identity to SEQ ID NO: 99 and an LC CDR1 sequence comprising or consisting of SEQ ID NO: 100 or an amino acid sequence with at least 70%, 80%, 85%, 90% or 95% sequence identity to SEQ ID NO: 100 and an LC CDR2 sequence comprising or consisting of SEQ ID NO: 101 or an amino acid sequence with at least 70%, 80%, 85%, 90% or 95% sequence identity to SEQ ID NO: 101 and an LC CDR3 sequence comprising or consisting of SEQ ID NO: 102 or an amino acid sequence with at least 70%, 80%, 85%, 90% or 95% sequence identity to SEQ ID NO: 102.

In one embodiment of the invention an isolated canine antibody or antigen binding fragment thereof which binds to canine PD-1 is provided wherein said antibody binds to one of the following epitopes of the extracellular domain of canine PD-1:
i) T76, Q83, E84, L128, P129, P130 according to SEQ ID NO: 2
ii) D58, D61, N74, T76, Y127 according to SEQ ID NO: 2
   wherein the epitope is determined using an epitope mapping technique,
   and wherein said antibody comprises an HC CDR1 sequence comprising or consisting of SEQ ID NO: 17 or an amino acid sequence which has 1, 2, 3, 4, or 5 amino acid differences compared to SEQ ID NO: 17 and an HC CDR2 sequence comprising or consisting of SEQ ID NO: 18 or an amino acid sequence which has 1, 2, 3, 4, 5, or 6 amino acid differences compared to SEQ ID NO: 18 and an HC CDR3 sequence comprising or consisting of SEQ ID NO: 19 or an amino acid sequence which has 1, 2, 3, 4, 5, 6, 7, 8, or 9 amino acid differences compared to SEQ ID NO: 19 and an LC CDR1 sequence comprising or consisting of SEQ ID NO: 20 or an amino acid sequence which has 1, 2, 3, 4, 5, 6, 7, 8, 9, 10, 11, 12, 13 or 14 amino acid differences compared to SEQ ID NO: 20 and an LC CDR2 sequence comprising or consisting of SEQ ID NO: 21 or an amino acid sequence which has 1, 2, 3, 4, or 5 amino acid differences compared to SEQ ID NO: 21 and an LC CDR3 sequence comprising or consisting of SEQ ID NO: 22 or an amino acid sequence which has 1, 2, 3, 4, 5, 6, 7, 8, 9, or 10 amino acid differences compared to SEQ ID NO: 22; or an HC CDR1 sequence comprising or consisting of SEQ ID NO: 207 or an amino acid sequence which has 1, or 2 amino acid differences compared to SEQ ID NO: 207 and an HC CDR2 sequence comprising or consisting of SEQ ID NO: 208 or an amino acid sequence which has 1, 2, 3, 4, or 5 amino acid differences compared to SEQ ID NO: 208 and an HC CDR3 sequence comprising or consisting of SEQ ID NO: 209 or an amino acid sequence which has 1, 2, 3, 4, 5, 6, 7, 8, 9, 10, 11, 12, 13, 14, 15, or 16 amino acid differences compared to SEQ ID NO: 209 and an LC CDR1 sequence comprising or consisting of SEQ ID NO: 210 or an amino acid sequence which has 1, 2 or 3 amino acid differences compared to SEQ ID NO: 210 and an LC CDR2 sequence comprising or consisting of SEQ ID NO: 211 or an amino acid sequence which has 1 or 2 amino acid differences compared to SEQ ID NO: 211 and an LC CDR3 sequence comprising or consisting of SEQ ID NO: 212 or an amino acid sequence which has 1, 2, 3, or 4 amino acid differences compared to SEQ ID NO: 212; or an HC CDR1 sequence comprising or consisting of SEQ ID NO: 97 or an amino acid sequence which has 1, 2, or 3 amino acid differences compared to SEQ ID NO: 97 and an HC CDR2 sequence comprising or consisting of SEQ ID NO: 98 or an amino acid sequence which has 1, 2, 3, or 4 amino acid differences compared to SEQ ID NO: 98 and an HC CDR3 sequence comprising or consisting of SEQ ID NO: 99 or an amino acid sequence which has 1, 2, 3, 4, 5, 6, 7, 8, 9, 10, 11, 12, 13, or 14 amino acid differences compared to SEQ ID NO: 99 and an LC CDR1 sequence comprising or consisting of SEQ ID NO: 100 or an amino acid sequence which has 1, 2, 3, 4, 5, 6, 7, 8, 9, 10, 11, 12, 13, 14 or 15 amino acid differences compared to SEQ ID NO: 100 and an LC CDR2 sequence comprising or consisting of SEQ ID NO: 101 or an amino acid sequence which has 1, 2, 3, 4, or 5 amino acid differences compared to SEQ ID NO: 101 and an LC CDR3 sequence comprising or consisting of SEQ ID NO: 102 or an amino acid sequence which has 1, 2, 3, 4, 5, 6, 7, 8, 9, or 10 amino acid differences compared to SEQ ID NO: 102.

In another related embodiment of the invention an isolated canine antibody or antigen binding fragment thereof which binds to canine PD-1 is provided, wherein said antibody binds to one of the following epitopes of the extracellular domain of canine PD-1:
i) T76, Q83, E84, L128, P129, P130 according to SEQ ID NO: 2
ii) D58, D61, N74, T76, Y127 according to SEQ ID NO: 2
   wherein the epitope is determined using an epitope mapping technique,
   and wherein said antibody or antigen-binding fragment has i) a HC CDR1 sequence comprising or consisting of SEQ ID NO: 17, a HC CDR2 sequence comprising or consisting of SEQ ID NO: 18, a HC CDR3 sequence comprising or consisting of SEQ ID NO: 19, a LC CDR1 sequence comprising or consisting of SEQ ID NO: 20, a LC CDR2 sequence comprising or consisting of SEQ ID NO: 21 and a LC CDR3 sequence comprising or consisting of SEQ ID NO: 22, ii) a HC CDR1 sequence comprising or consisting of SEQ ID NO: 47, a HC CDR2 sequence comprising or consisting of SEQ ID NO: 48, a HC CDR3 sequence comprising or consisting of SEQ ID NO: 49, a LC CDR1 sequence comprising SEQ ID NO: 50, a LC CDR2 sequence comprising or consisting of SEQ ID NO: 51 and a LC CDR3 sequence comprising or consisting of SEQ ID NO: 52, iii) a HC CDR1 sequence comprising SEQ ID NO: 57, a HC CDR2 sequence comprising SEQ ID NO: 58, a HC CDR3 sequence comprising SEQ ID NO: 59, a LC CDR1 sequence comprising SEQ ID NO: 60, a LC CDR2 sequence comprising SEQ ID NO: 61 and a LC CDR3 sequence comprising SEQ ID NO: 62, iv) a HC CDR1 sequence comprising or consisting of SEQ ID NO: 37, a HC CDR2 sequence comprising or consisting of SEQ ID NO: 38, a HC CDR3 sequence comprising or consisting of SEQ ID NO: 39, a LC CDR1 sequence comprising or consisting of SEQ ID NO: 40, a LC CDR2 sequence comprising or consisting of SEQ ID NO: 41 and a LC CDR3 sequence comprising or consisting of SEQ ID NO: 42, v) a HC CDR1 sequence comprising or consisting of SEQ ID NO: 87, a HC CDR2 sequence comprising or consisting of SEQ ID NO: 88, a HC CDR3 sequence comprising or consisting of SEQ ID NO: 89, a LC CDR1 sequence comprising or consisting of SEQ ID NO: 90, a LC CDR2 sequence comprising or consisting of SEQ ID NO: 91 and a LC CDR3 sequence comprising or consisting of SEQ ID NO: 92, vi) a HC CDR1 sequence comprising or consisting of SEQ ID NO: 137, a HC CDR2 sequence comprising or consisting of SEQ ID NO: 138, a HC CDR3 sequence comprising or consisting of SEQ ID NO: 139, a LC CDR1 sequence comprising or consisting of SEQ ID NO: 141, a LC CDR2 sequence comprising or consisting of SEQ ID NO: 142 and a LC CDR3 sequence comprising or consisting of SEQ ID NO: 143, vii) a HC CDR1 sequence comprising or consisting of SEQ ID NO: 157, a HC CDR2 sequence comprising or consisting of SEQ ID NO: 158, a HC CDR3 sequence comprising or consisting of SEQ ID NO: 159, a LC CDR1 sequence comprising or consisting of SEQ ID NO: 160, a LC CDR2 sequence comprising or consisting of SEQ ID NO: 161 and a LC CDR3 sequence comprising or consisting of SEQ ID NO: 162, viii) a HC CDR1 sequence comprising or consisting of SEQ ID NO: 177, a HC CDR2 sequence comprising or consisting of SEQ ID NO: 178, a HC CDR3 sequence comprising or consisting of SEQ ID NO: 179, a LC CDR1 sequence comprising or consisting of SEQ ID NO: 180, a LC CDR2 sequence comprising or consisting of SEQ ID NO: 181 and a LC CDR3 sequence comprising or consisting of SEQ ID NO: 182, ix) a HC CDR1 sequence comprising or consisting of SEQ ID NO: 187, a HC CDR2 sequence comprising or consisting of SEQ ID NO: 188, a HC CDR3 sequence comprising or consisting of SEQ ID NO: 189, a LC CDR1 sequence comprising or consisting of SEQ ID NO: 190, a LC CDR2 sequence comprising or consisting of SEQ ID NO: 191 and a LC CDR3 sequence comprising or consisting of SEQ ID NO: 192, x) a HC CDR1 sequence comprising or consisting of SEQ ID NO: 207, a HC CDR2 sequence comprising or consisting of SEQ ID NO: 208, a HC CDR3 sequence comprising or consisting of SEQ ID NO: 209, a LC CDR1 sequence comprising or consisting of SEQ ID NO: 210, a LC CDR2 sequence comprising or consisting of SEQ ID NO: 211 and a LC CDR3 sequence comprising or consisting of SEQ ID NO: 212, xi) a HC CDR1 sequence comprising or consisting of SEQ ID NO: 217, a HC CDR2 sequence comprising or consisting of SEQ ID NO: 218, a HC CDR3 sequence comprising or consisting of SEQ ID NO: 219, a LC CDR1 sequence comprising or consisting of SEQ ID NO: 220, a LC CDR2 sequence comprising or consisting of SEQ ID NO: 221 and a LC CDR3 sequence comprising or consisting of SEQ ID NO: 222, xii) a HC CDR1 sequence comprising or consisting of SEQ ID NO: 227, a HC CDR2 sequence comprising or consisting of SEQ ID NO: 228, a HC CDR3 sequence comprising or consisting of SEQ ID NO: 229, a LC CDR1 sequence comprising or consisting of SEQ ID NO: 230, a LC CDR2 sequence comprising or consisting of SEQ ID NO: 231 and a LC CDR3 sequence comprising or consisting of SEQ ID NO: 232, xiii) a HC CDR1 sequence comprising or consisting of SEQ ID NO: 237, a HC CDR2 sequence comprising or consisting of SEQ ID NO: 238, a HC CDR3 sequence comprising or consisting of SEQ ID NO: 239, a LC CDR1 sequence comprising or consisting of SEQ ID NO: 240, a LC CDR2 sequence comprising or consisting of SEQ ID NO: 241 and a LC CDR3 sequence comprising or consisting of SEQ ID NO: 242, xiv) a HC CDR1 sequence comprising or consisting of SEQ ID NO: 247, a HC CDR2 sequence comprising or consisting of SEQ ID NO: 248, a HC CDR3 sequence comprising or consisting of SEQ ID NO: 249, a LC CDR1 sequence comprising or consisting of SEQ ID NO: 250, a LC CDR2 sequence comprising or consisting of SEQ ID NO: 251 and a LC CDR3 sequence comprising or consisting of SEQ ID NO: 252, xv) a HC CDR1 sequence comprising or consisting of SEQ ID NO: 257, a HC CDR2 sequence comprising or consisting of SEQ ID NO: 258, a HC CDR3 sequence comprising or consisting of SEQ ID NO: 259, a LC CDR1 sequence comprising or consisting of SEQ ID NO: 260, a LC CDR2 sequence comprising or consisting of SEQ ID NO: 261 and a LC CDR3 sequence comprising or consisting of SEQ ID NO: 262, xvi) a HC CDR1 sequence comprising or consisting of SEQ ID NO: 267, a HC CDR2 sequence comprising or consisting of SEQ ID NO: 268, a HC CDR3 sequence comprising or consisting of SEQ ID NO: 269, a LC CDR1 sequence comprising or consisting of SEQ ID NO: 270, a LC CDR2 sequence comprising or consisting of SEQ ID NO: 271 and a LC CDR3 sequence comprising or consisting of SEQ ID NO: 271, xvii) a HC CDR1 sequence comprising or consisting of SEQ ID NO: 117, a HC CDR2 sequence comprising or consisting of SEQ ID NO: 118, a HC CDR3 sequence comprising or consisting of SEQ ID NO: 119, a LC CDR1 sequence comprising or consisting of SEQ ID NO: 120, a LC CDR2 sequence comprising or consisting of SEQ ID NO: 121 and a LC CDR3 sequence comprising or consisting of SEQ ID NO: 122, xviii) a HC CDR1 sequence comprising or consisting of SEQ ID NO: 127, a HC CDR2 sequence comprising or consisting of SEQ ID NO: 128, a HC CDR3 sequence comprising or consisting of SEQ ID NO: 129, a LC CDR1 sequence comprising or consisting of SEQ ID NO: 130, a LC CDR2 sequence comprising or consisting of SEQ ID NO: 131 and a LC CDR3 sequence comprising or consisting of SEQ ID NO: 132, xxix) a HC CDR1 sequence comprising or consisting of SEQ ID NO: 167, a HC CDR2 sequence comprising or consisting of SEQ ID NO: 168, a HC CDR3 sequence comprising or consisting of SEQ ID NO: 169, a LC CDR1 sequence comprising or consisting of SEQ ID NO: 170, a LC CDR2 sequence comprising or consisting of SEQ ID NO: 171 and a LC CDR3 sequence comprising or consisting of SEQ ID NO: 172, xx) a HC CDR1 sequence comprising or consisting of SEQ ID NO: 197, a HC CDR2 sequence comprising or consisting of SEQ ID NO: 198, a HC CDR3 sequence comprising or consisting of SEQ ID NO: 199, a LC CDR1 sequence comprising or consisting of SEQ ID NO: 200, a LC CDR2 sequence comprising or consisting of SEQ ID NO: 201 and a LC CDR3 sequence comprising or consisting of SEQ ID NO: 202, xxi) a HC CDR1 sequence comprising or consisting of SEQ ID NO: 97, a HC CDR2 sequence comprising or consisting of SEQ ID NO: 98, a HC CDR3 sequence comprising or consisting of SEQ ID NO: 99, a LC CDR1 sequence comprising or consisting of SEQ ID NO: 100, a LC CDR2 sequence comprising or consisting of SEQ ID NO: 101 and a LC CDR3 sequence comprising or consisting of SEQ ID NO: 102, xxii) a HC CDR1 sequence comprising or consisting of SEQ ID NO: 277, a HC CDR2 sequence comprising or consisting of SEQ ID NO: 278, a HC CDR3 sequence comprising or consisting of SEQ ID NO: 279, a LC CDR1 sequence comprising or consisting of SEQ ID NO: 280, a LC CDR2 sequence comprising or consisting of SEQ ID NO: 281 and a LC CDR3 sequence comprising or consisting of SEQ ID NO: 282, xxiii) a HC CDR1 sequence comprising or consisting of SEQ ID NO: 287, a HC CDR2 sequence comprising or consisting of SEQ ID NO: 288, a HC CDR3 sequence comprising or consisting of SEQ ID NO: 289, a LC CDR1 sequence comprising or consisting of SEQ ID NO: 290, a LC CDR2 sequence comprising or consisting of SEQ ID NO: 291 and a LC CDR3 sequence comprising or consisting of SEQ ID NO: 292, xxiv) a HC CDR1 sequence comprising or consisting of SEQ ID NO: 107, a HC CDR2 sequence comprising or consisting of SEQ ID NO: 108, a HC CDR3 sequence comprising or consisting of SEQ ID NO: 109, a LC CDR1 sequence comprising or consisting of SEQ ID NO: 110, a LC CDR2 sequence comprising or consisting of SEQ ID NO: 111 and a LC CDR3 sequence comprising or consisting of SEQ ID NO: 112, xxv) a HC CDR1 sequence comprising or consisting of SEQ ID NO: 147, a HC CDR2 sequence comprising or consisting of SEQ ID NO: 148, a HC CDR3 sequence comprising or consisting of SEQ ID NO: 149, a LC CDR1 sequence comprising or consisting of SEQ ID NO: 150, a LC CDR2 sequence comprising or consisting of SEQ ID NO: 151 and a LC CDR3 sequence comprising or consisting of SEQ ID NO: 152, xxvi) a HC CDR1 sequence comprising or consisting of SEQ ID NO: 7, a HC CDR2 sequence comprising or consisting of SEQ ID NO: 8, a HC CDR3 sequence comprising or consisting of SEQ ID NO: 9, a LC CDR1 sequence comprising or consisting of SEQ ID NO: 10, a LC CDR2 sequence comprising or consisting of SEQ ID NO: 11 and a LC CDR3 sequence comprising or consisting of SEQ ID NO: 12, xxvii) a HC CDR1 sequence comprising or consisting of SEQ ID NO: 27, a HC CDR2 sequence comprising or consisting of SEQ ID NO: 28, a HC CDR3 sequence comprising or consisting of SEQ ID NO: 29, a LC CDR1 sequence comprising or consisting of SEQ ID NO: 30, a LC CDR2 sequence comprising or consisting of SEQ ID NO: 31 and a LC CDR3 sequence comprising or consisting of SEQ ID NO: 32, xxviii) a HC CDR1 sequence comprising or consisting of SEQ ID NO: 67, a HC CDR2 sequence comprising or consisting of SEQ ID NO: 68, a HC CDR3 sequence comprising or consisting of SEQ ID NO: 69, a LC CDR1 sequence comprising or consisting of SEQ ID NO: 70, a LC CDR2 sequence comprising or consisting of SEQ ID NO: 71 and a LC CDR3 sequence comprising or consisting of SEQ ID NO: 72, xxix) a HC CDR1 sequence comprising or consisting of SEQ ID NO: 77, a HC CDR2 sequence comprising or consisting of SEQ ID NO: 78, a HC CDR3 sequence comprising or consisting of SEQ ID NO: 79, a LC CDR1 sequence comprising or consisting of SEQ ID NO: 80, a LC CDR2 sequence comprising or consisting of SEQ ID NO: 81 and a LC CDR3 sequence comprising or consisting of SEQ ID NO: 82.

In an embodiment of the invention the antibody or antigen-binding portion thereof which binds to one of the following epitopes of the extracellular domain of canine PD-1:
i) T76, Q83, E84, L128, P129, P130 according to SEQ ID NO: 2
ii) D58, D61, N74, T76, Y127 according to SEQ ID NO: 2
   wherein the epitope is determined using an epitope mapping technique, comprises a HC variable region sequence comprising SEQ ID NO: 4 or a sequence with at least 45%, 50%, 60%, 70%, 75%, 80%, 85% or 90% sequence identity thereto and a LC variable region sequence comprising SEQ ID NO: 6 or a sequence with at least 35%, 40%, 50%, 60%, 70%, 75%, 80%, 85%, 90% or 95% sequence identity thereto for example a LC variable region sequence comprising SEQ ID NO: 6.

In another related embodiment of the invention an antibody or antigen-binding portion thereof which binds to one of the following epitopes of the extracellular domain of canine PD-1:
i) an epitope comprising T76, Q83, E84, L128, P129, P130 according to SEQ ID NO: 2
ii) an epitope comprising D58, D61, N74, T76, Y127 according to SEQ ID NO: 2
   wherein the epitope is determined using an epitope mapping technique, is provided which has: a) a HC variable region sequence comprising SEQ ID NO: 14 or a sequence with at least 40%, 45%, 70%, 75%, 80%, 85% or 90% sequence identity thereto and a LC variable region sequence comprising SEQ ID NO: 16 or a sequence with at least 35%, 45%, 55%, 65%, 70%, 75%, 80%, 85% or 90% sequence identity thereto; or b) a HC variable region sequence comprising SEQ ID NO: 44 or a sequence with at least 40%, 50%, 60%, 70%, 75%, 80%, 85% or 90% sequence identity thereto and a LC variable region sequence comprising SEQ ID NO: 46 or a sequence with at least 35%, 45%, 55%, 65%, 70%, 75%, 80%, 85% or 90% sequence identity thereto; or c) a HC variable region sequence comprising SEQ ID NO: 54 or a sequence with at least 40%, 45%, 70%, 75%, 80%, 85% or 90% sequence identity thereto and a LC variable region sequence comprising SEQ ID NO: 56 or a sequence with at least 35%, 40%, 70%, 75%, 80%, 85% or 90% sequence identity thereto; or d) a HC variable region sequence comprising SEQ ID NO: 34 or a sequence with at least 45%, 65%, 70%, 75%, 80%, 85% or 90% sequence identity thereto and a LC variable region sequence comprising SEQ ID NO: 36 or a sequence with at least 35%, 40%, 70%, 75%, 80%, 85% or 90% sequence identity thereto; or e) a HC variable region sequence comprising SEQ ID NO: 84 or a sequence with at least 40%, 45%, 50%, 70%, 75%, 80%, 85% or 90% sequence identity thereto and a LC variable region sequence comprising SEQ ID NO: 86 or a sequence with at least 35%, 40%, 70%, 75%, 80%, 85% or 90% sequence identity thereto; or f) a HC variable region sequence comprising SEQ ID NO: 134 or a sequence with at least 45%, 50%, 70%, 75%, 80%, 85% or 90% sequence identity thereto and a LC variable region sequence comprising SEQ ID NO: 136 or a sequence with at least 35%, 40%, 70%, 75%, 80%, 85% or 90% sequence identity thereto; or g) a HC variable region sequence comprising SEQ ID NO: 154 or a sequence with at least 45%, 50%, 70%, 75%, 80%, 85% or 90% sequence identity thereto and a LC variable region sequence comprising SEQ ID NO: 156 or a sequence with at least 35%, 40%, 70%, 75%, 80%, 85% or 90% sequence identity thereto; or h) a HC variable region sequence comprising SEQ ID NO: 174 or a sequence with at least 45%, 50%, 70%, 75%, 80%, 85% or 90% sequence identity thereto and a LC variable region sequence comprising SEQ ID NO: 176 or a sequence with at least 35%, 40%, 70%, 75%, 80%, 85% or 90% sequence identity thereto; or i) a HC variable region sequence comprising SEQ ID NO: 184 or a sequence with at least 45%, 50%, 70%, 75%, 80%, 85% or 90% sequence identity thereto and a LC variable region sequence comprising SEQ ID NO: 186 or a sequence with at least 35%, 40%, 70%, 75%, 80%, 85% or 90% sequence identity thereto; or j) a HC variable region sequence comprising SEQ ID NO: 204 or a sequence with at least 40%, 45%, 70%, 75%, 80%, 85% or 90% sequence identity thereto and a LC variable region sequence comprising SEQ ID NO: 206 or a sequence with at least 35%, 40%, 70%, 75%, 80%, 85% or 90% sequence identity thereto; or k) a HC variable region sequence comprising SEQ ID NO: 214 or a sequence with at least 40%, 45%, 70%, 75%, 80%, 85% or 90% sequence identity thereto and a LC variable region sequence comprising SEQ ID NO: 216 or a sequence with at least 35%, 40%, 70%, 75%, 80%, 85% or 90% sequence identity thereto; or 1) a HC variable region sequence comprising SEQ ID NO: 224 or a sequence with at least 40%, 45%, 70%, 75%, 80%, 85% or 90% sequence identity thereto and a LC variable region sequence comprising SEQ ID NO: 226 or a sequence with at least 35%, 40%, 70%, 75%, 80%, 85% or 90% sequence identity thereto; or m) a HC variable region sequence comprising SEQ ID NO: 234 or a sequence with at least 45%, 50%, 70%, 75%, 80%, 85% or 90% sequence identity thereto and a LC variable region sequence comprising SEQ ID NO: 236 or a sequence with at least 35%, 40%, 70%, 75%, 80%, 85% or 90% sequence identity thereto; or n) a HC variable region sequence comprising SEQ ID NO: 244 or a sequence with at least 40%, 45%, 70%, 75%, 80%, 85% or 90% sequence identity thereto and a LC variable region sequence comprising SEQ ID NO: 246 or a sequence with at least 35%, 40%, 70%, 75%, 80%, 85% or 90% sequence identity thereto; or o) a HC variable region sequence comprising SEQ ID NO: 254 or a sequence with at least 40%, 45%, 70%, 75%, 80%, 85% or 90% sequence identity thereto and a LC variable region sequence comprising SEQ ID NO: 256 or a sequence with at least 35%, 40%, 70%, 75%, 80%, 85% or 90% sequence identity thereto; or p) a HC variable region sequence comprising SEQ ID NO: 264 or a sequence with at least 40%, 45%, 70%, 75%, 80%, 85% or 90% sequence identity thereto and a LC variable region sequence comprising SEQ ID NO: 266 or a sequence with at least 35%, 40%, 70%, 75%, 80%, 85% or 90% sequence identity thereto; or q) a HC variable region sequence comprising SEQ ID NO: 114 or a sequence with at least 40%, 45%, 70%, 75%, 80%, 85% or 90% sequence identity thereto and a LC variable region sequence comprising SEQ ID NO: 116 or a sequence with at least 35%, 40%, 70%, 75%, 80%, 85% or 90% sequence identity thereto; or r) a HC variable region sequence comprising SEQ ID NO: 124 or a sequence with at least 45%, 50%, 70%, 75%, 80%, 85% or 90% sequence identity thereto and a LC variable region sequence comprising SEQ ID NO: 126 or a sequence with at least 35%, 40%, 70%, 75%, 80%, 85% or 90% sequence identity thereto; or s) a HC variable region sequence comprising SEQ ID NO: 164 or a sequence with at least 45%, 70%, 75%, 80%, 85% or 90% sequence identity thereto and a LC variable region sequence comprising SEQ ID NO: 166 or a sequence with at least 35%, 40%, 70%, 75%, 80%, 85% or 90% sequence identity thereto; or t) a HC variable region sequence comprising SEQ ID NO: 194 or a sequence with at least 45%, 50%, 70%, 75%, 80%, 85% or 90% sequence identity thereto and a LC variable region sequence comprising SEQ ID NO: 196 or a sequence with at least 35%, 40%, 70%, 75%, 80%, 85% or 90% sequence identity thereto; or u) a HC variable region sequence comprising SEQ ID NO: 94 or a sequence with at least 45%, 50%, 70%, 75%, 80%, 85% or 90% sequence identity thereto and a LC variable region sequence comprising SEQ ID NO: 96 or a sequence with at least 35%, 40%, 70%, 75%, 80%, 85% or 90% sequence identity thereto; or v) a HC variable region sequence comprising SEQ ID NO: 274 or a sequence with at least 45%, 50%, 70%, 75%, 80%, 85% or 90% sequence identity thereto and a LC variable region sequence comprising SEQ ID NO: 276 or a sequence with at least 35%, 40%, 70%, 75%, 80%, 85% or 90% sequence identity thereto; or w) a HC variable region sequence comprising SEQ ID NO: 284 or a sequence with at least 45%, 50%, 70%, 75%, 80%, 85% or 90% sequence identity thereto and a LC variable region sequence comprising SEQ ID NO: 286 or a sequence with at least 35%, 40%, 70%, 75%, 80%, 85% or 90% sequence identity thereto; or x) a HC variable region sequence comprising SEQ ID NO: 104 or a sequence with at least 40%, 45%, 70%, 75%, 80%, 85% or 90% sequence identity thereto and a LC variable region sequence comprising SEQ ID NO: 106 or a sequence with at least 35%, 40%, 70%, 75%, 80%, 85% or 90% sequence identity thereto; or y) a HC variable region sequence comprising SEQ ID NO: 144 or a sequence with at least 45%, 50%, 70%, 75%, 80%, 85% or 90% sequence identity thereto and a LC variable region sequence comprising SEQ ID NO: 146 or a sequence with at least 35%, 40%, 70%, 75%, 80%, 85% or 90% sequence identity thereto; or z) a HC variable region sequence comprising SEQ ID NO: 4 or a sequence with at least 45%, 50%, 70%, 75%, 80%, 85% or 90% sequence identity thereto and a LC variable region sequence comprising SEQ ID NO: 6 or a sequence with at least 35%, 40%, 70%, 75%, 80%, 85% or 90% sequence identity thereto; or aa) a HC variable region sequence comprising SEQ ID NO: 24 or a sequence with at least 45%, 50%, 70%, 75%, 80%, 85% or 90% sequence identity thereto and a LC variable region sequence comprising SEQ ID NO: 26 or a sequence with at least 35%, 40%, 70%, 75%, 80%, 85% or 90% sequence identity thereto; or bb) a HC variable region sequence comprising SEQ ID NO: 64 or a sequence with at least 45%, 50%, 70%, 75%, 80%, 85% or 90% sequence identity thereto and a LC variable region sequence comprising SEQ ID NO: 66 or a sequence with at least 35%, 40%, 70%, 75%, 80%, 85% or 90% sequence identity thereto; or cc) a HC variable region sequence comprising SEQ ID NO: 74 or a sequence with at least 45%, 50%, 70%, 75%, 80%, 85% or 90% sequence identity thereto and a LC variable region sequence comprising SEQ ID NO: 76 or a sequence with at least 35%, 40%, 70%, 75%, 80%, 85% or 90% sequence identity thereto.

In one embodiment the antibody or antigen-binding portion thereof binds to an epitope of the extracellular domain of canine PD-1 extracellular domain comprising D58, D61, N74, T76, Y127 according to SEQ ID NO: 2, wherein the antibody comprises an HC CDR1 sequence comprising or consisting of SEQ ID NO: 17 or an amino acid sequence with at least 70%, 80%, 85%, 90% or 95% sequence identity to SEQ ID NO: 17 and an HC CDR2 sequence comprising or consisting of SEQ ID NO: 18 or an amino acid sequence with at least 70%, 80%, 85%, 90% or 95% sequence identity to SEQ ID NO: 18 and an HC CDR3 sequence comprising or consisting of SEQ ID NO: 19 or an amino acid sequence with at least 70%, 80%, 85%, 90% or 95% sequence identity to SEQ ID NO: 19 and an LC CDR1 sequence comprising or consisting of SEQ ID NO: 20 or an amino acid sequence with at least 70%, 80%, 85%, 90% or 95% sequence identity to SEQ ID NO: 20 and an LC CDR2 sequence comprising or consisting of SEQ ID NO: 21 or an amino acid sequence with at least 70%, 80%, 85%, 90% or 95% sequence identity to SEQ ID NO: 21 and an LC CDR3 sequence comprising or consisting of SEQ ID NO: 22 or an amino acid sequence with at least 70%, 80%, 85%, 90% or 95% sequence identity to SEQ ID NO: 22. Optionally said antibody or antigen binding portion may comprise a HC variable region sequence comprising SEQ ID NO: 14 or a sequence with at least 45%, 50%, 70%, 75%, 80%, 85% or 90% sequence identity thereto and a LC variable region sequence comprising SEQ ID NO: 16 or a sequence with at least 35%, 40%, 70%, 75%, 80%, 85% or 90% sequence identity thereto.

In one embodiment the antibody or antigen-binding portion thereof binds to an epitope of the extracellular domain of canine PD-1 extracellular domain comprising D58, D61, N74, T76, Y127 according to SEQ ID NO: 2, wherein the antibody comprises an HC CDR1 sequence comprising or consisting of SEQ ID NO: 17 or an amino acid sequence which has 1, 2, 3, 4, or 5 amino acid differences compared to SEQ ID NO: 17 and an HC CDR2 sequence comprising or consisting of SEQ ID NO: 18 or an amino acid sequence which has 1, 2, 3, 4, 5, or 6 amino acid differences compared to SEQ ID NO: 18 and an HC CDR3 sequence comprising or consisting of SEQ ID NO: 19 or an amino acid sequence which has 1, 2, 3, 4, 5, 6, 7, 8, or 9 amino acid differences compared to SEQ ID NO: 19 and an LC CDR1 sequence comprising or consisting of SEQ ID NO: 20 or an amino acid sequence which has 1, 2, 3, 4, 5, 6, 7, 8, 9, 10, 11, 12, 13 or 14 amino acid differences compared to SEQ ID NO: 20 and an LC CDR2 sequence comprising or consisting of SEQ ID NO: 21 or an amino acid sequence which has 1, 2, 3, 4, or 5 amino acid differences compared to SEQ ID NO: 21 and an LC CDR3 sequence comprising or consisting of SEQ ID NO: 22 or an amino acid sequence which has 1, 2, 3, 4, 5, 6, 7, 8, 9, or 10 amino acid differences compared to SEQ ID NO: 22;

In one embodiment the antibody or antigen-binding portion thereof binds to an epitope of the extracellular domain of canine PD-1 extracellular domain comprising T76, Q83, E84, L128, P129, P130 according to SEQ ID NO: 2 and said antibody or antigen binding portion comprises an HC CDR1 sequence comprising or consisting of SEQ ID NO: 27 or an amino acid sequence with at least 70%, 80%, 85%, 90% or 95% sequence identity to SEQ ID NO: 27 and an HC CDR2 sequence comprising or consisting of SEQ ID NO: 28 or an amino acid sequence with at least 70%, 80%, 85%, 90% or 95% sequence identity to SEQ ID NO: 28 and an HC CDR3 sequence comprising or consisting of SEQ ID NO: 29 or an amino acid sequence with at least 70%, 80%, 85%, 90% or 95% sequence identity to SEQ ID NO: 29 and an LC CDR1 sequence comprising or consisting of SEQ ID NO: 30 or an amino acid sequence with at least 70%, 80%, 85%, 90% or 95% sequence identity to SEQ ID NO: 30 and an LC CDR2 sequence comprising or consisting of SEQ ID NO: 31 or an amino acid sequence with at least 70%, 80%, 85%, 90% or 95% sequence identity to SEQ ID NO: 31 and an LC CDR3 sequence comprising or consisting of SEQ ID NO: 32 or an amino acid sequence with at least 70%, 80%, 85%, 90% or 95% sequence identity to SEQ ID NO: 32. Optionally said antibody or antigen binding portion may comprise a HC variable region sequence comprising SEQ ID NO: 24 or a sequence with at least 45%, 50%, 70%, 75%, 80%, 85% or 90% sequence identity thereto and a LC variable region sequence comprising SEQ ID NO: 26 or a sequence with at least 35%, 40%, 70%, 75%, 80%, 85% or 90% sequence identity thereto.

In one embodiment the antibody or antigen-binding portion thereof binds to an epitope of the extracellular domain of canine PD-1 extracellular domain comprising T76, Q83, E84, L128, P129, P130 according to SEQ ID NO: 2 and said antibody or antigen binding portion comprises an HC CDR1 sequence comprising or consisting of SEQ ID NO: 27 or an amino acid sequence which has 1, 2, 3, 4, or 5 amino acid differences compared to SEQ ID NO: 27 and an HC CDR2 sequence comprising or consisting of SEQ ID NO: 28 or an amino acid sequence which has 1, 2, 3, 4, 5, or 6 amino acid differences compared to SEQ ID NO: 28 and an HC CDR3 sequence comprising or consisting of SEQ ID NO: 29 or an amino acid sequence which has 1, 2, 3, 4, 5, 6, 7, 8, or 9 amino acid differences compared to SEQ ID NO: 29 and an LC CDR1 sequence comprising or consisting of SEQ ID NO: 30 or an amino acid sequence which has 1, 2, 3, 4, 5, 6, 7, 8, 9, 10, 11, 12, 13 or 14 amino acid differences compared to SEQ ID NO: 30 and an LC CDR2 sequence comprising or consisting of SEQ ID NO: 31 or an amino acid sequence which has 1, 2, 3, 4, or 5 amino acid differences compared to SEQ ID NO: 31 and an LC CDR3 sequence comprising or consisting of SEQ ID NO: 32 or an amino acid sequence with at least 70%, 80%, 85%, 90% or 95% sequence identity to SEQ ID NO: 32. amino acid sequence which has 1, 2, 3, 4, 5, 6, 7, 8, 9, or 10 amino acid differences compared to SEQ ID NO: 32.

The term PD-1 refers to the protein also known as CD279 (cluster of differentiation 279) is a protein on the surface of T and B cells. The antibodies and antigen binding portions thereof bind specifically to wild type canine PD-1 as defined in SEQ ID NO: 1 (nucleotide sequence) and SEQ ID NO: 2 (amino acid sequence). Unless otherwise stated, the term PD-1 as used herein refers to canine PD-1 or T and B cell antigen PD-1. In humans and canines, PD-1 is encoded by the PDCD1 gene.

Unless otherwise specified, the term PD-1 as used herein refers to canine PD-1. The terms “Programmed Death 1,” “Programmed Cell Death 1,” “Protein PD-1,” “PD-1,” PD1,” “PDCD1,” “hPD-1” and “hPD-1” are used interchangeably, and include variants, isoforms, species homologs of canine PD-1.

The terms “PD-1 binding molecule/protein/polypeptide/agent/moiety”, “PD-1 antigen binding molecule molecule/protein/polypeptide/agent/moiety”, “anti-PD-1 antibody”, “anti-PD-1 antibody or antigen binding portion thereof” all refer to a molecule capable of specifically binding to the canine PD-1 antigen. The binding reaction may be shown by standard methods, for example with reference to a negative control test using an antibody of unrelated specificity. The term “PD-1 binding molecule/agent” includes a PD-1 binding protein.

An antibody or antigen binding portion thereof of the invention, including a multispecific, e.g. bispecific or trispecific, binding agent described herein, “which binds” or is “capable of binding” an antigen of interest, that is canine PD-1, is one that binds the antigen with sufficient affinity such that the antibody or antigen binding portion thereof is useful as a therapeutic agent in targeting a cell or tissue expressing the antigen PD-1 as described herein.

Binding molecules of the invention, including the antibodies and multivalent or multispecific binding agents described herein, bind specifically to canine PD-1. In other words, binding to the PD-1 antigen is measurably different from a non-specific interaction. As demonstrated in the examples, the antibodies of the invention do not cross react with mouse PD-1. Further as demonstrated in the examples, the antibodies of the invention do not cross react with human PD-1. Preferably, the antibodies of the invention bind to canine PD-1.

The term “specific binding” or “specifically binds to” or is “specific for” a particular polypeptide or an epitope on a particular polypeptide target as used herein can be exhibited, for example, by a molecule having a KD for the target of at least about 10⁻⁶ M, alternatively at least about 10⁻⁷ M, alternatively at least about 10⁻⁸ M, alternatively at least about 10⁻⁹ M, alternatively at least about 10⁻¹⁰ M, alternatively at least about 10⁻¹¹ M, alternatively at least about 10⁻¹² M, alternatively at least about 10⁻¹³ M or lower. In one embodiment, the term “specific binding” as used herein can be exhibited by a molecule having a KD for the target of at least about 10⁻⁸ M, alternatively at least about 10⁻⁹ M, alternatively at least about 10⁻¹⁰ M, alternatively at least about 10⁻¹¹ M, alternatively at least about 10⁻¹² M, alternatively at least about 10⁻¹³ M or lower. In one embodiment, the term “specific binding” refers to binding of at least single or double digit nanomolar or picomolar. In one embodiment, the term “specific binding” refers to binding where a molecule binds to a particular polypeptide or epitope on a particular polypeptide without substantially binding to any other polypeptide or polypeptide epitope. In an embodiment the KD of the binding molecules of the invention for the target is determined using a monovalent and/or a bivalent binding assay for example the monovalent and bivalent binding assays described in Example 9. In one embodiment, the term “specific binding” as used herein can be exhibited by a molecule having a KD for the target of at least about 10⁻⁸ M, alternatively at least about 10⁻⁹ M, alternatively at least about 10⁻¹⁰ M, alternatively at least about 10⁻¹¹ M, alternatively at least about 10⁻¹² M, alternatively at least about 10⁻¹³ M or lower when determined by a bivalent binding assay. The binding molecules may have a KD for the target of between about 10⁻⁸ M to 10⁻¹³ M, 10⁻⁹ M to 10⁻¹³ M, 10⁻¹⁰ M to 10⁻¹³ M, 10⁻¹¹ M to 10⁻¹³ M, 10⁻¹² M to 10⁻¹³ M, when determined by a bivalent binding assay. In an embodiment the binding molecules may have a KD for the target of between about 10⁻¹⁰ M to 10⁻¹³ M when determined by a bivalent binding assay. In one embodiment, the term “specific binding” as used herein can be exhibited by a molecule having a KD for the target of at least about 10⁻⁸ M, alternatively at least about 10⁻⁹ M, alternatively at least about 10⁻¹⁰ M, alternatively at least about 10⁻¹¹ M, alternatively at least about 10⁻¹² M, alternatively at least about 10⁻¹³ M or lower when determined by a monovalent binding assay. The binding molecules may have a KD for the target of between about 10⁻⁸ M to 10⁻¹³ M, 10⁻⁹ M to 10⁻¹³ M, 10⁻¹⁰ M to 10⁻¹³ M, 10⁻¹¹ M to 10⁻¹³ M, 10⁻¹² M to 10⁻¹³ M, when determined by a monovalent binding assay. In an embodiment the binding molecules may have a KD for the target of between about 10⁻⁸ M to 10⁻¹¹ M when

determined by a monovalent binding assay. The term “antibody” as used herein broadly refers to any immunoglobulin (Ig) molecule, or antigen binding portion thereof, comprised of four polypeptide chains, two heavy (H) chains and two light (L) chains, or any functional fragment, mutant, variant, or derivation thereof, which retains the essential epitope binding features of an Ig molecule. Such mutant, variant, or derivative antibody formats are known in the art.

In a full-length antibody, each heavy chain is comprised of a heavy chain variable region or domain (abbreviated herein as HCVR) and a heavy chain constant region. The heavy chain constant region is comprised of three domains, CH1, CH2 and CH3. Each light chain is comprised of a light chain variable region or domain (abbreviated herein as LCVR) and a light chain constant region. The light chain constant region is comprised of one domain, CL.

The heavy chain and light chain variable regions can be further subdivided into regions of hypervariability, termed complementarity determining regions (CDR), interspersed with regions that are more conserved, termed framework regions (FR). Each heavy chain and light chain variable region is composed of three CDRs and four FRs, arranged from amino-terminus to carboxy-terminus in the following order: FR1, CDR1, FR2, CDR2, FR3, CDR3, FR4.

Immunoglobulin molecules can generally be of any type ((e.g., IgG, IgE, IgM, IgD, IgA and IgY), class (e.g., IgG 1, IgG2, IgG 3, IgG4, IgA1 and IgA2), or subclass.

In canine, there are four IgG heavy chains referred to as A, B, C, and D. These heavy chains represent four different subclasses of dog IgG, which are referred to as IgG-A, IgG-B, IgG-C and IgG-D. The DNA and amino acid sequences of these four heavy chains were first identified by Tang et al. (Vet. Immunol. Immunopathol. 80: 259-270 (2001)). The amino acid and DNA sequences for these heavy chains are also available from the GenBank data bases (IgGA: accession number AAL35301.1, IgGB: accession number AAL35302.1, IgGC: accession number AAL35303.1, IgGD: accession number AAL35304.1). Canine antibodies also contain two types of light chains, kappa and lambda (GenBank accession number kappa light chain amino acid sequence ABY 57289.1, GenBank accession number ABY 55569.1). The antibodies herein may have a lambda or kappa light chain. In one embodiment, the light chain is a lambda light chain.

The term “CDR” refers to the complementarity-determining region within antibody variable sequences. There are three CDRs in each of the variable regions of the heavy chain and the light chain, which are designated CDR1, CDR2 and CDR3, for each of the variable regions. The term “CDR set” refers to a group of three CDRs that occur in a single variable region capable of binding the antigen. The exact boundaries of these CDRs can be defined differently according to different systems known in the art.

The Kabat Complementarity Determining Regions (CDRs) are based on sequence variability and are the most commonly used (Kabat et al., (1971) Ann. NY Acad. Sci. 190:382-391 and Kabat, et al., (1991) Sequences of Proteins of Immunological Interest, Fifth Edition, U.S. Department of Health and Human Services, NIH Publication No. 91-3242). Chothia refers instead to the location of the structural loops (Chothia and Lesk J. Mol. Biol. 196:901-917 (1987)). The Kabat numbering system is generally used when referring to a residue in the variable domain (approximately residues 1-107 of the light chain and residues 1-113 of the heavy chain). Another system is the ImMunoGeneTics (IMGT) numbering scheme. The IMGT numbering scheme is described in Lefranc et al., Dev. Comp. Immunol., 29, 185-203 (2005). The IMGT numbering scheme is used herein unless otherwise specified.

In another embodiment, the invention pertains to an isolated monoclonal antibody, or an antigen-binding portion thereof, comprising a heavy chain variable region that is the product of or derived from a canine IGHV3-5 VH gene, a canine IGHV3-19 VH gene, or a canine IGHV4-1 VH gene, wherein the antibody specifically binds canine PD-1. In another embodiment, the invention pertains to an isolated monoclonal antibody, or an antigen-binding portion thereof, comprising a light chain variable region that is the product of or derived from a canine IGKV2-9 VK gene, or a canine IGKV2-5 VK gene; or a canine IGLV3-3 VL gene, or a canine IGLV3-11 VL gene, wherein the antibody specifically binds canine PD-1.

The antibody to PD-1 according to the invention may be a canine, humanized, chimeric antibody, felinized or caninized antibody.

A “chimeric antibody” is a recombinant protein that contains the variable domains including the complementarity determining regions (CDRs) of an antibody derived from one species, preferably a rodent or human antibody, while the constant domains of the antibody molecule are derived from those of a canine antibody.

As used herein, the term “caninized antibody” refers to forms of recombinant antibodies that contain sequences from both canine and non-canine (e.g., murine) antibodies. In general, a caninized antibody will comprise substantially all of at least one or more typically, two variable domains in which all or substantially all of the hypervariable loops correspond to those of a non-canine immunoglobulin, and all or substantially all of the framework (FR) regions (and typically all or substantially all of the remaining frame) are those of a canine immunoglobulin sequence. A caninized antibody may comprise both the three heavy chain CDRs and the three light chain CDRS from a murine or human antibody together with a canine frame or a modified canine frame. A modified canine frame comprises one or more amino acids changes that can further optimize the effectiveness of the caninized antibody, e.g., to increase its binding to its target. The non-canine sequences, e.g., of the hypervariable loops, may further be compared to canine sequences and as many residues changed to be as similar to authentic canine sequences as possible.

As used herein, the term “felinized antibody” refers to forms of recombinant antibodies that contain sequences from both feline and non-feline (e.g., canine) antibodies. In general, the felinized antibody will comprise substantially all of at least one or more typically, two variable domains in which all or substantially all of the hypervariable loops correspond to those of a non-feline immunoglobulin, and all or substantially all of the framework (FR) regions (and typically all or substantially all of the remaining frame) are those of a feline immunoglobulin sequence. A felinized antibody may comprise both the three heavy chain CDRs and the three light chain CDRS from a murine or human antibody together with a feline frame or a modified feline frame. A modified feline frame comprises one or more amino acids changes that can further optimize the effectiveness of the felinized antibody, e.g., to increase its binding to its target. The non-feline sequences, e.g., of the hypervariable loops, may further be compared to feline sequences and as many residues changed to be as similar to authentic feline sequences as possible.

A “speciated” antibody (e.g. humanized, caninized, chimeric, felinized) is one which has been engineered to render it similar to antibodies of the target species. In one embodiment, a “speciated” antibody is greater than about 80%, 85% or 90% similar to antibodies of the target species.

In contrast, fully canine antibodies of the present invention have canine variable regions and do not include full or partial CDRs or FRs from another species. Advantageously, fully canine antibodies as described herein have been obtained from transgenic mice comprising canine immunoglobulin sequences. Antibodies produced in these immunised mice are developed through in vivo B cell signalling and development to allow for natural affinity maturation including in vivo V(D)J recombination, in vivo junctional diversification, in vivo pairing of heavy and light chains and in vivo hypermutation. Fully canine antibodies produced in this way generate antibodies with optimal properties for developability, minimizing lengthy lead optimization prior to production at scale. Advantageously, such fully canine antibodies present the lowest possible risk of immunogenicity when introduced into a patient animal which, in turn, facilitates a repeated dosing regime. Given that ex vivo mAb engineering runs the risk of introducing development liabilities, immunogenicity, and reduced affinity (as outlined above), fully canine antibodies of the present invention are, therefore, most likely to be efficacious therapies in a clinical context.

In one embodiment, the antibody or antibody fragment is canine. In one embodiment, the antibody is fully canine.

In one embodiment, the antibody or antibody fragment is cross-reactive with another species. In one embodiment, the canine antibody cross-reacts with feline PD-1 (GenBank ID 100135770). In another embodiment, the canine antibody that cross-reacts with canine PD-1 is felinized. The antibody or antibody fragment that cross reacts with feline PD-1 may have any of the features as described herein. In an embodiment the antibody or antibody fragment that cross reacts with feline PD-1 comprises an HC CDR1 sequence comprising or consisting of SEQ ID NO: 207 or an amino acid sequence with at least 70%, 80%, 85%, 90% or 95% sequence identity to SEQ ID NO: 207 and an HC CDR2 sequence comprising or consisting of SEQ ID NO: 208 or an amino acid sequence with at least 70%, 80%, 85%, 90% or 95% sequence identity to SEQ ID NO: 208 and an HC CDR3 sequence comprising or consisting of SEQ ID NO: 209 or an amino acid sequence w with at least 70%, 80%, 85%, 90% or 95% sequence identity to SEQ ID NO: 209 and an LC CDR1 sequence comprising or consisting of SEQ ID NO: 210 or an amino acid sequence with at least 70%, 80%, 85%, 90% or 95% sequence identity to SEQ ID NO: 210 and an LC CDR2 sequence comprising or consisting of SEQ ID NO: 211 or an amino acid sequence with at least 70%, 80%, 85%, 90% or 95% sequence identity to SEQ ID NO: 211 and an LC CDR3 sequence comprising or consisting of SEQ ID NO: 212 or an amino acid sequence with at least 70%, 80%, 85%, 90% or 95% sequence identity to SEQ ID NO: 212.

The term “antigen binding site” refers to the part of the antibody or antibody fragment that comprises the area that specifically binds to an antigen. An antigen binding site may be provided by one or more antibody variable domains. Preferably, an antigen binding site is comprised within the associated VH and VL of an antibody or antibody fragment.

The terms antigen binding portion or antigen binding fragment or portion or fragment of an antibody as used here are used interchangeably. In an embodiment of the invention an antibody or antigen-binding portion thereof which binds to canine PD-1 is provided wherein said antigen-binding portion thereof is an scFv, Fv, heavy chain or a single domain antibody, e.g. a VH single domain antibody.

An antibody fragment is a functional portion of a full-length antibody, for example as F(ab′)2, Fab, Fv, sFv and the like. Functional fragments of a full-length antibody retain the target specificity of a full-length antibody. Recombinant functional antibody fragments, such as Fab (Fragment, antibody), scFv (single chain variable chain fragments) and single domain antibodies (dAbs) have therefore been used to develop therapeutics as an alternative to therapeutics based on mAbs.

An “Fv” is the minimum antibody fragment which contains a complete antigen-recognition and -binding site. This fragment consists of a dimer of one heavy- and one light-chain variable region domain in tight, non-covalent association. From the folding of these two domains emanate six hypervariable loops (3 loops each from the H and L chain) that contribute the amino acid residues for antigen binding and confer antigen binding specificity to the antibody. However, even a single variable domain (or half of an Fv comprising only three HVRs specific for an antigen) has the ability to recognize and bind antigen, although at a lower affinity than the entire binding site. “Single-chain Fv” also abbreviated as “sFv” or “scFv” are antibody fragments that comprise the VH and VL antibody domains connected into a single polypeptide chain.

scFv fragments (˜25 kDa) consist of the two variable domains, VH and VL. Naturally, VH and VL domain are non-covalently associated via hydrophobic interaction and tend to dissociate. However, stable fragments can be engineered by linking the domains with a hydrophilic flexible linker to create a single chain Fv (scFv).

The smallest antigen binding fragment is the single variable fragment, namely the variable heavy (VH) or variable light (VL) chain domain. VH and VL domains respectively are capable of binding to an antigen. Binding to a light chain/heavy chain partner respectively or indeed the presence of other parts of the full antibody is not required for target binding. The antigen-binding entity of an antibody, reduced in size to one single domain (corresponding to the VH or VL domain), is generally referred to as a “single domain antibody” or “immunoglobulin single variable domain”. A single domain antibody (˜12 to 15 kDa) has thus either the VH or VL domain.

In one aspect, the invention relates to an isolated single domain antibody, an isolated variable single domain or an isolated immunoglobulin single variable domain wherein said isolated single domain antibody, isolated variable single domain or isolated immunoglobulin single variable domain binds to canine PD-1 and blocks the interaction of PD-1 and PD-L1 and/or PD-L2.

The terms “single domain antibody, variable single domain or immunoglobulin single variable domain (ISV)” are all well known in the art and describe the single variable fragment of an antibody that binds to a target antigen. These terms are used interchangeably herein. As explained below, embodiments of the various aspects of the invention relate to single heavy chain variable domain antibodies/immunoglobulin heavy chain single variable domains which bind a PD-1 antigen in the absence of light chain. Canine heavy chain single variable domain antibodies are thus within the scope of the invention.

Thus, in some embodiments, the isolated binding agents/molecules of the invention comprise or consist of at least one single domain antibody wherein said domain is a canine heavy chain variable domain. Thus, in one aspect, the binding agents of the invention comprise or consist of at least one canine immunoglobulin single variable heavy chain domain; they are devoid of VL domains.

The term “isolated” single domain antibody refers to a single domain antibody that is substantially free of other single domain antibodies, antibodies or antibody fragments having different antigenic specificities. Moreover, an isolated single domain antibody may be substantially free of other cellular material and/or chemicals.

In an embodiment of the invention an isolated canine antibody or antigen-binding portion thereof which binds to PD-1 is provided wherein said antibody or antigen-binding portion thereof competes with PD-L1 and/or PD-L2.

In a related embodiment of the invention an isolated canine antibody or antigen-binding portion thereof which binds to PD-1 is provided wherein said antibody or antigen-binding portion thereof blocks the interaction of PD-1 with PD-L1 and/or PD-L2 and/or prevents a cellular response associated with the interaction of PD-1 with PD-L1 and/or PD-L2.

A “blocking antibody or antibody” or a “neutralizing antibody or antibody”, as used herein refers to an antibody whose binding to PD-1 results in inhibition of at least one biological activity of PD-1. For example, an antibody of the invention may prevent or block PD-1 binding to PD-L1 and/or PD-L2. In one embodiment, the antibody of the invention blocks PD-1 binding to PD-L1. In one embodiment, the antibody of the invention blocks PD-1 binding to PD-L2.

Each single VH domain antibody comprises three CDRs and four FRs, arranged from amino-terminus to carboxy-terminus in the following order: FR1-CDR1-FR2-CDR2-FR3-CDR3-FR4. Thus, in one embodiment of the invention, the domain is a human variable heavy chain (VH) domain with the following formula FR1-CDR1-FR2-CDR2-FR3-CDR3-FR4.

Modifications to the C or N-terminal VH framework sequence may be made to the antibodies of the invention to improve their properties. For example, the VH domain may comprise C or N-terminal extensions or deletions.

The term “monoclonal antibody” as used herein refers to an antibody obtained from a population of substantially homogeneous antibodies, i.e., the individual antibodies comprising the population are identical except for possible naturally occurring mutations and/or post-translation modifications (e.g., isomerizations, amidations, carbohydrate addition) that may be present in minor amounts. Such monoclonal antibodies may be derived from a single B or plasma cell. Monoclonal antibodies are highly specific, being directed against a single antigenic site. In contrast to polyclonal antibody preparations which typically include different antibodies directed against different determinants (epitopes), each monoclonal antibody is directed against a single determinant on the antigen. In addition to their specificity, the monoclonal antibodies are advantageous in that they are synthesized by the hybridoma culture, uncontaminated by other immunoglobulins.

The term “antigen binding site” refers to the part of the antibody or antibody fragment that comprises the area that specifically binds to an antigen. An antigen binding site may be provided by one or more antibody variable domains. An antigen binding site is typically comprised within the associated VH and VL of an antibody or antibody fragment.

The term “epitope” or “antigenic determinant” refers to a site on the surface of an antigen (to which an immunoglobulin, antibody or antibody fragment, specifically binds. Generally, an antigen has several or many different epitopes and reacts with many different antibodies. The term specifically includes linear epitopes and conformational epitopes. Epitopes within protein antigens can be formed both from contiguous amino acids (usually a linear epitope) or non-contiguous amino acids juxtaposed by tertiary folding of the protein (usually a conformational epitope). Epitopes formed from contiguous amino acids are typically, but not always, retained on exposure to denaturing solvents, whereas epitopes formed by tertiary folding are typically lost on treatment with denaturing solvents. An epitope typically includes at least 3, 4, 5, 6, 7, 8, 9, 10, 11, 12, 13, 14 or 15 amino acids in a unique spatial conformation. Methods for determining what epitopes are bound by a given antibody or antibody fragment (i.e., epitope mapping) are well known in the art and include, for example, immunoblotting and immunoprecipitation assays, wherein overlapping or contiguous peptides from are tested for reactivity with a given antibody or antibody fragment.

An antibody binds “essentially the same epitope” as a reference antibody, when the two antibodies recognize identical or sterically overlapping epitopes. The most widely used and rapid methods for determining whether two epitopes bind to identical or sterically overlapping epitopes are competition assays, which can be configured in different formats, using either labelled antigen or labelled antibody. The epitope may or may not be a three-dimensional surface feature of the antigen. In some embodiments, an antibody may, for example, bind to a monomer/single subunit and block formation of an active form. Suitably the antibodies bind to the extracellular domain of PD-1.

Proteolytic digestion of antibodies releases different fragments termed Fv (Fragment variable), Fab (Fragment antigen binding) and Fc (Fragment crystallisation). The Fc fragment comprises the carboxy-terminal portions of both H chains held together by disulfides. The constant domains of the Fc fragment are responsible for mediating the effector functions of an antibody.

The invention extends to antigen binding portions or antigen binding fragments of the antibody. The terms “binding portion” and “fragment” are used interchangeably herein. An antibody fragment is a portion of an antibody, for example a F(ab′)2, Fab, Fv, scFv, heavy chain, light chain, variable heavy (VH), variable light (VL) chain domain and the like. Functional fragments of a full-length antibody retain the target specificity of a full antibody. Recombinant functional antibody fragments, such as Fab (Fragment, antibody), scFv (single chain variable chain fragments) and single domain antibodies (dAbs) have therefore been used to develop therapeutics as an alternative to therapeutics based on mAbs.

The invention also extends to antibody mimetics that comprise a sequence as described herein.

An “Fv” is the minimum antibody fragment which contains a complete antigen-recognition and -binding site. This fragment consists of a dimer of one heavy- and one light-chain variable region domain in tight, non-covalent association. From the folding of these two domains emanate six hypervariable loops (3 loops each from the H and L chain) that contribute the amino acid residues for antigen binding and confer antigen binding specificity to the antibody. However, even a single variable domain (or half of an Fv comprising only three HVRs specific for an antigen) has the ability to recognize and bind antigen, although at a lower affinity than the entire binding site.

“Single-chain Fv” also abbreviated as “sFv” or “scFv” are antibody fragments scFv fragments (˜25 kDa) that consist of the two variable domains, VH and VL connected into a single polypeptide chain. Naturally, VH and VL domains are non-covalently associated via hydrophobic interactions and tend to dissociate. However, stable fragments can be engineered by linking the domains with a hydrophilic flexible linker to create a single chain Fv (scFv).

The smallest antigen binding fragment is the single variable fragment, namely the variable heavy (VH) or variable light (VL) chain domain. VH and VL domains respectively are capable of binding to an antigen. Binding to a light chain/heavy chain partner respectively or indeed the presence of other parts of the full antibody is not required for target binding. The antigen-binding entity of an antibody, reduced in size to one single domain (corresponding to the VH or VL domain), is generally referred to as a “single domain antibody” or “immunoglobulin single variable domain”. A single domain antibody (˜12 to 15 kDa) has thus either the VH or VL domain, i.e. it does not have other parts of a full antibody. The term “dAb” for “domain antibodies” generally refers to a single immunoglobulin variable domain (VH, VHH or VL) polypeptide that specifically binds antigen.

The term “isolated” refers to a moiety that is isolated from its natural environment. For example, the term “isolated” refers to an antibody or fragment thereof that is substantially free of other antibodies, antibodies or antibody fragments. Moreover, an isolated antibody may be substantially free of other cellular material and/or chemicals.

As used herein, the term “homology” or “identity” generally refers to the percentage of amino acid residues in a sequence that are identical with the residues of the reference polypeptide with which it is compared, after aligning the sequences and in some embodiments after introducing gaps, if necessary, to achieve the maximum percent homology, and not considering any conservative substitutions as part of the sequence identity. Thus, the percent homology between two amino acid sequences is equivalent to the percent identity between the two sequences. Neither N- or C-terminal extensions, tags or insertions shall be construed as reducing identity or homology. Methods and computer programs for the alignment are well known. The percent identity between two amino acid sequences can be determined using well known mathematical algorithms.

By “amino acid” herein is meant one of the 20 naturally occurring amino acids or any non-natural analogues that may be present at a specific, defined position. Amino acid encompasses both naturally occurring and synthetic amino acids. Although in most cases, when the protein is to be produced recombinantly, only naturally occurring amino acids are used.

As used herein, a “substitution of an amino acid residue” with another amino acid residue in an amino acid sequence of heterodimeric protein or polypeptide as described herein (an antibody for example), is equivalent to “replacing an amino acid residue” with another amino acid residue and denotes that a particular amino acid residue at a specific position in the original (e.g. wild type/germline) amino acid sequence has been replaced by (or substituted for) by a different amino acid residue. This can be done using standard techniques available to the skilled person, e.g. using recombinant DNA technology. The amino acids are changed relative to the native (wild type/germline) sequence as found in nature in the wild type (wt), but may be made in IgG molecules that contain other changes relative to the native sequence. By “wild type” or “WT” or “native” herein is meant an amino acid sequence or a nucleotide sequence that is found in nature, including allelic variations. A WT protein, polypeptide, antibody or immunoglobulin has an amino acid sequence or a nucleotide sequence that has not been intentionally modified.

The antibody or antigen-binding portion thereof according to the invention has one or more of the following properties:
a) binds to canine PD-1;
b) blocks or reduces the functional interaction of PD-1 with PD-L1 and/or PD-L2;
c) shows PD-1 blockade in demonstrated in the examples;
d) increase the secretion of IFN-γ from canine PBMC as shown in the examples;
e) binds canine PD-1 on activated canine T cells as shown in the examples;
f) is capable of binding to cells expressing canine PD-1;
g) provides good stability as shown in the examples;
h) do not cross react with human PD-1;
i) has a half-life of between 1 and 20 days;
j) is capable of increasing or upregulating T cell activation; and/or
k) is capable of upregulating one or more genes involved in T cell proliferation, activation and/or differentiation, such as IFGGC1, CD3D, ICOS, CXCL10, CD80, CCR5, IL-7 and/or INF-gamma, and/or GZMB, PRF1, CD28, CXCR3, CTLA-4, CD8A, CD8B, TBX21, GATA3, IL2RA, CD40LG and/or IRF1.

These properties can be measured by methods known in the art, such the methods disclosed in the examples.

For example, blocking the functional interaction of PD-1 with PD-L1 and/or PD-L2 means that the antibody reduces or abolishes binding of PD-1 to PD-L1 and/or PD-L2 such that that signalling through these pathways is abolished or reduced.

The antibody or antigen binding portion thereof according to the invention has a favourable half-life. In some embodiments the antibody or antigen binding portion thereof demonstrates a half-life of 1 to 20 days, 1 to 19 days, 1 to 18 days, 1 to 17 days, 1 to 16 days, 1 to 15 days, 1 to 14 days, 1 to 13 days, 1 to 12 days, 1 to 11 days, 1 to 10 days, 2 to 10 days, 3 to 10 days, 4 to 10 days, 5 to 10 days, 1 to 9 days, 1 to 8 days, 1 to 7 days, 1 to 6 days, 1 to 5 days, 5 to 20 days, 5 to 19 days, 5 to 18 days, 5 to 17 days, 5 to 16 days, 5 to 15 days, 5 to 14 days, 5 to 13 days, 5 to 12 days, 5 to 11 days, 5 to 10 days, 10 to 20 days, 10 to 19 days, 10 to 18 days, 10 to 17 days, 10 to 16 days, 10 to 15 days. In some embodiments the antibody or antigen binding portion thereof demonstrates a half-life in the range of 10 to 15 days for example 10 days, 11 days, 12 days, 13 days, 14 days, 15 days.

Increasing or upregulating T cell activation may be assessed by comparing the level of T cell activation to a reference value. The reference value may be the level of T cell activation from a subject prior to treatment with an antibody of the invention. The reference value may be the level of T cell activation from a healthy subject. The reference value may be the level of T cell activation from a diseased subject. The diseased subject may or may not have received therapy.

T cell activation may be measured using any suitable method known in the art. Examples of suitable methods to determine T cell activation are provided herein in Example 12 and Example 20. In an embodiment, increased secretion of IFN-γ may indicate increased T cell activation. In an embodiment T cell activation may be determined by upregulation of certain genes linked to T cell activation, for example upregulation of one or more of IFGGC1, CD3D, ICOS, CXCL10, CD80, CCR5, IL-7 and/or INF-gamma.

Upregulation of one or more genes selected from IFGGC1, CD3D, ICOS, CXCL10, CD80, CCR5, IL-7 and/or INF-gamma may be assessed by comparing the level of said gene e.g., expression level to a reference value. The reference value may be the level of one or more of said genes from a subject prior to treatment with an antibody of the invention. The reference value may be the level of level of one or more of said genes from a healthy subject. The reference value may be the level of one or more of said genes from a diseased subject. The diseased subject may or may not have received therapy.

The term “IFGGC1” refers to the gene encoding interferon-inducible GTPase. The term “CD3D” refers to the gene encoding CD3 Delta Subunit Of T-Cell Receptor Complex. The term “ICOS” refers to the gene that encodes inducible T-cell costimulator receptor. The term “CXCL10” refers to the gene that encodes the C—X—C motif chemokine ligand 10. The term “CD80” refers to the gene that encodes the cluster of differentiation 80. The term “CCR5” refers to the gene that encodes C—C chemokine receptor type 5.

In one embodiment, the antibody is PMX126, PMX127, PMX128, PMX129, PMX130, PMX131, PMX132, PMX133, PMX138, PMX140, PMX142, PMX143, PMX145, PMX146, PMX147, PMX148, PMX149, PMX150, PMX151, PMX136, PMX137, PMX141, PMX144, PMX134, PMX152, PMX153, PMX135, PMX139, PMX125, or a fragment thereof as shown in the examples and sequence information. The VH CDR amino acid sequences, VL CDR amino acid sequences, VH amino acid sequences and VL amino acid sequences for PMX molecules as shown in Table 2 are specifically within the scope of the invention.

In one embodiment, the antibody or antigen-binding portion thereof comprises an Fc region, for example a canine Fc region, for example a canine IgGB Fc region.

Also within the scope of the invention are variants of the antibodies and antigen binding portions as described above.

A variant of an antibody or antigen binding portion thereof (e.g. a VH or a VL) as described herein has at least 80%, 90%, 91%, 92%, 93%, 94%, 95%, 96%, 97%, 98% or 99% sequence identity to the non-variant molecule. In one embodiment, sequence identity is at least 95%. In one embodiment, the modification is a conservative sequence modification.

As used herein, the term “conservative sequence modifications” is intended to refer to amino acid modifications that do not significantly affect or alter the binding characteristics of the antibody containing the amino acid sequence. Such conservative modifications include amino acid substitutions, additions and deletions. Modifications can be introduced into an antibody or antigen binding portion thereof of the invention by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within the CDR regions of an antibody of the invention can be replaced with other amino acid residues from the same side chain family and the altered antibody can be tested for retained function (i.e., PD-1 binding) using the functional assays described herein.

Thus, these amino acid changes can typically be made without altering the biological activity, function, or other desired property of the polypeptide, such as its affinity or its specificity for antigen. In general, single amino acid substitutions in nonessential regions of a polypeptide do not substantially alter biological activity. Furthermore, substitutions of amino acids that are similar in structure or function are less likely to disrupt the polypeptides' biological activity. Abbreviations for the amino acid residues that comprise polypeptides and peptides described herein, and conservative substitutions for these amino acid residues are shown in Table 1 below.

TABLE 1
Amino Acid Residues and Examples of Conservative
Amino Acid Substitutions.

| Original residue | Conservative |
|---|---|
| Three letter code, single letter code | substitution |
| Alanine, Ala, A | Gly, Ser |
| Arginine, Arg, R | Lys, His |
| Asparagine, Asn, N | Gln, His |
| Aspartic acid Asp, D | Glu, Asn |
| Cysteine, Cys, C | Ser, Ala |
| Glutamine, Gln, Q | Asn |
| Glutamic acid, Glu, E | Asp, Gln |
| Glycine, Gly, G | Ala |
| Histidine, His, H | Asn, Gln |
| Isoleucine, Ile, I | Leu, Val |
| Leucine, Leu, L | Ile, Val |
| Lysine, lys, K | Ar, His |
| Methionine, Met, M | Leu, Ile, Tyr |
| Phenylalanine, Phe, F | Tyr, Met, Leu |
| Proline, Pro, P | Ala |
| Serine, Ser, S | Thr |
| Threonine, Thr, T | Ser |
| Tryptophan, Trp, W | Tyr, Phe |
| Tyrosine, Tyr, Y | Try, Phe |
| Valine, Val, V | Ile, Leu |

In some embodiments, the invention provides an antibody or antigen binding portion thereof that is a variant of an antibody or antigen binding portion thereof compared to a sequence selected from any one of the amino acids sequences from SEQ ID NO: 3 to SEQ ID NO: 292 that comprises one or more sequence modification and has improvements in one or more of a property such as binding affinity, specificity, thermostability, expression level, effector function, glycosylation, reduced immunogenicity, or solubility as compared to the unmodified antibody or fragment thereof. Modifications for altered effector function activity are described, for example, in WO2023012496 and WO2021165417.

In some embodiments, the invention provides an antibody or antigen binding portion thereof that is a variant of an antibody or antigen binding portion thereof compared to a sequence selected from any one of the amino acids sequences from SEQ ID NO: 3 to SEQ ID NO: 292 that comprises one or more sequence modification and has improvements in half-life as compared to the unmodified antibody or antigen binding portion thereof. Suitable sequence modifications for improving half-life are known in the art and are described for example in WO2018073185, WO 2020082048, WO2020116560, WO2020142625, WO2021212081, WO2021212081 and WO2022067233. Where the antibody or antigen-binding portion thereof comprises a half life extending sequence modifications the half life of the molecule may demonstrate a half-life of 1 to 20 days, 1 to 19 days, 1 to 18 days, 1 to 17 days, 1 to 16 days, 1 to 15 days, 1 to 14 days, 1 to 13 days, 1 to 12 days, 1 to 11 days, 1 to 10 days, 2 to 10 days, 3 to 10 days, 4 to 10 days, 5 to 10 days, 1 to 9 days, 1 to 8 days, 1 to 7 days, 1 to 6 days, 1 to 5 days, 5 to 20 days, 5 to 19 days, 5 to 18 days, 5 to 17 days, 5 to 16 days, 5 to 15 days, 5 to 14 days, 5 to 13 days, 5 to 12 days, 5 to 11 days, 5 to 10 days, 10 to 20 days, 10 to 19 days, 10 to 18 days, 10 to 17 days, 10 to 16 days, or 10 to 15 days.

Suitable methods for measuring properties which may suggest that the antibody can be successfully developed at scale include first purification using chromatography, such as affinity chromatography chromatography (Protein A: MabSelect Sure LX), anion exchange chromatography (Capto Q), cation exchange chromatography (Capto S) and buffer exchange (G-25 Fine), followed by assessment of whether the antibody remains intact (e.g. using SDS PAGE analysis to determine molecular weight, HPLC-SEC to calculate % of monomers, assess aggregation, and thermostability (Tm) studies.

In an embodiment of the invention an antibody or antigen-binding portion thereof which binds to PD-1 is provided wherein said antibody or antigen-binding portion thereof is conjugated to a therapeutic moiety such as a drug, an enzyme or a toxin. In one embodiment, the therapeutic moiety is a toxin, for example a cytotoxic radionuclide, chemical toxin or protein toxin. In a further related embodiment of the invention an antibody or antigen-binding portion thereof which binds to PD-1 is provided wherein said therapeutic moiety is a second antibody or antigen-binding portion thereof. In a related embodiment of the invention the second antibody or antigen-binding portion thereof binds to a different target. In a further related embodiment the different target may be one or more of the following: LAG-3, OX40L, CD2, CD27, CDS, ICAM-1, LFA-1 (CD11a/CD18), ICOS (CD278), 4-1BB (CD137), GITR, CD30, CD40, BAFFR, HVEM, CD7, LIGHT, NKG2C, SLAMF7, NKp80, CD160, B7-H3 or CD83 ligand, CD3, CD8, CD28, CD4 or ICAM-1.

In another embodiment of the invention an antibody or antigen-binding portion thereof which binds to PD-1 is provided wherein said antibody or antigen-binding portion thereof is conjugated to a further moiety selected from a half-life extending moiety, label, cytotoxin, liposome, nanoparticle or radioisotope. In a related embodiment of the invention the half-life extending moiety is selected from: albumin binding moiety, a transferrin binding moiety, a polyethylene glycol molecule, a recombinant polyethylene glycol molecule, human serum albumin, a fragment of human serum albumin, an albumin binding peptide or single domain antibody that binds to human serum albumin. It will be apparent to the skilled person that other half-life extending moieties may be conjugated to the antibody or antigen-binding portion thereof according to the invention. Where the antibody or antigen-binding portion thereof comprises a half life extending moiety the half life of the molecule may demonstrate a half-life of 1 to 20 days, 1 to 19 days, 1 to 18 days, 1 to 17 days, 1 to 16 days, 1 to 15 days, 1 to 14 days, 1 to 13 days, 1 to 12 days, 1 to 11 days, 1 to 10 days, 2 to 10 days, 3 to 10 days, 4 to 10 days, 5 to 10 days, 1 to 9 days, 1 to 8 days, 1 to 7 days, 1 to 6 days, 1 to 5 days, 5 to 20 days, 5 to 19 days, 5 to 18 days, 5 to 17 days, 5 to 16 days, 5 to 15 days, 5 to 14 days, 5 to 13 days, 5 to 12 days, 5 to 11 days, 5 to 10 days, 10 to 20 days, 10 to 19 days, 10 to 18 days, 10 to 17 days, 10 to 16 days, 10 to 15 days. Where the antibody or antigen-binding portion thereof comprises a half-life extending moiety the half-life of the molecule may demonstrate a half-life in the range of 10 to 15 days for example 10 days, 11 days, 12 days, 13 days, 14 days, or 15 days.

In another aspect of the invention a pharmaceutical composition comprising an antibody or antigen-binding portion thereof according to any preceding embodiment is provided. The pharmaceutical composition may be used in the treatment of disease. In one embodiment the disease is a cancer or tumor.

The pharmaceutical composition according to the invention may comprise the antibody or antigen-binding portion thereof as described herein and optionally a pharmaceutically acceptable carrier. The term pharmaceutical composition as used herein refers to a composition that is used to treat a companion animal, that is for veterinary use, i.e. a veterinary composition. In preferred embodiments, the animal that is treated is a dog.

The pharmaceutical composition may optionally comprise a pharmaceutically acceptable carrier. Antibodies, protein or construct or the pharmaceutical composition can be administered by any convenient route, including but not limited to oral, topical, parenteral, sublingual, rectal, vaginal, ocular, intranasal, pulmonary, intradermal, intravitreal, intramuscular, intraperitoneal, intravenous, subcutaneous, intracerebral, transdermal, transmucosal, by inhalation, or topical, particularly to the ears, nose, eyes, or skin or by inhalation.

Parenteral administration includes, for example, intravenous, intramuscular, intraarterial, intraperitoneal, intranasal, rectal, intravesical, intradermal, topical or subcutaneous administration. Preferably, the compositions are administered parenterally.

The pharmaceutically acceptable carrier or vehicle can be particulate, so that the compositions are, for example, in tablet or powder form. The term “carrier” refers to a diluent, adjuvant or excipient, with which a drug antibody conjugate of the present invention is administered. Such pharmaceutical carriers can be liquids, such as water and oils, including those of petroleum, animal, vegetable or synthetic origin, such as peanut oil, soybean oil, mineral oil, sesame oil and the like. The carriers can be saline, gum acacia, gelatin, starch paste, talc, keratin, colloidal silica, urea, and the like. In addition, auxiliary, stabilizing, thickening, lubricating and coloring agents can be used. In one embodiment, when administered to an animal, the antigen binding domain, or antibody of the present invention or compositions and pharmaceutically acceptable carriers are sterile. Water is a preferred carrier when the drug antibody conjugates of the present invention are administered intravenously. Saline solutions and aqueous dextrose and glycerol solutions can also be employed as liquid carriers, particularly for injectable solutions. Suitable pharmaceutical carriers also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present compositions, if desired, can also contain minor amounts of wetting or emulsifying agents, or pH buffering agents.

The pharmaceutical composition of the invention can be in the form of a liquid, e.g., a solution, emulsion or suspension. The liquid can be useful for delivery by injection, infusion (e.g., IV infusion) or sub-cutaneously. When intended for oral administration, the composition is preferably in solid or liquid form, where semi-solid, semi-liquid, suspension and gel forms are included within the forms considered herein as either solid or liquid.

As a solid composition for oral administration, the composition can be formulated into a powder, granule, compressed tablet, pill, capsule, chewing gum, wafer or the like form. Such a solid composition typically contains one or more inert diluents. In addition, one or more of the following can be present: binders such as carboxymethylcellulose, ethyl cellulose, microcrystalline cellulose, or gelatin; excipients such as starch, lactose or dextrins, disintegrating agents such as alginic acid, sodium alginate, corn starch and the like; lubricants such as magnesium stearate; glidants such as colloidal silicon dioxide; sweetening agents such as sucrose or saccharin; a flavoring agent such as peppermint, methyl salicylate or orange flavoring; and a coloring agent. When the composition is in the form of a capsule (e. g. a gelatin capsule), it can contain, in addition to materials of the above type, a liquid carrier such as polyethylene glycol, cyclodextrin or a fatty oil.

The composition can be in the form of a liquid, e. g. an elixir, syrup, solution, emulsion or suspension. The liquid can be useful for oral administration or for delivery by injection. When intended for oral administration, a composition can comprise one or more of a sweetening agent, preservatives, dye/colorant and flavor enhancer. In a composition for administration by injection, one or more of a surfactant, preservative, wetting agent, dispersing agent, suspending agent, buffer, stabilizer and isotonic agent can also be included.

Compositions can take the form of one or more dosage units. In specific embodiments, it can be desirable to administer the composition locally to the area in need of treatment, or by intravenous injection or infusion.

In another aspect of the invention a method for of treating a disease in a canine subject in need thereof is provided, the method comprising administering an effective amount of the antibody or antigen-binding portion thereof of any previous embodiment of the invention. The method may be used in the treatment of disease. In one embodiment the disease is a cancer or tumour.

In another aspect, the invention relates to an antibody or antigen binding portion thereof as described herein for use in treating a disease in a canine subject. In one embodiment the disease is a cancer or tumour.

In another aspect, the invention relates to the use of an antibody or antigen binding portion thereof as described herein in the manufacture of a medicament for the treatment of a cancer or tumour.

Cancers that may be treated by the antibody or antigen-binding portion thereof, the pharmaceutical composition and/or the method according to the invention are selected from: bone cancer, pancreatic cancer, skin cancer, cancer of the head or neck, cutaneous or intraocular malignant melanoma, oral melanoma, uterine cancer, ovarian cancer, rectal cancer, cancer of the anal region, stomach cancer, testicular cancer, breast cancer, brain cancer, carcinoma of the fallopian tubes, carcinoma of the endometrium, carcinoma of the cervix, carcinoma of the vagina, carcinoma of the vulva, cancer of the esophagus, cancer of the small intestine, cancer of the endocrine system, cancer of the thyroid gland, cancer of the parathyroid gland, cancer of the adrenal gland, kidney cancer, sarcoma of soft tissue, cancer of the urethra, cancer of the bladder, renal cancer, lung cancer, non-small cell lung cancer, thymoma, urothelial carcinoma leukemia, prostate cancer, mesothelioma, adrenocortical carcinoma, lymphomas, such as such as Hodgkin's disease, non-Hodgkin's, gastric cancer, and multiple myelomas. Canine cancers that may be targets for the antibody or antigen-binding portion thereof, the pharmaceutical composition and/or the method according to the invention are selected from: mammary carcinoma (cancer of tissues lining internal organs), osteosarcoma (bone cancer), melanoma (skin cancer) and hemangiosarcoma (cancer of the blood vessel lining).

In a further related embodiment of the invention the antibody or antigen-binding portion thereof, pharmaceutical composition or method of the previous embodiments is provided wherein these embodiments further comprises separately administering another therapeutic agent to the subject. In a related embodiment the other therapeutic agent is a radiotoxic agent, an immunosuppressant, an immunological modulating agent, or an antibody or antibody fragment thereof. In related embodiments the immunological modulating agent is a cytokine, a chemokine or an anti-cancer therapy. A therapeutic agent is a compound or molecule which is useful in the treatment of a disease. Examples of therapeutic agents include antibodies, antibody fragments, drugs, toxins, nucleases, hormones, immunomodulators, pro-apoptotic agents, anti-angiogenic agents, boron compounds, photoactive agents or dyes and radioisotopes. An antibody molecule includes a full antibody or fragment thereof (e.g., a Fab, F(ab′)2, Fv, a single chain Fv fragment (scFv) or a single domain antibody, for example a VH domain, or antibody mimetic protein. The anti-cancer therapy may include a therapeutic agent or radiation therapy and includes gene therapy, viral therapy, RNA therapy bone marrow transplantation, nanotherapy, targeted anti-cancer therapies or oncolytic drugs. Examples of other therapeutic agents include other checkpoint inhibitors, antineoplastic agents, immunogenic agents, attenuated cancerous cells, tumor antigens, antigen presenting cells such as dendritic cells pulsed with tumor-derived antigen or nucleic acids, immune stimulating cytokines (e.g., IL-2, IFNa2, GM-CSF), targeted small molecules and biological molecules (such as components of signal transduction pathways, e.g. modulators of tyrosine kinases and inhibitors of receptor tyrosine kinases, and agents that bind to tumor-specific antigens, including EGFR antagonists), an anti-inflammatory agent, a cytotoxic agent, a radiotoxic agent, or an immunosuppressive agent and cells transfected with a gene encoding an immune stimulating cytokine (e.g., GM-CSF), chemotherapy. In one embodiment, the antibody is used in combination with surgery.

In one embodiment, the antibody or pharmaceutical composition of the invention is administered together with an immunomodulator, a checkpoint modulator, an agent involved in T-cell activation, a tumor microenvironment modifier (TME) or a tumour-specific target. For example, the immunomodulator can be an inhibitor of an immune checkpoint molecule selected from an inhibitor of one or more of PD-1, PD-L1, PD-L2, CTLA-4, TIM-3, LAG-3, CEACAM, VISTA, BTLA, TIGIT, LAIR1, CD160, 2B4 or TGFR beta, OX40, OX40L, CD2, CD27, CDS, ICAM-1, LFA-1 (CD11a/CD18), ICOS (CD278), 4-1BB (CD137), GITR, CD30, CD40, BAFFR, HVEM, CD7, LIGHT, NKG2C, SLAMF7, NKp80, CD160, B7-H3 or CD83 ligand, CD3, CD8, CD28, CD4 or ICAM-1. In another embodiment, the immunomodulator can be an activator of a costimulatory molecule selected from an agonist of one or more of LAG-3, OX40, OX40L, CD2, CD27, CDS, ICAM-1, LFA-1 (CD11a/CD18), ICOS (CD278), 4-1BB (CD137), GITR, CD30, CD40, BAFFR, HVEM, CD7, LIGHT, NKG2C, SLAMF7, NKp80, CD160, B7-H3 or CD83 ligand, CD3, CD8, CD28, CD4 or ICAM-1.

In one embodiment, the PD-1 inhibitor is an anti-PD-1 antibody chosen from Nivolumab®, Pembrolizumab®, Pidilizumab® or Gilvetmab®.

In one embodiment of the present invention, the composition is administered concurrently with a chemotherapeutic agent or with radiation therapy. In another specific embodiment, the chemotherapeutic agent or radiation therapy is administered prior or subsequent to administration of the composition of the present invention, preferably at least an hour, five hours, 12 hours, a day, a week, a month, more preferably several months (e. g. up to three months), prior or subsequent to administration of composition of the present invention.

The antibody or antigen-binding portion thereof or pharmaceutical composition of the invention may be administered at the same time or at a different time as the other therapy or therapeutic compound or therapy, e.g., simultaneously, separately or sequentially. In one embodiment, the other therapy may be any additional PD-1, PD-L1 or PD-L2 therapy.

The amount of the therapeutic that is effective/active in the treatment of a particular disorder or condition will depend on the nature of the disorder or condition and can be determined by standard clinical techniques. In addition, in vitro or in vivo assays can optionally be employed to help identify optimal dosage ranges. The precise dose to be employed in the compositions will also depend on the route of administration, and the seriousness of the disease or disorder, and should be decided according to the judgment of the practitioner and each patient's circumstances. Factors like age, body weight, sex, diet, time of administration, rate of excretion, condition of the host, drug combinations, reaction sensitivities and severity of the disease shall be taken into account.

Typically, the amount is at least about 0.01% of an antibody, antigen binding domain or fragment thereof of the present invention by weight of the composition. When intended for oral administration, this amount can be varied to range from about 0.1% to about 80% by weight of the composition. Preferred oral compositions can comprise from about 4% to about 50% of the antibody or fragment thereof of the present invention by weight of the composition.

In an embodiment the antibody or antigen binding portion thereof is provided at a dose of 0.1 to 50 mg/kg, 0.1 to 40 mg/kg, 0.1 to 30 mg/kg, 0.1 to 20 mg/kg, 0.1 to 10 mg/kg, 0.1 to 5 mg/kg, 0.2 to 50 mg/kg, 0.3 to 50 mg/kg, 0.4 to 50 mg/kg, 0.5 to 50 mg/kg, 0.6 to 50 mg/kg, 0.2 to 40 mg/kg, 0.3 to 40 mg/kg, 0.4 to 40 mg/kg, 0.5 to 40 mg/kg, 0.6 to 40 mg/kg, 0.2 to 30 mg/kg, 0.3 to 30 mg/kg, 0.4 to 30 mg/kg, 0.5 to 30 mg/kg, 0.6 to 30 mg/kg, 0.2 to 20 mg/kg, 0.3 to 20 mg/kg, 0.4 to 20 mg/kg, 0.5 to 20 mg/kg, 0.6 to 20 mg/kg, 0.2 to 10 mg/kg, 0.3 to 10 mg/kg, 0.4 to 10 mg/kg, 0.5 to 10 mg/kg, 0.6 to 10 mg/kg. In some embodiments the antibody or antigen binding portion thereof is provided at a dose of approximately 3 mg/kg. In some embodiments the antibody or antigen binding portion thereof is provided at a dose of approximately 0.6 mg/kg.

Preferred compositions of the present invention are prepared so that a parenteral dosage unit contains from about 0.01% to about 2% by weight of the antibody, antigen binding domain or fragment thereof of the present invention.

For administration by injection, such as intravenous or sub-cutaneous injection, the composition can comprise from about typically about 0.01 mg/kg to about 250 mg/kg, for example 0.1 mg/kg to about 250 mg/kg of the subject's body weight, for example, between about 0.1 mg/kg and about 20 mg/kg of the animal's body weight, and more preferably about 1 mg/kg to about 10 mg/kg of the animal's body weight, although less than 0.1 mg/kg is also envisaged. In one embodiment, the composition is administered at a dose of about 0.5 to 30 mg/kg, e.g., about 5 to 25 mg/kg, about 10 to 20 mg/kg, about 0.5 to 5 mg/kg, about 0.5 to 2.5 mg/kg, about 0.5 to 2.0 mg/kg or about 2 or 3 mg/kg. In one embodiment, the composition is administered at a dose of 2 to 50 mg/ml. In one embodiment, the composition is administered at a dose of 0.5 mg/ml to 2.5 mg/ml, or 0.5 mg/ml to 5 mg/ml. The dosing schedule can vary from e.g., once a week to once every 2, 3, 4 weeks, or up to 8 weeks between doses. In one embodiment, the composition is administered at a dose of 0.5 mg/ml to 2.5 mg/ml every three to four weeks, e.g. 0.5 mg/ml or 2.5 mg/ml every three to four weeks. In some embodiments the dose is chosen so as to give prolonged depletion of PD-1 positive cells to allow a three to four week interval between doses. Multiple doses may be administered, suitably up to about 6 or more repeat doses.

In one embodiment, post-treatment, the subject has at least 7 days, or at least 14 days, or at least 21 days, or at least 28 days, or at least 40 days, or at least 50 days, or at least 60 days disease progression-free. In one embodiment, post-treatment, the subject has at least 7 days, or at least 14 days, or at least 21 days, or at least 28 days, or at least 40 days, or at least 50 days, or at least 60 days disease progression-free.

In one embodiment, the number of days of survival, the number of disease-free days, or the number of disease-progression free days is at least 2 months, or at least 3 months, or at least 4 months, e.g. at least 5 months, such as at least 6 months.

In one embodiment, the number of days of survival, the number of disease-free days, or the number of disease-progression free days is at least 9 months, 200 days, 300 days or 3 years or more. In one embodiment, it is least one, two, three or more years. The invention provides methods of treating or preventing PD-1-mediated diseases or disorders in a companion animal, e.g., a dog, comprising administering an effective amount of an antibody, antigen binding domain or fragment of the present invention to the animal in need thereof.

As used herein, “treat”, “treating” or “treatment” means inhibiting or relieving a disease or disorder. For example, treatment can include a postponement of development of the symptoms associated with a disease or disorder, and/or a reduction in the severity of such symptoms that will, or are expected, to develop with said disease. The terms include ameliorating existing symptoms, preventing additional symptoms, and ameliorating or preventing the underlying causes of such symptoms. Thus, the terms denote that a beneficial result is being conferred on at least some of the mammals, e.g., canine patients, being treated. Many medical treatments are effective for some, but not all, patients that undergo the treatment. In treatment of B cell lymphomas, for example, ameliorating symptoms can be assessed by measuring lymph nodes after treatment and observing a reduction in lymph node size as an indication of successful treatment.

The term “subject” or “patient” refers to a dog, which is the object of treatment, observation, or experiment. For the avoidance of doubt, the treatment of humans is excluded.

In another aspect the invention provides a nucleic acid sequence that encodes an antibody or antibody antigen-binding portion thereof according to any previous embodiment of the invention. Such a DNA sequence may be selected from: SEQ ID NOs. 3 and 5, or SEQ ID NOs. 13 and 15, or SEQ ID NOs. 23 and 25, or SEQ ID NOs 33 and 35, or SEQ ID NOs. 43 and 45, or SEQ ID NOs. 53 and 55, or SEQ ID NOs. 63 and 65, or SEQ ID NOs. 73 and 75, or SEQ ID NOs. 83 and 85, or SEQ ID NOs. 93 and 95, or SEQ ID NOs. 103 and 105, or SEQ ID NOs. 113 and 115, or SEQ ID NOs. 123 and 125, or SEQ ID NOs. 133 and 135, or SEQ ID NOs. 143 and 145, or SEQ ID NOs. 153 and 155, or SEQ ID NOs. 163 and 165, or SEQ ID NOs. 173 and 175, or SEQ ID NOs. 183 and 185, or 193 and 195, or 203 and 205, or SEQ ID NOs. 213 and 215, or SEQ ID NOs. 223 and 225, or SEQ ID NOs. 233 and 235, or SEQ ID NOs. 243 and 245, or SEQ ID NOs. 253 and 255, or SEQ ID NOs. 263 and 265, or SEQ ID NOs. 273 and 275, or SEQ ID NOs. 283 and 285 or a sequence with at least 75%, 80%, 90% or 95% sequence identity thereto.

In another aspect the invention provides a vector comprising a nucleic acid sequence as described above.

In another aspect the invention provides a host cell comprising the nucleic acid according to any previous embodiment or a vector as described above.

In another aspect, the invention provides a kit comprising an antibody or antigen-binding portion thereof according to any previous embodiment or a pharmaceutical composition according to a previous embodiment. In a related embodiment the kit further comprises a reagent for the detection of the antibody or antigen-binding portion thereof as described above.

In another aspect the invention provides a kit for detecting PD-1 for diagnosis, prognosis or monitoring disease comprising an antibody or antigen-binding portion thereof of the invention. Such a kit may contain other components, packaging, instructions, or material to aid in the detection of PD-1 protein. The kit may include a labeled antibody or antigen-binding portion thereof of the invention as described above and one or more compounds for detecting the label.

The invention in another aspect provides an antibody or antigen-binding portion thereof of the invention packaged in lyophilized form, or packaged in an aqueous medium.

In another aspect t the invention provides a method for making a canine antibody that binds PD-1 comprising culturing the isolated host cell of the invention and recovering said antibody.

In a further aspect the invention provides a method for making a canine antibody that binds PD-1 comprising the steps of
a) immunising a transgenic mouse that expresses a nucleic acid construct comprising canine heavy chain V genes and canine light chain V genes with PD-1 antigen,
b) generating a library of antibodies from said mouse and
c) isolating an antibody from said library.

In another further embodiment the invention provides a method for detecting a PD-1 protein or an extracellular domain of a PD-1 protein in a biological sample from a canine subject, comprising contacting a biological sample with the antibody or antigen-binding portion thereof of the invention wherein said antibody or antigen-binding portion thereof is linked to a detectable label. The biological sample is one or more of a biopsy, tissue, blood, serum, plasma, or lymphatic fluid sample. The method may be carried out in vivo, in vitro or ex vivo.

In another aspect the invention provides a method of inhibiting tumor growth or metastasis comprising contacting a tumor cell with an effective amount of the antibody or antigen-binding portion thereof according to the invention or pharmaceutical composition according to the invention.

In a yet further aspect the invention provides a method of killing a tumor cell expressing PD-1, comprising contacting the cell with the antibody or antigen-binding portion thereof of the invention or pharmaceutical composition according to the invention, such that killing of the cell expressing PD-1 occurs.

In one embodiment the tumor cell is a canine tumor cell.

In a further embodiment the invention provides a binding agent comprising the antibody or antigen-binding portion thereof according to the invention wherein said antibody or antigen-binding portion thereof is linked to a second antibody or antigen-binding portion thereof that binds to a second target. In yet a further embodiment the binding agent is selected from any one of OX40L, CD2, CD27, CDS, ICAM-1, LFA-1 (CD11a/CD18), ICOS (CD278), 4-1BB (CD137), GITR, CD30, CD40, BAFFR, HVEM, CD7, LIGHT, NKG2C, SLAMF7, NKp80, CD160, B7-H3 or CD83 ligand, CD3, CD8, CD28, CD4 or ICAM-1.

The invention also relates to the following non-limiting clauses:

1. An isolated canine antibody or antigen-binding portion thereof which binds to one of the following epitopes of the extracellular domain of canine PD-1:
i) an epitope comprising T76, Q83, E84, L128, P129, P130 according to SEQ ID NO: 2
ii) an epitope comprising D58, D61, N74, T76, Y127 according to SEQ ID NO: 2
   wherein the epitope is determined using an epitope mapping technique.

2. The antibody or antigen binding portion thereof according to clause 1, wherein said antibody comprises
a) an HC CDR1 sequence comprising or consisting of SEQ ID NO: 17 or an amino acid sequence with at least 70%, 80%, 85%, 90% or 95% sequence identity to SEQ ID NO: 17 and an HC CDR2 sequence comprising or consisting of SEQ ID NO: 18 or an amino acid sequence with at least 70%, 80%, 85%, 90% or 95% sequence identity to SEQ ID NO: 18 and an HC CDR3 sequence comprising or consisting of SEQ ID NO: 19 or an amino acid sequence with at least 70%, 80%, 85%, 90% or 95% sequence identity to SEQ ID NO: 19 and an LC CDR1 sequence comprising or consisting of SEQ ID NO: 20 or an amino acid sequence with at least 70%, 80%, 85%, 90% or 95% sequence identity to SEQ ID NO: 20 and an LC CDR2 sequence comprising or consisting of SEQ ID NO: 21 or an amino acid sequence with at least 70%, 80%, 85%, 90% or 95% sequence identity to SEQ ID NO: 21 and an LC CDR3 sequence comprising or consisting of SEQ ID NO: 22 or an amino acid sequence with at least 70%, 80%, 85%, 90% or 95% sequence identity to SEQ ID NO: 22; or
b) an HC CDR1 sequence comprising or consisting of SEQ ID NO: 27 or an amino acid sequence with at least 70%, 80%, 85%, 90% or 95% sequence identity to SEQ ID NO: 27 and an HC CDR2 sequence comprising or consisting of SEQ ID NO: 28 or an amino acid sequence with at least 70%, 80%, 85%, 90% or 95% sequence identity to SEQ ID NO: 28 and an HC CDR3 sequence comprising or consisting of SEQ ID NO: 29 or an amino acid sequence with at least 70%, 80%, 85%, 90% or 95% sequence identity to SEQ ID NO: 29 and an LC CDR1 sequence comprising or consisting of SEQ ID NO: 30 or an amino acid sequence with at least 70%, 80%, 85%, 90% or 95% sequence identity to SEQ ID NO: 30 and an LC CDR2 sequence comprising or consisting of SEQ ID NO: 31 or an amino acid sequence with at least 70%, 80%, 85%, 90% or 95% sequence identity to SEQ ID NO: 31 and an LC CDR3 sequence comprising or consisting of SEQ ID NO: 32 or an amino acid sequence with at least 70%, 80%, 85%, 90% or 95% sequence identity to SEQ ID NO: 32; or
c) an HC CDR1 sequence comprising or consisting of SEQ ID NO: 207 or an amino acid sequence with at least 70%, 80%, 85%, 90% or 95% sequence identity to SEQ ID NO: 207 and an HC CDR2 sequence comprising or consisting of SEQ ID NO: 208 or an amino acid sequence with at least 70%, 80%, 85%, 90% or 95% sequence identity to SEQ ID NO: 208 and an HC CDR3 sequence comprising or consisting of SEQ ID NO: 209 or an amino acid sequence w with at least 70%, 80%, 85%, 90% or 95% sequence identity to SEQ ID NO: 209 and an LC CDR1 sequence comprising or consisting of SEQ ID NO: 210 or an amino acid sequence with at least 70%, 80%, 85%, 90% or 95% sequence identity to SEQ ID NO: 210 and an LC CDR2 sequence comprising or consisting of SEQ ID NO: 211 or an amino acid sequence with at least 70%, 80%, 85%, 90% or 95% sequence identity to SEQ ID NO: 211 and an LC CDR3 sequence comprising or consisting of SEQ ID NO: 212 or an amino acid sequence with at least 70%, 80%, 85%, 90% or 95% sequence identity to SEQ ID NO: 212; or
d) an HC CDR1 sequence comprising or consisting of SEQ ID NO: 97 or an amino acid sequence with at least 70%, 80%, 85%, 90% or 95% sequence identity to SEQ ID NO: 97 and an HC CDR2 sequence comprising or consisting of SEQ ID NO: 98 or an amino acid sequence with at least 70%, 80%, 85%, 90% or 95% sequence identity to SEQ ID NO: 98 and an HC CDR3 sequence comprising or consisting of SEQ ID NO: 99 or an amino acid sequence with at least 70%, 80%, 85%, 90% or 95% sequence identity to SEQ ID NO: 99 and an LC CDR1 sequence comprising or consisting of SEQ ID NO: 100 or an amino acid sequence with at least 70%, 80%, 85%, 90% or 95% sequence identity to SEQ ID NO: 100 and an LC CDR2 sequence comprising or consisting of SEQ ID NO: 101 or an amino acid sequence with at least 70%, 80%, 85%, 90% or 95% sequence identity to SEQ ID NO: 101 and an LC CDR3 sequence comprising or consisting of SEQ ID NO: 102 or an amino acid sequence with at least 70%, 80%, 85%, 90% or 95% sequence identity to SEQ ID NO: 102.

3. The antibody or antigen-binding portion thereof according to clause 1 or 2, wherein said antibody comprises
a) an HC CDR1 sequence comprising or consisting of SEQ ID NO: 17 or an amino acid sequence which has 1, 2, 3, 4, or 5 amino acid differences compared to SEQ ID NO: 17 and an HC CDR2 sequence comprising or consisting of SEQ ID NO: 18 or an amino acid sequence which has 1, 2, 3, 4, 5, or 6 amino acid differences compared to SEQ ID NO: 18 and an HC CDR3 sequence comprising or consisting of SEQ ID NO: 19 or an amino acid sequence which has 1, 2, 3, 4, 5, 6, 7, 8, or 9 amino acid differences compared to SEQ ID NO: 19 and an LC CDR1 sequence comprising or consisting of SEQ ID NO: 20 or an amino acid sequence which has 1, 2, 3, 4, 5, 6, 7, 8, 9, 10, 11, 12, 13 or 14 amino acid differences compared to SEQ ID NO: 20 and an LC CDR2 sequence comprising or consisting of SEQ ID NO: 21 or an amino acid sequence which has 1, 2, 3, 4, or 5 amino acid differences compared to SEQ ID NO: 21 and an LC CDR3 sequence comprising or consisting of SEQ ID NO: 22 or an amino acid sequence which has 1, 2, 3, 4, 5, 6, 7, 8, 9, or 10 amino acid differences compared to SEQ ID NO: 22; or
b) an HC CDR1 sequence comprising or consisting of SEQ ID NO: 207 or an amino acid sequence which has 1, or 2 amino acid differences compared to SEQ ID NO: 207 and an HC CDR2 sequence comprising or consisting of SEQ ID NO: 208 or an amino acid sequence which has 1, 2, 3, 4, or 5 amino acid differences compared to SEQ ID NO: 208 and an HC CDR3 sequence comprising or consisting of SEQ ID NO: 209 or an amino acid sequence which has 1, 2, 3, 4, 5, 6, 7, 8, 9, 10, 11, 12, 13, 14, 15, or 16 amino acid differences compared to SEQ ID NO: 209 and an LC CDR1 sequence comprising or consisting of SEQ ID NO: 210 or an amino acid sequence which has 1, 2 or 3 amino acid differences compared to SEQ ID NO: 210 and an LC CDR2 sequence comprising or consisting of SEQ ID NO: 211 or an amino acid sequence which has 1 or 2 amino acid differences compared to SEQ ID NO: 211 and an LC CDR3 sequence comprising or consisting of SEQ ID NO: 212 or an amino acid sequence which has 1, 2, 3, or 4 amino acid differences compared to SEQ ID NO: 212; or
c) an HC CDR1 sequence comprising or consisting of SEQ ID NO: 97 or an amino acid sequence which has 1, 2, or 3 amino acid differences compared to SEQ ID NO: 97 and an HC CDR2 sequence comprising or consisting of SEQ ID NO: 98 or an amino acid sequence which has 1, 2, 3, or 4 amino acid differences compared to SEQ ID NO: 98 and an HC CDR3 sequence comprising or consisting of SEQ ID NO: 99 or an amino acid sequence which has 1, 2, 3, 4, 5, 6, 7, 8, 9, 10, 11, 12, 13, or 14 amino acid differences compared to SEQ ID NO: 99 and an LC CDR1 sequence comprising or consisting of SEQ ID NO: 100 or an amino acid sequence which has 1, 2, 3, 4, 5, 6, 7, 8, 9, 10, 11, 12, 13, 14 or 15 amino acid differences compared to SEQ ID NO: 100 and an LC CDR2 sequence comprising or consisting of SEQ ID NO: 101 or an amino acid sequence which has 1, 2, 3, 4, or 5 amino acid differences compared to SEQ ID NO: 101 and an LC CDR3 sequence comprising or consisting of SEQ ID NO: 102 or an amino acid sequence which has 1, 2, 3, 4, 5, 6, 7, 8, 9, or 10 amino acid differences compared to SEQ ID NO: 102.

4. An isolated canine antibody or antigen-binding portion thereof which binds canine PD-1 wherein said antibody comprises
a) an HC CDR1 sequence comprising or consisting of SEQ ID NO: 17 or an amino acid sequence which has 1, 2, 3, 4, or 5 amino acid differences compared to SEQ ID NO: 17 and an HC CDR2 sequence comprising or consisting of SEQ ID NO: 18 or an amino acid sequence which has 1, 2, 3, 4, 5, or 6 amino acid differences compared to SEQ ID NO: 18 and an HC CDR3 sequence comprising or consisting of SEQ ID NO: 19 or an amino acid sequence which has 1, 2, 3, 4, 5, 6, 7, 8, or 9 amino acid differences compared to SEQ ID NO: 19 and an LC CDR1 sequence comprising or consisting of SEQ ID NO: 20 or an amino acid sequence which has 1, 2, 3, 4, 5, 6, 7, 8, 9, 10, 11, 12, 13 or 14 amino acid differences compared to SEQ ID NO: 20 and an LC CDR2 sequence comprising or consisting of SEQ ID NO: 21 or an amino acid sequence which has 1, 2, 3, 4, or 5 amino acid differences compared to SEQ ID NO: 21 and an LC CDR3 sequence comprising or consisting of SEQ ID NO: 22 or an amino acid sequence which has 1, 2, 3, 4, 5, 6, 7, 8, 9, or 10 amino acid differences compared to SEQ ID NO: 22; or
b) an HC CDR1 sequence comprising or consisting of SEQ ID NO: 207 or an amino acid sequence which has 1, or 2 amino acid differences compared to SEQ ID NO: 207 and an HC CDR2 sequence comprising or consisting of SEQ ID NO: 208 or an amino acid sequence which has 1, 2, 3, 4, or 5 amino acid differences compared to SEQ ID NO: 208 and an HC CDR3 sequence comprising or consisting of SEQ ID NO: 209 or an amino acid sequence which has 1, 2, 3, 4, 5, 6, 7, 8, 9, 10, 11, 12, 13, 14, 15, or 16 amino acid differences compared to SEQ ID NO: 209 and an LC CDR1 sequence comprising or consisting of SEQ ID NO: 210 or an amino acid sequence which has 1, 2 or 3 amino acid differences compared to SEQ ID NO: 210 and an LC CDR2 sequence comprising or consisting of SEQ ID NO: 211 or an amino acid sequence which has 1 or 2 amino acid differences compared to SEQ ID NO: 211 and an LC CDR3 sequence comprising or consisting of SEQ ID NO: 212 or an amino acid sequence which has 1, 2, 3, or 4 amino acid differences compared to SEQ ID NO: 212; or
c) an HC CDR1 sequence comprising or consisting of SEQ ID NO: 97 or an amino acid sequence which has 1, 2, or 3 amino acid differences compared to SEQ ID NO: 97 and an HC CDR2 sequence comprising or consisting of SEQ ID NO: 98 or an amino acid sequence which has 1, 2, 3, or 4 amino acid differences compared to SEQ ID NO: 98 and an HC CDR3 sequence comprising or consisting of SEQ ID NO: 99 or an amino acid sequence which has 1, 2, 3, 4, 5, 6, 7, 8, 9, 10, 11, 12, 13, or 14 amino acid differences compared to SEQ ID NO: 99 and an LC CDR1 sequence comprising or consisting of SEQ ID NO: 100 or an amino acid sequence which has 1, 2, 3, 4, 5, 6, 7, 8, 9, 10, 11, 12, 13, 14 or 15 amino acid differences compared to SEQ ID NO: 100 and an LC CDR2 sequence comprising or consisting of SEQ ID NO: 101 or an amino acid sequence which has 1, 2, 3, 4, or 5 amino acid differences compared to SEQ ID NO: 101 and an LC CDR3 sequence comprising or consisting of SEQ ID NO: 102 or an amino acid sequence which has 1, 2, 3, 4, 5, 6, 7, 8, 9, or 10 amino acid differences compared to SEQ ID NO: 102.

5. The antibody or antigen-binding portion thereof according to any preceding clause wherein said antibody or antigen-binding portion thereof has
i) a HC CDR1 sequence comprising or consisting of SEQ ID NO: 17, a HC CDR2 sequence comprising or consisting of SEQ ID NO: 18, a HC CDR3 sequence comprising or consisting of SEQ ID NO: 19, a LC CDR1 sequence comprising or consisting of SEQ ID NO: 20, a LC CDR2 sequence comprising or consisting of SEQ ID NO: 21 and a LC CDR3 sequence comprising or consisting of SEQ ID NO: 22,
ii) a HC CDR1 sequence comprising or consisting of SEQ ID NO: 27, a HC CDR2 sequence comprising or consisting of SEQ ID NO: 28, a HC CDR3 sequence comprising or consisting of SEQ ID NO: 29, a LC CDR1 sequence comprising or consisting of SEQ ID NO: 30, a LC CDR2 sequence comprising or consisting of SEQ ID NO: 31 and a LC CDR3 sequence comprising or consisting of SEQ ID NO: 32
iii) a HC CDR1 sequence comprising or consisting of SEQ ID NO: 7, a HC CDR2 sequence comprising or consisting of SEQ ID NO: 8, a HC CDR3 sequence comprising or consisting of SEQ ID NO: 9, a LC CDR1 sequence comprising or consisting of SEQ ID NO: 10, a LC CDR2 sequence comprising or consisting of SEQ ID NO: 11 and a LC CDR3 sequence comprising or consisting of SEQ ID NO: 12,
iv) a HC CDR1 sequence comprising or consisting of SEQ ID NO: 37, a HC CDR2 sequence comprising or consisting of SEQ ID NO: 38, a HC CDR3 sequence comprising or consisting of SEQ ID NO: 39, a LC CDR1 sequence comprising or consisting of SEQ ID NO: 40, a LC CDR2 sequence comprising or consisting of SEQ ID NO: 41 and a LC CDR3 sequence comprising or consisting of SEQ ID NO: 42
v) a HC CDR1 sequence comprising or consisting of SEQ ID NO: 47, a HC CDR2 sequence comprising or consisting of SEQ ID NO: 48, a HC CDR3 sequence comprising or consisting of SEQ ID NO: 49, a LC CDR1 sequence comprising SEQ ID NO: 50, a LC CDR2 sequence comprising or consisting of SEQ ID NO: 51 and a LC CDR3 sequence comprising or consisting of SEQ ID NO: 52,
vi) a HC CDR1 sequence comprising SEQ ID NO: 57, a HC CDR2 sequence comprising SEQ ID NO: 58, a HC CDR3 sequence comprising SEQ ID NO: 59, a LC CDR1 sequence comprising SEQ ID NO: 60, a LC CDR2 sequence comprising SEQ ID NO: 61 and a LC CDR3 sequence comprising SEQ ID NO: 62,
vii) a HC CDR1 sequence comprising SEQ ID NO: 67, a HC CDR2 sequence comprising SEQ ID NO: 68, a HC CDR3 sequence comprising SEQ ID NO: 69, a LC CDR1 sequence comprising SEQ ID NO: 70, a LC CDR2 sequence comprising SEQ ID NO: 71 and a LC CDR3 sequence comprising SEQ ID NO: 72,
viii) a HC CDR1 sequence comprising SEQ ID NO: 77, a HC CDR2 sequence comprising SEQ ID NO: 78, a HC CDR3 sequence comprising SEQ ID NO: 79, a LC CDR1 sequence comprising SEQ ID NO: 80, a LC CDR2 sequence comprising SEQ ID NO: 81 and a LC CDR3 sequence comprising SEQ ID NO: 82,
ix) a HC CDR1 sequence comprising or consisting of SEQ ID NO: 87, a HC CDR2 sequence comprising or consisting of SEQ ID NO: 88, a HC CDR3 sequence comprising or consisting of SEQ ID NO: 89, a LC CDR1 sequence comprising or consisting of SEQ ID NO: 90, a LC CDR2 sequence comprising or consisting of SEQ ID NO: 91 and a LC CDR3 sequence comprising or consisting of SEQ ID NO: 92,
x) a HC CDR1 sequence comprising or consisting of SEQ ID NO: 97, a HC CDR2 sequence comprising or consisting of SEQ ID NO: 98, a HC CDR3 sequence comprising or consisting of SEQ ID NO: 99, a LC CDR1 sequence comprising or consisting of SEQ ID NO: 100, a LC CDR2 sequence comprising or consisting of SEQ ID NO: 101 and a LC CDR3 sequence comprising or consisting of SEQ ID NO: 102,
xi) a HC CDR1 sequence comprising or consisting of SEQ ID NO: 107, a HC CDR2 sequence comprising or consisting of SEQ ID NO: 108, a HC CDR3 sequence comprising or consisting of SEQ ID NO: 109, a LC CDR1 sequence comprising or consisting of SEQ ID NO: 110, a LC CDR2 sequence comprising or consisting of SEQ ID NO: 111 and a LC CDR3 sequence comprising or consisting of SEQ ID NO: 112,
xii) a HC CDR1 sequence comprising or consisting of SEQ ID NO: 117, a HC CDR2 sequence comprising or consisting of SEQ ID NO: 118, a HC CDR3 sequence comprising or consisting of SEQ ID NO: 119, a LC CDR1 sequence comprising or consisting of SEQ ID NO: 120, a LC CDR2 sequence comprising or consisting of SEQ ID NO: 12 and a LC CDR3 sequence comprising or consisting of SEQ ID NO: 122,
xiii) a HC CDR1 sequence comprising or consisting of SEQ ID NO: 127, a HC CDR2 sequence comprising or consisting of SEQ ID NO: 128, a HC CDR3 sequence comprising or consisting of SEQ ID NO: 129, a LC CDR1 sequence comprising or consisting of SEQ ID NO: 130, a LC CDR2 sequence comprising or consisting of SEQ ID NO: 131 and a LC CDR3 sequence comprising or consisting of SEQ ID NO: 132,
xiv) a HC CDR1 sequence comprising or consisting of SEQ ID NO: 137, a HC CDR2 sequence comprising or consisting of SEQ ID NO: 138, a HC CDR3 sequence comprising or consisting of SEQ ID NO: 139, a LC CDR1 sequence comprising or consisting of SEQ ID NO: 140, a LC CDR2 sequence comprising or consisting of SEQ ID NO: 141 and a LC CDR3 sequence comprising or consisting of SEQ ID NO: 142,
xv) a HC CDR1 sequence comprising or consisting of SEQ ID NO: 147, a HC CDR2 sequence comprising or consisting of SEQ ID NO: 148, a HC CDR3 sequence comprising or consisting of SEQ ID NO: 149, a LC CDR1 sequence comprising or consisting of SEQ ID NO: 150, a LC CDR2 sequence comprising or consisting of SEQ ID NO: 151 and a LC CDR3 sequence comprising or consisting of SEQ ID NO: 152,
xvi) a HC CDR1 sequence comprising or consisting of SEQ ID NO: 157, a HC CDR2 sequence comprising or consisting of SEQ ID NO: 158, a HC CDR3 sequence comprising or consisting of SEQ ID NO: 159, a LC CDR1 sequence comprising or consisting of SEQ ID NO: 160, a LC CDR2 sequence comprising or consisting of SEQ ID NO: 161 and a LC CDR3 sequence comprising or consisting of SEQ ID NO: 162,
xvii) a HC CDR1 sequence comprising or consisting of SEQ ID NO: 167, a HC CDR2 sequence comprising or consisting of SEQ ID NO: 168, a HC CDR3 sequence comprising or consisting of SEQ ID NO: 169, a LC CDR1 sequence comprising or consisting of SEQ ID NO: 170, a LC CDR2 sequence comprising or consisting of SEQ ID NO: 171 and a LC CDR3 sequence comprising or consisting of SEQ ID NO: 172,
xviii) a HC CDR1 sequence comprising or consisting of SEQ ID NO: 177, a HC CDR2 sequence comprising or consisting of SEQ ID NO: 178, a HC CDR3 sequence comprising or consisting of SEQ ID NO: 179, a LC CDR1 sequence comprising or consisting of SEQ ID NO: 180, a LC CDR2 sequence comprising or consisting of SEQ ID NO: 181 and a LC CDR3 sequence comprising or consisting of SEQ ID NO: 182,
xix) a HC CDR1 sequence comprising or consisting of SEQ ID NO: 187, a HC CDR2 sequence comprising or consisting of SEQ ID NO: 188, a HC CDR3 sequence comprising or consisting of SEQ ID NO: 189, a LC CDR1 sequence comprising or consisting of SEQ ID NO: 190, a LC CDR2 sequence comprising or consisting of SEQ ID NO: 191 and a LC CDR3 sequence comprising or consisting of SEQ ID NO: 192,
xx) a HC CDR1 sequence comprising or consisting of SEQ ID NO: 197, a HC CDR2 sequence comprising or consisting of SEQ ID NO: 198, a HC CDR3 sequence comprising or consisting of SEQ ID NO: 199, a LC CDR1 sequence comprising or consisting of SEQ ID NO: 200, a LC CDR2 sequence comprising or consisting of SEQ ID NO: 201 and a LC CDR3 sequence comprising or consisting of SEQ ID NO: 202,
xxi) a HC CDR1 sequence comprising or consisting of SEQ ID NO: 207, a HC CDR2 sequence comprising or consisting of SEQ ID NO: 208, a HC CDR3 sequence comprising or consisting of SEQ ID NO: 209, a LC CDR1 sequence comprising or consisting of SEQ ID NO: 210, a LC CDR2 sequence comprising or consisting of SEQ ID NO: 211 and a LC CDR3 sequence comprising or consisting of SEQ ID NO: 212,
xxii) a HC CDR1 sequence comprising or consisting of SEQ ID NO: 217, a HC CDR2 sequence comprising or consisting of SEQ ID NO: 218, a HC CDR3 sequence comprising or consisting of SEQ ID NO: 219, a LC CDR1 sequence comprising or consisting of SEQ ID NO: 220, a LC CDR2 sequence comprising or consisting of SEQ ID NO: 221 and a LC CDR3 sequence comprising or consisting of SEQ ID NO: 222,
xxiii) a HC CDR1 sequence comprising or consisting of SEQ ID NO: 227, a HC CDR2 sequence comprising or consisting of SEQ ID NO: 228, a HC CDR3 sequence comprising or consisting of SEQ ID NO: 229, a LC CDR1 sequence comprising or consisting of SEQ ID NO: 230, a LC CDR2 sequence comprising or consisting of SEQ ID NO: 231 and a LC CDR3 sequence comprising or consisting of SEQ ID NO: 232,
xxiv) a HC CDR1 sequence comprising or consisting of SEQ ID NO: 237, a HC CDR2 sequence comprising or consisting of SEQ ID NO: 238, a HC CDR3 sequence comprising or consisting of SEQ ID NO: 239, a LC CDR1 sequence comprising or consisting of SEQ ID NO: 240, a LC CDR2 sequence comprising or consisting of SEQ ID NO: 241 and a LC CDR3 sequence comprising or consisting of SEQ ID NO: 242,
xxv) a HC CDR1 sequence comprising or consisting of SEQ ID NO: 247, a HC CDR2 sequence comprising or consisting of SEQ ID NO: 248, a HC CDR3 sequence comprising or consisting of SEQ ID NO: 249, a LC CDR1 sequence comprising or consisting of SEQ ID NO: 250, a LC CDR2 sequence comprising or consisting of SEQ ID NO: 251 and a LC CDR3 sequence comprising or consisting of SEQ ID NO: 252,
xxvi) a HC CDR1 sequence comprising or consisting of SEQ ID NO: 257, a HC CDR2 sequence comprising or consisting of SEQ ID NO: 258, a HC CDR3 sequence comprising or consisting of SEQ ID NO: 259, a LC CDR1 sequence comprising or consisting of SEQ ID NO: 260, a LC CDR2 sequence comprising or consisting of SEQ ID NO: 261 and a LC CDR3 sequence comprising or consisting of SEQ ID NO: 262,
xxvii) a HC CDR1 sequence comprising or consisting of SEQ ID NO: 267, a HC CDR2 sequence comprising or consisting of SEQ ID NO: 268, a HC CDR3 sequence comprising or consisting of SEQ ID NO: 269, a LC CDR1 sequence comprising or consisting of SEQ ID NO: 270, a LC CDR2 sequence comprising or consisting of SEQ ID NO: 271 and a LC CDR3 sequence comprising or consisting of SEQ ID NO: 271,
xxviii) a HC CDR1 sequence comprising or consisting of SEQ ID NO: 277, a HC CDR2 sequence comprising or consisting of SEQ ID NO: 278, a HC CDR3 sequence comprising or consisting of SEQ ID NO: 279, a LC CDR1 sequence comprising or consisting of SEQ ID NO: 280, a LC CDR2 sequence comprising or consisting of SEQ ID NO: 281 and a LC CDR3 sequence comprising or consisting of SEQ ID NO: 282, or
xxix) a HC CDR1 sequence comprising or consisting of SEQ ID NO: 287, a HC CDR2 sequence comprising or consisting of SEQ ID NO: 288, a HC CDR3 sequence comprising or consisting of SEQ ID NO: 289, a LC CDR1 sequence comprising or consisting of SEQ ID NO: 290, a LC CDR2 sequence comprising or consisting of SEQ ID NO: 291 and a LC CDR3 sequence comprising or consisting of SEQ ID NO: 292.

6. The antibody or antigen-binding portion thereof according to a preceding clause wherein said antibody or antigen-binding portion thereof comprises a HC variable region sequence comprising SEQ ID NO: 4 or a sequence with at least 45%, 50%, 60%, 70%, 75%, 80%, 85% or 90% sequence identity thereto and a LC variable region sequence comprising SEQ ID NO: 6 or a sequence with at least 35%, 40%, 50%, 60%, 70%, 75%, 80%, 85%, 90% or 95% sequence identity thereto for example a LC variable region sequence comprising SEQ ID NO: 6.

7. The antibody or antigen-binding portion thereof according to a preceding clause wherein said antibody or antigen-binding portion thereof has
a) a HC variable region sequence comprising SEQ ID NO: 4 or a sequence with at least 45%, 50%, 70%, 75%, 80%, 85% or 90% sequence identity thereto and a LC variable region sequence comprising SEQ ID NO: 6 or a sequence with at least 35%, 40%, 70%, 75%, 80%, 85% or 90% sequence identity thereto; or
b) a HC variable region sequence comprising SEQ ID NO: 14 or a sequence with at least 40%, 45%, 70%, 75%, 80%, 85% or 90% sequence identity thereto and a LC variable region sequence comprising SEQ ID NO: 16 or a sequence with at least 35%, 45%, 55%, 65%, 70%, 75%, 80%, 85% or 90% sequence identity thereto; or
c) a HC variable region sequence comprising SEQ ID NO: 24 or a sequence with at least 45%, 50%, 70%, 75%, 80%, 85% or 90% sequence identity thereto and a LC variable region sequence comprising SEQ ID NO: 6 or a sequence with at least 35%, 40%, 70%, 75%, 80%, 85% or 90% sequence identity thereto; or
d) a HC variable region sequence comprising SEQ ID NO: 34 or a sequence with at least 45%, 50%, 70%, 75%, 80%, 85% or 90% sequence identity thereto and a LC variable region sequence comprising SEQ ID NO: 6 or a sequence with at least 35%, 40%, 70%, 75%, 80%, 85% or 90% sequence identity thereto; or
e) a HC variable region sequence comprising SEQ ID NO: 44 or a sequence with at least 40%, 50%, 60%, 70%, 75%, 80%, 85% or 90% sequence identity thereto and a LC variable region sequence comprising SEQ ID NO: 46 or a sequence with at least 35%, 45%, 55%, 65%, 70%, 75%, 80%, 85% or 90% sequence identity thereto; or
f) a HC variable region sequence comprising SEQ ID NO: 54 or a sequence with at least 40%, 45%, 70%, 75%, 80%, 85% or 90% sequence identity thereto and a LC variable region sequence comprising SEQ ID NO: 56 or a sequence with at least 35%, 40%, 70%, 75%, 80%, 85% or 90% sequence identity thereto; or
g) a HC variable region sequence comprising SEQ ID NO: 64 or a sequence with at least 45%, 65%, 70%, 75%, 80%, 85% or 90% sequence identity thereto and a LC variable region sequence comprising SEQ ID NO: 36 or a sequence with at least 35%, 40%, 70%, 75%, 80%, 85% or 90% sequence identity thereto; or
h) a HC variable region sequence comprising SEQ ID NO: 74 or a sequence with at least 45%, 50%, 70%, 75%, 80%, 85% or 90% sequence identity thereto and a LC variable region sequence comprising SEQ ID NO: 6 or a sequence with at least 35%, 40%, 70%, 75%, 80%, 85% or 90% sequence identity thereto; or
i) a HC variable region sequence comprising SEQ ID NO: 84 or a sequence with at least 40%, 45%, 50%, 70%, 75%, 80%, 85% or 90% sequence identity thereto and a LC variable region sequence comprising SEQ ID NO: 86 or a sequence with at least 35%, 40%, 70%, 75%, 80%, 85% or 90% sequence identity thereto; or
j) a HC variable region sequence comprising SEQ ID NO: 94 or a sequence with at least 45%, 50%, 70%, 75%, 80%, 85% or 90% sequence identity thereto and a LC variable region sequence comprising SEQ ID NO: 6 or a sequence with at least 35%, 40%, 70%, 75%, 80%, 85% or 90% sequence identity thereto; or
k) a HC variable region sequence comprising SEQ ID NO: 104 or a sequence with at least 45%, 50%, 70%, 75%, 80%, 85% or 90% sequence identity thereto and a LC variable region sequence comprising SEQ ID NO: 6 or a sequence with at least 35%, 40%, 70%, 75%, 80%, 85% or 90% sequence identity thereto; or
l) a HC variable region sequence comprising SEQ ID NO: 114 or a sequence with at least 45%, 50%, 70%, 75%, 80%, 85% or 90% sequence identity thereto and a LC variable region sequence comprising SEQ ID NO: 6 or a sequence with at least 35%, 40%, 70%, 75%, 80%, 85% or 90% sequence identity thereto; or
m) a HC variable region sequence comprising SEQ ID NO: 124 or a sequence with at least 45%, 50%, 70%, 75%, 80%, 85% or 90% sequence identity thereto and a LC variable region sequence comprising SEQ ID NO: 6 or a sequence with at least 35%, 40%, 70%, 75%, 80%, 85% or 90% sequence identity thereto; or
n) a HC variable region sequence comprising SEQ ID NO: 134 or a sequence with at least 45%, 50%, 70%, 75%, 80%, 85% or 90% sequence identity thereto and a LC variable region sequence comprising SEQ ID NO: 136 or a sequence with at least 35%, 40%, 70%, 75%, 80%, 85% or 90% sequence identity thereto; or
o) a HC variable region sequence comprising SEQ ID NO:144 or a sequence with at least 45%, 50%, 70%, 75%, 80%, 85% or 90% sequence identity thereto and a LC variable region sequence comprising SEQ ID NO: 6 or a sequence with at least 35%, 40%, 70%, 75%, 80%, 85% or 90% sequence identity thereto; or
p) a HC variable region sequence comprising SEQ ID NO: 154 or a sequence with at least 45%, 50%, 70%, 75%, 80%, 85% or 90% sequence identity thereto and a LC variable region sequence comprising SEQ ID NO: 156 or a sequence with at least 35%, 40%, 70%, 75%, 80%, 85% or 90% sequence identity thereto; or
q) a HC variable region sequence comprising SEQ ID NO: 164 or a sequence with at least 45%, 50%, 70%, 75%, 80%, 85% or 90% sequence identity thereto and a LC variable region sequence comprising SEQ ID NO: 176 or a sequence with at least 35%, 40%, 70%, 75%, 80%, 85% or 90% sequence identity thereto; or
r) a HC variable region sequence comprising SEQ ID NO: 174 or a sequence with at least 45%, 50%, 70%, 75%, 80%, 85% or 90% sequence identity thereto and a LC variable region sequence comprising SEQ ID NO: 6 or a sequence with at least 35%, 40%, 70%, 75%, 80%, 85% or 90% sequence identity thereto; or
s) a HC variable region sequence comprising SEQ ID NO: 184 or a sequence with at least 45%, 50%, 70%, 75%, 80%, 85% or 90% sequence identity thereto and a LC variable region sequence comprising SEQ ID NO: 186 or a sequence with at least 35%, 40%, 70%, 75%, 80%, 85% or 90% sequence identity thereto; or
t) a HC variable region sequence comprising SEQ ID NO: 194 or a sequence with at least 45%, 50%, 70%, 75%, 80%, 85% or 90% sequence identity thereto and a LC variable region sequence comprising SEQ ID NO: 6 or a sequence with at least 35%, 40%, 70%, 75%, 80%, 85% or 90% sequence identity thereto; or
u) a HC variable region sequence comprising SEQ ID NO: 204 or a sequence with at least 40%, 45%, 70%, 75%, 80%, 85% or 90% sequence identity thereto and a LC variable region sequence comprising SEQ ID NO: 206 or a sequence with at least 35%, 40%, 70%, 75%, 80%, 85% or 90% sequence identity thereto; or
v) a HC variable region sequence comprising SEQ ID NO: 214 or a sequence with at least 40%, 45%, 70%, 75%, 80%, 85% or 90% sequence identity thereto and a LC variable region sequence comprising SEQ ID NO: 216 or a sequence with at least 35%, 40%, 70%, 75%, 80%, 85% or 90% sequence identity thereto; or
w) a HC variable region sequence comprising SEQ ID NO: 224 or a sequence with at least 40%, 45%, 70%, 75%, 80%, 85% or 90% sequence identity thereto and a LC variable region sequence comprising SEQ ID NO: 226 or a sequence with at least 35%, 40%, 70%, 75%, 80%, 85% or 90% sequence identity thereto; or
x) a HC variable region sequence comprising SEQ ID NO: 234 or a sequence with at least 45%, 50%, 70%, 75%, 80%, 85% or 90% sequence identity thereto and a LC variable region sequence comprising SEQ ID NO: 236 or a sequence with at least 35%, 40%, 70%, 75%, 80%, 85% or 90% sequence identity thereto; or
y) a HC variable region sequence comprising SEQ ID NO: 244 or a sequence with at least 40%, 45%, 70%, 75%, 80%, 85% or 90% sequence identity thereto and a LC variable region sequence comprising SEQ ID NO: 246 or a sequence with at least 35%, 40%, 70%, 75%, 80%, 85% or 90% sequence identity thereto; or
z) a HC variable region sequence comprising SEQ ID NO: 254 or a sequence with at least 40%, 45%, 70%, 75%, 80%, 85% or 90% sequence identity thereto and a LC variable region sequence comprising SEQ ID NO: 256 or a sequence with at least 35%, 40%, 70%, 75%, 80%, 85% or 90% sequence identity thereto; or
aa) a HC variable region sequence comprising SEQ ID NO: 264 or a sequence with at least 40%, 45%, 70%, 75%, 80%, 85% or 90% sequence identity thereto and a LC variable region sequence comprising SEQ ID NO: 266 or a sequence with at least 35%, 40%, 70%, 75%, 80%, 85% or 90% sequence identity thereto; or
bb) a HC variable region sequence comprising SEQ ID NO: 274 or a sequence with at least 45%, 50%, 70%, 75%, 80%, 85% or 90% sequence identity thereto and a LC variable region sequence comprising SEQ ID NO: 276 or a sequence with at least 35%, 40%, 70%, 75%, 80%, 85% or 90% sequence identity thereto; or
cc) a HC variable region sequence comprising SEQ ID NO: 284 or a sequence with at least 45%, 50%, 70%, 75%, 80%, 85% or 90% sequence identity thereto and a LC variable region sequence comprising SEQ ID NO: 286 or a sequence with at least 35%, 40%, 70%, 75%, 80%, 85% or 90% sequence identity thereto, or
dd) a HC variable region sequence comprising SEQ ID NO: 24 or a sequence with at least 45%, 50%, 70%, 75%, 80%, 85% or 90% sequence identity thereto and a LC variable region sequence comprising SEQ ID NO: 6 or a sequence with at least 35%, 40%, 70%, 75%, 80%, 85% or 90% sequence identity thereto.

8. The antibody or antigen-binding portion thereof according to a preceding clause wherein said antigen-binding portion thereof is an scFv, Fv, heavy chain or single domain antibody.

9. An isolated canine antibody or antigen-binding portion thereof according to a preceding clause that binds to canine PD-1 wherein said antibody or antigen-binding portion thereof that competes with PD-L1 and/or PD-L2.

10. The antibody or antigen-binding portion thereof according to a preceding clause wherein said antibody or antigen binding portion thereof blocks the interaction of PD-1 with PD-L1 and/or PD-L2 and/or prevents a cellular response associated with the interaction of PD-1 with PD-L1 and/or PD-L2.

11. The antibody or antigen-binding portion thereof according to a preceding clause wherein said antibody or antigen-binding portion thereof is conjugated to a therapeutic moiety.

12. The antibody or antigen-binding portion thereof according to clause 11 wherein said therapeutic moiety is a second antibody or antigen-binding portion thereof.

13. The antibody or antigen-binding portion thereof according to clause 12 wherein said second antibody or antigen-binding portion thereof binds to a different target.

14. The antibody or antigen-binding portion thereof according to a preceding clause wherein said antibody or antigen-binding portion thereof is conjugated to a further moiety selected from a half-life extending moiety, label, cytotoxin, liposome, nanoparticle or radioisotope.

15. The antibody or antigen-binding portion thereof according to clause 14 wherein the half-life extending moiety is selected from: albumin binding moiety, a transferrin binding moiety, a polyethylene glycol molecule, a recombinant polyethylene glycol molecule, human serum albumin, a fragment of human serum albumin, an albumin binding peptide or single domain antibody that binds to human serum albumin.

16. A pharmaceutical composition comprising an antibody or antigen-binding portion thereof according to a preceding clause.

17. The antibody or antigen-binding portion thereof according to any of clauses 1 to 15 or the pharmaceutical composition according to clause 16 for use in the treatment of disease.

18. A method of treating a disease in a canine subject in need thereof comprising administering an effective amount of the antibody or antigen-binding portion thereof of any one of clauses 1 to 15 or a pharmaceutical composition according to clause 16.

19. The antibody or antigen-binding portion thereof or the pharmaceutical composition according to clause 17 or the method of clause 18 wherein the disease is a cancer or tumor.

20. The antibody or antigen-binding portion thereof or the pharmaceutical composition according to clause 17 or 19 or the method of clause 18 or 19 further comprising separately administering another therapeutic agent to the subject.

21. The antibody or antigen-binding portion thereof or the pharmaceutical composition, or the method according to clause 20 wherein the therapeutic agent is a cytotoxic agent or a radiotoxic agent, an immunosuppressant, an immunological modulating agent, or an antibody or antibody fragment thereof.

22. The antibody or antigen-binding portion thereof or the pharmaceutical composition, or the method according to clause 21 wherein the immunological modulating agent is a cytokine or a chemokine.

23. A nucleic acid sequence that encodes an antibody or antibody antigen-binding portion thereof according to any one of clauses 1 to 15.

24. The nucleic acid sequence according to clause 23 comprising a sequence selected from: SEQ ID NO: 3 and SEQ ID NO: 5, or SEQ ID NO: 13 and SEQ ID NO: 15, or SEQ ID NO: 23 and SEQ ID NO: 25, or SEQ ID NO: 33 and SEQ ID NO: 35, or SEQ ID NO: 43 and SEQ ID NO: 45, or SEQ ID NO: 53 and SEQ ID NO: 55, or SEQ ID NO: 63 and SEQ ID NO: 65, or SEQ ID NO: 73 and SEQ ID NO: 75, or SEQ ID NO: 83 and SEQ ID NO: 85, or SEQ ID NO: 93 and SEQ ID NO: 95, or SEQ ID NO: 103 and SEQ ID NO: 105, or SEQ ID NO: 113 and SEQ ID NO: 115, or SEQ ID NO: 123 and SEQ ID NO: 125, or SEQ ID NO: 133 and SEQ ID NO: 135, or SEQ ID NO: 143 and SEQ ID NO: 145, or SEQ ID NO: 153 and SEQ ID NO: 155, or SEQ ID NO: 163 and SEQ ID NO: 165, or SEQ ID NO: 173 and SEQ ID NO: 175, or SEQ ID NO: 183 and SEQ ID NO: 185, or SEQ ID NO: 193 and SEQ ID NO: 195, or SEQ ID NO: 203 and SEQ ID NO: 205, or SEQ ID NO: 213 and SEQ ID NO: 215, or SEQ ID NO: 223 and SEQ ID NO: 225, or SEQ ID NO: 233 and SEQ ID NO: 235, or SEQ ID NO: 243 and SEQ ID NO: 245, or SEQ ID NO: 253 and SEQ ID NO: 255, or SEQ ID NO: 263 and SEQ ID NO: 265, or SEQ ID NO: 273 and SEQ ID NO: 275, or SEQ ID NO: 283 and SEQ ID NO: 285 or a sequence having at least 75% sequence identity to any one of the forgoing sequences.

25. A vector comprising a nucleic acid sequence according to any one of clauses 23 to 24.

26. A host cell comprising the nucleic acid sequence according to any one of clauses 23 to 24 or a vector of clause 25.

27. A kit comprising an antibody or antigen-binding portion thereof according to any one of clauses 1 to 15 or a pharmaceutical composition according to clause 16.

28. The kit according to clause 27 further comprising a reagent for the detection of the antibody or antigen-binding portion thereof.

29. A method for making a canine antibody that binds PD-1 comprising culturing the isolated host cell of clause 26 and recovering said antibody.

30. A method for making a canine antibody that binds PD-1 comprising the steps of
a) immunising a transgenic mouse that expresses a nucleic acid construct comprising canine heavy chain V genes and canine light chain V genes with PD-1 antigen,
b) generating a library of antibodies from said mouse and
c) isolating an antibody from said library.

31. A method for detecting a PD-1 protein or an extracellular domain of a PD-1 protein in a biological sample from a canine subject, comprising contacting a biological sample with the antibody or antigen-binding portion thereof of any one of clauses 1 to 15 wherein said antibody or antigen-binding portion thereof is linked to a detectable label.

32. The method of clause 31, wherein the biological sample is a biopsy, tissue, blood, serum, plasma, or lymphatic fluid sample.

33. A method of inhibiting tumor growth or metastasis comprising contacting a tumor cell with an effective amount of the antibody or antigen-binding portion thereof according to any one of clauses 1 to 15 or pharmaceutical composition according to clause 16.

34. A method of killing a tumor cell expressing PD-1, comprising contacting the cell with the antibody of any one of clauses 1 to 15 or pharmaceutical composition according to clauses 16, such that killing of the cell expressing PD-1 occurs.

35. The method of clause 34 wherein the tumor cell is a canine tumor cell.

36. A binding agent comprising the antibody or antigen-binding portion thereof according to any one of clauses 1 to 15 wherein said antibody or antigen-binding portion thereof is linked to a second antibody or antigen-binding portion thereof that binds to a second target.

37. The binding agent according to clause 36 wherein the second target binding agent is selected from any one of the list comprising LAG-3, OX40, OX40L, CD2, CD27, CDS, ICAM-1, LFA-1 (CD11a/CD18), ICOS (CD278), 4-1BB (CD137), GITR, CD30, CD40, BAFFR, HVEM, CD7, LIGHT, NKG2C, SLAMF7, NKp80, CD160, B7-H3 or CD83 ligand, CD3, CD8, CD28, CD4 or ICAM-1.

38. An immunoconjugate comprising the antibody or antigen-binding portion thereof according to any one of clauses 1 to 15 or the binding agent of clause 36 or 37.

39. A method of modulating an immune response comprising administering an antibody or fragment thereof according to any one of clauses 1 to 15, or a pharmaceutical composition according to clause 16 or a binding agent according to any one of clauses 36 to 37, or an immunoconjugate according to any one of clauses 38 to 39.

40. A combination therapy comprising an antibody or fragment thereof according to any one of clauses 1 to 15, or a pharmaceutical composition according to clause 16 or a binding agent according to any one of clauses 36 to 37, or an immunoconjugate according to any one of clauses 38 to 39 and a further therapeutic moiety.

41. A combination therapy according to clause 40 wherein the further therapeutic moiety is an antibody optionally an antibody that binds an immunooncology target, or a chemotherapy agent.

TABLE 2
Sequence IDs of amino acid and nucleotide
sequences for each anti-PD-1 antibody
included within the specification.

| SEQ ID | | |
|---|---|---|
| NO: | Description | Sequence |
| 3 | PMX125 VH_NT | GAGGTGCAGCTGGTGGAGTCTGGGGGAGACCTGGTGAAGCCTGGGGGGTCCCTG |
| | | AGACTTTCCTGTGTGGCCTCTGGATTCTCCTTCAGTAGATACCACATGAGCTGGG |
| | | TCCGCCAGGCTCCAGGGAAGGGACTTCAGTGGGTCGCATACATTATCAGTGGTG |
| | | GAATTACCACAAGCTATGCAGACGCTGTGAAGGGCCGATTCACCATCTCCAGAG |
| | | ACAACGCCAAGAACACGCTGTTTCTTCAGATGAACAGCCTGAGAGCCGAGGACA |
| | | CGGCCGTGTATTACTGTGCGACGTACGGTAGTTATTTTTTTGACAAGTGGGGCCA |
| | | GGGAACCCTGGTCACCGTCTCCTCAG |
| 4 | PMX125 VH_AA | EVQLVESGGDLVKPGGSLRLSCVASGFSFSRYHMSWVRQAPGKGLQWVAYIISGGI |
| | | TTSYADAVKGRFTISRDNAKNTLFLQMNSLRAEDTAVYYCATYGSYFFDKWGQGT |
| | | LVTVSS |
| 5 | PMX125 VL_NT | GATATTGTCATGACACAGACCCCACTGTCCCTGTCTGTCAGCCCTGGAGAGACTG |
| | | CCTCCATCTCTTGCAAGGCCAGTCAGAGCCTCCTGCACAGTGATGGAAAGTCGT |
| | | ATTTGAATTGGTTCCGACAGAAGCCAGGCCAGTCTCCACAGCGTTTGATCTATAG |
| | | GGTCTCCAACAGAGACCCTGGGGTCCCAGACAGGTTCAGTGGCAGCGGGTCAGG |
| | | GACAGATTTCACCCTGAGAATCAGCAGAGTGGAGGCTGACGATACTGGACTTTA |
| | | TTACTGCGGGCAAGTTATACAAGATCCGACGTTCGGCGCAGGAACCAAGGTGGA |
| | | GCTCAAAC |
| 6 | PMX125 VL_AA | DIVMTQTPLSLSVSPGETASISCKASQSLLHSDGKSYLNWFRQKPGQSPQRLIYRVSN |
| | | RDPGVPDRFSGSGSGTDFTLRISRVEADDTGLYYCGQVIQDPTFGAGTKVELK |
| 7 | PMX125 CDR1H | GFSFSRY |
| 8 | PMX125 CDR2H | ISGGIT |
| 9 | PMX125 CDR3H | CATYGSYFFDKW |
| 10 | PMX125 CDR1L | CKASQSLLHSD |
| 11 | PMX125 CDR2L | PQRLIYR |
| 12 | PMX125 CDR3L | CGQVIQDPTF |
| 13 | PMX126 VH_NT | GAGGTGCAGCTGGTGGAGTCTGGGGGAGACCTGGTGAAGCCTGCGGGGTCCCTG |
| | | AGACTGTCCTGTTTGGCCTCTGGATTCACCTTCAGTAGCTACAGCATGAGCTGGG |
| | | TCCGCCAGGCTCCTGAGAAGGGGCTGCAGTCGGTCGCGGGTATTAACAGCGATG |
| | | GAGATAGAACATACTACACAGACGCTGTGAAGGGCCGATTCACCATCTCCAGAG |
| | | ACAACGCCAAGAGCACAGTGTATCTGCAGATGAACAGCCTGCGAGCCGAGGAC |
| | | ACGGCCATGTATTATTGTGTACGGGGGTTTTCCTCTTTCAATGCTTTTGGTTACTG |
| | | GGGCCAGGGCACCCTGGTCACTGTCTCCTCAG |
| 14 | PMX126 VH_AA | EVQLVESGGDLVKPAGSLRLSCLASGFTFSSYSMSWVRQAPEKGLQSVAGINSDGD |
| | | RTYYTDAVKGRFTISRDNAKSTVYLQMNSLRAEDTAMYYCVRGFSSFNAFGYWGQ |
| | | GTLVTVSS |
| 15 | PMX126 VL_NT | GATATTGTCATGACACAGACCCCACTGTCCCTGTCTGTCAGTCCTGGAGAGACTG |
| | | CCTCCATCTCTTGCAAGGCCAGTCAGAGCCTCCTGCACAGTGATGGAAACACGT |
| | | ATTTGAGTTGGTTCCGACAGAAGCCAGGCCAGTCTCCACACCGTTTGATCTATGC |
| | | GGTCTCCCACAGACACCCTGGGGTCCCAGACAGGTTCAGTGGCAGCGGGTCAGG |
| | | GACAGATTTCACCCTGAGAATCAGCAGAGTGGAGGCTGACGATGCTGGACTTTA |
| | | TTACTGCGGACAAGTTATACAAGATCCGTGGACGTTCGGAGCAGGAACCAAGGT |
| | | GGAACTCAAGC |
| 16 | PMX126 VL_AA | DIVMTQTPLSLSVSPGETASISCKASQSLLHSDGNTYLSWFRQKPGQSPHRLIYAVSH |
| | | RHPGVPDRFSGSGSGTDFTLRISRVEADDAGLYYCGQVIQDPWTFGAGTKVELK |
| 17 | PMX126 CDR1H | GFTFSSY |
| 18 | PMX126 CDR2H | NSDGDR |
| 19 | PMX126 CDR3H | CVRGFSSFNAFGYW |
| 20 | PMX126 CDR1L | CKASQSLLHSD |
| 21 | PMX126 CDR2L | PHRLIYA |
| 22 | PMX126 CDR3L | CGQVIQDPWTF |
| 23 | PMX127 VH_NT | GAGGTGCAGCTGGTGGAGTCTGGGGGAGACCTGGTGAAGCCTGGGGGGTCCCTG |
| | | AGACTTTCCTGTGTGGCCTCTGGATTCTCCTTCAGTAGATACCACATGAGCTGGG |
| | | TCCGCCAGGCTCCAGGGAAGGGACTTCAGTGGGTCGCATACATTGTCAGTGGTG |
| | | GAATTTCCACAAGCTATGCAGACGCTGTGAAGGGCCGATTCACCATCTCCAGAG |
| | | ACAACGCCAAGAACACGCTGTTTCTTCAGATGAACAGCCTGAGAGCCGAGGACA |
| | | CGGCCGTGTATTACTGTGCGACGTACGGTAGTTATTTTTTTGACACCTGGGGCCA |
| | | GGGAACCCTGGTCACCGTCTCCTCAG |
| 24 | PMX127 VH_AA | EVQLVESGGDLVKPGGSLRLSCVASGFSFSRYHMSWVRQAPGKGLQWVAYIVSGGI |
| | | STSYADAVKGRFTISRDNAKNTLFLQMNSLRAEDTAVYYCATYGSYFFDTWGQGT |
| | | LVTVSS |
| 25 | PMX127 VL_NT | GATATTGTCATGACACAGACCCCACTGTCCCTGTCTGTCAGCCCTGGAGAGACTG |
| | | CCTCCATTTTTTGTAAGGCCAGTCAGAGCCTCCTGCACAGTGATGGAAAGTCGTA |
| | | TTTGAATTGGTTCCGACAGAAGCCAGGCCAGTCTCCACAGCGTTTGATCTATAGG |
| | | GTCTCCAACAGGGACCCTGGGGTCCCAGACAGGTTCAGTGGCAGCGGGTCAGGG |
| | | ACAGATTTCACCCTGAGAATCAGCAGAGTGGAGGCTGACGATACTGGACTTTAT |
| | | TACTGCGGGCAAGTTATACAAGATCCGACGTTCGGCGCAGGAACCAAGGTGGAG |
| | | CTCAAAC |
| 26 | PMX127 VL_AA | DIVMTQTPLSLSVSPGETASIFCKASQSLLHSDGKSYLNWFRQKPGQSPQRLIYRVSN |
| | | RDPGVPDRFSGSGSGTDFTLRISRVEADDTGLYYCGQVIQDPTFGAGTKVELK |
| 27 | PMX127 CDR1H | GFSFSRY |
| 28 | PMX127 CDR2H | VSGGIS |
| 29 | PMX127 CDR3H | CATYGSYFFDTW |
| 30 | PMX127 CDR1L | CKASQSLLHSD |
| 31 | PMX127 CDR2L | PQRLIYR |
| 32 | PMX127 CDR3L | CGQVIQDPTF |
| 33 | PMX128 VH_NT | CAGGTGCAGCTGGTGGAGTCTGGGGGAGACCTGGTGAAGCCGGGGGGGTCCCT |
| | | GAGACTTTCCTGTGTGGCCTCTGGATTCCCCTTCAGTAGCTTCCACATGACCTGG |
| | | GTCCGCCAGGTTCCTGGGATGGGGCTTCAGTGGGTCGCATACGTTAAGACTGGT |
| | | GGAAGTAGTACAAGCTATGCAGACGCTGTGAAGGGCCGATTCACCATCTCCAGA |
| | | GACAATGCCAAGAACACACTGTATCTTCACATGAATAGCCTGAGAGCCGAGGAC |
| | | ACGGCCTTGTATTACTGTGCGACTGGGGTGGTCCCCACTACGGTAGCTTCTTCCT |
| | | ACTGGGGCCAGGGAACCCTGGTCACCGTCTCCTCAG |
| 34 | PMX128 VH_AA | QVQLVESGGDLVKPGGSLRLSCVASGFPFSSFHMTWVRQVPGMGLQWVAYVKTG |
| | | GSSTSYADAVKGRFTISRDNAKNTLYLHMNSLRAEDTALYYCATGVVPTTVASSY |
| | | WGQGTLVTVSS |
| 35 | PMX128 VL_NT | GATATTGTCATGACACAGACCCCACTGTCCCTGTCCGTCAGCCCTGGAGAGCCG |
| | | GCCTCCATCTCCTGCAAGGCCAGTCAGAGCCTCCTGCACAGTAGTGGGTACACC |
| | | TATTTGTATTGGTTCCGACAGAAGCCAGGCCAGTCTCCACAGCGTTTGGTCTATA |
| | | AGGTCGTCTACAGAGACCCTGGGGTCCCAGACAGGTTCAGTGGCAGCGGGTCAG |
| | | GGACAGATTTCACCCTGAGAATCAGCAGAGTGGAGGCTGATGATGCTGGAGTTT |
| | | ATTACTGCGGGCAAGGTATAAAAGATCCGTTCACTTTTGGTCAAGGGACCAAAC |
| | | TGGAGATCAAAC |
| 36 | PMX128 VL_AA | DIVMTQTPLSLSVSPGEPASISCKASQSLLHSSGYTYLYWFRQKPGQSPQRLVYKVV |
| | | YRDPGVPDRFSGSGSGTDFTLRISRVEADDAGVYYCGQGIKDPFTFGQGTKLEIK |
| 37 | PMX128 CDR1H | GFPFSSF |
| 38 | PMX128 CDR2H | KTGGSS |
| 39 | PMX128 CDR3H | CATGVVPTTVASSYW |
| 40 | PMX128 CDR1L | CKASQSLLHSS |
| 41 | PMX128 CDR2L | PQRLVYK |
| 42 | PMX128 CDR3L | CGQGIKDPFTF |
| 43 | PMX129 VH_NT | GAACTCACACTGCAGGAGTCAGGGCCAGGATTAGTGAAGCCCTCACAGACCCTC |
| | | TCTCTCACCTGTGTTGTGTCCGGAGGCTCCGTCACCAACATATTCTACTGGAACT |
| | | GGATCCGTCAGCGCCCTAAGAGGGGACTGGAATGGATGGGATACTGGTCAGGTT |
| | | ACACACACTACAACCCGGCATTCCAGGGACGCCTCTCCATCGATGCTGACACGG |
| | | CCCAGAACCACTTCTCCCTGCAGCTGAGCTCCATGACCACCGAGGACACGGCCG |
| | | TGTATTATTGTGTTAGAGGATATATATGGACCCCAGATTATGACTACTGGGGCCA |
| | | GGGAATCCTGGTCACCGTCTCCTCAG |
| 44 | PMX129 VH_AA | ELTLQESGPGLVKPSQTLSLTCVVSGGSVTNIFYWNWIRQRPKRGLEWMGYWSGYT |
| | | HYNPAFQGRLSIDADTAQNHFSLQLSSMTTEDTAVYYCVRGYIWTPDYDYWGQGIL |
| | | VTVSS |
| 45 | PMX129 VL_NT | GATATTGTCATGACACAGACCCCACTGTCCCTGTCCGTCAGCCCTGGAGAGCCG |
| | | GCCTCCATCGCCTGCAAGGCCAGTCAGAGCCTCCTGCATAGTAATGGGAACACC |
| | | TATTTGTATTGGTTCCGACAGAAGCCAGGCCAGTCTCCACAGCGTTTGATCTATA |
| | | AGGTCTCCAACAGAGACCCTGGGGTCCCGGATAGGTTCAGTGGCAGCGGGTCAG |
| | | GGACAGATTTCACCCTGAGAATCACCAGAGTGGAGGCTGATGATGCTGGAGTTT |
| | | ATTACTGCGGGCAAGGTATACAAGATCCGTGGACGTTCGGAGAAGGAACCAAG |
| | | GTGGAGCTCAAAC |
| 46 | PMX129 VL_AA | DIVMTQTPLSLSVSPGEPASIACKASQSLLHSNGNTYLYWFRQKPGQSPQRLIYKVSN |
| | | RDPGVPDRFSGSGSGTDFTLRITRVEADDAGVYYCGQGIQDPWTFGEGTKVELK |
| 47 | PMX129 CDR1H | GGSVT |
| 48 | PMX129 CDR2H | GYWSGY |
| 49 | PMX129 CDR3H | CVRGYIWTPDYDYW |
| 50 | PMX129 CDR1L | CKASQSLLHSN |
| 51 | PMX129 CDR2L | PQRLIYK |
| 52 | PMX129 CDR3L | CGQGIQDPWTF |
| 53 | PMX130 VH_NT | GAGGTGCAGCTGGTGGAGTCTGGGGGAGACCTGGTGAAGCCTGCGGGGTCCCTG |
| | | AGACTGTCCTGTTTGGCCTCTGGATTCACCTTCAGTAGCTACAGCATGAGCTGGG |
| | | TCCGCCAGGCTCCTGAGAAGGGGCTGCACTTGGTCGCAGGTATCAACAGCGATG |
| | | GAGATAGAACATACTACAAAGACGCTGTGAAGGGCCGATTCACCATCTCCAGAG |
| | | ACAACGCCAAGAGTATAGTGTATCTCCAGATGAATAGCCTGCGAGTCGAGGACA |
| | | CGGCCATGTATTACTGTGTACGGGGGTTTTCCTCTTTCAATGGTCTTGGTTACTG |
| | | GGGCCAGGGCACCCTGGTCACTGTCTCCTCAG |
| 54 | PMX130 VH_AA | EVQLVESGGDLVKPAGSLRLSCLASGFTFSSYSMSWVRQAPEKGLHLVAGINSDGD |
| | | RTYYKDAVKGRFTISRDNAKSIVYLQMNSLRVEDTAMYYCVRGFSSFNGLGYWGQ |
| | | GTLVTVSS |
| 55 | PMX130 VL_NT | GATGTTGTCATGACACAGACCCCACTGTCCCTGTCTGTCAGCCCTGGAGAGACTG |
| | | CCTCCATCTCTTGCAAGGCCAGTCAGAGCCTCCTCCACAGTGATGGAAACACGT |
| | | ATTTGACTTGGTTCCGACAGAAGCCAGGCCAGTCTCCACACCGTTTGGTCTATGC |
| | | GGTCTCCCACAGACACCCTGGGGTCCCAGACAGGTTCAGTGGCAGCGGGTCAGG |
| | | GACAGATTTCACCCTGAGAATAAGCAGAGTGGAGGCTGACGATACTGGACTTTA |
| | | TTACTGCGGACAAGTTATACACGATCCGTGGACGTTCGGAGCAGGAACCAAGGT |
| | | GGAGCTCAAAC |
| 56 | PMX130 VL_AA | DVVMTQTPLSLSVSPGETASISCKASQSLLHSDGNTYLTWFRQKPGQSPHRLVYAVS |
| | | HRHPGVPDRFSGSGSGTDFTLRISRVEADDTGLYYCGQVIHDPWTFGAGTKVELK |
| 57 | PMX130 CDR1H | GFTFSSY |
| 58 | PMX130 CDR2H | NSDGDR |
| 59 | PMX130 CDR3H | CVRGFSSFNGLGYW |
| 60 | PMX130 CDR1L | CKASQSLLHSD |
| 61 | PMX130 CDR2L | PHRLVYA |
| 62 | PMX130 CDR3L | CGQVIHDPWTF |
| 63 | PMX131 VH_NT | GAGGTGCAGCTGGTGGAGTCTGGGGGAGACCTGGTGAAGCCTGGGGGGTCCCTG |
| | | AGACTTTCCTGTGTGGCCTCTGGATTCACCTTCAGTTTCTACCTCCTTAACTGGGT |
| | | CCGCCAGGCTCCAGGGAAGGGACTTCAATGGGTCGCATACATTAACAGTGGTGG |
| | | AAGTACCACATACTATGCAGACGCTGTGAAGGGCCGATTCACCATCTCCAGAGA |
| | | CAACGCCAAGAACACTCTCTATCTTCAGATGAACAGCCTGAGAGCCGAGGACAC |
| | | GGCCGTATACTACTGTGCGTCTGGGGGTACTTGGGGTTTTTTTGACTACTGGGGC |
| | | CAGGGAACCCTGGTCTCCGTCTCCTCAG |
| 64 | PMX131 VH_AA | EVQLVESGGDLVKPGGSLRLSCVASGFTFSFYLLNWVRQAPGKGLQWVAYINSGGS |
| | | TTYYADAVKGRFTISRDNAKNTLYLQMNSLRAEDTAVYYCASGGTWGFFDYWGQ |
| | | GTLVSVSS |
| 65 | PMX131 VL_NT | TCCTATGTGCTGTCTCAGCCGCCATCAGTGACTGTGACTCTGAGGCAGACGGCCC |
| | | GCCTCACCTGTGGGGGAGACAGCATTGGAAGTAAAAGTATTGAATGGTACCAGC |
| | | AGAAGCCGGGCCAGCCCCCCGTGCTCATTATCTATGGTGAAAGTAGCAGGCCGT |
| | | CAGGGATCCCTGAGCGATTCTCCGGCGCCAACTCGGGGAACACGGCCACCCTGA |
| | | CCATCAGCGGGGCCCTGGCCGAGGACGAGGCTGACTATTACTGCCAGGTGTGGG |
| | | ACAGCAGTACTTGGGTATTCGGTGAAGGGACCCAGCTGACCGTCCTCG |
| 66 | PMX131 VL_AA | SYVLSQPPSVTVTLRQTARLTCGGDSIGSKSIEWYQQKPGQPPVLIIYGESSRPSGIPE |
| | | RFSGANSGNTATLTISGALAEDEADYYCQVWDSSTWVFGEGTQLTVL |
| 67 | PMX131 CDR1H | GFTFSFY |
| 68 | PMX131 CDR2H | NSGGST |
| 69 | PMX131 CDR3H | CASGGTWGFFDYW |
| 70 | PMX131 CDR1L | GGDSIGSKSIE |
| 71 | PMX131 CDR2L | GESSRPS |
| 72 | PMX131 CDR3L | CQVWDSSTWVF |
| 73 | PMX132 VH_NT | GAGGTGCACCTGGTGGAGTCTGGGGGAGACCTGGTGAAGCCGGGGGGGTCCCT |
| | | GAGACTTTCCTGTGTGGCCTCTGGATTCACCTTCAGAAACTATTACATGAGTTGG |
| | | GTCCGCCAAGGTCCAGGGAAGGGGCTTCAGTGGGTCGCATACATCAACAGTGGT |
| | | GGAATTAGTACAAGATATGCAGACGCTGTGAAGGGCCGATTCACCATCTCCAGA |
| | | GACAACGCCAAGAATACGGTGTATCTTCAGATGAACAGCCTGAGAGTCGAGGAC |
| | | ACGGCCGTGTATTACTGTGCGACCTACGGTAGTTATTACTACGACTACTGGGGCC |
| | | AGGGAACCCTGGTCACCGTCACCTCAG |
| 74 | PMX132 VH_AA | EVHLVESGGDLVKPGGSLRLSCVASGFTFRNYYMSWVRQGPGKGLQWVAYINSGG |
| | | ISTRYADAVKGRFTISRDNAKNTVYLQMNSLRVEDTAVYYCATYGSYYYDYWGQG |
| | | TLVTVTS |
| 75 | PMX132 VL_NT | GATATTGTCATGACACAGACCCCACTGTCCCTGTCTGTCAGCCCTGGAGAGACTG |
| | | CCTCCATCTCTTGCAAGGCCAGTCAGAGCCTCCTGCACAATGATGACAACACGT |
| | | ATTTGAATTGGTTCCGACAGAGGCCAGGCCAGTCTCCACAGCGTTTGATCTATAG |
| | | GGTCTCCAACAGAGACCCTGGGGTCCCAGACAGGTTCAGTGGCAGCGGGTCAGG |
| | | GACAGATTTCACCCTGAAAATCAGCAGAGTGGAGGCTGACGATACTGGAGTTTA |
| | | TTACTGCGGGCAAGTTATACAAGATCCTACGTTTGGCAAAGGGACACATCTGGA |
| | | GATTAAAC |
| 76 | PMX132 VL_AA | DIVMTQTPLSLSVSPGETASISCKASQSLLHNDDNTYLNWFRQRPGQSPQRLIYRVSN |
| | | RDPGVPDRFSGSGSGTDFTLKISRVEADDTGVYYCGQVIQDPTFGKGTHLEIK |
| 77 | PMX132 CDR1H | GFTFRNY |
| 78 | PMX132 CDR2H | NSGGIS |
| 79 | PMX132 CDR3H | CATYGSYYYDYW |
| 80 | PMX132 CDR1L | CKASQSLLHND |
| 81 | PMX132 CDR2L | PQRLIYR |
| 82 | PMX132 CDR3L | CGQVIQDPTF |
| 83 | PMX133 VH_NT | GATCTCACACTGCAGGAGTCAGGGCCAGGACTAGTGAAGCCCTCACAGACCCTC |
| | | TCTCTCACCTGTGTTGTGTCCGGAGGCTCCGTCACCAACATATTCTACTGGAACT |
| | | GGATCCGTCAGCGCCCTAATAGGGGACTGGAATGGATGGGGTACTGGTCAGGTT |
| | | ACACACACTACAACCCGGCATTCCAGGGACGCATCTCCATCGATGCTGACACGG |
| | | CCCAGAACCACTTCTCCCTGCAGCTGAGCTCCATGACCACCGAGGACACGGCCG |
| | | TATATTACTGTCTAAGAGGATATATATGGACCCCAGATTATGACTACTGGGGCC |
| | | AGGGAATCCTGGTCACCGTCTCCTCAG |
| 84 | PMX133 VH_AA | DLTLQESGPGLVKPSQTLSLTCVVSGGSVTNIFYWNWIRQRPNRGLEWMGYWSGY |
| | | THYNPAFQGRISIDADTAQNHFSLQLSSMTTEDTAVYYCLRGYIWTPDYDYWGQGI |
| | | LVTVSS |
| 85 | PMX133 VL_NT | GATATTGTCATGACACAGACCCCACTGTCCCTGTCCGTCAGTCCTGGAGAGCCG |
| | | GCCTCCATCGCCTGTAAGGCCAGTCAGAGCCTCCTGCACAGTAATGGGAACACC |
| | | TATTTGTATTGGTTCCGACAGAAGCCAGGCCAGTCTCCACAGCGTTTGATCTATA |
| | | AGGTCTCCAACAGAGACCCTGGGGTCCCGGATAGGTTCAGTGGCAGCGGGTCAG |
| | | GGACAGATTTCACCCTGAGAATCACCAGAGTGGAGGCTGATGATGCTGGAGTTT |
| | | ATTACTGCGGGCAAGGTATACAAGATCCGTGGACGTTCGGAGCAGGAACCAAG |
| | | GTGGGGCTCAAAC |
| 86 | PMX133 VL_AA | DIVMTQTPLSLSVSPGEPASIACKASQSLLHSNGNTYLYWFRQKPGQSPQRLIYKVSN |
| | | RDPGVPDRFSGSGSGTDFTLRITRVEADDAGVYYCGQGIQDPWTFGAGTKVGLK |
| 87 | PMX133 CDR1H | GGSVT |
| 88 | PMX133 CDR2H | GYWSGY |
| 89 | PMX133 CDR3H | CLRGYIWTPDYDYW |
| 90 | PMX133 CDR1L | CKASQSLLHSN |
| 91 | PMX133 CDR2L | PQRLIYK |
| 92 | PMX133 CDR3L | CGQGIQDPWTF |
| 93 | PMX134 VH_NT | GAGGTACAACTGGTGGAGTCTGGGGGAGACCTAGTGAAGCCGGGGGGGTCCCT |
| | | GAGACTTTCCTGTGTGGCCTCTGGATTCACTTTCAATACTTACCACATGAGCTGG |
| | | GTCCGCCAGGCTCCAGGGAAGGGGCTTCAGTGGGTCGCATACATTACCAGTGGT |
| | | GGAAGTAGTACAAGCCATGCAGACGCTGTGAAGGGCCGATTCACCATCTCCAGA |
| | | GACAACGCCAAGAAAACGCTGTATCTTCAGATGAACAGCCTGAGAGCCGAGGA |
| | | CACGGCCGTGTATTACTGTGCGACTTATGGTAGTTATTTCTTTGACTCTTGGGGC |
| | | CAGGGAACCCTGGTCACCGTCTCCTCAG |
| 94 | PMX134 VH_AA | EVQLVESGGDLVKPGGSLRLSCVASGFTFNTYHMSWVRQAPGKGLQWVAYITSGG |
| | | SSTSHADAVKGRFTISRDNAKKTLYLQMNSLRAEDTAVYYCATYGSYFFDSWGQG |
| | | TLVTVSS |
| 95 | PMX134 VL_NT | AGTGGGGATATTGTCATGACACAGACCCCACTGTCCCTGTCTGTCAGCCCTGGCG |
| | | AGACTGCCTCCATCTCTTGCAAGGCCAGTCAGAGCCTCCTGCACATTGATGGAA |
| | | ACACGTATTTGAATTGGTTCCGACAGAAGCCAGGCCAGTCTCCACAGCGTTTGA |
| | | TCTATAGGGTCTCCACCAGAGACCCTGGGGTCCCAGACAGGTTCAGTGGCAGCG |
| | | GGTCAGGGACAGATTTCACCCTGAGAATCAGCAGAGTGGAGGCTGACGATACTG |
| | | GAGTTTATTACTGCGGGCAAGTTATACAAGATCCTACGTTCGGCCAAGGGACCA |
| | | AGGTGGAGATCAAAC |
| 96 | PMX134 VL_AA | SGDIVMTQTPLSLSVSPGETASISCKASQSLLHIDGNTYLNWFRQKPGQSPQRLIYRV |
| | | STRDPGVPDRFSGSGSGTDFTLRISRVEADDTGVYYCGQVIQDPTFGQGTKVEIK |
| 97 | PMX134 CDR1H | GFTFNTY |
| 98 | PMX134 CDR2H | TSGGSS |
| 99 | PMX134 CDR3H | CATYGSYFFDSW |
| 100 | PMX134 CDR1L | ISCKASQSLLH |
| 101 | PMX134 CDR2L | QSPQRLI |
| 102 | PMX134 CDR3L | CGQVIQDPTF |
| 103 | PMX135 VH_NT | GAGGTACAACTGGTGGAGTCTGGGGGAGACCTAGTGAAGCCGGGGGGGTCCCT |
| | | GAGACTTTCCTGTGTGGCCTCTGGATTCACTTTCAATACTTACCACATGAGCTGG |
| | | GTCCGCCAGGCTCCAGGGAAGGGGCTTCAGTGGGTCGCATACATTACCAGTGGT |
| | | GGAAGTAGTACAAGTCATGCAGACGCTGTGAAGGGCCGATTCACTATCTCCAGA |
| | | GACAACGCCAAGAAAACGCTGTATCTTCAGATGAACAACCTGAGAGCCGAGGA |
| | | CACGGCCGTGTATTACTGTGCGACTTATGGTAGTTATTTCTTTGACTCTTGGGGC |
| | | CAGGGAACCCTGGTCACCGTCTCCTCAG |
| 104 | PMX135 VH_AA | EVQLVESGGDLVKPGGSLRLSCVASGFTFNTYHMSWVRQAPGKGLQWVAYITSGG |
| | | SSTSHADAVKGRFTISRDNAKKTLYLQMNNLRAEDTAVYYCATYGSYFFDSWGQG |
| | | TLVTVSS |
| 105 | PMX135 VL_NT | AGTGGGGATATTGTCATGACACAGACCCCACTGTCCCTGCCTGTCAGCCCTGGC |
| | | GAGACTGCCTCCATCTCTTGTAAGGCCAGTCAGAGCCTCCTGCACATTGATGGA |
| | | AACACGTATTTGAATTGGTTCCGACAGAAGCCAGGCCAGTCTCCACAGCGTTTG |
| | | ATCTATAGGGTCTCCACCAGAGACCCTGGGGTCCCAGACAGGTTCAGTGGCAGC |
| | | GGGTCAGGGACAGATTTCACCCTGAGAATCAGCAGAGTGGAGGCTGACGATACT |
| | | GGAGTTTATTACTGCGGGCAAGTTATACAAGATCCTACGTTCGGCCAAGGGACC |
| | | AAGGTGGAGATCAAAC |
| 106 | PMX135 VL_AA | SGDIVMTQTPLSLPVSPGETASISCKASQSLLHIDGNTYLNWFRQKPGQSPQRLIYRV |
| | | STRDPGVPDRFSGSGSGTDFTLRISRVEADDTGVYYCGQVIQDPTFGQGTKVEIK |
| 107 | PMX135 CDR1H | GFTFNTY |
| 108 | PMX135 CDR2H | TSGGSS |
| 109 | PMX135 CDR3H | CATYGSYFFDSW |
| 110 | PMX135 CDR1L | ISCKASQSLLH |
| 111 | PMX135 CDR2L | QSPQRLI |
| 112 | PMX135 CDR3L | CGQVIQDPTF |
| 113 | PMX136 VH_NT | GAGGTGCCACTGGTGGAGTCTGGGGGAGACCTGGTGAAGCCTGGGGGGTCCCTG |
| | | AGACTTTCCTGTCTGGCCTCTGGATTCACCTTCAGTAGCTACCACATGAGCTGGG |
| | | TCCGCCAGGCTCCAGGGAAGGGGCTTCAGTGGGTCGCATACATTAACAGTGGTG |
| | | GAAATATCATAAGCTATGTAGACGCTGTGAAGGGCCGATTCACCATCTCCAGAG |
| | | ACAACGCCAAGAACACGCTGTATCTTCAGATGAACAGCCTGAGAGTCGAGGACA |
| | | CGGCCGTTTATTACTGTGCGAGTCACTTCCTTAGCTACTTCTGGGGCCAGGGAAC |
| | | CCTGGTCACCGTCTCCTCAG |
| 114 | PMX136 VH_AA | EVPLVESGGDLVKPGGSLRLSCLASGFTFSSYHMSWVRQAPGKGLQWVAYINSGGN |
| | | IISYVDAVKGRFTISRDNAKNTLYLQMNSLRVEDTAVYYCASHFLSYFWGQGTLVT |
| | | VSS |
| 115 | PMX136 VL_NT | TCCTATGTGCTGTCTCAGCCGCCATCAGCGACTGTGACTCTGAGGCAGACGGCCC |
| | | GCCTCACCTGTGGGGGAGACAGCATTGGAAGAAAAAGTGTTGAATGGTACCAAC |
| | | AGAAGCCGGGCCAGCCCCCCGTGCTCATTATCTATGGTGATAGCAGCAGGCCGT |
| | | CAGGGATCCCTGAGCGATTCTCCGGCGCCAACTCGGGGAACACGGCCACCCTGA |
| | | CCATCAGCGGGGCCCTGGCCGAGGACGAGGCTGACTATTACTGCCAGGTGTGGG |
| | | ACAGCAGTACTAAGGCTAGGGTATTCGGTGAAGGGACCCAGCTGACCGTCCTCG |
| 116 | PMX136 VL_AA | SYVLSQPPSATVTLRQTARLTCGGDSIGRKSVEWYQQKPGQPPVLIIYGDSSRPSGIP |
| | | ERFSGANSGNTATLTISGALAEDEADYYCQVWDSSTKARVFGEGTQLTVL |
| 117 | PMX136 CDR1H | GFTFSSY |
| 118 | PMX136 CDR2H | NSGGNI |
| 119 | PMX136 CDR3H | CASHFLSYFW |
| 120 | PMX136 CDR1L | GGDSIGRKSVE |
| 121 | PMX136 CDR2L | GDSSRPS |
| 122 | PMX136 CDR3L | CQVWDSSTKARVF |
| 123 | PMX137 VH_NT | GAGGTGCACCTGGTGGAGTCTGGGGGAGACCTGGTGAGGCCGGGGGGGTCCCT |
| | | GAGACTTTCCTGTGTGGCCTCTGGATTCACCTTCAGTAGCTACCACATGAGCTGG |
| | | GTCCGCCAGGCTCCAGGGAAGGGGCTTCAGTGGGTCGCATACATTAGCAATGAT |
| | | GGAAGAATCATAAGTTATGCAGACGCTGTGAAGGGCCGATTCACCATCTCCAGA |
| | | GACAACGCCAAGAACACGTTGTATCTTCAGATGAACAGCCTGAACGTCGAGGAC |
| | | ACGGCCGTGTATTACTGTGCGAGTCACTTCGATAGTTACTTCTGGGGCCAGGGA |
| | | ACCCTGGTCACCGTCTCCTCAG |
| 124 | PMX137 VH_AA | EVHLVESGGDLVRPGGSLRLSCVASGFTFSSYHMSWVRQAPGKGLQWVAYISNDG |
| | | RIISYADAVKGRFTISRDNAKNTLYLQMNSLNVEDTAVYYCASHFDSYFWGQGTLV |
| | | TVSS |
| 125 | PMX137 VL_NT | TCCTATGTGCTGTCTCAGCCGCCATCAGCGACTGTGACTCTGAGGCAGACGGCCC |
| | | GCCTCACCTGTGGGGGAGACAGCATTGGACGTAAAAGTGTTGAATGGTACCAGC |
| | | AGAAGCCGGGCCAGCCCCCCGTGCTCATTATATATGGTGATAGTAGTAGGCCGT |
| | | CAGGGATCCCTGAGCGATTCTCCGGCGCCAACTCGGGGAACACGGCCACCCTGA |
| | | CCATCAGCGGGGCCCTGGCCGAGGACGAGGCGGACTATTACTGCCAGGTGTGGG |
| | | ACAGCAGAACTAAGGCTAGGGTATTCGGTGAAGGGACCCAGCTGACCGTCCTCG |
| 126 | PMX137 VL_AA | SYVLSQPPSATVTLRQTARLTCGGDSIGRKSVEWYQQKPGQPPVLIIYGDSSRPSGIP |
| | | ERFSGANSGNTATLTISGALAEDEADYYCQVWDSRTKARVFGEGTQLTVL |
| 127 | PMX137 CDR1H | GFTFSSY |
| 128 | PMX137 CDR2H | SNDGRI |
| 129 | PMX137 CDR3H | CASHFDSYFW |
| 130 | PMX137 CDR1L | GGDSIGRKSVE |
| 131 | PMX137 CDR2L | GDSSRPS |
| 132 | PMX137 CDR3L | CQVWDSRTKARVF |
| 133 | PMX138 VH_NT | GAGGTGCAGCTGGTGGAGTCTGGGGGAGACCTGGTGAAGCCTGGGGGGTCCCTG |
| | | AGACTTTCCTGTTTGGCCTCTGGATTCACCTTCAGTAACTTCCACATGAACTGGG |
| | | TCCGCCAGGCTCCAGGGAAGGGGCTTCAGTGGGTCGCATTCGTAAACGTTGGTG |
| | | GAATTAGAACAGCCTATGCAGACGGTGTGAAGGGCCGATTCACCATCTCCAGAG |
| | | ACGACGCCAAAAACACGCTCTATCTTCAGATGGACAGCCTGCGAGCCGACGACA |
| | | CGGCCGTATATTACTGTGCGAGGGCTTTCCCGGGGGACTACTGGGGCCAGGGAA |
| | | CCCTGGTCACCGTCTCCTCAG |
| 134 | PMX138 VH_AA | EVQLVESGGDLVKPGGSLRLSCLASGFTFSNFHMNWVRQAPGKGLQWVAFVNVGG |
| | | IRTAYADGVKGRFTISRDDAKNTLYLQMDSLRADDTAVYYCARAFPGDYWGQGTL |
| | | VTVSS |
| 135 | PMX138 VL_NT | TCCTATGTGCTGTCTCAGCCGCCATCAGCGACTGTGACTCTGAGGCAGACGGCCC |
| | | GCCTCACCTGTGGGGGAGACAGCATTGGGAGAAAAAGTGTTGAATGGTATCAGC |
| | | AGAAGCCGGGCCAGCCCCCCGTGCTCATTATCTTTGGTGATAGCAGCAGGCCGT |
| | | CAGGGATCCCTGAGCGATTCTCCGGCGCCAACTCGGGGAACACGGCCACCCTGA |
| | | CCATCAGCGGGGCCCTGGCCGAGGACGAGGCTGACTATTACTGCCAGGTGTGGG |
| | | ACAGCAGTACTAAGGCTTGGGTATTCGGTGAAGGGACCCAGCTGACCGTCCTCG |
| 136 | PMX138 VL_AA | SYVLSQPPSATVTLRQTARLTCGGDSIGRKSVEWYQQKPGQPPVLIIFGDSSRPSGIPE |
| | | RFSGANSGNTATLTISGALAEDEADYYCQVWDSSTKAWVFGEGTQLTVL |
| 137 | PMX138 CDR1H | GFTFSNF |
| 138 | PMX138 CDR2H | NVGGIR |
| 139 | PMX138 CDR3H | CARAFPGDYW |
| 140 | PMX138 CDR1L | GGDSIGRKSVE |
| 141 | PMX138 CDR2L | GDSSRPS |
| 142 | PMX138 CDR3L | CQVWDSSTKAWVF |
| 143 | PMX139 VH_NT | GAGGTACAACTGGTGGAGTCTGGGGGAGACCTAGTGAAGCCGGGGGGGTCCCT |
| | | GAGACTTTCCTGTGTGGCCTCTGGATTCACTTTCAATACTTACCACATGAGCTGG |
| | | GTCCGCCAGACTCCAGGGAAGGGGCTTCAGTGGGTCGCATACATTACCAGTGGT |
| | | GGAATTAGTACAAGCCATTCAGACGCTGTGAAGGGCCGATTCACCATCTCCAGA |
| | | GACAACGCCAAGAAAACGCTATATCTTCAGATGAACAGCCTGAGAGCCGAGGA |
| | | CACGGCCGTGTATTACTGTGCGACTTATGGTAGTTATTTCTTTGACTCTTGGGGC |
| | | CAGGGAACCCTGGTCACCGTCTCCTCAG |
| 144 | PMX139 VH_AA | EVQLVESGGDLVKPGGSLRLSCVASGFTFNTYHMSWVRQTPGKGLQWVAYITSGGI |
| | | STSHSDAVKGRFTISRDNAKKTLYLQMNSLRAEDTAVYYCATYGSYFFDSWGQGTL |
| | | VTVSS |
| 145 | PMX139 VL_NT | AGTGGGGATATTGTCATGACACAGACCCCACTGTCCCTGTCTGTCAGCCCTGGCG |
| | | AGACTGCCTCCATCTCTTGCAAGGCCAGTCAGAGCCTCCTGCACATTGATGGAA |
| | | ACACGTATTTGAATTGGTTCCGACAGAAGCCAGGCCAGTCTCCACAGCGTTTGA |
| | | TCTATAGGGTCTCCACCAGAGACCCTGGGGTCCCAGACAGGTTCAGTGGCAGCG |
| | | GGTCAGGGACAGATTTCACCCTGAGAATCAGCAGAGTGGAGGCTGACGATACTG |
| | | GAGTTTATTACTGCGGGCAAGTTATACAAGATCCTACGTTCGGCCAAGGGACCA |
| | | AGGTGGAGATCAAAC |
| 146 | PMX139 VL_AA | SGDIVMTQTPLSLSVSPGETASISCKASQSLLHIDGNTYLNWFRQKPGQSPQRLIYRV |
| | | STRDPGVPDRFSGSGSGTDFTLRISRVEADDTGVYYCGQVIQDPTFGQGTKVEIK |
| 147 | PMX139 CDR1H | GFTFNTY |
| 148 | PMX139 CDR2H | TSGGIS |
| 149 | PMX139 CDR3H | CATYGSYFFDSW |
| 150 | PMX139 CDR1L | ISCKASQSLLH |
| 151 | PMX139 CDR2L | QSPQRLI |
| 152 | PMX139 CDR3L | CGQVIQDPTF |
| 153 | PMX140 VH_NT | GAGGTGCAGCTGGTGGAGTCTGGGGGAGACCTGGTGAAGCCTGGGGGGTCCCTG |
| | | AGACTTTCCTGTTTGGCCTCTGGATTCACCTTCAGTAACTTCCACATGAACTGGG |
| | | TCCGCCAGGCTCCAGGGAAGGGGCTTCAGTGGGTCGCATTCGTAAACGTTGGTG |
| | | GAATTAGAACAGCCTATGCAGACGGTGTGAAGGGCCGATTCACCATCTCCAGAG |
| | | ACGACGCCAAAAACACGCTCTATCTTCAGATGGACAGCCTGCGAGCCGACGACA |
| | | CGGCCGTATATTACTGTGCGAGGGCTTTCCCGGGGGACTACTGGGGCCAGGGAA |
| | | CCCTGGTCACCGTCTCCTCAG |
| 154 | PMX140 VH_AA | EVQLVESGGDLVKPGGSLRLSCLASGFTFSNFHMNWVRQAPGKGLQWVAFVNVGG |
| | | IRTAYADGVKGRFTISRDDAKNTLYLQMDSLRADDTAVYYCARAFPGDYWGQGTL |
| | | VTVSS |
| 155 | PMX140 VL_NT | TCCTATGTGCTGTCTCAGCCGCCATCAGCGACTGTGACTCTGAGGCAGACGGCCC |
| | | GCCTCACCTGTGGGGGAGACAGCATTGGGAGAAAAAGTGTTGAATGGTATCAGC |
| | | AGAAGCCGGGCCAGCCCCCCGTGCTCATTATCTTTGGTGATAGCAGCAGGCCGT |
| | | CAGGGATCCCTGAGCGATTCTCCGGCGCCAACTCGGGGAACACGGCCACCCTGA |
| | | CCATCAGCGGGGCCCTGGCCGAGGACGAGGCTGACTATTACTGCCAGGTGTGGG |
| | | ACAGCAATACTAAGGCTTGGGTATTCGGTGAAGGGACCCAGCTGACCGTCCTCG |
| 156 | PMX140 VL_AA | SYVLSQPPSATVTLRQTARLTCGGDSIGRKSVEWYQQKPGQPPVLIIFGDSSRPSGIPE |
| | | RFSGANSGNTATLTISGALAEDEADYYCQVWDSNTKAWVFGEGTQLTVL |
| 157 | PMX140 CDR1H | GFTFSNF |
| 158 | PMX140 CDR2H | NVGGIR |
| 159 | PMX140 CDR3H | CARAFPGDYW |
| 160 | PMX140 CDR1L | GGDSIGRKSVE |
| 161 | PMX140 CDR2L | GDSSRPS |
| 162 | PMX140 CDR3L | CQVWDSNTKAWVF |
| 163 | PMX141 VH_NT | GAGGTGCAACTGGTGGAGTCTGGGGGAGACCTGGTGAAGCCTGGGGGGTCCCTG |
| | | AGACTTTCCTGTGTGGCCTCTGGATTCACCTTCAGTACCTACCACATGAGCTGGG |
| | | TCCGCCAGGCTCCAGGGAGGGGACTTCAGTGGGTCGCATACATTAACAGTGGTG |
| | | GAACTATGATAAGCTATACAGACACTGTGAAGGGCCGATTCACCATCTCCAGAG |
| | | ACGACGCCAAGAGCACGCTGTATCTTCAAATGAACAGCCTGAGAGCCGAGGAC |
| | | ACGGCCGTTTATTACTGTGCGAGTCACTTCAGTAGCTACTACTGGGGCCAGGGA |
| | | ACCCTGGTCACCGTCTCCTCAG |
| 164 | PMX141 VH_AA | EVQLVESGGDLVKPGGSLRLSCVASGFTFSTYHMSWVRQAPGRGLQWVAYINSGG |
| | | TMISYTDTVKGRFTISRDDAKSTLYLQMNSLRAEDTAVYYCASHFSSYYWGQGTLV |
| | | TVSS |
| 165 | PMX141 VL_NT | TCCTATGTGCTGTCTCAGCCGCCATCAGCGACTGTGACTCTGAAACAGACGGCCC |
| | | GCCTCACCTGTGGGGGAGACAGCATTGGAAGGAAAAGTGTTGAATGGTACCAGC |
| | | AGAAGCCGGGCCAGCCCCCCGTGCTCATTATCTATGGTGATAGCAGCAGGCCGT |
| | | CAGGGATCCCTGAGCGATTCTCCGGCGCCAACTCGGGGAACACGGCCACCCTGA |
| | | CCATCAGCGGGGCCCTGGCCGAGGACGAGGCTGACTATTACTGCCAGGTGTGGG |
| | | ACAGCAGTAGTAAGGCTAGGGTTTTCGGTGAAGGGACCCAGCTGACCGTCCTCG |
| 166 | PMX141 VL_AA | SYVLSQPPSATVTLKQTARLTCGGDSIGRKSVEWYQQKPGQPPVLIIYGDSSRPSGIP |
| | | ERFSGANSGNTATLTISGALAEDEADYYCQVWDSSSKARVFGEGTQLTVL |
| 167 | PMX141 CDR1H | GFTFSTY |
| 168 | PMX141 CDR2H | NSGGTM |
| 169 | PMX141 CDR3H | CASHFSSYYW |
| 170 | PMX141 CDR1L | GGDSIGRKSVE |
| 171 | PMX141 CDR2L | GDSSRPS |
| 172 | PMX141 CDR3L | CQVWDSSSKARVF |
| 173 | PMX142 VH_NT | GAGGTGCAGCTGGTGGAGTCTGGGGGAGACCTGGTGAAGCCTGGGGGGTCCCTG |
| | | AGACTTTCCTGTTTGGCCTCTGGATTCACCTTCAGTAACTTCCACATGAACTGGG |
| | | TCCGCCAGGCTCCAGGGAAGGGGCTTCAGTGGGTCGCATTCGTAAACGTTGGTG |
| | | GAATTAGAACAGCCTATGCAGACGCTGTGAAGGGCCGATTCACCATCTCCAGAG |
| | | ACGACGCCAAAAACACGCTCTATCTTCAGATGGACAGCCTGCGAGCCGACGACA |
| | | CGGCCGTATATTACTGTGCGAGGGCATTCCCGGGGGACTACTGGGGCCAGGGAA |
| | | TTCTGGTCACCGTCTCCTCAG |
| 174 | PMX142 VH_AA | EVQLVESGGDLVKPGGSLRLSCLASGFTFSNFHMNWVRQAPGKGLQWVAFVNVGG |
| | | IRTAYADAVKGRFTISRDDAKNTLYLQMDSLRADDTAVYYCARAFPGDYWGQGIL |
| | | VTVSS |
| 175 | PMX142 VL_NT | TCCTATGTGCTGTCTCAGCCGCCATCAGCGACTGTGACTCTGAGGCAGACGGCCC |
| | | GCCTCACCTGTGGGGGAGACAGCATTGGGAGAAAAAGTGTTGAATGGTATCAGC |
| | | AGAAGCCGGGCCAGCCCCCCGTGCTCATTATCTTTGGTGATAGCAGCAGGCCGT |
| | | CAGGGATCCCTGAGCGATTCTCCGGCGCCAACTCGGGGAACACGGCCACCCTGA |
| | | CCATCAGCGGGGCCCTGGCCGAGGACGAGGCTGACTATTACTGCCAGGTGTGGG |
| | | ACAGCAGTACTAAGGCTTGGGTATTCGGTGAAGGGACCCAGCTGACCGTCCTCG |
| 176 | PMX142 VL_AA | SYVLSQPPSATVTLRQTARLTCGGDSIGRKSVEWYQQKPGQPPVLIIFGDSSRPSGIPE |
| | | RFSGANSGNTATLTISGALAEDEADYYCQVWDSSTKAWVFGEGTQLTVL |
| 177 | PMX142 CDR1H | GFTFSNF |
| 178 | PMX142 CDR2H | NVGGIR |
| 179 | PMX142 CDR3H | CARAFPGDYW |
| 180 | PMX142 CDRIL | GGDSIGRKSVE |
| 181 | PMX142 CDR2L | GDSSRPS |
| 182 | PMX142 CDR3L | CQVWDSSTKAWVF |
| 183 | PMX143 VH_NT | GAGGTGCAGCTGGTGGAGTCTGGGGGAGACCTGGTGAAGCCTGGGGGGTCCCTG |
| | | AGACTTTCCTGTTTGGCCTCTGGATTCACCTTCAGTAACTTCCACATGAACTGGG |
| | | TCCGCCAGGCTCCAGGGAAGGGGCTTCAGTGGGTCGCATTCGTAAACGTTGGTG |
| | | GAATTAGAACAGCCTATGCAGACGCTGTGAAGGGCCGATTCACCATCTCCAGAG |
| | | ACGACGCCAAAAACACGCTCTATCTTCAGATGGACAGCCTGCGAGCCGACGACA |
| | | CGGCCATATATTACTGTGCGAGGGCATTCCCGGGGGACTACTGGGGCCAGGGAA |
| | | CCCTGGTCACCGTCTCCTCAG |
| 184 | PMX143 VH_AA | EVQLVESGGDLVKPGGSLRLSCLASGFTFSNFHMNWVRQAPGKGLQWVAFVNVGG |
| | | IRTAYADAVKGRFTISRDDAKNTLYLQMDSLRADDTAIYYCARAFPGDYWGQGTL |
| | | VTVSS |
| 185 | PMX143 VL_NT | TCCTATGTGCTGTCTCAGCCGCCATCAGCGACTGTGACTCTGAGGCAGACGGCCC |
| | | GCCTCACCTGTGGGGGAGACAGCATTGGGAGAAAAAGTGTTGAATGGTATCAGC |
| | | AGAAGCCGGGCCAGCCCCCCGTGCTCATTATCTTTGGTGATAGCAGCAGGCCGT |
| | | CAGGGATCCCTGAGCGATTCTCCGGCGCCAACTCGGGGAACACGGCCACCCTGA |
| | | CCATCAGCGGGGCCCTGGCCGAGGACGAGGCTGACTATTACTGCCAGGTGTGGG |
| | | ACAGCAGTACTAAGGCTTGGGTATTCGGTGAAGGGACCCAGCTGACCGTCCTCG |
| 186 | PMX143 VL_AA | SYVLSQPPSATVTLRQTARLTCGGDSIGRKSVEWYQQKPGQPPVLIIFGDSSRPSGIPE |
| | | RFSGANSGNTATLTISGALAEDEADYYCQVWDSSTKAWVFGEGTQLTVL |
| 187 | PMX143 CDR1H | GFTFSNF |
| 188 | PMX143 CDR2H | NVGGIR |
| 189 | PMX143 CDR3H | CARAFPGDYW |
| 190 | PMX143 CDR1L | GGDSIGRKSVE |
| 191 | PMX143 CDR2L | GDSSRPS |
| 192 | PMX143 CDR3L | CQVWDSSTKAWVF |
| 193 | PMX144 VH_NT | GAGGTGCAACTGGTGGAGTCTGGGGGAGACCTGGTGAAGCCTGGGGGGTCCCTG |
| | | AGACTTTCCTGTGTGGCCTCTGGATTCACCTTCAGTACCTACCACATGAGCTGGG |
| | | TCCGCCAGGCTCCAGGGAAGGGGCTTCAGTGGGTCGCATACATTAGCAGTGATG |
| | | GAAGAATCATAAGTTATGCAGACGCTGTGAAGGGCCGATTCACCATCTCCAGAG |
| | | ACAACGCCAAGAAAATGTTGTATCTTCAGATGAACAGCCTGAGCGTCGAGGACA |
| | | CGGCCGTATATTACTGTGCGAGTCACTTCGCTAGTTACTTCTGGGGCCAGGGAAC |
| | | CCTGGTCACCGTCTCCTCAG |
| 194 | PMX144 VH_AA | EVQLVESGGDLVKPGGSLRLSCVASGFTFSTYHMSWVRQAPGKGLQWVAYISSDG |
| | | RIISYADAVKGRFTISRDNAKKMLYLQMNSLSVEDTAVYYCASHFASYFWGQGTLV |
| | | TVSS |
| 195 | PMX144 VL_NT | TCCTATGTGCTGTCTCAGCCGCCATCAGCGACTGTGACTCTGAGGCAGACGGCCC |
| | | GCCTCACCTGTGGGGGAGACAGCATTGGACGTAAAAGTGTTGAATGGTACCAGC |
| | | AGAAGCCGGGCCAGCCCCCCGTGCTCATTATATATGGTGATAGTAGTAGGCCGT |
| | | CAGGGATCCCTGAGCGATTCTCCGGCGCCAACTCGGGGAACACGGCCACCCTGA |
| | | CCATCAGCGGGGCCCTGGCCGAGGACGAGGCTGACTATTACTGCCAGGTGTGGG |
| | | ACAGCAGAACTAAGGCTAGGGTATTCGGTGAAGGGACCCAGCTGACCGTCCTCG |
| 196 | PMX144 VL_AA | SYVLSQPPSATVTLRQTARLTCGGDSIGRKSVEWYQQKPGQPPVLIIYGDSSRPSGIP |
| | | ERFSGANSGNTATLTISGALAEDEADYYCQVWDSRTKARVFGEGTQLTVL |
| 197 | PMX144 CDR1H | GFTFSTY |
| 198 | PMX144 CDR2H | SSDGRI |
| 199 | PMX144 CDR3H | CASHFASYFW |
| 200 | PMX144 CDR1L | GGDSIGRKSVE |
| 201 | PMX144 CDR2L | GDSSRPS |
| 202 | PMX144 CDR3L | CQVWDSRTKARVF |
| 203 | PMX145 VH_NT | GAGGTGCAACTGGTGGAGTCTGGGGGAGACCTGGTGAAGTCGGGGGGGTCCCT |
| | | GAGACTTTCCTGTGTGGCCTCTGGATTCACCTTCAATACCTATTCTATGAACTGG |
| | | GTCCGCCAGGCTCCAGGGAAGGGGCTTCAGTGGGTCGCACGCATTAAAAGTAGT |
| | | GGAAGTAGCACAACCTATGCAGACGCTGTGAAGGGCCGATTCACCGTCTCCAGA |
| | | GACATCGCCAAGAACACTCTATTTCTTCAGATGAACAGCCTGAGAGCCGAGGAC |
| | | ACGGCCCTGTATTACTGTGCGAGTGAACCGGGGGCACGGTACGAAATATACTTT |
| | | GTGGAGAGTTTCTTTGACTACTGGGGCCAGGGAACCCTGGTCACCGTCTCCTCAG |
| 204 | PMX145 VH_AA | EVQLVESGGDLVKSGGSLRLSCVASGFTFNTYSMNWVRQAPGKGLQWVARIKSSG |
| | | SSTTYADAVKGRFTVSRDIAKNTLFLQMNSLRAEDTALYYCASEPGARYEIYFVESF |
| | | FDYWGQGTLVTVSS |
| 205 | PMX145 VL_NT | TCCTATGTGCTGACACAGCTGCCATCCAAAAATGTGACCCTGAAGCAGCCGGCC |
| | | CACATCACCTGTGGGGGAGACAACATTGGAAGTAAAAGTGTTCACTGGTACCAG |
| | | CAGAAGCTGGGCCAGGCCCCTGTACTGATTATCTATTATGATAGCAGCAGGCCG |
| | | ACAGGGATCCCTGAGCGATTCTCCGGCGCCAACTCGGGGAACACGGCCACCCTG |
| | | ACCATCAGCGGGGCCCTGGCCGAGGACGAGGCTGACTATTACTGCCAGGTGTGG |
| | | GACAGCAGTGCTCAGGCTTGGATATTCGGTGAAGGGACCCAGCTGACCGTCCTC |
| | | G |
| 206 | PMX145 VL_AA | SYVLTQLPSKNVTLKQPAHITCGGDNIGSKSVHWYQQKLGQAPVLIIYYDSSRPTGIP |
| | | ERFSGANSGNTATLTISGALAEDEADYYCQVWDSSAQAWIFGEGTQLTVL |
| 207 | PMX145 CDR1H | GFTFNTY |
| 208 | PMX145 CDR2H | KSSGSS |
| 209 | PMX145 CDR3H | CASEPGARYEIYFVESFFDYW |
| 210 | PMX145 CDR1L | GGDNIGSKSVH |
| 211 | PMX145 CDR2L | YDSSRPT |
| 212 | PMX145 CDR3L | CQVWDSSAQAWIF |
| 213 | PMX146 VH_NT | GAGGTGCAGATGGTGGAGTCTGGGGGAGACCTGGTGAAGCCGGGGGGGTCCCT |
| | | AAGACTTTCCTGTGTGGCCTCTGGATTCACCTTCAGTAACTACCACATGAACTGG |
| | | GTCCGCCAGGCTCCAGGGAAGGGGCTTCAGTGGGTCGCATACATTAACAGAGGT |
| | | GGAAGTAGGACAAGCTATGTAGACGCTGTGAAGGGCCGATTCACCATCTCCAGA |
| | | GACAACGCCAGGAACACGCTGTTTCTTCAGATGAACAGCCTGAGAGCCGAGGAC |
| | | ACGGCCGTGTATTACTGTGCGAGTGAACCGGGGGCACGATATGATATATACTAC |
| | | GTGGAGAGTTTCTTTGAGTCCTGGGGCCAGGGAACCCTGGTCACCGTCTCCTCAG |
| 214 | PMX146 VH_AA | EVQMVESGGDLVKPGGSLRLSCVASGFTFSNYHMNWVRQAPGKGLQWVAYINRG |
| | | GSRTSYVDAVKGRFTISRDNARNTLFLQMNSLRAEDTAVYYCASEPGARYDIYYVE |
| | | SFFESWGQGTLVTVSS |
| 215 | PMX146 VL_NT | TCCTATGTGCTGACACAGCTGCCATCCAAAAATGTGACCCTGAAGCAGCCGGCC |
| | | CACATCACCTGTGGGGGAGACAACATTGGAAGTAAAAGTGTTCACTGGTACCAG |
| | | CAGAAGCTGGGCCAGGCCCCTGTACTGATTATCTATTATGATAGCAGCAGGCCG |
| | | ACAGGGATCCCTGAGCGATTCTCCGGCGCCAACTCGGGGAACACGGCCACCCTG |
| | | ACCATCAGCGGGGCCCTGGCCGAGGACGAGGCTGACTATTACTGCCAGGTGTGG |
| | | GACAGCAGTGCTAAGGCTTGGATATTCGGTGAAGGGACCCAGCTGACCGTCCTC |
| | | G |
| 216 | PMX146 VL_AA | SYVLTQLPSKNVTLKQPAHITCGGDNIGSKSVHWYQQKLGQAPVLIIYYDSSRPTGIP |
| | | ERFSGANSGNTATLTISGALAEDEADYYCQVWDSSAKAWIFGEGTQLTVL |
| 217 | PMX146 CDR1H | GFTFSNY |
| 218 | PMX146 CDR2H | NRGGSR |
| 219 | PMX146 CDR3H | CASEPGARYDIYYVESFFESW |
| 220 | PMX146 CDR1L | GGDNIGSKSVH |
| 221 | PMX146 CDR2L | YDSSRPT |
| 222 | PMX146 CDR3L | CQVWDSSAKAWIF |
| 223 | PMX147 VH_NT | GAGGTGCAGCTGGTGGAGTCTGGGGGAGACCTGGTGAAGTCGGGGGGGTCCCT |
| | | GAGACTTTCCTGTGTGGCCTCTGGATTCACCTTCAGTACCTACTCTATGAACTGG |
| | | GTCCGCCAGGCTCCAGGGAAGGGGCTTCAGTGGGTCGCACGCATTAATAGTGGT |
| | | GGAAGTAGCACAAGCTATGCAGACGCTGTGAAGGGCCGATTCACCGTCTCCAGA |
| | | GACATCGCCAAGAACACTCTATTTCTTCAGATGAACAGCCTGAGAGCCGAGGAC |
| | | ACGGCCCTGTATTACTGTGCGAGTGAACCGGGGGCACGGTACGAAATATACTTT |
| | | GTGGAGAGTTTCTTTGACTACTGGGGCCAGGGAACCCTGGTCACCGTCTCCTCAG |
| 224 | PMX147 VH_AA | EVQLVESGGDLVKSGGSLRLSCVASGFTFSTYSMNWVRQAPGKGLQWVARINSGG |
| | | SSTSYADAVKGRFTVSRDIAKNTLFLQMNSLRAEDTALYYCASEPGARYEIYFVESF |
| | | FDYWGQGTLVTVSS |
| 225 | PMX147 VL_NT | TCCTATGTGCTGACACAGCTGCCATCCAAAAATGTGACCCTGAAGCAGCCGGCC |
| | | CACATCACCTGTGGGGGAGACAACATTGGAAGTAAAAGTGTTCACTGGTACCAG |
| | | CAGAAGCTGGGCCAGGCCCCTGTATTGATTATCTATTATGATAGCAGCAGGCCG |
| | | ACAGGGATCCCTGAGCGATTCTCCGGCGCCAACTCGGGGAACACGGCCACCCTG |
| | | ACCATCAGCGGGGCCCTGGCCGAGGACGAGGCTGACTATTACTGCCAGGTGTGG |
| | | GACAGCAGTGCTAAGGCTTGGATATTCGGTGAAGGGACCCAGCTGACCGTCCTC |
| | | G |
| 226 | PMX147 VL_AA | SYVLTQLPSKNVTLKQPAHITCGGDNIGSKSVHWYQQKLGQAPVLIIYYDSSRPTGIP |
| | | ERFSGANSGNTATLTISGALAEDEADYYCQVWDSSAKAWIFGEGTQLTVL |
| 227 | PMX147 CDR1H | GFTFSTY |
| 228 | PMX147 CDR2H | NSGGSS |
| 229 | PMX147 CDR3H | CASEPGARYEIYFVESFFDYW |
| 230 | PMX147 CDR1L | GGDNIGSKSVH |
| 231 | PMX147 CDR2L | YDSSRPT |
| 232 | PMX147 CDR3L | CQVWDSSAKAWIF |
| 233 | PMX148 VH_NT | GAGGTGCAGCTGGTGGAGTCTGGGGGAGACCTGGTGAAGCCGGGGGGGTCCCT |
| | | GAGACTTTCCTGTGTGGCCTCTGGATTCACCTTCAGTACCTACCACATGAACTGG |
| | | GTCCGCCAGGCTCCAGGGAAGGGGCTTCAGTGGGTCGCACACATTAACAGAAGT |
| | | GGAAGTATTACAAGTTATGTGGACGCTGTGAAGGACCGATTCACCATCTCCAGA |
| | | GACAACGCCAAGAACACGCTGTATCTTCAGCTGAACAGCCTGAGAGCCGAGGAC |
| | | ACGGCCGTGTATTACTGTGCGAGTGAACCGGGGGCACGGTACGATATATACTAC |
| | | GTGGAGAGTTTCTTTGACTACTGGGGCCAGGGAACCCTGGTCACCGTCTCCTCAG |
| 234 | PMX148 VH_AA | EVQLVESGGDLVKPGGSLRLSCVASGFTFSTYHMNWVRQAPGKGLQWVAHINRSG |
| | | SITSYVDAVKDRFTISRDNAKNTLYLQLNSLRAEDTAVYYCASEPGARYDIYYVESF |
| | | FDYWGQGTLVTVSS |
| 235 | PMX148 VL_NT | TCCTATGTGCTGACACAGCTGCCAGCCAAAAATGTGACCCTGAAGCAGGCGGCC |
| | | CACATCACCTGTGGGGGAGACAACATTGGAAGTAAAAGTGTTCACTGGTACCAG |
| | | CAGAAGCTGGGCCAGGCCCCTGTACTGATTATCTATTATGATAGCAGCAGGCCG |
| | | ACAGGGATCCCTGAGCGATTCTCCGGCGCCAACTCGGGGAACACGGCCACCCTG |
| | | ACCATCAGCGGGGCCCTGGCCGAGGACGAGGCTGACTATTACTGCCAGGTGTGG |
| | | GACAGCAGTGCTAAGGCTTGGATATTCGGTGAAGGGACCCAGCTGACCGTCCTC |
| | | G |
| 236 | PMX148 VL_AA | SYVLTQLPAKNVTLKQAAHITCGGDNIGSKSVHWYQQKLGQAPVLIIYYDSSRPTGI |
| | | PERFSGANSGNTATLTISGALAEDEADYYCQVWDSSAKAWIFGEGTQLTVL |
| 237 | PMX148 CDR1H | GFTFSTY |
| 238 | PMX148 CDR2H | NRSGSI |
| 239 | PMX148 CDR3H | CASEPGARYDIYYVESFFDYW |
| 240 | PMX148 CDR1L | GGDNIGSKSVH |
| 241 | PMX148 CDR2L | YDSSRPT |
| 242 | PMX148 CDR3L | CQVWDSSAKAWIF |
| 243 | PMX149 VH_NT | GAGGTGCAACTGGTGGAGTCTGGGGGAGACCTGGTGAAGTCGGGGGGGTCCCT |
| | | GAGACTCTCCTGTGTGGCCTCTGGATTCACCTTCAGTACCTACTCTATGAACTGG |
| | | GTCCGCCAGGCTCCAGGGAAGGGGCTTCAGTGGGTCGCACGCATTAAAAGTAGT |
| | | GGAAGTAGTACAAGCTATGCAGACGCTGTGAAGGGCCGATTCACCGTCTCCAGA |
| | | GACATCGCCAAGAACACTCTATTTCTTCAGATGAACAGCCTGAGAGCCGAGGAC |
| | | ACGGCCCTGTATTTCTGTGCGAGTGAACCGGGGGCACGGTACGAAATATACTTT |
| | | GTGGAGAGTTTCTTTGACTACTGGGGCCAGGGAACCCTGGTCACCGTCTCCTCAG |
| 244 | PMX149 VH_AA | EVQLVESGGDLVKSGGSLRLSCVASGFTFSTYSMNWVRQAPGKGLQWVARIKSSGS |
| | | STSYADAVKGRFTVSRDIAKNTLFLQMNSLRAEDTALYFCASEPGARYEIYFVESFF |
| | | DYWGQGTLVTVSS |
| 245 | PMX149 VL_NT | TCCTATGTGCTGACACAGCTGCCATCCAAAAATGTGACCCTGAAGCAGCCGGCC |
| | | CACATCACCTGTGGGGGAGACAACATTGGAAGTAAAAGTGTTCACTGGTACCAG |
| | | CAGAAGCTGGGCCAGGCCCCTGTACTGATTATCTATTATGATAGCAGCAGGCCG |
| | | ACAGGGATCCCTGAGCGATTCTCCGGCGCCAACTCGGGGAACACGGCCACCCTG |
| | | ACCATCAGCGGGGCCCTGGCCGAGGACGAGGCTGACTATTACTGCCAGGTGTGG |
| | | GACAGCAGTGCTCAGGCTTGGATATTCGGTGAAGGGACCCAGCTGACCGTCCTC |
| | | G |
| 246 | PMX149 VL_AA | SYVLTQLPSKNVTLKQPAHITCGGDNIGSKSVHWYQQKLGQAPVLIIYYDSSRPTGIP |
| | | ERFSGANSGNTATLTISGALAEDEADYYCQVWDSSAQAWIFGEGTQLTVL |
| 247 | PMX149 CDR1H | GFTFSTY |
| 248 | PMX149 CDR2H | KSSGSS |
| 249 | PMX149 CDR3H | CASEPGARYEIYFVESFFDYW |
| 250 | PMX149 CDR1L | GGDNIGSKSVH |
| 251 | PMX149 CDR2L | YDSSRPT |
| 252 | PMX149 CDR3L | CQVWDSSAQAWIF |
| 253 | PMX150 VH_NT | GAGGTGCAACTGGTGGAGTCTGGGGGAGACCTGGTGAAGTCGGGGGGGTCCCT |
| | | GAGACTTTCCTGTGTGGCCTCTGGATTCACCTTCAGTACCTACTCTATGAACTGG |
| | | GTCCGCCAGGCTCCAGGGAAGGGGCTTCAGTGGGTCGCACGCATTAAAAGTAGT |
| | | GGAAGTAGCACAAGCTATGCAGACGCTGTGAAGGGCCGATTCACCGTCTCCAGA |
| | | GACATCGCCAAGAACACTCTATTTCTTCAGATGAACAGCCTGAGAGCCGAGGAC |
| | | ACGGCCTTCTATTACTGTGCGAGTGAACCGGGGGCACGGTACGAAATATACTTT |
| | | GTGGAGAGTTTCTTTGACTACTGGGGCCAGGGAACCCTGGTCACCGTCTCCTCAG |
| 254 | PMX150 VH_AA | EVQLVESGGDLVKSGGSLRLSCVASGFTFSTYSMNWVRQAPGKGLQWVARIKSSGS |
| | | STSYADAVKGRFTVSRDIAKNTLFLQMNSLRAEDTAFYYCASEPGARYEIYFVESFF |
| | | DYWGQGTLVTVSS |
| 255 | PMX150 VL_NT | TCCTATGTGCTGACACAGCTGCCATCCAAAAATGTGACCCTGAAGCAGCCGGCC |
| | | CACATCACCTGTGGGGGAGACAACATTGGAAGTAAAAGTGTTCACTGGTACCAG |
| | | CAGAAGCTGGGCCAGGCCCCTATACTGATTATCTATTATGATATCAGCAGGCCG |
| | | ACAGGGATCCCTGAGCGATTCTCCGGCGCCAAGTCGGGGAACACGGCCACCCTG |
| | | ACCATCAGCGGGGCCCTGGCCGAGGACGAGGCTGACTATTACTGCCAGGTGTGG |
| | | GACAGCAGTGCTCAGGCTTGGATATTCGGTGAAGGGACCCAGCTGACCGTCCTC |
| | | G |
| 256 | PMX150 VL_AA | SYVLTQLPSKNVTLKQPAHITCGGDNIGSKSVHWYQQKLGQAPILIIYYDISRPTGIPE |
| | | RFSGAKSGNTATLTISGALAEDEADYYCQVWDSSAQAWIFGEGTQLTVL |
| 257 | PMX150 CDR1H | GFTFSTY |
| 258 | PMX150 CDR2H | KSSGSS |
| 259 | PMX150 CDR3H | CASEPGARYEIYFVESFFDYW |
| 260 | PMX150 CDR1L | GGDNIGSKSVH |
| 261 | PMX150 CDR2L | YDISRPT |
| 262 | PMX150 CDR3L | CQVWDSSAQAWIF |
| 263 | PMX151 VH_NT | GAGGTGCAACTGGTGGAGTCTGGGGGAGACCTGGTGAAGTCGGGGGGGTCCCT |
| | | GAGACTTTCCTGTGTGGCCTCTGGATTCACCTTCAATACCTATTCTATGAACTGG |
| | | GTCCGCCAGGCTCCAGGGAAGGGGCTTCAGTGGGTCGCACGCATTAAAAGTAGT |
| | | GGAAGTAGCACAACCTATGCAGACGCTGTGAAGGGCCGATTCACCGTCTCCAGA |
| | | GACATCGCCAAGAACACTCTATTTCTTCAGATGAACAGCCTGAGAGCCGAGGAC |
| | | ACGGCCCTGTATTACTGTGCGAGTGAACCGGGGGCACGGTACGAAATATACTTT |
| | | GTGGAGAGTTTCTTTGACTACTGGGGCCAGGGAACCCTGGTCACCGTCTCCTCAG |
| 264 | PMX151 VH_AA | EVQLVESGGDLVKSGGSLRLSCVASGFTFNTYSMNWVRQAPGKGLQWVARIKSSG |
| | | SSTTYADAVKGRFTVSRDIAKNTLFLQMNSLRAEDTALYYCASEPGARYEIYFVESF |
| | | FDYWGQGTLVTVSS |
| 265 | PMX151 VL_NT | TCCTATGTGCTGACACAGCTGCCATCCAAAAGTGTGACCCTGAAGCAGCCGGCC |
| | | CACATCACCTGTGGGGGAGACAACATTGGAAGTAAAAGTGTTCACTGGTACCAG |
| | | CAGAAGCTGGGCCAGGCCCCTGTACTGATTATCTATTATGATAGCAGCAGGCCG |
| | | ACAGGGATCCCTGAGCGATTCTCCGGCGCCAACTCGGGGAACACGGCCACCCTG |
| | | ACCATCAGCGGGGCCCTGGCCGAGGACGAGGCTGACTATTACTGCCAGGTGTGG |
| | | GACAGCAGTGCTCAGGCTTGGATATTCGGTGAAGGGACCCAGCTGACCGTCCTC |
| | | G |
| 266 | PMX151 VL_AA | SYVLTQLPSKSVTLKQPAHITCGGDNIGSKSVHWYQQKLGQAPVLIIYYDSSRPTGIP |
| | | ERFSGANSGNTATLTISGALAEDEADYYCQVWDSSAQAWIFGEGTQLTVL |
| 267 | PMX151 CDR1H | GFTFNTY |
| 268 | PMX151 CDR2H | KSSGSS |
| 269 | PMX151 CDR3H | CASEPGARYEIYFVESFFDYW |
| 270 | PMX151 CDR1L | GGDNIGSKSVH |
| 271 | PMX151 CDR2L | YDSSRPT |
| 272 | PMX151 CDR3L | CQVWDSSAQAWIF |
| 273 | PMX152 VH_NT | GAGGTGCAGCTGGTGGAGTCTGGGGGAGACCTGGTGAAGCCTGGGGGGTCCCTG |
| | | AGACTTTCCTGTGTGGCCTCTGGATTCACCTTCAGTAGATACTACATGAACTGGG |
| | | TCCGCCAGGCTCCAGGGAAGGGGCCTCAGTGGGTCGCATACATTAACAATGCTG |
| | | GAAGTGATAGAAATTATGTAGACGCTGTGAAGGGCCGATTCACCATCTCCAGAG |
| | | ACAACGCCAAGAACACGCTGTACCTTCAGATGAACAGCCTGAGAGCCGAGGAC |
| | | ACGGCCGTGTATTATTGTGCGAGTGAAATAAGGGGGGTTGTAGTAGTTGGACAT |
| | | CTTGACTACTGGGGCCAGGGAACCCTGGTCACCGTCTCCTCAG |
| 274 | PMX152 VH_AA | EVQLVESGGDLVKPGGSLRLSCVASGFTFSRYYMNWVRQAPGKGPQWVAYINNAG |
| | | SDRNYVDAVKGRFTISRDNAKNTLYLQMNSLRAEDTAVYYCASEIRGVVVVGHLD |
| | | YWGQGTLVTVSS |
| 275 | PMX152 VL_NT | TCCTATGTGCTGACACAGCTGCCATCCAAAAGTGTGACCCTGAACCAGCCGGCC |
| | | CACATCACCTGTGGGGGAGACAACATTGGAAGTAAAAGTGTTCACTGGTACCAG |
| | | CAGAAGCTGGGCCAGGCCCCTGTACTGATTATCTATTATGATAGCATCAGGCCG |
| | | ACAGGGATCCCTGAGCGATTCTCCGGCGCCACCTCGGGGAACACGGCCACCCTG |
| | | ACCATCAGCGGGGCCCTGGCCGAGGACGAGGCTGACTATTACTGCCAGGTGTGG |
| | | GACAGCAGTGCTAAGGCTTGGGTATTCGGTGAAGGGACCCAGCTGACCGTCCTC |
| | | G |
| 276 | PMX152 VL_AA | SYVLTQLPSKSVTLNQPAHITCGGDNIGSKSVHWYQQKLGQAPVLIIYYDSIRPTGIP |
| | | ERFSGATSGNTATLTISGALAEDEADYYCQVWDSSAKAWVFGEGTQLTVL |
| 277 | PMX152 CDR1H | GFTFSRY |
| 278 | PMX152 CDR2H | NNAGSD |
| 279 | PMX152 CDR3H | CASEIRGVVVVGHLDYW |
| 280 | PMX152 CDR1L | GGDNIGSKSVH |
| 281 | PMX152 CDR2L | YDSIRPT |
| 282 | PMX152 CDR3L | CQVWDSSAKAWVF |
| 283 | PMX153 VH_NT | GAGGTGCAGCTGGTGGAGTCTGGGGGAGACCTGGTGAAGCCTGGGGGGTCCCTG |
| | | AGACTTTCCTGTGTGGCCTCTGGATTCACCTTCAGTAGATACTACATGAACTGGG |
| | | TCCGCCAGGCTCCAGGGAAGGGGCCTCAGTGGGTCGCATACATTAACAATGGTG |
| | | GAAGTGATAGAAATTATGTAGACGCTGTGAAGGGCCGATTCACCATCTCCAGAG |
| | | ACAACGCCAAGAACACGCTGTACCTTCAGATGAACAGCCTGAGAGCCGAGGAC |
| | | ACGGCCGTGTATTATTGTGCGAGTGAAATAAGGGGGGTTGTAGTAGTTGGACAT |
| | | CTTGACTACTGGGGCCAGGGAACCCTGGTCACCGTCTCCTCAG |
| 284 | PMX153 VH_AA | EVQLVESGGDLVKPGGSLRLSCVASGFTFSRYYMNWVRQAPGKGPQWVAYINNGG |
| | | SDRNYVDAVKGRFTISRDNAKNTLYLQMNSLRAEDTAVYYCASEIRGVVVVGHLD |
| | | YWGQGTLVTVSS |
| 285 | PMX153 VL_NT | TCCTATGTGCTGACACAGCTGCCATCCAAAAATGTGACCCTGAACCAGCCGGCC |
| | | CACATCACCTGTGGGGGAGACAACATTGGAAGTAAAAGTGTTCACTGGTACCAG |
| | | CAGAAGCTGGGCCAGGCCCCTGTACTGATTATCTATTATGATAGCATCAGGCCG |
| | | ACAGGGATCCCTGAGCGATTCTCCGGCGCCACCTCGGGGAACACGGCCACCCTG |
| | | ACCATCAGCGGGGCCCTGGCCGAGGACGAGGCTGACTATTACTGCCAGGTGTGG |
| | | GACAGCAGTGCTAAGGCTTGGGTATTCGGTGAAGGGACCCAGCTGACCGTCCTC |
| | | G |
| 286 | PMX153 VL_AA | SYVLTQLPSKNVTLNQPAHITCGGDNIGSKSVHWYQQKLGQAPVLIIYYDSIRPTGIP |
| | | ERFSGATSGNTATLTISGALAEDEADYYCQVWDSSAKAWVFGEGTQLTVL |
| 287 | PMX153 CDR1H | GFTFSRY |
| 288 | PMX153 CDR2H | NNGGSD |
| 289 | PMX153 CDR3H | CASEIRGVVVVGHLDYW |
| 290 | PMX153 CDR1L | GGDNIGSKSVH |
| 291 | PMX153 CDR2L | YDSIRPT |
| 292 | PMX153 CDR3L | CQVWDSSAKAWVF |
| 293 | IgGB_Def2 | ASTTAPSVFPLAPSCGSTSGSTVALACLVSGYFPEPVTVSWNSGSLTSGVHTFPSVLQ |
| | | SSGLYSLSSMVTVPSSRWPSETFTCNVAHPASKTKVDKPVPKRENGRVPRPPDCPKC |
| | | PAPESLGGPSVFIFPPKPKDTLLIARTPEVTCVVVDLGREDPEVQISWFVDGKQMQTA |
| | | KTQPREEQFNGTYRVVSVLPIGHQDWLKGKQFTCKVNHIGLPSPIERTISKARGQAH |
| | | QPSVYVLPPSREELSKNTVSLTCLIKDFFPPDIDVEWQSNGQQEPESKYRTTPPQLDE |
| | | DGSYFLYSKLSVDKSRWQRGDTFICAVMHEALHNHYTQKSLSHSPGK |
| 294 | Lambda_5_ | GQPKASPSVTLFPPSSEELGANKATLVCLISDFYPSGVTVAWKADGSPITQGVETTKP |
| | constant | SKQSNNKYAASSYLSLTPDKWKSHSSFSCLVTHEGSTVEKKVAPAECS |
| 295 | Kappa_constant | RNDAQPAVYLFQPSPDQLHTGSASVVCLLNSFYPKDINVKWKVDGVIQDTGIQESV |
| | | TEQDKDSTYSLSSTLTMSSTEYLSHELYSCEITHKSLPSTLIKSFQRSECQRVD |

The invention is further illustrated in the following non-limiting examples.

### EXAMPLES

### Example 1: Canine PD-1, PD-L1 and PD-L2 Sequences

Canine PD-1 (ENSCAFG00000013184), PD-L1 (ENSCAFG00000002120) and PD-L2 (ENSCAFG00000002121) coding sequences were synthesised (and where appropriate, codon-optimised) according to the sequences predicted from the genes found in the canine reference genome assembly (CanFam3.1) found in the Ensembl genome browser after verifying sequence homology with well-annotated human and mouse orthologs. The coding sequences of canine PD-1, PD-L1 and PD-L2 were confirmed by RNA-sequencing following standard approaches.

### Example 2: Expression of Canine PD-1 in HEK Cells & MEF Cells

Human embryonic kidney (HEK) 293 cells were grown on 90 mm round tissue culture plates as monolayers in DMEM/F12 (Life Technologies, California, US) supplemented with 10% fetal bovine serum (FBS; Sigma Aldrich) at 37° C. in a humidified atmosphere containing 5% CO₂. HEK293 cells were transfected with either PD-1, PD-L1 or PD-L2 using Lipofectamine LTX with Plus reagent (Life technologies, California, USA) according to the manufacturer's recommendations. Stable integration of the plasmid's expression cassette was ensured by using PiggyBac transposon elements in the expression vector and by including 1% w/w transposase-encoding plasmid along with the expression plasmid. Stably transfected cells were selected with 3 μg/mL puromycin 48 h after transfection for a total of 6 days.

Mouse embryonic fibroblasts (MEF) were grown on 90 mm round tissue culture plates as monolayers in DMEM-high glucose with L-glutamine (Life Technologies) supplemented with 10% FBS and 1×MEM nonessential amino acids (Gibco) at 37° C. in a moist atmosphere and 5% CO₂. Cells were transfected with PD-1 using Lipofectamine LTX with Plus reagent (Life technologies, California, USA) according to the manufacturer's recommendations. Stable integration of the plasmid's expression cassette was ensured by using PiggyBac transposon elements in the expression vector and by including 1% w/w transposase-encoding plasmid along with the expression plasmid. Stably transfected cells were selected with 3 μg/mL puromycin 48 h after transfection for a total of 6 days.

### Example 3: Expression of Recombinant Soluble Canine PD-1, PD-L1 and PD-L2 Proteins

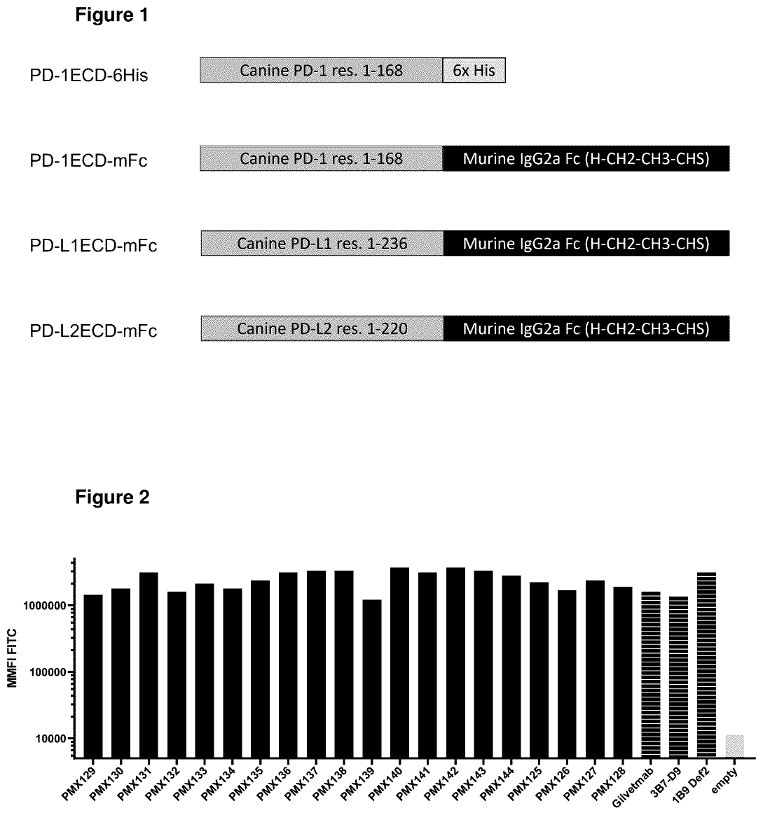

Chinese hamster ovary (CHO) cells, grown in F17 medium (Gibco) supplemented with 4 mM L-glutamine in a humidified environment at 37° C. with 8% CO₂, were stably transfected with expression plasmids encoding PD-1 ECD-mFc, PD-1 ECD-6His, PD-L1 ECD-mFc and PD-L2 ECD-mFc (ECD=extracellular domain). PD-1 ECD, PD-L1 ECD and PD-L2 ECD refer to the predicted extracellular domains of PD-1, PD-L1 and PD-L2, respectively. The “-mFc” suffix refers to the Fc portion of murine IgG2a comprising the genetic annotations Hinge-Constant Heavy2-Constant Heavy3-Constant Heavy Secreted (H-CH2-CH3-CHS) and “6His” refers to a hexahistidine tag (FIG. 1).

Stable integration of the plasmid's expression cassette was ensured by using PiggyBac transposon elements in the expression vector and by including 1% w/w transposase-encoding plasmid along with the expression plasmid. 3 E+06 cells were transfected with 2 μg expression plasmid encoding the appropriate protein and 20 ng transposes-expressing plasmid. Briefly, the DNA was mixed with polyethylenimine (PEI, 25 kDa linear, PolySciences) at a 1:3 ratio (w/w) of DNA:PEI in 50 μL Opti-MEM and incubated for 15 mins at room temperature, then added dropwise to the cell culture.

After 24 h, cells were selected with 25 μg/mL puromycin for a total of 8 days. After selection, cells were seeded at 3×10⁶ in 100 mL culture media and incubated at 32° C., 8% CO₂ with shaking at 150 rpm. 4% HyClone Cell Boost 7a supplement+0.4% HyClone Cell Boost 7b supplement+1% glucose was added to the media on days 1, 4, 7 and 10. Culture supernatants were collected on day 12. Supernatants were harvested by centrifuging at 1000 g for 15 mins, followed by a 0.2 μm filtration step. Proteins were purified from supernatant by affinity chromatography using standard protein A affinity chromatography (for Fc-tagged proteins) or Ni-NTA IMAC approaches (6His-tagged proteins) using an AKTA FPLC system (Cytiva). In all cases, proteins were immediately buffer-exchanged into PBS and concentrated using 10 kDa MWCO Amicon spin filter devices. After four rounds of buffer-exchange, proteins were 0.2 μm filtered and quantified by absorbance at 280 nm.

### Example 4: Immunisation

Ky9™ mice, a transgenic mouse platform capable of generating antibodies with canine variable domains, for example substantially as described in WO2018/189520 and WO2020/074874, were immunised.

The transgenic mice have been modified compared to wild type mice by insertion of immunoglobulin heavy (IGH) chain and light chain (IGL) variable (V) region genes, IGH D region genes and IGH J region genes from a dog into a mouse which allows the production of antibody heavy chains that comprise a variable antibody region originating from the expression of canine DNA in the mouse, in combination with a constant region.

The constant region may be the rodent immunoglobulin (IG) constant region, resulting in production of chimeric heavy chains carrying a canine variable region and a murine constant region. Information concerning, or the nucleic acid comprising, the variable region of such chimeric antibody chains may be used to generate fully canine antibodies, for therapeutic use in dogs for example.

Mice were immunised in a prime boost regime by intraperitoneal (I.P) administration of 2.5 E+06 MEF cells stably expressing PD-1, formulated in 50% PBS and 50% Sigma Adjuvant system adjuvant (Sigma Aldrich). Spleens and mesenteric lymph nodes were harvested 10 days following the final boost. Alternatively, mice were immunised with recombinant canine PD-1 extracellular domain protein (PD-1 ECD-mFc) three or four times at 2 or 3-week intervals. Protein immunogen was delivered at two sites simultaneously: intraperitoneally and sub-cutaneously formulated in 50% PBS and 50% Sigma Adjuvant system adjuvant (Sigma Aldrich). Spleens and mesenteric lymph nodes were harvested 4 days following the final boost.

Determination of serum titres: Mice were bled prior to immunisation and 10 days following the first boost. Blood was collected and serum-separated using microvette 200 Z-gel tubes (Starstedt AG & Co. KG). Serum titres of PD-1-specific IgG was evaluated by measuring the binding of dilutions of serum to PD-1-expressing HEK293 cells. Immune sera were serially diluted in FACS buffer and incubated for 1 hour at 4° C. with 1×105 HEK293 cells stably expressing PD-1, then washed in 200 μL FACS buffer. Specific IgG was detected with a trio of BB700-conjugated monoclonal antibodies against murine isotypes IgG1, IgG2a, IgG2b (BD OptiBuild™, Becton Dickinson), diluted 1:400 for 1 hour at 4° C. before being washed in 200 μL FACS buffer. HEK293 cell geometric mean fluorescence intensity was calculated by flow cytometry using a Beckton-Coulter Cytoflex. Data were analyzed in FlowJo version 10.6.1. Pre-immune sera at a 1:100 dilution was used to set the background threshold.

### Example 5: Isolation of Antibody Producing Cells & Identifying Antibody Sequence of Candidate Molecules

Tissue isolation: Spleens and mesenteric lymph nodes were harvested from immunised mice. Splenocytes were prepared by cutting the spleen into pieces and forcing these through a 45 μm cell strainer (Falcon) while rinsing with RPMI-1640 (Lonza, Basel, CH)+10% FBS on ice. A similar process was used for lymphocytes from lymph nodes. Cells were pelleted at 300 g for 5 min, before being resuspended in PBS+2% FBS+1 mM EDTA if directly used for flow sorting or being frozen at −150° C. in 90% FBS+10% DMSO for later usage.

Cell Sorting: Prior to antigen-specific cell sorting, T/erythroid cells were depleted using biotinylated anti-CD90.2 and anti-ter119 antibodies followed by binding to streptavidin rapidspheres according to the manufacturer's instructions (StemCell Technologies UK). Following this, bulk cell sorting was performed using a BD FACSAria Fusion flow sorter. The markers CD19, IgM and IgD were used to identify class-switched B cells and CD138 (Syndecan-1)+CD267 (TACI) were used to mark plasmablast and plasma cell populations within the spleen+lymph node cells (SP+LN). Within the class-switched B cell population, cells expressing BCRs specific for canine PD-1 were detected using amine-labelled AlexaFluo647-conjugated PD-1 ECD-mFC fusion proteins were sorted by FACS. Markers to identify unwanted cell populations and dead cells (CD8a; CD4; Fixable viability dye) were included in all staining panels to exclude these cells.

Sorted cells were prepared for antibody profiling using the 10× Genomics Chromium Single Cell Immune Profiling system and the V(D)J Kit (10× Genomics) according to the manufacturer's instructions. Nucleotide sequences of expressed antibodies were determined by Illumina sequencing.

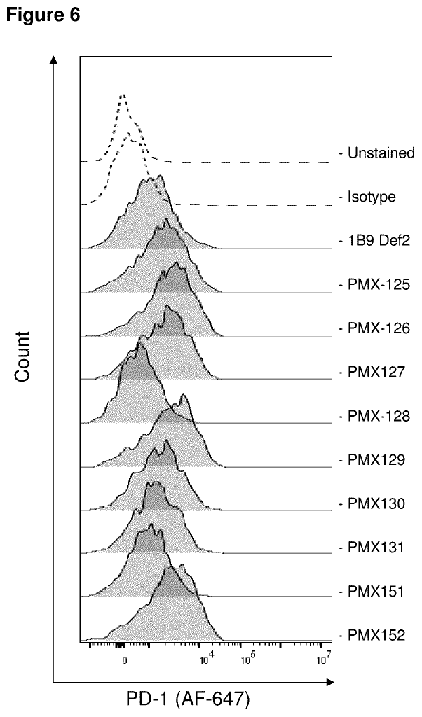

The variable immunoglobulin region comprises a VDJ region of an immunoglobulin nucleotide sequence for heavy genes and a VJ region of an immunoglobulin nucleotide sequence for IgK and IgA. Within a clonal family there are subfamilies with shared mutations within their V(D)J segments that arise during immunoglobulin gene recombination and somatic hypermutation. Different clonal families that display unique V(D)J segment usage usually exhibit different binding characteristics. During recombination and hypermutation, cells whose antibodies have a higher affinity for an antigen were selected, and if low affinity clones from the same lineage have a neutralising function, the affinity usually increases with further mutations; for example, a clustered family is shown in FIG. 6 of WO2015/040401.

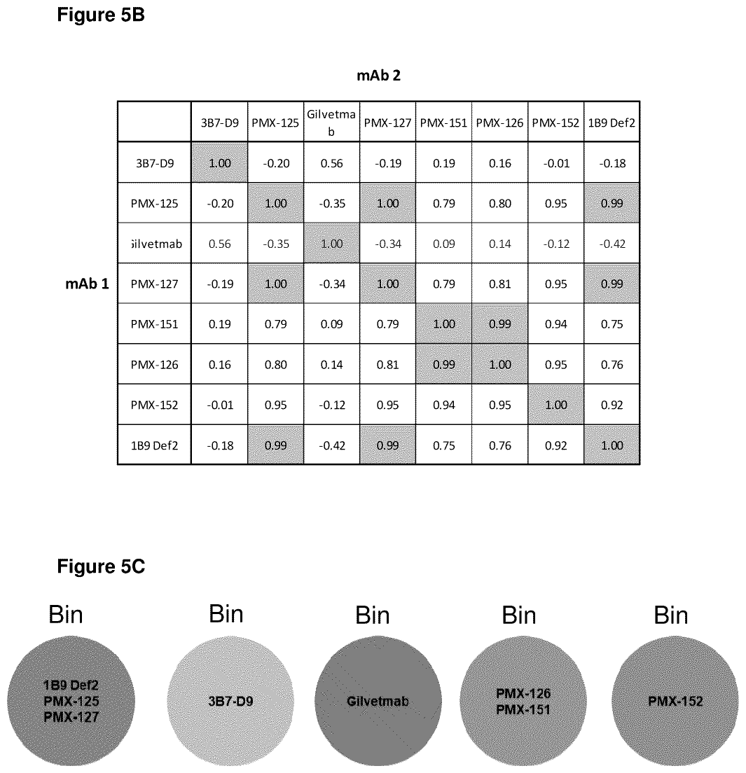

A clonal family is generally defined by related immunoglobulin heavy chain and/or light chain V(D)J sequences of two or more clonal cells. Related immunoglobulin heavy chain V(D)J sequences can be identified by their shared usage of V(D)J gene segments. An example of the analysis of antibody sequences of sorted Ag-specific single B-cells is shown in FIG. 5 of WO2015/040401, and shows antibody sequences that are arranged by heavy-chain V-gene family usage, and clustered to generate the displayed phylogenetic trees. From phylogenetic trees such as these, candidate clones were selected.

### Example 6: Recombinant Expression of Monoclonal Antibodies

The heavy chain and light chain variable region sequence of selected candidate clones were synthesised and cloned into expression vectors containing sequences encoding an effector-deficient dog IgGB constant heavy region (see WO2023012496), and the wild-type dog lambda constant 5 light region, or the wild-type dog kappa constant light region. The expression vectors encoding the heavy chain and light chain were co-transfected into CHO cells to obtain stable expression. For antibody production, 3×10⁶ selected CHO cells were seeded in 3 mL culture media and incubated at 32° C., 8% CO₂ with shaking at 200 rpm. 4% HyClone Cell Boost 7a supplement+0.4% HyClone Cell Boost 7b supplement+1% glucose was added to the media on days 1, 4, 7 and 10. Culture supernatants were collected on day 12 and the IgG concentration determined by assaying for protein A binding using surface plasmon resonance (Biacore 8K, Cytiva)

### Example 7: Flow Cytometry Cell Binding Assay

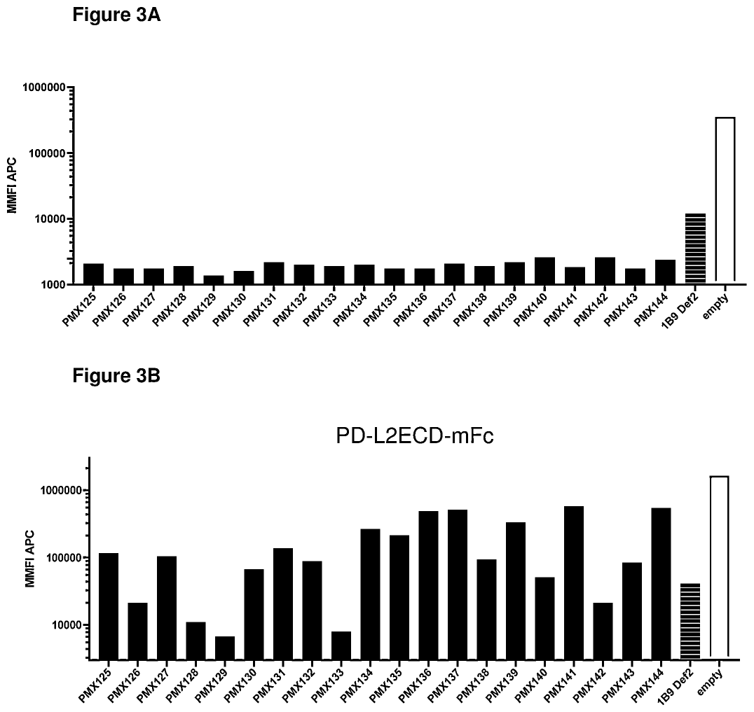

As PD-1 is a transmembrane protein, the most conformationally correct protein is embedded in the plasma membrane. Binding to plasma membrane-embedded protein can be measured by measuring binding to cells stably transfected with canine PD-1 by flow cytometry. 1 E+05 HEK293 cells stably transfected with canine PD-1 were incubated in CHO supernatant containing IgG at 2 μg/mL for one hour at 4° C. Cells were washed in 200 μL FACS buffer twice before being incubated with a 1:300 dilution of Goat anti-dog H+L-FITC conjugated secondary antibody (Abcam) for 30 mins at 4° C. in the dark. Cells were then washed once in 200 μL FACS buffer and resuspended in 200 μL FACS buffer and acquired on a BD Accuri C6 Plus flow cytometer. Results shown in FIG. 2 indicate mAbs PMX125-144 all bind canine full-length PD-1 in transmembrane form to a comparable degree to benchmark anti-canine PD-1 binding mAbs Gilvetmab, 3B7-D and 1B9 Def2. Gilvetmab is a canine PD-1 binding IgGB Kappa antibody whose full-length heavy and light chain amino acid sequences are described in: WHO Drug Information, Vol. 30, No. 4, 2016 (https://www.antibodysociety.org/wordpress/wp-content/uploads/2017/07/INN-2017-Reference-26.pdf). 3B7-D9 is a PD-1 binding IgGD Kappa antibody whose variable regions are described in US2017/0158764A1 (Variable heavy SEQ ID NO 2, variable light SEQ ID NO 3). Here, its light chain variable region is grafted onto a wild-type canine kappa constant (aka IGKC) sequence and its heavy chain variable region is grafted onto wild-type IgGD canine constant (aka IGHG4). 1B9 Def2 is an IgGB Def2 Kappa antibody whose variable regions are described in WO2020/103885A1 (Variable heavy SEQ ID NO 50, variable light SEQ ID NO 52). Here, its light chain variable region is grafted onto a wild-type canine kappa constant sequence (aka IGKC) and its heavy chain variable sequence is grafted onto PetMedix's “Def2” modified IgGB constant Fc domain which is described in WO2023012496.

### Example 8: PD-L1/PD-L2 Blocking Assays Using Flow Cytometry

Blockade of the interaction between PD-1 and its ligands PD-L1 and PD-L2 is the major mechanism of action of checkpoint inhibitors targeting this axis. It is assumed that in vitro blockade of this interaction is a pre-requisite for in vivo function.

### Flow Cytometry-Based Ligand Blocking Assay

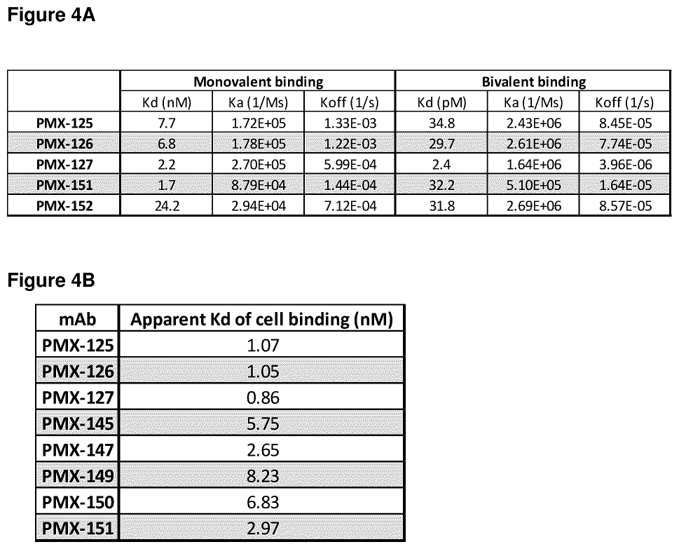

1 E+05 HEK293 cells stably transfected with canine PD-1 were incubated in CHO supernatant containing 2 μg/mL of recombinant IgG for one hour at 4° C. Cells were then washed with 200 μL FACS buffer twice before incubating the cells with 1 μg/mL PD-L1 ECD-mFc or PDL2 ECD-mFc labelled with phycoerythrin for 1 hour at 4° C. Cells were then washed once in 200 μL FACS buffer and resuspended in 200 μL FACS buffer and acquired on a Beckman-Coulter CytoFlex flow cytometer. Data shown in FIG. 3A and FIG. 3B indicate mAbs PMX125-PMX144 all potently block PD-L1 binding to PD-1 and that a subset also block PD-L2 binding to PD-1, more potently than control antibody 1B9 Def2. A subset of PD-L1 blockers also block PD-L2 binding to PD-1, although only PMX126, PMX128, PMX129, PMX133 and PMX142 more potently than 1B9 Def2.

### Example 9: Affinity Assays—SPR & Flow Cytometry

### SPR-Based Affinity Assay

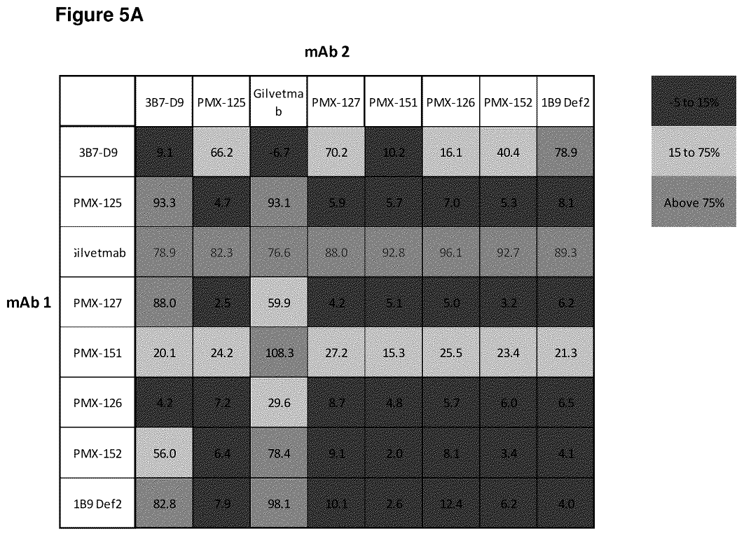

SPR-based monomeric affinity assays were conducted as multi-cycle kinetic experiments. Around 100 RU of purified monoclonal antibody was captured on Fc2 of a series S protein A chip (Cytiva). After capture, monomeric PD-1 ECD-6His protein was injected at 2-fold dilutions from 100 nM to 0.13 nM at 30 μL/min with an association time of 150 s and a dissociation time of 700 s. SPR-based bivalent affinity assays were conducted as multi-cycle kinetic experiments. Around 170 RU of purified PD-1 ECD-mFc protein was captured on Fc2 of a series S CM5 chip (Cytiva). After capture, monoclonal antibodies were injected at 2-fold dilutions from 11.1 nM to 0.04 nM at 30 μL/min with an association time of 180 s and a dissociation time of 1000 s. At the end of each cycle, the chip surface was regenerated with a 120 s pulse of 10 mM Glycine pH 1.9 at 30 μL/min. The data were analysed using Biacore Insight package (Cytiva) by fitting to a global 1:1 binding model with kinetic analysis. The mAbs analysed has Ks values in the single-digit (double digit for PMX-152) nanomolar range when assayed in monovalent mode and in the single or double digit picomolar range when assayed in bivalent mode (FIG. 4A).

### Flow Cytometry-Based Apparent Affinity Assay

5 E+04 HEK cells stably expressing PD-1 were incubated in 250 μL FACS buffer containing purified mAb diluted in 3-fold concentrations ranging from 300 nM to 10 pM for 2 hours at 4° C. Cells were then washed twice in 200 μL FACS buffer and incubated in a 1:300 dilution of Goat anti-dog H+L-FITC conjugated secondary antibody (Abcam) for 30 mins at 4° C. in the dark. Cells were then washed once in 200 μL FACS buffer and resuspended in 200 μL FACS buffer and acquired on a BD Accuri C6 Plus flow cytometer. Apparent affinities were then derived by reporting the concentration at which signal is 50% maximal after fitting the data to a “[Agonist] vs. response” 4-parameter logistic curve in Prism 8.3.0 (GraphPad). The resulting EC₅₀ values (apparent affinity) measured were in the single digit nanomolar range for all mAbs analysed (FIG. 4B)

### Example 10: Epitope Binning

Epitope binning was performed with an “in tandem” SPR-based approach. Approximately 200 RU of PD-1 ECD-mFc was amine coupled at pH 5.0 to Fc2 on a CM5 chip, according to standard manufacturer's recommendations (Cytiva). The in tandem assay consisted of injecting in “Dual” mode mAb 1 at 200 nM over the chip for 300 s immediately followed by mAb 2 at 200 nM for 300 s at a flow rate of 10 μL/min. Low ligand densities, along with a high mAb 1 concentration (200 nM) and long injections (300 s) ensured ligand saturation. A strong signal following mAb2 injection indicates a non-competing binding mode in the mAb pair whereas little to no signal following mAb2 injection indicates a competing binding mode in the mAb pair. Following the end of the cycle, the chip surface was regenerated with a 120 s pulse of 10 mM Glycine HCl pH 1.9 at 30 μL/min. The running buffer used was HBS-EP+ (Cytiva) and the data were collected on a Biacore 8K instrument (Cytiva). Gilvetmab (WHO Drug Information, Vol. 30, No. 4, 2016), 3B7-D9 (US2017/0158764A1) and 1B9 Def2 (WP2020/103885A1) are three mAbs which bind canine PD-1. FIG. 5A shows the fraction of mAb2 binding when mAb1 was prebound to saturating conditions. Values lower than 15% indicate complete blockade of a pair of mAbs whereas values above 75% indicate negligible blockade of mAb 2 binding by mAb 1. Analysing the competition relationships between mAbs, permits in some cases the distinction of binding epitopes of mAbs which cross-compete on a one-to-one basis. Correlating the relative binding profile of each mAb in the matrix (column of relative binding values) with that of each other mAb, when appropriately thresholded, can highlight non-obvious binding epitope similarities between mAbs (FIG. 5B). These results suggest that PMX126 and PMX151 access an epitope which, although overlapping with the binding epitope of previously characterised mAbs 1B9 Def2, Gilvetmab and 3B7-D9, can be shown to be distinct, however the binding epitope of PMX125 and PM127 cannot be resolved from that of 1B9 Def2, using this limited set of mAbs (FIG. 5B and FIG. 5C)

### Example 11: Binding to Activated Canine T Cells

Canine PBMCs isolated from healthy dogs were cultured in 96-well round bottom plates at a concentration of 1×10⁵ cells per well in complete RPMI medium (RPMI-1640+10% FBS+1 U/mL penicillin+10 μg/mL streptomycin). Cells were stimulated with concanavalin A (ConA) at 1 μg/ml for 96 hours to upregulate PD-1 expression before being collected for flow cytometry binding analysis. For the assessment of PD-1 antibody binding on activated T cells, PD-1 antibodies were directly conjugated using to Alexa Fluor® 647. Cells were resuspended in FACS buffer and incubated with the commercial canine PE-conjugated CD5 (clone YKIX322.3, BioRad) to identify T cells and the Alexa 647-conjugated PD-1 antibodies at a final concentration of 5 μg/mL for 30 min at 4° C. was used to detect PD-1. Fixable Viability Dye eFluor 780 (eBioscience) was used to label and exclude dead cells. Results shown in FIG. 6 indicate that all mAbs tested bind canine PD-1 on ConA-activated canine T cells

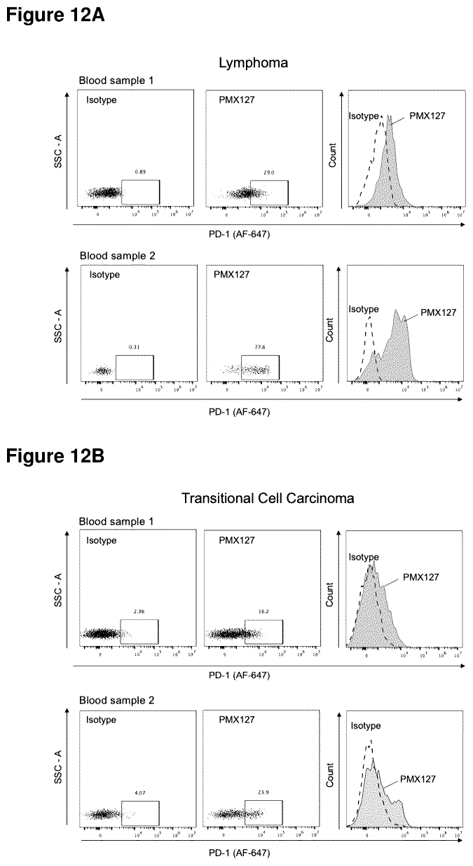

The ability of PD-1 antibody to bind to PD-1 on T cells of cancer-bearing dogs was also evaluated as cancer samples are thought to have higher levels of PD-1/PD-L1. Samples from dogs with cancer were provided by Oncology Services of the Queen Mother Hospital for Animals at the Royal Veterinary College (RVC), UK. For this analysis, peripheral blood leukocytes were isolated from canine blood following red blood cell lysis (eBioscience). Cells were stained with anti-canine PE-conjugated CD5 (clone YKIX322.3, BioRad) and Alexa 647-conjugated PMX127 antibody as previously described and analysed by flow cytometry. For assessment of PD-1 expression by T cells, lymphocytes were gated initially by forward and side-scatter properties, followed by CD5⁺ population. Gates for analysis of PD-1⁺ cells were based on binding of isotype control antibody by the CD5⁺ population. Results shown in FIG. 12A and FIG. 12B demonstrated the ability of PD-1 antibody to detect PD-1 expression by canine CD5⁺ T cells

### Example 12: Functional In Vitro Assay IFN-γ Secretion Assay

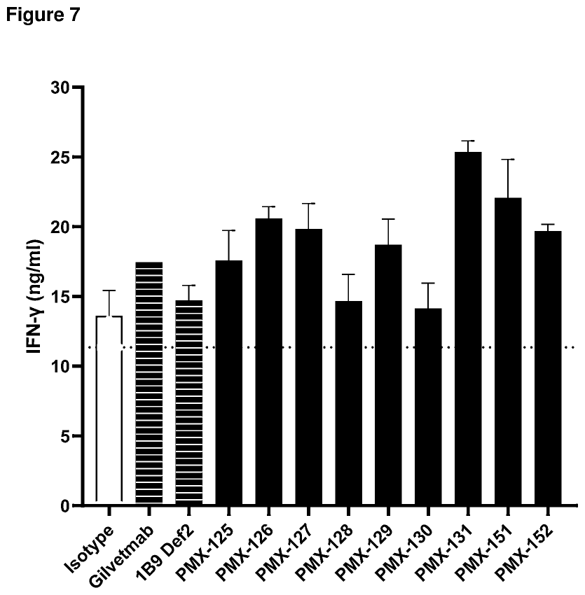

Blood from healthy beagle dogs was collected in EDTA tubes. Peripheral blood mononuclear cells (PBMC) were isolated from blood following density centrifugation with Ficoll-Paque PLUS 1.077 g/mL (Cytiva) in Leucosep tubes (Greiner Bio-One). Isolated PBMCs were washed twice, resuspended in complete RPMI-1640 medium (Lonza) and plated in triplicate into 96-well round bottom plates at a concentration of 2.5×10⁵ cells per well. Cells were stimulated with concanavalin A (ConA) at 1 μg/ml and incubated for 96 hours. Antibodies were added at a final concentration of 10 μg/mL at the time of stimulation. For IFN-γ secretion measurement, supernatants were collected after 96 hours incubation and IFN-γ levels were quantified by sandwich ELISA using a canine IFN-gamma ELISABASIC kit (Mabtech AB) according to the manufacturer's recommended protocol. For the evaluation of T cell proliferation, following the 96-hour conA stimulation, cells were pelleted at 400 g for 5 mins, resuspended in PBS+3% FBS+2 mM EDTA (FACS buffer) and stained with anti-dog CD5-RPE (Bio-Rad), Fixable viability dye eFluor 780 (Invitrogen) and anti-mouse/rat Ki-67 eFluor 450 (Invitrogen) for 30 mins at 4° C. Cells were pelleted and washed twice in FACS buffer and acquired on a Beckman-Coulter Cytoflex flow cytometer. Lymphocytes were first gated on using FSC and SSC profiling and percentages of live, CD5⁺, Ki-67⁺ cells were assessed in each sample in triplicate. Data was analyzed in FlowJo version 10.6.1. Results shown in FIG. 7 indicate that several mAbs are able to significantly increase the secretion of IFN-γ from canine PBMC, an indicator of increased T cell activation. PMX126, PM127, PM131, PMX151 and PM152 were able to increase T cell activation above the levels observed for Gilvetmab and 1B9 Def2.

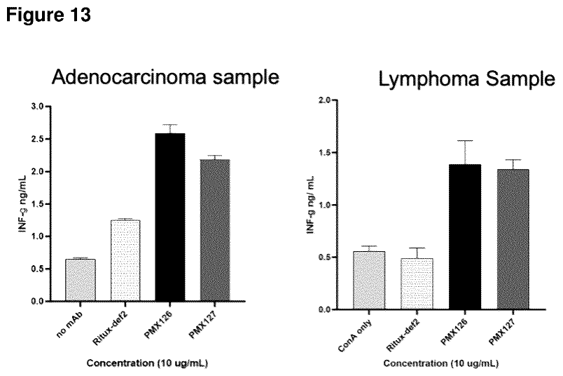

The effects of PD-1 blockade on INF-gamma production in blood samples from dogs (obtained from RVC as above) with cancer were evaluated. Canine leukocytes isolated from dogs with cancer were stimulated with ConA at 1 mg/ml in the presence of PMX126 or PMX127 and cultures incubated for 96 h. Controls included cells incubated without antibody, and an irrelevant IgG isotype-matched antibody. After 96 hours of incubation, the supernatants were collected, and the concentrations of IFN-gamma in the supernatants were measured by ELISA (R&D Systems, USA). An Increase in IFN-gamma production was observed with both of PMX126 and PMX127 antibodies compared to controls (FIG. 13).

### Example 13: Caninized Cell-Based Assay PD-1 Signalling Reporter Assay

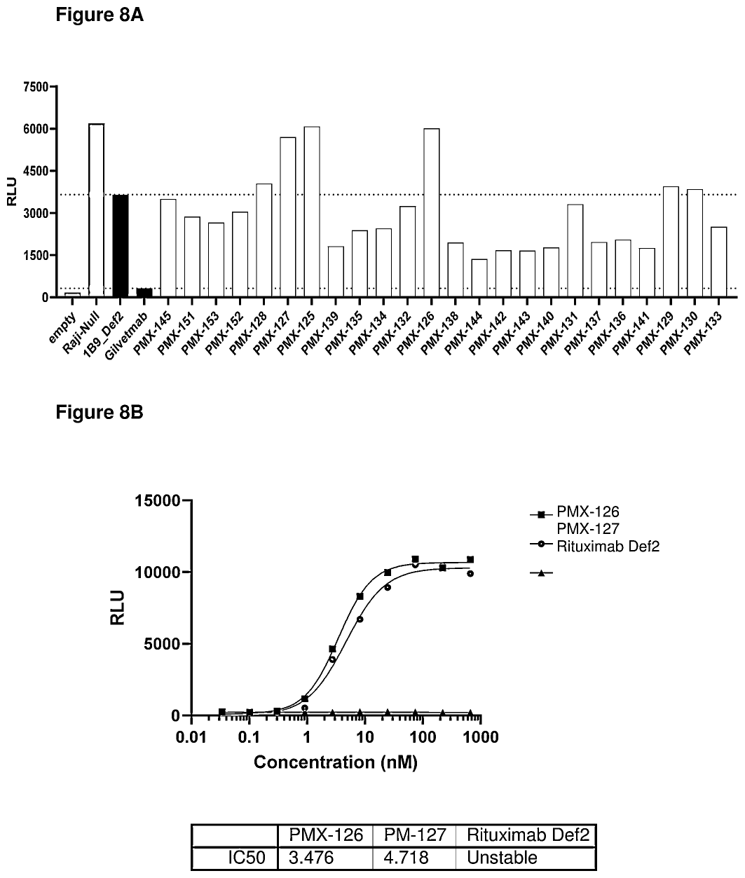

To evaluate the immunological activity of the antibody candidates, we tested their ability to disrupt the PD-1/PD-L1 inhibitory signalling in a bioluminescent reporter PD-1/PD-L1 cell-based assay adapted from commercially available cell lines (Invivogen, France). In this assay, NFAT-luc reporter Jurkat T cells were genetically engineered to express chimeric dog (extracellular domain)/human (transmembrane and intracellular domain) PD-1 instead of full-length human PD-1 and Raji antigen-presenting B cells were engineered to overexpress full-length canine PD-L1. Raji-Null cells expressing no PD-L1 were used as positive control for the bioluminescent signalling. Briefly, 1×10⁵ chimeric PD-1 NFAT-luc Jurkat cells were co-cultured with 5×10⁴ canine PD-L1 Raji-APC cells per well in a total volume of 200 μL in the presence of anti-canine PD-1 antibody candidates at 3 μg/mL or a control antibody. Co-cultures were incubated overnight at 37° C. and a bioluminescence measurement was performed by adding the luciferase detection reagent QUANTI-Luc gold (Invivogen, France) according to the manufacturer's instructions. Results shown in FIG. 8A,B indicate that all mAbs assayed had the ability to inhibit PD-1 signalling by interfering with the canine PD-1/PD-L1 interaction. In this assay, PMX125, PMX126 and PMX127 led to a marked increased in signalling blockade compared to both Gilvetmab and 1B9 Def2.

### Example 14: In Vivo Assessment: MC-38 Tumour Treatment in Caninised Mouse Model

Transgenic C57BL/6 mice carrying a chimeric PD-1 bearing a murine transmembrane and intracellular domain (coding residues 170VIG-WPL288) and a canine extracellular domain composed of exon 2 and part of exon 3 (coding residues 27SPD-QGL169) were created by methods known by those skilled in the art and named C57BL/6^{cPD-1/cPD-1}. A PD-L1-positive murine colorectal cancer cell line named MC-38 is routinely used for the assessment of human checkpoint inhibitors. This cell line can be stably transfected using PiggyBac transposase system to constitutively express the canine PD-L1 ortholog (MC-38^{cPD-1+}). In the transgenic C57BL/6^{cPD-1/cPD-1} mice, the MC-38^{cPD-1+} cell line is able to grow at the same rate as MC-38. Three days after sub-cutaneous inoculation of 2.5 E5 MC-38^{cPD-1+} cells in a 50% v/v formulation of PBS and Matrigel hESC (Corning) or when tumours reach 150 mm³, a weekly intraperitoneal administration of 2.5 mg/kg anti-canine PD-1 mAb is made. Tumour size is monitored by measuring the tumour in two dimensions and calculating the volume is derived using the formula: Tumour volume=((width)²×length)/2.

### Example 15: Epitope Mapping

To determine the exact binding site of monoclonal antibodies to a protein including interacting residues, epitope mapping can be carried out. Several methods are available including X-ray crystallography, cross-linking mass spectrometry, hydrogen-deuterium-exchange mass spectrometry (HDX-MS) or alanine scanning mutagenesis, methods for which are all known to those skilled in the art. Alanine scanning mutagenesis consists of sequentially mutating residues to alanine, measuring the interaction strength to the variants compared to the wild-type protein and deducing the target residues which contribute significantly to binding. Alanine is chosen for its small sidechain composed of just a methyl group and its standard Ramachandran plot features making it unlikely to significantly affect the local alpha carbon structure around the mutation.

### Example 16: In Vivo Dog Studies

Lead candidate antibodies are administered by intravenous infusion into healthy Beagles at 3 or 0.6 mg/kg of bodyweight. Pharmacokinetics (pK) are monitored with a direct ELISA assay using the target of interest. A linear pK relationship with an elimination half-life of approximately 8-15 days is anticipated.

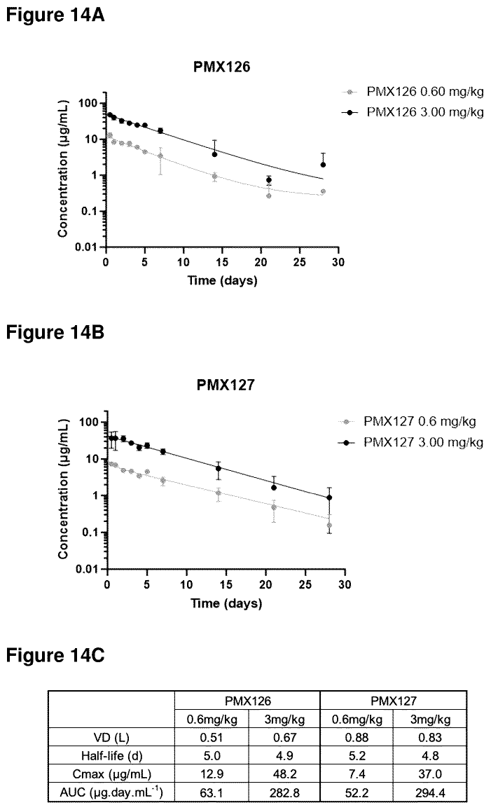

Canine PD-1 antibody plasma concentrations were detected using ELISA. For this assay, ELISA 96-well plates (Thermofisher) were coated overnight 4° C. with 5 μg/mL of canine PD-1 Fc-tag protein produced in-house. After washing and blocking steps, serially diluted dog plasma (12-point dilution from 250 μg/mL starting concentration) and goat anti-dog IgG HRP-conjugated secondary antibodies (Bethyl labs) were added. Canine PD-1 plasma concentrations were determined using SuperSignal ELISA Pico Chemiluminescent Substrate (Thermofisher). The quantification of bound PMX126 and PMX127 antibodies in plasma was determined by measuring the optical densities absorption at 450 nm on a CLARIOstar microplate reader. Plasma concentrations of PMX126 and PMX127 antibodies were calculated from the standard curve using a one-phase decay model in GraphPad Prism software. Linear pK and a half-life of 5 days were observed. (FIGS. 14A, B, C).

Safety and tolerability are the other major readouts. In particular immune-related adverse effects (irAEs) are monitored closely by a veterinarian and full blood counts and liver enzyme activity tests are performed. In multi-dose studies, anti-drug immunogenicity is monitored closely by measuring the presence of anti-drug antibodies (ADAs) using standard approaches including acid-dissociation or ionic dissociation ELISA-based ADA assays which have been described previously^{1,2}.

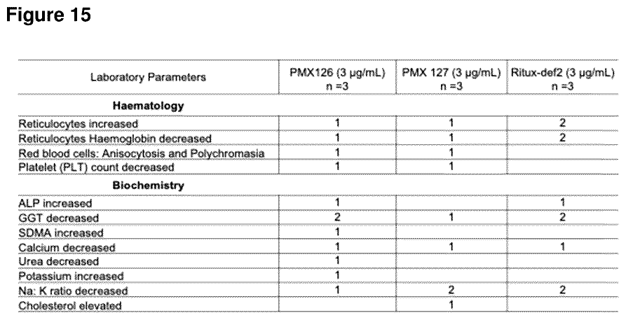

In an exploratory single-dose target animal safety and pK study, animals were monitored for adverse events during the in-life phase. Clinical observations of animals, weight monitoring, haematology, and serum biochemistry analyses were performed before treatment and then weekly throughout the study period for all animals. Adverse events were categorized using the Veterinary Cooperative Oncology Group (VCOG)—Common Terminology Criteria for Adverse Events (VCOG-CTCAE v2) following investigational therapy in dogs and cats. Single intravenous administration of PMX126 and PMX127 to healthy beagles at doses of 0.6 and 3 mg/kg was well tolerated with no effects on body weight or clinical observations. Clinical pathology analysis showed only mild and transient alterations that may not have been related to treatment (FIG. 15)

### Example 17: Alanine Scanning Mutagenesis

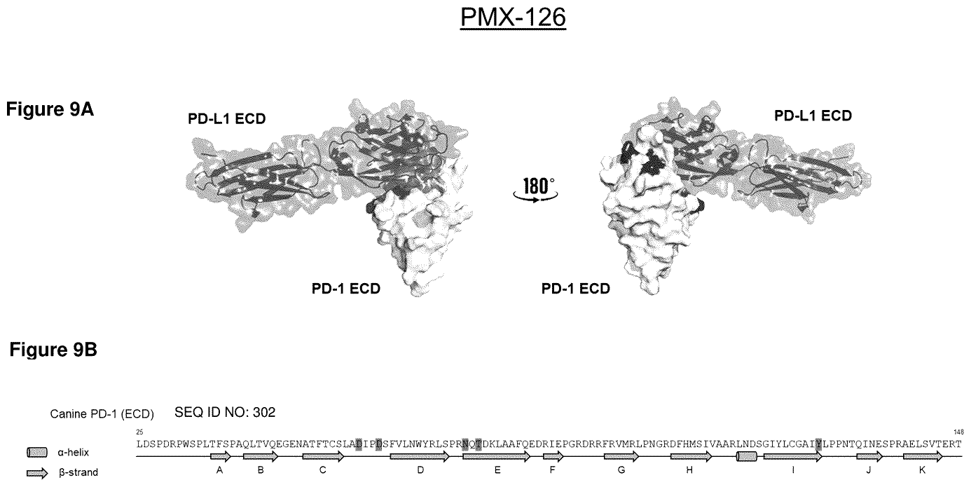

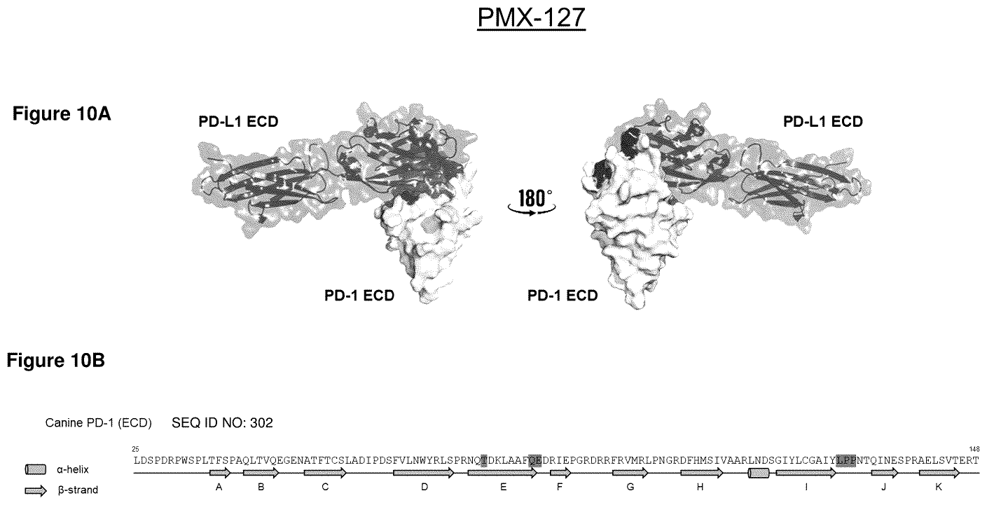

Alanine scanning experiments were carried out by creating a panel of canine PD-1 mutant plasmids in which each position of the predicted extracellular domain (L25-L166) contains a single codon substitution coding for the amino acid alanine. Each mutant plasmid and the wild-type plasmid were separately transfected into HEK293 cells to generate 134 stable cell lines using PiggyBac transposase technology coupled with drug-selection of the transgene. All cell lines were continuously cultured under selective pressure. To control for variable expression levels of the PD-1 mutants across the 134 cell lines, PD-1 binding mAbs which fail to block PD-L1 binding were identified to use as PD-1 expression controls (IM84-187 and IM84-223). The inability of IM84-187 and IM84-223 to interfere with the binding to PD-1 of PMX-126 and PMX-127 was extensively validated by real-time competition assays by SPR as well as endpoint competition assays by flow cytometry. Test mAbs PMX-126 and PMX-127 and expression control mAbs IM84-187 and IM84-223 were directly labelled with AlexaFluor-647 and AlexaFluor-555, respectively, using standard amine coupling chemistry. In each experiment, one test mAb (either PMX-126 or PMX-127) and one expression control mAb (IM84-187 or IM187-223) were used simultaneously to co-stain each mutant stable cell line at a concentration of 0.9 μg/mL (test mAbs) and 4.1 μg/mL (expression control mAbs). Binding to each stable cell line was measured by flow cytometry. Cell lines which showed sub-proportional binding of the test mAb to the cell line in comparison to the expression control mAb was inferred to be involved in the binding of the test mAb. In practice, this was determined by a non-diagonal downward shift on a 2D scatterplot of AlexaFluor-647 (ordinate) and AlexaFluor-555 (abscissa). Proportional signal intensity shifts of both the AlexaFluor-647 and AlexaFluor-555 signals were attributed to differing amounts of membrane-bound PD-1 on the cell surface of the different stable cell lines; this could be caused by factors such as transgene integration copy number or location or the expression level of the mutant PD-1 proteins. Using this method, several residue positions which interact with PMX-126 (D58, D61, N74, T76 and Y127) and PMX-127 (T76, Q83, E84, L128, P129 and P130) were identified and are shown on structural representations of PD-1/PD-L1 extracellular domain structural representations (FIG. 9A,B and FIG. 10A,B).

### Example 18: Cross-Reactivity to Human PD-1

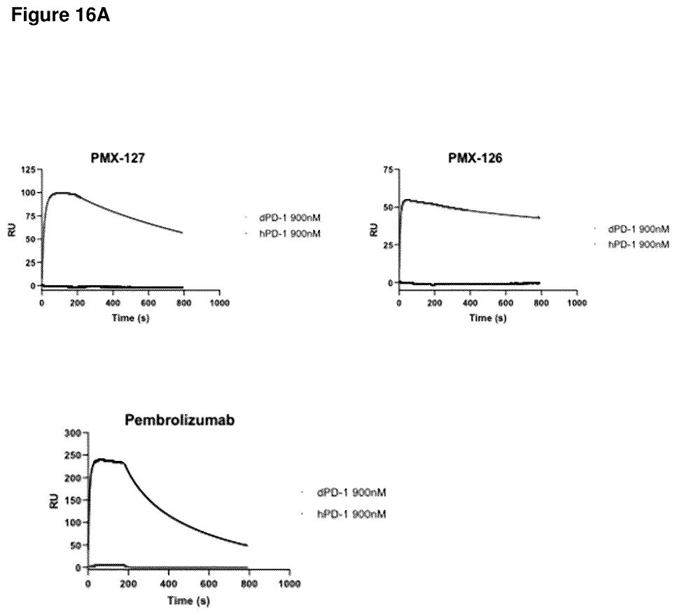

Cross-reactivity of the anti-canine mAbs PMX-126 and PMX-127 to human PD-1 protein was tested by SPR and by ELISA. In the SPR-based assay, approximately 300 RU of antibodies PMX-126 and PMX-127 were captured on flow cell 2 of a series S sensor chip protein A (Cytiva) and a 180 s injection of recombinant canine PD-1-ECD-6His protein (produced in-house) or a recombinant human PD-1-ED-6His protein (Bio-Techne, item BP164049-1 mg) at a concentration of 900 nM and a flow rate of 30 μL/min was made. The assay was also performed by capturing Pembrolizumab analogue (Biosynth AG) to ensure the human PD-1 ECD-6His protein was correctly folded and active. Neither PMX-126 or PMX-127 cross-reacted with human PD-1 ECD-6His protein and Pembrolizumab bound only to human PD-1 ECD-6His protein (FIG. 16A). The ELISA assay was performed by coating Maxisorp plates (Nunc) with 100 μL recombinant canine PD-1-ECD-6His protein or 100 μL recombinant human PD-1-ECD-6His protein, both at 3 μg/mL overnight at 4° C. Plates were then washed twice with PBS-Tween20, blocked with 2% Albumin Fraction V in PBS for 1 h, washed again with PBS-Tween20 and incubated for 1 h at room temperature with 100 μL of a 12-point 3-fold dilution series of unlabelled mAb with a top concentration of 100 μg/mL. Antibody binding was detected using anti-canine IgG-HRP conjugate (anti-human IgG-HRP conjugate for Pembrolizumab) diluted 1:8000 and the ELISA was developed with 100 μL TMB substrate solution (Thermo Fisher). The reaction was stopped with 50 μL 1 M H₂SO₄ and plates were read at 450 nm. Neither PMX-126 or PMX-127 cross-reacted with human PD-1 ECD-6His protein, even up to a concentration of 100 μg/mL (FIG. 16B).

### Example 19: Receptor Occupancy

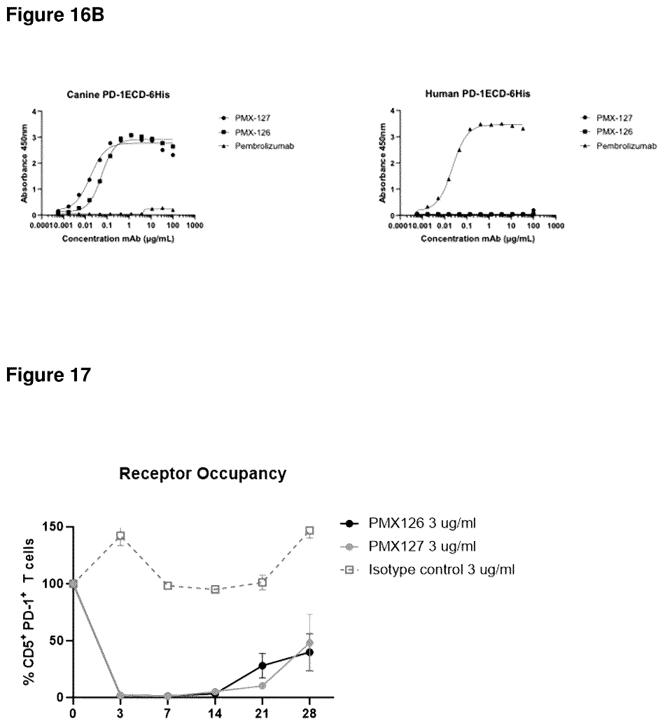

Receptor occupancy (RO) of PD-1 antibodies on T cells were determined using a flow cytometry assay to identify unoccupied PD-1 receptor in PBMC samples of healthy beagles treated with PMX-126, PMX-127 or a control antibody. After isolating PBMCs using a previously described method, PBMC samples from dogs treated with PMX-126 were incubated with PE-conjugated mouse anti-dog CD5 (Biorad) and Alexa 647-conjugated PMX-126 or anti-canine IgG isotype control for 30 minutes at 4° C. in the dark. PBMC samples from dogs treated with PMX127 were incubated with PE-conjugated mouse anti-dog CD5, and Alexa 647-conjugated PMX-127 or anti-canine IgG isotype control. The samples were centrifuged at 400×g for 3 minutes at 4° C. and washed twice in PBS. The binding of PMX-126 or PMX-127 antibodies to PD-1 molecules on T cells was detected by flow cytometry (FIG. 17).

### Example 20: Differential Expression in Genes Associated with T Cell Function

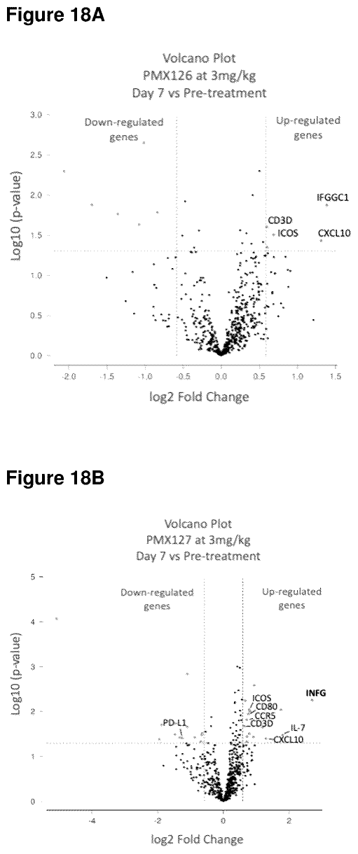

Transcriptomic analysis was performed to evaluate changes in gene expression profiles in PBMC samples of healthy beagles following treatment with PMX-126 and PMX-127 antibodies. RNA was isolated from PBMCs using Quick-DNA/RNA Miniprep Plus Kit (Zymo Research) per the manufacturer's protocol and quantified using a Nanodrop spectrophotometer and a Bioanalyser. Samples were processed in the nCounter Core Facility at University College London (UCL), UK, for detection of genes involved in T cell activation and function using the nCounter Canine 10 Panel (NanoString Technologies). Data was analysed using Rosalind software provided by NanoString. Criteria for significantly differentially expressed genes were chosen based on a log 2 fold-change less than or greater than 1.5, and p<0.05 comparing Day 07 samples to pre-treatment samples. Data was visualized using volcano plots, heatmaps, and histograms for specific genes. Groupwise comparisons were conducted using pre-treatment samples compared to Day 07 samples from each antibody group (PMX126, PMX127 and isotype-matched control). Transcriptomic analysis revealed increased T cell activation signatures following treatment with PMX-126 and PMX-127 (FIG. 18A and FIG. 18B). In particular upregulation of IFGGC1, CD3D, ICOS, CXCL10, CD80, CCR5, IL-7 and INF-gamma was observed.

### REFERENCES

1. Patton, A., Mullenix, M. C., Swanson, S. J. and Koren, E., 2005. An acid dissociation bridging ELISA for detection of antibodies directed against therapeutic proteins in the presence of antigen. Journal of immunological methods, 304(1-2), pp. 189-195.
2. Jordan, G., Pöhler, A., Guilhot, F., Zaspel, M. and Staack, R. F., 2020. High ionic strength dissociation assay (HISDA) for high drug tolerant immunogenicity testing. Bioanalysis, 12(12), pp. 857-866.

PD1 SEQUENCES
Wildtype canine PD-1 nucleotide sequence
(SEQ ID NO: 1):
ATGGGGAGCCGGGGGGGGCCCTGGCCGCTCGTCTGGGCCGTGCTGCAG
CTGGGCTGGTGGCCAGGATGGCTCCTAGACTCCCCTGACAGGCCCTGG
AGCCCGCTCACCTTCTCCCCGGCGCAGCTCACGGTGCAGGAGGGAGAG
AACGCCACGTTCACCTGCAGCCTGGCCGACATCCCCGACAGCTTCGTG
CTCAACTGGTACCGCCTGAGCCCCCGCAACCAGACGGACAAGCTGGCC
GCCTTCCAGGAGGACCGCATCGAGCCGGGCCGGGACAGGCGCTTCCGC
GTCACGCGGCTGCCCAACGGGGGGACTTCCACATGAGCATCGTCGCTG
CGCGCCTCAACGACAGCGGCATCTACCTGTGCGGGGCCATCTACCTGC
CCCCCAACACACAGATCAACGAGAGTCCCCGCGCAGAGCTCTCCGTGA
CGGAGAGAACCCTGGAGCCCCCCACACAGAGCCCCAGCCCCCCACCCA
GACTCAGCGGCCAGTTGCAGGGGCTGGTCATCGGCGTCACGAGCGTGC
TGGTGGGTGTCCTGCTACTGCTGCTGCTGACCTGGGTCCTGGCCGCTG
TCTTCCCCAGGGCCACCCGAGGTGCCTGTGTGTGCGGGAGCGAGGACG
AGCCTCTGAAGGAGGGCCCCGATGCAGCGCCCGTCTTCACCCTGGACT
ACGGGGAGCTGGACTTCCAGTGGCGAGAGAAGACGCCGGAGCCCCCGG
CGCCCTGTGCCCCGGAGCAGACCGAGTATGCCACCATCGTCTTCCCGG
GCAGGCCGGCGTCCCCGGGCCGCAGGGCCTCGGCCAGCAGCCTGCAGG
GAGCCCAGCCTCCGAGCCCCGAGGACGGACCCGGCCTGTGGCCCCTC
Wildtype canine PD-1 amino acid sequence
(SEQ ID NO: 2):
MGSRRGPWPLVWAVLQLGWWPGWLLDSPDRPWSPLTFSPAQLTVQEGE
NATFTCSLADIPDSFVLNWYRLSPRNQTDKLAAFQEDRIEPGRDRRFR
VTRLPNGRDFHMSIVAARLNDSGIYLCGAIYLPPNTQINESPRAELSV
TERTLEPPTQSPSPPPRLSGQLQGLVIGVTSVLVGVLLLLLLTWVLAA
VFPRATRGACVCGSEDEPLKEGPDAAPVFTLDYGELDFQWREKTPEPP
APCAPEQTEYATIVFPGRPASPGRRASASSLQGAQPPSPEDGPGLWPL
Wildtype canine PD-1 extracellular domain amino
acid sequence (SEQ ID NO: 302):
LDSPDRPWSPLTFSPAQLTVQEGENATFTCSLADIPDSFVLNWYRLSP
RNQTDKLAAFQEDRIEPGRDRRFRVTRLPNGRDFHMSIVAARLNDSGI
YLCGAIYLPPNTQINESPRAELSVTERT
PET002 control antibodies:
Gilvetmab heavy chain (SEQ ID NO: 296)
EVQLVQSGGDLVKPGGSVRLSCVASGFNIKNTYMHWVRQAPGKGLQWI
GRIAPANVDTKYAPKFQGKATISADTAKNTAYMQLNSLRAEDTAVYYC
VLIYYDYDGDIDVWGQGTLVTVSSASTTAPSVFPLAPSCGSTSGSTVA
LACLVSGYFPEPVTVSWNSGSLTSGVHTFPSVLQSSGLYSLSSMVTVP
SSRWPSETFTCNVAHPASKTKVDKPVPKRENGRVPRPPDCPKCPAPEM
LGGPSVFIFPPKPKDTLLIARTPEVTCVVVALDPEDPEVQISWFVDGK
QMQTAKTQPREEQFAGTYRVVSVLPIGHQDWLKGKQFTCKVNNKALPS
PIERTISKARGQAHQPSVYVLPPSREELSKNTVSLTCLIKDFFPPDID
VEWQSNGQQEPESKYRTTPPQLDEDGSYFLYSKLSVDKSRWQRGDTFI
CAVMHEALHNHYTQESLSHSPGK
Gilvetmab Light chain (SEQ ID NO: 297)
DIVMTQTPLSLSVSLGEPASISCHASQNINVWLSWYRQKPGQIPQLLI
YKASHLHTGVPDRFSGSGSGTDFTLRISRVEADDAGVYYCQQGQSWPL
TFGQGTKVEIKRNDAQPAVYLFQPSPDQLHTGSASVVCLLNSFYPKDI
NVKWKVDGVIQDTGIQESVTEQDSKDSTYSLSSTLTMSSTEYLSHELY
SCEITHKSLPSTLIKSFQRSECQRVD
3B7-D9 heavy chain (SEQ ID NO: 298)
EVQLVETGGGLVQPGRSLKLSCVASGFTFNNYWMSWTRQAPGKGLEWV
ASITNSGVSTYYPDSVKGRFTISRDNAQNTLYLQMNSLRSEDTATYFC
TSALNWGYWYFDFWGPGTMVTVSSASTTAPSVFPLAPSCGSTSGSTVA
LACLVSGYFPEPVTVSWNSGSLTSGVHTFPSVLQSSGLYSLSSTVTVP
SSRWPSETFTCNVVHPASNTKVDKPVPKESTCKCISPCPVPESLGGPS
VFIFPPKPKDILRITRTPEITCVVLDLGREDPEVQISWFVDGKEVHTA
KTQPREQQFNSTYRVVSVLPIEHQDWLTGKEFKCRVNHIGLPSPIERT
ISKARGQAHQPSVYVLPPSPKELSSSDTVTLTCLIKDFFPPEIDVEWQ
SNGQPEPESKYHTTAPQLDEDGSYFLYSKLSVDKSRWQQGDTFTCAVM
HEALQNHYTDLSLSHSPGK
3B7-D9 light chain (SEQ ID NO: 299)
DIVMTQTPSSQAVSAGEKVTMSCKSSQSLLYSENKKNYLAWYQRKPGQ
SPKLLIYWASTRESGVPDRFIGSGSGTDFTLTISSVQAEDLAVYYCQQ
YYNFPLTFGSGTKLEIKRNDAQPAVYLFQPSPDQLHTGSASVVCLLNS
FYPKDINVKWKVDGVIQDTGIQESVTEQDKDSTYSLSSTLTMSSTEYL
SHELYSCEITHKSLPSTLIKSFQRSECQRVD
1B9 Def2 Heavy chain (SEQ ID NO: 300)
EVQLVQSGAEVKKPGASVKVSCKASGYTFTSFWMNWVRLAPGAGLEWI
GRVDPYDSETHYNQKFKDRAILTVDTSTSTAYMELSSLRAGDIAVYYC
ATQFGFSWLAYWGQGTLVTVSAASTTAPSVFPLAPSCGSTSGSTVALA
CLVSGYFPEPVTVSWNSGSLTSGVHTFPSVLQSSGLYSLSSMVTVPSS
RWPSETFTCNVAHPASKTKVDKPVPKRENGRVPRPPDCPKCPAPESLG
GPSVFIFPPKPKDTLLIARTPEVTCVVVDLGREDPEVQISWFVDGKQM
QTAKTQPREEQFNGTYRVVSVLPIGHQDWLKGKQFTCKVNHIGLPSPI
ERTISKARGQAHQPSVYVLPPSREELSKNTVSLTCLIKDFFPPDIDVE
WQSNGQQEPESKYRTTPPQLDEDGSYFLYSKLSVDKSRWQRGDTFICA
VMHEALHNHYTQKSLSHSPGK
1B9 Def2 Light chain (SEQ ID NO: 301)
EIVMTQSPGSLAGSAGESVSINCKSSQSLLYSSNQKNYLAWYQQKPGE
SPKLLIYWASTRESGVPDRFSGSGSGTDFTLTINNLQAEDVGVYYCQQ
YYSNPYTFGQGTKLEIKRNDAQPAVYLFQPSPDQLHTGSASVVCLLNS
FYPKDINVKWKVDGVIQDTGIQESVTEQDSKDSTYSLSSTLTMSSTEY
LSHELYSCEITHKSLPSTLIKSFQRSECQRVD

1-41. (canceled)

42. An isolated canine antibody or antigen-binding portion thereof which binds to one of the following epitopes of the extracellular domain of canine PD-1:
i) an epitope comprising T76, Q83, E84, L128, P129, P130 according to SEQ ID NO: 2
ii) an epitope comprising D58, D61, N74, T76, Y127 according to SEQ ID NO: 2 wherein the epitope is determined using an epitope mapping technique.

43. The antibody or antigen binding portion thereof of claim 42, wherein said antibody comprises
a) a heavy chain (HC) complementarity determining region CDR1 sequence comprising SEQ ID NO: 17 or an amino acid sequence with at least 70%, 80%, 85%, 90% or 95% sequence identity to SEQ ID NO: 17 and an HC CDR2 sequence comprising SEQ ID NO: 18 or an amino acid sequence with at least 70%, 80%, 85%, 90% or 95% sequence identity to SEQ ID NO: 18 and an HC CDR3 sequence comprising SEQ ID NO: 19 or an amino acid sequence with at least 70%, 80%, 85%, 90% or 95% sequence identity to SEQ ID NO: 19 and a light chain (LC) CDR1 sequence comprising SEQ ID NO: 20 or an amino acid sequence with at least 70%, 80%, 85%, 90% or 95% sequence identity to SEQ ID NO: 20 and an LC CDR2 sequence comprising SEQ ID NO: 21 or an amino acid sequence with at least 70%, 80%, 85%, 90% or 95% sequence identity to SEQ ID NO: 21 and an LC CDR3 sequence comprising SEQ ID NO: 22 or an amino acid sequence with at least 70%, 80%, 85%, 90% or 95% sequence identity to SEQ ID NO: 22;
b) an HC CDR1 sequence comprising SEQ ID NO: 27 or an amino acid sequence with at least 70%, 80%, 85%, 90% or 95% sequence identity to SEQ ID NO: 27 and an HC CDR2 sequence comprising SEQ ID NO: 28 or an amino acid sequence with at least 70%, 80%, 85%, 90% or 95% sequence identity to SEQ ID NO: 28 and an HC CDR3 sequence comprising SEQ ID NO: 29 or an amino acid sequence with at least 70%, 80%, 85%, 90% or 95% sequence identity to SEQ ID NO: 29 and an LC CDR1 sequence comprising SEQ ID NO: 30 or an amino acid sequence with at least 70%, 80%, 85%, 90% or 95% sequence identity to SEQ ID NO: 30 and an LC CDR2 sequence comprising SEQ ID NO: 31 or an amino acid sequence with at least 70%, 80%, 85%, 90% or 95% sequence identity to SEQ ID NO: 31 and an LC CDR3 sequence comprising SEQ ID NO: 32 or an amino acid sequence with at least 70%, 80%, 85%, 90% or 95% sequence identity to SEQ ID NO: 32;
c) an HC CDR1 sequence comprising SEQ ID NO: 207 or an amino acid sequence with at least 70%, 80%, 85%, 90% or 95% sequence identity to SEQ ID NO: 207 and an HC CDR2 sequence comprising SEQ ID NO: 208 or an amino acid sequence with at least 70%, 80%, 85%, 90% or 95% sequence identity to SEQ ID NO: 208 and an HC CDR3 sequence comprising SEQ ID NO: 209 or an amino acid sequence w with at least 70%, 80%, 85%, 90% or 95% sequence identity to SEQ ID NO: 209 and an LC CDR1 sequence comprising SEQ ID NO: 210 or an amino acid sequence with at least 70%, 80%, 85%, 90% or 95% sequence identity to SEQ ID NO: 210 and an LC CDR2 sequence comprising SEQ ID NO: 211 or an amino acid sequence with at least 70%, 80%, 85%, 90% or 95% sequence identity to SEQ ID NO: 211 and an LC CDR3 sequence comprising SEQ ID NO: 212 or an amino acid sequence with at least 70%, 80%, 85%, 90% or 95% sequence identity to SEQ ID NO: 212; or
d) an HC CDR1 sequence comprising SEQ ID NO: 97 or an amino acid sequence with at least 70%, 80%, 85%, 90% or 95% sequence identity to SEQ ID NO: 97 and an HC CDR2 sequence comprising SEQ ID NO: 98 or an amino acid sequence with at least 70%, 80%, 85%, 90% or 95% sequence identity to SEQ ID NO: 98 and an HC CDR3 sequence comprising SEQ ID NO: 99 or an amino acid sequence with at least 70%, 80%, 85%, 90% or 95% sequence identity to SEQ ID NO: 99 and an LC CDR1 sequence comprising SEQ ID NO: 100 or an amino acid sequence with at least 70%, 80%, 85%, 90% or 95% sequence identity to SEQ ID NO: 100 and an LC CDR2 sequence comprising SEQ ID NO: 101 or an amino acid sequence with at least 70%, 80%, 85%, 90% or 95% sequence identity to SEQ ID NO: 101 and an LC CDR3 sequence comprising SEQ ID NO: 102 or an amino acid sequence with at least 70%, 80%, 85%, 90% or 95% sequence identity to SEQ ID NO: 102.

44. The antibody or antigen-binding portion thereof of claim 42, wherein said antibody comprises
a) an HC CDR1 sequence comprising SEQ ID NO: 17 or an amino acid sequence which has 1, 2, 3, 4, or 5 amino acid differences compared to SEQ ID NO: 17 and an HC CDR2 sequence comprising SEQ ID NO: 18 or an amino acid sequence which has 1, 2, 3, 4, 5, or 6 amino acid differences compared to SEQ ID NO: 18 and an HC CDR3 sequence comprising SEQ ID NO: 19 or an amino acid sequence which has 1, 2, 3, 4, 5, 6, 7, 8, or 9 amino acid differences compared to SEQ ID NO: 19 and an LC CDR1 sequence comprising SEQ ID NO: 20 or an amino acid sequence which has 1, 2, 3, 4, 5, 6, 7, 8, 9, 10, 11, 12, 13 or 14 amino acid differences compared to SEQ ID NO: 20 and an LC CDR2 sequence comprising SEQ ID NO: 21 or an amino acid sequence which has 1, 2, 3, 4, or 5 amino acid differences compared to SEQ ID NO: 21 and an LC CDR3 sequence comprising SEQ ID NO: 22 or an amino acid sequence which has 1, 2, 3, 4, 5, 6, 7, 8, 9, or 10 amino acid differences compared to SEQ ID NO: 22;
b) an HC CDR1 sequence comprising SEQ ID NO: 207 or an amino acid sequence which has 1, or 2 amino acid differences compared to SEQ ID NO: 207 and an HC CDR2 sequence comprising SEQ ID NO: 208 or an amino acid sequence which has 1, 2, 3, 4, or 5 amino acid differences compared to SEQ ID NO: 208 and an HC CDR3 sequence comprising SEQ ID NO: 209 or an amino acid sequence which has 1, 2, 3, 4, 5, 6, 7, 8, 9, 10, 11, 12, 13, 14, 15, or 16 amino acid differences compared to SEQ ID NO: 209 and an LC CDR1 sequence comprising SEQ ID NO: 210 or an amino acid sequence which has 1, 2 or 3 amino acid differences compared to SEQ ID NO: 210 and an LC CDR2 sequence comprising SEQ ID NO: 211 or an amino acid sequence which has 1 or 2 amino acid differences compared to SEQ ID NO: 211 and an LC CDR3 sequence comprising SEQ ID NO: 212 or an amino acid sequence which has 1, 2, 3, or 4 amino acid differences compared to SEQ ID NO: 212;
c) an HC CDR1 sequence comprising SEQ ID NO: 97 or an amino acid sequence which has 1, 2, or 3 amino acid differences compared to SEQ ID NO: 97 and an HC CDR2 sequence comprising SEQ ID NO: 98 or an amino acid sequence which has 1, 2, 3, or 4 amino acid differences compared to SEQ ID NO: 98 and an HC CDR3 sequence comprising SEQ ID NO: 99 or an amino acid sequence which has 1, 2, 3, 4, 5, 6, 7, 8, 9, 10, 11, 12, 13, or 14 amino acid differences compared to SEQ ID NO: 99 and an LC CDR1 sequence comprising SEQ ID NO: 100 or an amino acid sequence which has 1, 2, 3, 4, 5, 6, 7, 8, 9, 10, 11, 12, 13, 14 or 15 amino acid differences compared to SEQ ID NO: 100 and an LC CDR2 sequence comprising SEQ ID NO: 101 or an amino acid sequence which has 1, 2, 3, 4, or 5 amino acid differences compared to SEQ ID NO: 101 and an LC CDR3 sequence comprising SEQ ID NO: 102 or an amino acid sequence which has 1, 2, 3, 4, 5, 6, 7, 8, 9, or 10 amino acid differences compared to SEQ ID NO: 102.

45. An isolated canine antibody or antigen-binding portion thereof which binds canine PD-1 wherein said antibody comprises
a) an HC CDR1 sequence comprising SEQ ID NO: 17 or an amino acid sequence which has 1, 2, 3, 4, or 5 amino acid differences compared to SEQ ID NO: 17 and an HC CDR2 sequence comprising SEQ ID NO: 18 or an amino acid sequence which has 1, 2, 3, 4, 5, or 6 amino acid differences compared to SEQ ID NO: 18 and an HC CDR3 sequence comprising SEQ ID NO: 19 or an amino acid sequence which has 1, 2, 3, 4, 5, 6, 7, 8, or 9 amino acid differences compared to SEQ ID NO: 19 and an LC CDR1 sequence comprising SEQ ID NO: 20 or an amino acid sequence which has 1, 2, 3, 4, 5, 6, 7, 8, 9, 10, 11, 12, 13 or 14 amino acid differences compared to SEQ ID NO: 20 and an LC CDR2 sequence comprising SEQ ID NO: 21 or an amino acid sequence which has 1, 2, 3, 4, or 5 amino acid differences compared to SEQ ID NO: 21 and an LC CDR3 sequence comprising SEQ ID NO: 22 or an amino acid sequence which has 1, 2, 3, 4, 5, 6, 7, 8, 9, or 10 amino acid differences compared to SEQ ID NO: 22;
b) an HC CDR1 sequence comprising SEQ ID NO: 207 or an amino acid sequence which has 1, or 2 amino acid differences compared to SEQ ID NO: 207 and an HC CDR2 sequence comprising SEQ ID NO: 208 or an amino acid sequence which has 1, 2, 3, 4, or 5 amino acid differences compared to SEQ ID NO: 208 and an HC CDR3 sequence comprising SEQ ID NO: 209 or an amino acid sequence which has 1, 2, 3, 4, 5, 6, 7, 8, 9, 10, 11, 12, 13, 14, 15, or 16 amino acid differences compared to SEQ ID NO: 209 and an LC CDR1 sequence comprising SEQ ID NO: 210 or an amino acid sequence which has 1, 2 or 3 amino acid differences compared to SEQ ID NO: 210 and an LC CDR2 sequence comprising SEQ ID NO: 211 or an amino acid sequence which has 1 or 2 amino acid differences compared to SEQ ID NO: 211 and an LC CDR3 sequence comprising SEQ ID NO: 212 or an amino acid sequence which has 1, 2, 3, or 4 amino acid differences compared to SEQ ID NO: 212; or
c) an HC CDR1 sequence comprising SEQ ID NO: 97 or an amino acid sequence which has 1, 2, or 3 amino acid differences compared to SEQ ID NO: 97 and an HC CDR2 sequence comprising SEQ ID NO: 98 or an amino acid sequence which has 1, 2, 3, or 4 amino acid differences compared to SEQ ID NO: 98 and an HC CDR3 sequence comprising SEQ ID NO: 99 or an amino acid sequence which has 1, 2, 3, 4, 5, 6, 7, 8, 9, 10, 11, 12, 13, or 14 amino acid differences compared to SEQ ID NO: 99 and an LC CDR1 sequence comprising SEQ ID NO: 100 or an amino acid sequence which has 1, 2, 3, 4, 5, 6, 7, 8, 9, 10, 11, 12, 13, 14 or 15 amino acid differences compared to SEQ ID NO: 100 and an LC CDR2 sequence comprising SEQ ID NO: 101 or an amino acid sequence which has 1, 2, 3, 4, or 5 amino acid differences compared to SEQ ID NO: 101 and an LC CDR3 sequence comprising SEQ ID NO: 102 or an amino acid sequence which has 1, 2, 3, 4, 5, 6, 7, 8, 9, or 10 amino acid differences compared to SEQ ID NO: 102.

46. The antibody or antigen-binding portion thereof of claim 42, wherein said antibody or antigen-binding portion thereof has
i) a HC CDR1 sequence comprising SEQ ID NO: 17, a HC CDR2 sequence comprising SEQ ID NO: 18, a HC CDR3 sequence comprising SEQ ID NO: 19, a LC CDR1 sequence comprising SEQ ID NO: 20, a LC CDR2 sequence comprising SEQ ID NO: 21 and a LC CDR3 sequence comprising SEQ ID NO: 22;
ii) a HC CDR1 sequence comprising SEQ ID NO: 27, a HC CDR2 sequence comprising SEQ ID NO: 28, a HC CDR3 sequence comprising SEQ ID NO: 29, a LC CDR1 sequence comprising SEQ ID NO: 30, a LC CDR2 sequence comprising SEQ ID NO: 31 and a LC CDR3 sequence comprising SEQ ID NO: 32;
iii) a HC CDR1 sequence comprising SEQ ID NO: 7, a HC CDR2 sequence comprising SEQ ID NO: 8, a HC CDR3 sequence comprising SEQ ID NO: 9, a LC CDR1 sequence comprising SEQ ID NO: 10, a LC CDR2 sequence comprising SEQ ID NO: 11 and a LC CDR3 sequence comprising SEQ ID NO: 12;
iv) a HC CDR1 sequence comprising SEQ ID NO: 37, a HC CDR2 sequence comprising SEQ ID NO: 38, a HC CDR3 sequence comprising SEQ ID NO: 39, a LC CDR1 sequence comprising SEQ ID NO: 40, a LC CDR2 sequence comprising SEQ ID NO: 41 and a LC CDR3 sequence comprising SEQ ID NO: 42;
v) a HC CDR1 sequence comprising SEQ ID NO: 47, a HC CDR2 sequence comprising SEQ ID NO: 48, a HC CDR3 sequence comprising SEQ ID NO: 49, a LC CDR1 sequence comprising SEQ ID NO: 50, a LC CDR2 sequence comprising SEQ ID NO: 51 and a LC CDR3 sequence comprising SEQ ID NO: 52;
vi) a HC CDR1 sequence comprising SEQ ID NO: 57, a HC CDR2 sequence comprising SEQ ID NO: 58, a HC CDR3 sequence comprising SEQ ID NO: 59, a LC CDR1 sequence comprising SEQ ID NO: 60, a LC CDR2 sequence comprising SEQ ID NO: 61 and a LC CDR3 sequence comprising SEQ ID NO: 62;
vii) a HC CDR1 sequence comprising SEQ ID NO: 67, a HC CDR2 sequence comprising SEQ ID NO: 68, a HC CDR3 sequence comprising SEQ ID NO: 69, a LC CDR1 sequence comprising SEQ ID NO: 70, a LC CDR2 sequence comprising SEQ ID NO: 71 and a LC CDR3 sequence comprising SEQ ID NO: 72;
viii) a HC CDR1 sequence comprising SEQ ID NO: 77, a HC CDR2 sequence comprising SEQ ID NO: 78, a HC CDR3 sequence comprising SEQ ID NO: 79, a LC CDR1 sequence comprising SEQ ID NO: 80, a LC CDR2 sequence comprising SEQ ID NO: 81 and a LC CDR3 sequence comprising SEQ ID NO: 82;
ix) a HC CDR1 sequence comprising SEQ ID NO: 87, a HC CDR2 sequence comprising SEQ ID NO: 88, a HC CDR3 sequence comprising SEQ ID NO: 89, a LC CDR1 sequence comprising SEQ ID NO: 90, a LC CDR2 sequence comprising SEQ ID NO: 91 and a LC CDR3 sequence comprising SEQ ID NO: 92;
x) a HC CDR1 sequence comprising SEQ ID NO: 97, a HC CDR2 sequence comprising SEQ ID NO: 98, a HC CDR3 sequence comprising SEQ ID NO: 99, a LC CDR1 sequence comprising SEQ ID NO: 100, a LC CDR2 sequence comprising SEQ ID NO: 101 and a LC CDR3 sequence comprising SEQ ID NO: 102;
xi) a HC CDR1 sequence comprising SEQ ID NO: 107, a HC CDR2 sequence comprising SEQ ID NO: 108, a HC CDR3 sequence comprising SEQ ID NO: 109, a LC CDR1 sequence comprising SEQ ID NO: 110, a LC CDR2 sequence comprising SEQ ID NO: 111 and a LC CDR3 sequence comprising SEQ ID NO: 112;
xii) a HC CDR1 sequence comprising SEQ ID NO: 117, a HC CDR2 sequence comprising SEQ ID NO: 118, a HC CDR3 sequence comprising SEQ ID NO: 119, a LC CDR1 sequence comprising SEQ ID NO: 120, a LC CDR2 sequence comprising SEQ ID NO: 12 and a LC CDR3 sequence comprising SEQ ID NO: 122;
xiii) a HC CDR1 sequence comprising SEQ ID NO: 127, a HC CDR2 sequence comprising SEQ ID NO: 128, a HC CDR3 sequence comprising SEQ ID NO: 129, a LC CDR1 sequence comprising SEQ ID NO: 130, a LC CDR2 sequence comprising SEQ ID NO: 131 and a LC CDR3 sequence comprising SEQ ID NO: 132;
xiv) a HC CDR1 sequence comprising SEQ ID NO: 137, a HC CDR2 sequence comprising SEQ ID NO: 138, a HC CDR3 sequence comprising SEQ ID NO: 139, a LC CDR1 sequence comprising SEQ ID NO: 140, a LC CDR2 sequence comprising SEQ ID NO: 141 and a LC CDR3 sequence comprising SEQ ID NO: 142;
xv) a HC CDR1 sequence comprising SEQ ID NO: 147, a HC CDR2 sequence comprising SEQ ID NO: 148, a HC CDR3 sequence comprising SEQ ID NO: 149, a LC CDR1 sequence comprising SEQ ID NO: 150, a LC CDR2 sequence comprising SEQ ID NO: 151 and a LC CDR3 sequence comprising SEQ ID NO: 152;
xvi) a HC CDR1 sequence comprising SEQ ID NO: 157, a HC CDR2 sequence comprising SEQ ID NO: 158, a HC CDR3 sequence comprising SEQ ID NO: 159, a LC CDR1 sequence comprising SEQ ID NO: 160, a LC CDR2 sequence comprising SEQ ID NO: 161 and a LC CDR3 sequence comprising SEQ ID NO: 162;
xvii) a HC CDR1 sequence comprising SEQ ID NO: 167, a HC CDR2 sequence comprising SEQ ID NO: 168, a HC CDR3 sequence comprising SEQ ID NO: 169, a LC CDR1 sequence comprising SEQ ID NO: 170, a LC CDR2 sequence comprising SEQ ID NO: 171 and a LC CDR3 sequence comprising SEQ ID NO: 172;
xviii) a HC CDR1 sequence comprising SEQ ID NO: 177, a HC CDR2 sequence comprising SEQ ID NO: 178, a HC CDR3 sequence comprising SEQ ID NO: 179, a LC CDR1 sequence comprising SEQ ID NO: 180, a LC CDR2 sequence comprising SEQ ID NO: 181 and a LC CDR3 sequence comprising SEQ ID NO: 182;
xix) a HC CDR1 sequence comprising SEQ ID NO: 187, a HC CDR2 sequence comprising SEQ ID NO: 188, a HC CDR3 sequence comprising SEQ ID NO: 189, a LC CDR1 sequence comprising SEQ ID NO: 190, a LC CDR2 sequence comprising SEQ ID NO: 191 and a LC CDR3 sequence comprising SEQ ID NO: 192;
xx) a HC CDR1 sequence comprising SEQ ID NO: 197, a HC CDR2 sequence comprising SEQ ID NO: 198, a HC CDR3 sequence comprising SEQ ID NO: 199, a LC CDR1 sequence comprising SEQ ID NO: 200, a LC CDR2 sequence comprising SEQ ID NO: 201 and a LC CDR3 sequence comprising SEQ ID NO: 202;
xxi) a HC CDR1 sequence comprising SEQ ID NO: 207, a HC CDR2 sequence comprising SEQ ID NO: 208, a HC CDR3 sequence comprising SEQ ID NO: 209, a LC CDR1 sequence comprising SEQ ID NO: 210, a LC CDR2 sequence comprising SEQ ID NO: 211 and a LC CDR3 sequence comprising SEQ ID NO: 212;
xxii) a HC CDR1 sequence comprising SEQ ID NO: 217, a HC CDR2 sequence comprising SEQ ID NO: 218, a HC CDR3 sequence comprising SEQ ID NO: 219, a LC CDR1 sequence comprising SEQ ID NO: 220, a LC CDR2 sequence comprising SEQ ID NO: 221 and a LC CDR3 sequence comprising SEQ ID NO: 222;
xxiii) a HC CDR1 sequence comprising SEQ ID NO: 227, a HC CDR2 sequence comprising SEQ ID NO: 228, a HC CDR3 sequence comprising SEQ ID NO: 229, a LC CDR1 sequence comprising SEQ ID NO: 230, a LC CDR2 sequence comprising SEQ ID NO: 231 and a LC CDR3 sequence comprising SEQ ID NO: 232;
xxiv) a HC CDR1 sequence comprising SEQ ID NO: 237, a HC CDR2 sequence comprising SEQ ID NO: 238, a HC CDR3 sequence comprising SEQ ID NO: 239, a LC CDR1 sequence comprising SEQ ID NO: 240, a LC CDR2 sequence comprising SEQ ID NO: 241 and a LC CDR3 sequence comprising SEQ ID NO: 242;
xxv) a HC CDR1 sequence comprising SEQ ID NO: 247, a HC CDR2 sequence comprising SEQ ID NO: 248, a HC CDR3 sequence comprising SEQ ID NO: 249, a LC CDR1 sequence comprising SEQ ID NO: 250, a LC CDR2 sequence comprising SEQ ID NO: 251 and a LC CDR3 sequence comprising SEQ ID NO: 252;
xxvi) a HC CDR1 sequence comprising SEQ ID NO: 257, a HC CDR2 sequence comprising SEQ ID NO: 258, a HC CDR3 sequence comprising SEQ ID NO: 259, a LC CDR1 sequence comprising SEQ ID NO: 260, a LC CDR2 sequence comprising SEQ ID NO: 261 and a LC CDR3 sequence comprising SEQ ID NO: 262;
xxvii) a HC CDR1 sequence comprising SEQ ID NO: 267, a HC CDR2 sequence comprising SEQ ID NO: 268, a HC CDR3 sequence comprising SEQ ID NO: 269, a LC CDR1 sequence comprising SEQ ID NO: 270, a LC CDR2 sequence comprising SEQ ID NO: 271 and a LC CDR3 sequence comprising SEQ ID NO: 272;
xxviii) a HC CDR1 sequence comprising SEQ ID NO: 277, a HC CDR2 sequence comprising SEQ ID NO: 278, a HC CDR3 sequence comprising SEQ ID NO: 279, a LC CDR1 sequence comprising SEQ ID NO: 280, a LC CDR2 sequence comprising SEQ ID NO: 281 and a LC CDR3 sequence comprising SEQ ID NO: 282; or
xxix) a HC CDR1 sequence comprising SEQ ID NO: 287, a HC CDR2 sequence comprising SEQ ID NO: 288, a HC CDR3 sequence comprising SEQ ID NO: 289, a LC CDR1 sequence comprising SEQ ID NO: 290, a LC CDR2 sequence comprising SEQ ID NO: 291 and a LC CDR3 sequence comprising SEQ ID NO: 292.

47. The antibody or antigen-binding portion thereof of claim 42, wherein said antibody or antigen-binding portion thereof comprises a HC variable region sequence comprising SEQ ID NO: 4 or a sequence with at least 45%, 50%, 60%, 70%, 75%, 80%, 85% or 90% sequence identity thereto and a LC variable region sequence comprising SEQ ID NO: 6 or a sequence with at least 35%, 40%, 50%, 60%, 70%, 75%, 80%, 85%, 90% or 95% sequence identity thereto for example a LC variable region sequence comprising SEQ ID NO: 6.

48. The antibody or antigen-binding portion thereof of claim 42, wherein said antibody or antigen-binding portion thereof has
a) a HC variable region sequence comprising SEQ ID NO: 4 or a sequence with at least 45%, 50%, 70%, 75%, 80%, 85% or 90% sequence identity thereto and a LC variable region sequence comprising SEQ ID NO: 6 or a sequence with at least 35%, 40%, 70%, 75%, 80%, 85% or 90% sequence identity thereto;
b) a HC variable region sequence comprising SEQ ID NO: 14 or a sequence with at least 40%, 45%, 70%, 75%, 80%, 85% or 90% sequence identity thereto and a LC variable region sequence comprising SEQ ID NO: 16 or a sequence with at least 35%, 45%, 55%, 65%, 70%, 75%, 80%, 85% or 90% sequence identity thereto;
c) a HC variable region sequence comprising SEQ ID NO: 24 or a sequence with at least 45%, 50%, 70%, 75%, 80%, 85% or 90% sequence identity thereto and a LC variable region sequence comprising SEQ ID NO: 6 or a sequence with at least 35%, 40%, 70%, 75%, 80%, 85% or 90% sequence identity thereto;
d) a HC variable region sequence comprising SEQ ID NO: 34 or a sequence with at least 45%, 50%, 70%, 75%, 80%, 85% or 90% sequence identity thereto and a LC variable region sequence comprising SEQ ID NO: 6 or a sequence with at least 35%, 40%, 70%, 75%, 80%, 85% or 90% sequence identity thereto;
e) a HC variable region sequence comprising SEQ ID NO: 44 or a sequence with at least 40%, 50%, 60%, 70%, 75%, 80%, 85% or 90% sequence identity thereto and a LC variable region sequence comprising SEQ ID NO: 46 or a sequence with at least 35%, 45%, 55%, 65%, 70%, 75%, 80%, 85% or 90% sequence identity thereto;
f) a HC variable region sequence comprising SEQ ID NO: 54 or a sequence with at least 40%, 45%, 70%, 75%, 80%, 85% or 90% sequence identity thereto and a LC variable region sequence comprising SEQ ID NO: 56 or a sequence with at least 35%, 40%, 70%, 75%, 80%, 85% or 90% sequence identity thereto;
g) a HC variable region sequence comprising SEQ ID NO: 64 or a sequence with at least 45%, 65%, 70%, 75%, 80%, 85% or 90% sequence identity thereto and a LC variable region sequence comprising SEQ ID NO: 36 or a sequence with at least 35%, 40%, 70%, 75%, 80%, 85% or 90% sequence identity thereto;
h) a HC variable region sequence comprising SEQ ID NO: 74 or a sequence with at least 45%, 50%, 70%, 75%, 80%, 85% or 90% sequence identity thereto and a LC variable region sequence comprising SEQ ID NO: 6 or a sequence with at least 35%, 40%, 70%, 75%, 80%, 85% or 90% sequence identity thereto;
i) a HC variable region sequence comprising SEQ ID NO: 84 or a sequence with at least 40%, 45%, 50%, 70%, 75%, 80%, 85% or 90% sequence identity thereto and a LC variable region sequence comprising SEQ ID NO: 86 or a sequence with at least 35%, 40%, 70%, 75%, 80%, 85% or 90% sequence identity thereto;
j) a HC variable region sequence comprising SEQ ID NO: 94 or a sequence with at least 45%, 50%, 70%, 75%, 80%, 85% or 90% sequence identity thereto and a LC variable region sequence comprising SEQ ID NO: 6 or a sequence with at least 35%, 40%, 70%, 75%, 80%, 85% or 90% sequence identity thereto;
k) a HC variable region sequence comprising SEQ ID NO: 104 or a sequence with at least 45%, 50%, 70%, 75%, 80%, 85% or 90% sequence identity thereto and a LC variable region sequence comprising SEQ ID NO: 6 or a sequence with at least 35%, 40%, 70%, 75%, 80%, 85% or 90% sequence identity thereto;
l) a HC variable region sequence comprising SEQ ID NO: 114 or a sequence with at least 45%, 50%, 70%, 75%, 80%, 85% or 90% sequence identity thereto and a LC variable region sequence comprising SEQ ID NO: 6 or a sequence with at least 35%, 40%, 70%, 75%, 80%, 85% or 90% sequence identity thereto;
m) a HC variable region sequence comprising SEQ ID NO: 124 or a sequence with at least 45%, 50%, 70%, 75%, 80%, 85% or 90% sequence identity thereto and a LC variable region sequence comprising SEQ ID NO: 6 or a sequence with at least 35%, 40%, 70%, 75%, 80%, 85% or 90% sequence identity thereto;
n) a HC variable region sequence comprising SEQ ID NO: 134 or a sequence with at least 45%, 50%, 70%, 75%, 80%, 85% or 90% sequence identity thereto and a LC variable region sequence comprising SEQ ID NO: 136 or a sequence with at least 35%, 40%, 70%, 75%, 80%, 85% or 90% sequence identity thereto;
o) a HC variable region sequence comprising SEQ ID NO:144 or a sequence with at least 45%, 50%, 70%, 75%, 80%, 85% or 90% sequence identity thereto and a LC variable region sequence comprising SEQ ID NO: 6 or a sequence with at least 35%, 40%, 70%, 75%, 80%, 85% or 90% sequence identity thereto;
p) a HC variable region sequence comprising SEQ ID NO: 154 or a sequence with at least 45%, 50%, 70%, 75%, 80%, 85% or 90% sequence identity thereto and a LC variable region sequence comprising SEQ ID NO: 156 or a sequence with at least 35%, 40%, 70%, 75%, 80%, 85% or 90% sequence identity thereto;
q) a HC variable region sequence comprising SEQ ID NO: 164 or a sequence with at least 45%, 50%, 70%, 75%, 80%, 85% or 90% sequence identity thereto and a LC variable region sequence comprising SEQ ID NO: 176 or a sequence with at least 35%, 40%, 70%, 75%, 80%, 85% or 90% sequence identity thereto;
r) a HC variable region sequence comprising SEQ ID NO: 174 or a sequence with at least 45%, 50%, 70%, 75%, 80%, 85% or 90% sequence identity thereto and a LC variable region sequence comprising SEQ ID NO: 6 or a sequence with at least 35%, 40%, 70%, 75%, 80%, 85% or 90% sequence identity thereto;
s) a HC variable region sequence comprising SEQ ID NO: 184 or a sequence with at least 45%, 50%, 70%, 75%, 80%, 85% or 90% sequence identity thereto and a LC variable region sequence comprising SEQ ID NO: 186 or a sequence with at least 35%, 40%, 70%, 75%, 80%, 85% or 90% sequence identity thereto;
t) a HC variable region sequence comprising SEQ ID NO: 194 or a sequence with at least 45%, 50%, 70%, 75%, 80%, 85% or 90% sequence identity thereto and a LC variable region sequence comprising SEQ ID NO: 6 or a sequence with at least 35%, 40%, 70%, 75%, 80%, 85% or 90% sequence identity thereto;
u) a HC variable region sequence comprising SEQ ID NO: 204 or a sequence with at least 40%, 45%, 70%, 75%, 80%, 85% or 90% sequence identity thereto and a LC variable region sequence comprising SEQ ID NO: 206 or a sequence with at least 35%, 40%, 70%, 75%, 80%, 85% or 90% sequence identity thereto;
v) a HC variable region sequence comprising SEQ ID NO: 214 or a sequence with at least 40%, 45%, 70%, 75%, 80%, 85% or 90% sequence identity thereto and a LC variable region sequence comprising SEQ ID NO: 216 or a sequence with at least 35%, 40%, 70%, 75%, 80%, 85% or 90% sequence identity thereto;
w) a HC variable region sequence comprising SEQ ID NO: 224 or a sequence with at least 40%, 45%, 70%, 75%, 80%, 85% or 90% sequence identity thereto and a LC variable region sequence comprising SEQ ID NO: 226 or a sequence with at least 35%, 40%, 70%, 75%, 80%, 85% or 90% sequence identity thereto;
x) a HC variable region sequence comprising SEQ ID NO: 234 or a sequence with at least 45%, 50%, 70%, 75%, 80%, 85% or 90% sequence identity thereto and a LC variable region sequence comprising SEQ ID NO: 236 or a sequence with at least 35%, 40%, 70%, 75%, 80%, 85% or 90% sequence identity thereto;
y) a HC variable region sequence comprising SEQ ID NO: 244 or a sequence with at least 40%, 45%, 70%, 75%, 80%, 85% or 90% sequence identity thereto and a LC variable region sequence comprising SEQ ID NO: 246 or a sequence with at least 35%, 40%, 70%, 75%, 80%, 85% or 90% sequence identity thereto;
z) a HC variable region sequence comprising SEQ ID NO: 254 or a sequence with at least 40%, 45%, 70%, 75%, 80%, 85% or 90% sequence identity thereto and a LC variable region sequence comprising SEQ ID NO: 256 or a sequence with at least 35%, 40%, 70%, 75%, 80%, 85% or 90% sequence identity thereto;
aa) a HC variable region sequence comprising SEQ ID NO: 264 or a sequence with at least 40%, 45%, 70%, 75%, 80%, 85% or 90% sequence identity thereto and a LC variable region sequence comprising SEQ ID NO: 266 or a sequence with at least 35%, 40%, 70%, 75%, 80%, 85% or 90% sequence identity thereto;
bb) a HC variable region sequence comprising SEQ ID NO: 274 or a sequence with at least 45%, 50%, 70%, 75%, 80%, 85% or 90% sequence identity thereto and a LC variable region sequence comprising SEQ ID NO: 276 or a sequence with at least 35%, 40%, 70%, 75%, 80%, 85% or 90% sequence identity thereto;
cc) a HC variable region sequence comprising SEQ ID NO: 284 or a sequence with at least 45%, 50%, 70%, 75%, 80%, 85% or 90% sequence identity thereto and a LC variable region sequence comprising SEQ ID NO: 286 or a sequence with at least 35%, 40%, 70%, 75%, 80%, 85% or 90% sequence identity thereto; or
dd) a HC variable region sequence comprising SEQ ID NO: 24 or a sequence with at least 45%, 50%, 70%, 75%, 80%, 85% or 90% sequence identity thereto and a LC variable region sequence comprising SEQ ID NO: 26 or a sequence with at least 35%, 40%, 70%, 75%, 80%, 85% or 90% sequence identity thereto.

49. The antibody or antigen-binding portion thereof of claim 42, wherein said antigen-binding portion thereof is an scFv, Fv, heavy chain or single domain antibody.

50. An isolated canine antibody or antigen-binding portion thereof of claim 42 that binds to canine PD-1 wherein said antibody or antigen-binding portion thereof that competes with PD-L1 and/or PD-L2.

51. The antibody or antigen-binding portion thereof of claim 42, wherein said antibody or antigen binding portion thereof blocks the interaction of PD-1 with PD-L1 and/or PD-L2 and/or prevents a cellular response associated with the interaction of PD-1 with PD-L1 and/or PD-L2.

52. The antibody or antigen-binding portion thereof of claim 42, wherein said antibody or antigen-binding portion thereof is conjugated to a therapeutic moiety.

53. The antibody or antigen-binding portion thereof of claim 52, wherein said therapeutic moiety is a second antibody or antigen-binding portion thereof.

54. The antibody or antigen-binding portion thereof of claim 53, wherein said second antibody or antigen-binding portion thereof binds to a different target.

55. The antibody or antigen-binding portion thereof of claim 42, wherein said antibody or antigen-binding portion thereof is conjugated to a further moiety selected from a half-life extending moiety, label, cytotoxin, liposome, nanoparticle or radioisotope.

56. The antibody or antigen-binding portion thereof of claim 55, wherein the half-life extending moiety is selected from: albumin binding moiety, a transferrin binding moiety, a polyethylene glycol molecule, a recombinant polyethylene glycol molecule, human serum albumin, a fragment of human serum albumin, an albumin binding peptide or single domain antibody that binds to human serum albumin.

57. A pharmaceutical composition comprising an antibody or antigen-binding portion thereof of claim 42.

58. A method of treating cancer or a tumor in a canine subject in need thereof comprising administering an effective amount of the antibody or antigen-binding portion thereof of claim 42.

59. A nucleic acid sequence that encodes an antibody or antibody antigen-binding portion thereof of claim 42.

60. A vector comprising a nucleic acid sequence of claim 59.

61. A host cell comprising the nucleic acid sequence of claim 59.

62. A kit comprising an antibody or antigen-binding portion thereof of claim 42 and instructions for use.

63. A method for making a canine antibody that binds PD-1 comprising culturing the isolated host cell of claim 61 and recovering said antibody.

64. A method for detecting a PD-1 protein or an extracellular domain of a PD-1 protein in a biological sample from a canine subject, comprising contacting a biological sample with the antibody or antigen-binding portion thereof of claim 42 wherein said antibody or antigen-binding portion thereof is linked to a detectable label.

65. A method of inhibiting tumor growth or metastasis comprising contacting a tumor cell with an effective amount of the antibody or antigen-binding portion thereof of claim 42.

66. A method of killing a tumor cell expressing PD-1, comprising contacting the cell with the antibody or antigen binding portion of claim 42, such that killing of the cell expressing PD-1 occurs.

67. A method of modulating an immune response comprising administering an antibody or antigen binding portion thereof of claim 42.

68. A combination therapy comprising an antibody or antigen binding portion thereof of claim 42 and a further therapeutic moiety.
